SEQUENCE LISTING (209 sequences)

SEQ ID NO: 1 - DNA, 115469 bp, Canis familiaris
  misc_feature (10684)..(10929): n is a, c, g, or t
ttactggtacacgaccttgacgcagaaggcttcttcatttttgtcttcgtggtcattgatgtaaatcagaatctcctcctccccggggctctgccgaggtgcgaaccacaggccgatggtgtaggtctctcctcccccaatctggaagcaagcatcagatcctagccctactgcagccctgctctccatgccagcatggtcaacacatgcaaacccaggctgacagagcagggtaaaccgaggctctggcccccactgctgccaggcacctgaggttctgaacacaacctccccgtgttacaggacaccactgctctgcccacgcctcaggggagattcacagctgaagaaacaccttccaatgacagacagactgccatctgctgtactgtcgttaaaatacttgcaacagagaaaaggctaacatgcaggacacttgcgaataaaatcgctgataaactgcagggtaaattggccaccagaagccctgtgacagtgcccagatgtgggggccctagccgcatgggcagaactgctgagagaggggtggccgcccctactcccagtaccaccggtgcagatcagcactgccccctcccggggctctggagacccctgtgcctcggggcccctgggacatgccgaggctgggtgtcccacatgcagtgcgcacaccctgtacgcagcgcttcccgcacctggaaggagtcctctctgaactgcagcaggtcagggtggtcgctgtacaggtgataggtcctctgggaggggtaagggttggtgtaggtgatccgcttgttggcgcctctgcctttccctgcagccagtgtgatctcgaaagcctgtgtgggcaggggtgtcagccttcctcagcctgctctgcacgtacagcccgacctatgtctccatagacatgacaagacacccgccgagggggctcactgtacaggagcaacccccgcccccacccccaggccatagactgcggaggagactcggtcacaggaccaggagacagcgaggtgacacagcacccataccacacacgacccagcaggttcccctccagaccttggagatgagaggcgggcggcaggagaggcacacgagccatgaggccaccagctggtggtagtccacgtcgaccaggttgaggtggatgaaccggctccctgccctgcggggcctcacaccgacatgcaggtcctgcaccccatgtggtggcagcacgaacacacctttggggtcagtctttcaaaacaacagcagagaggcgggtcagtatgcagtccctcatgctgtgcagaggggttccctgaacacctgcagccagtgcacgagaatcccaagggacctcagctattcagaggcagggggcacgtggcctgcaggaagtcagaaggggaccgagtgacaatgccaggtctgaagccagacgcagccacacagtcagggttgcagtgcatctcagtccgcatcaccacccgcatgggcaagtgcccctccatgtggcaacagtccccttagcaccagagctgcctcagcagagggaaatcttaacaggctccagctgtaagcttcagcaaggagtctgcattgccctcattacccagacgcatagttgtggctaaattaattccaaaattctattaatatttaccttccaataattctaaaaattggcctatagggtttatatgtcactcattccatgatggcagatccaataaaaggcagtttgaatgaagactctgccaagagtcttaaaggagctgtgagccgatcctcagccccacactggctgcagccagtctccacaccgtccgtgccccaggtggggtgaggatggggagaggccagagaacacccagcgacctaccttcagctcctgagggtgtgaggtgaaagcctgcactctcctaaccgtccgtgtccctcgaaggacaagggataggcgtgtccgctggcctgtgacacaggagacgtccacacgctgcaaggagtggaggtagacctgccatatttggatgggtgtcgccagccaacgatccctgtgtaggacagaaaccgtgatcaatggtggtgaggacagcagcactgaccaggaagaggccaagaaggtggccaggacaactggggcaggctggagcccagctgccatcatccctgcagccaccttccccactctgaaggcccactgtgactttgcaccacaacaccctgtgtgagtggtaatgtgggcttctaccagcaacaaaacagtcccaaaccaggtggtctgggacaaagcctgtggaaagagtgccgtggaagagtatgtgggttcagaggccacccagacctgagctcacaggccccgtcctagctgcctggtgctggaggccactggcccttccctggaaatggggctcgataaccctgcccctcgggcttctccacacggccagagaccagaaccgagcatggctacacctgccccaatcccagatgtcctattcagaagcccccagcctaccccagttttaaaagatcctcaagggggacacctgggtggctcagcagttgagtgtgtgcctttggctcagggagtgatcccagggccctgggatcaagttccacatcagtctccctgcagggagcctgcttctccctctgcctatgtctctgtccctctgtgtatctctcatgaataaataaataaaatctttaaaaaaaaaaaaaaaagaagatcctcaagggactggaagaggagacttcccagaacaaggtaaaggaacagggttatttcaactgagtgggagaggcaagaaggcgatgtgatcaccatctcaggcacggaggatgaagactgccaccctccctgtcccctgaacggagccaggcactgctctggggcagacgccaccccaagttcctttccttggtgggctctccatgcctctgcaagccgccgggccactctgatccgatggcgtcctttgtgaggcccctgccagccagccgtggagtctcacctcccgtggctgaaggccccacgacaatgtgccccatcaggaactcagcatgagcccaggcggcctctgggatgctgtggctgcctcccagtcccgcccaccctccagacctcccaggtttgggctcacagactcacgcgtaaacggcgacaaagaagtctctgctctctgggcttggaccgctggccaccttcaggaacacgtcccgaggctccctggggccctggcggacatgggcgccctgagaacaccacctgcctcccccccaagcccctggcaggtgaaagcgggcaggaactggctccctccctcccaacaggtgttgcattttcagtattaggcagaattcgtttttttcttcactgcaaaaaagaaaatcattaacttcttgatttcatattgagttcaataattatattctgcaggagattttatccagtttcctaaccagtcataacataattattgcctttattctttacacaatggcaattaataagtggcttaattaccactcactttattcactttgcttcatgcctttttttttttttttcatttgaattcaggtaatccaagaactgatctgccagcagaaaagatgaaaactcatgcataaagctcccacaaggatgtcaaaatagccccttagcaccttagaaagacaccacttaactcaaaagccgccgtgaagaggcctcaggaggccaggggctcagatgaccaggcactgactgggagacgctcggcagaacttaccacattctgggtctcgcagatgacgttggggtcgctacatcggacatagactgggggctcctcaccaggtcttcccacgggagcacctgctgcagggaatatcatagcaaacgtcttctggacaggcccgggccacatgatgttgctggcccatcacaggtaccccacagacaccagaagacagcaatgccacccttcctactgtcccgcccacgtgggcactggcactcccgccctaagccacacccgcaggcacttatccgaagagagtgaccaaaagtaaagagcggggggggctatggtgcagggcgccagacacggggacgctgctgcactctaaagcaccagaccagaagcagacaagtgacgcggcagcagaaggcgcgagcgcactccaggcacagccctcggggccgtgggctttaggaagtgggattagccagccaagggcataccctcgcatctcagaggacgcgaaaaggtcaccatttcaaaccataagggcgggtggcagactttacagatgggtcacaggttgggataagagcatcacaggagaatgtgatgatgccacttgtcaccctgagggaaaaacctttagccatctgtggaacctgcaaaatgccagccgcagggacagatgggacctgcctggaagcgtgtgccagggaggcaggcggatggccttcttcaggaaggtgagctctgggtggtagaagcggaaaacctggtccaccacatggggctgggtctccacggttaggcaaagcacggcgatgggcttaccgccactcccccggaacaagacctgccaggagagcagaggttgccagcatcagggctggcacgtgggctcatgaagagctgcctgcaccaagccagcatccacagggcccagtccccccaccccccgcaccgtgacgtgggaagcacaggtgttgtcagaaatggtgggctgaatctgcagcagcaggtccccacagaggggatggcacccagcacagggactgcatgtggacatgttcccgtgacacacatgcctcaaatgtgtcagcgctcaatttgacagccccaatgtcaaatactccttcagccaccgacccatctcccaacatgcctgccatgtgcatgctgggaagctgcccagagggtctgatgttaacctctccaagtcctcatgggtggtagtggagcacaagctctttaattctgaacctaaacaagaaaggctagagatacaaacttcctttctaaaaaaaaaaaaaaaaagttactgggacacctgagtggctcagtcagttaagcatccaactcttggtttcaactcagggtcatgagatggagccccacggcgggctctgcactcagcaaggagtctgcttgagattctctctgcccctgcagccctcccaagtgcatgcacatgcatgctctctctctcctccgcccctttctctctctctctctctcaaaaataaaaaatccttttaaaagaaaaaagaaagagagaaagggaaagaacgggtatctccctctggccctgagccctccaggccctgcatgaggccaggcatgcagcctgacacccaaggcaaccttcccacagcaacagaacccggtctgcaaaccttccacacgggtaagacgttcttctgctctggtacgttttattgtgaaacctttcagatgtacatatgtaaaaagctctatgtataagagcatgtttaagtcataaatcataacaggacagccagcaggagcccaaatccccacttgggatgaacccaccatccacactgttgagctctgggtgccccaccagccccccaccaaggaagccacccttcaagattctgtgggcctggtcccatcttttcttggctgtcttcccacctttggatttcacgcaaggtcacacttcacctgaaaggtcttcccccatcacatgtgctcccttggctgggagagaagctcccacaaacacacccaccaccccatcatctgtgccttgacactggccacatttgcaccgctagtctgagacttcgggtctaacaggagcacgtgtgtgttcagctgctatgcccactgcagaccaggcttctgccccaccccacccacctgctctcatggctgggcaccccctcagtccacatgtctgctccccctggccactcctggaagtgcagggaagagccaggcttgggaggacaaggactctctggtcagcctcacccagtgacccgccccggtccagcagtccctcccaagtggggcttgggtcctggggcttatttcccacactacagacccagcaccaacaggtgacaccctccttcagatggagtcttgggctcaaccgagtcctctagcacaagaaggggcagacagccacttcctgacatttccaccaccttaaaagcttgggagtgctggttttacccttccagtgacctggttagcacgttagcagttaacctaggtatcagtgtcagagagcgggcaggtctagcgtccaatgcctgtcacaggagaagcagcaaacctgaaaccacgtgttcataatgagactccaaaaacatgacctcctctggaggttgctagggtatagtttattatcttgaaaactgataaataaaatcaaagaatacaacattgtgatgcctgggtggctccgcagttgagcatctgcctttcactcaggtcgtgatcccagtcccaggatcaagttccacatcgggctccttgcatctccctctgcctatgtctctgcctctctctgtgtctctcatgaataaataaaatcttaaaaaaaaaaaaaaaagaatccaacatctatcctgactttctgatataacatatatttcagggcaaccaaactgctggtggggaaattctcattcattgccaaatcacagctaacaagtacagaaagaatgaaaatagttgaatgtccccatttcccagggtgaaaatgagcacaggcaatgaacccgaggtagaccacaaacacggggagccagggtcagtggaccccaactcacacacaccaaacatgagaagtgagcagtggggacaccaggagattcagaaggcctgctgctgaccagggcagggacagcggcatgtactaggctgaaagtcctcatcggccagggacacatcctggggtcattatgggtgaactcatgagctctctggggtttgccttcagactccagatccatatatggcaaatgtaccaggctctcagattccactgatagccctacatagagagcggacctactccttgaccaaggcaaagctgtgcctctacagtaagtgaaatacagcaggttccactagagatggggccgtgggcacaagctgacaccaccgtgatgtgggaacacaggcctgcaattgggaaccgtttcttatctgggacccactaataaaacccattctcagggggcagggacaaggcaaagtcttgttgcttttgtttttactttcaaactacttcacaaaggcatttgtctacccaaaaaagagcttcagaaattcctaaaaacaagtgtcctaagctctccctcaggatatcctaaaggcaggctgcaggtgtgtgtgcaaacatgccctccctcccaggcagtaatgaaggtttccagaagtaactgcacgccactcccactggtgctatgggatgtgggatgtggtggacggggtcagagggctagtctagccccctgtgtggtggccacgtgctgggacagctgtcctcccagggccatggggcaggcacgcagaccaccaaaggctacaaggcggccagtggctggggataggcttccatgcaggacacccacccagaggccataccaacatcgcctgaagcagtcactgccctatcaggacatgaaaagtccaccctggtaggaggtgcaataaaggacagagggtcaggtcctaggggcctgaggaacaacagaaggcaggaatgcttcaccgggtctgctcagaggacacctggctctcaggcaaaaccccaggagtgtctgggatcagagatctcctaccccttaccttggcatgcttagtgggcattgcactggatttctggggcaatccggtatccgtgtccttctcagatccgagctcagcagagggctgtgagaaaccaatcggtcatgagatgggagtccgagggtgatggccactacctgcaggacatctctttgtaggaacagctgccaccagggcagagggctcccgtgcgccgtctcatgggttctcactgggtggctgtctgggtgaccactgtcatgaaccagactcacagccccaaagagggaacaggggtggccatgaactatgaaggctcactggacatgtaactggctctgcctgaagtcaaagacctgacgtcttcactgtctttacgatctcttatcccaaaagtgcatctaaagagcctgttgaggattctgtgtgtctctgagagctcctcaagcccatttaagtgacaaccatcctacagaactgactccaaaagctcaggatgaaatgcatgccctgcacccttacccacccatcccaccagtcacacgggcatgtaccactcacacccatcactggaaacctccacgcctgcatttccagcagaggagcaaatgctacagaagacgtgggcacacagcattaactagtgaacacaaaatgcagcaaagtttcaacccatcatctcttctaacgtaagggagaggtcctatacccagagcactcagaggagacagtccagcctttcctgaagcccaatgcataggggctgggagagcaggtgacctgctgaaggctaacgggtctgagcagcacagggggacacatggaaatgccccactattatttagccaaatgggatttgcccacgtgcctgacaagtccatctggcattggaagaatggcctccccacctgcgccagggccagttgccctacagagaaggtctggtatttgaaggggatgtgagcggtctctcgtgggcgcaggaagatctgcggggcaaggctgccccgcagatggaacatgtcctcctccaccggcgtgtgcagcttggtcgcgtctttgaagtatctccactcctgactgtcctggatgatgctagatagcaggggagacacagcactcccagtgagtcctccgcccgcccactcagagctggcacctaggaccaggggtgaaagccaggaccagtgggaccccttcagggatgagagcagcccggctttcagggagaacctgtagacacccccagggaccctgtcggggctgccactcaagcctacctgagctcaggattgtccacctcaatggtcactgtgtgctgtgtgttgtgggggttcctaagggcaaactcgaagaactcggcagtgcccagtgtgacgtggattgtgtgcatcgtggtgatggccaggctcagcgcactggtgatgctctctgccttcgtacgctcccgataggcagcaatgacctgcaggtcacgtgcgtgctgtgcgcggatattctgctgaatctgtgggacacaaatgccaaaccaggccaccatcatggggctggctctccttctcaagggctgtttggccacctcccccactcccatgctgaataccaaatttcttctggaatcaccacctcctccttccccagacacccgggttggatgggactggcccatcctcaggtccagaggacagtaaacgggtctacacggcactcctggctccaatcagccctgcagtggtcttccctgtagaaggtggcatctgccctgcaggctcccaagagactacaaccacccatgaggccacctcttcccatgcctcaaggctggcatcagcatgatggggaattagaagcaaaggtttctaacaaaagggggggaaatgacctcaaagctactaagactagttatgtgctcattggccaggtccccagtgtggcgccgggagccccgtctgtgagcaggcgcctgtgtaacctgagccaggccacggtccagacagctggcacgctgccttagggggctcagccacagctcagcctgcggtccttcatccacctccttcctccatgccctgattcactgggcttggccaacacaaagtgttctagaggctggagactggcttaggtttcctgtgtgacttacactgaaaggtgaggtccttggaggctgccctgatctaggacctaggaggggatgccccgtgctggggaatgagccaggccccgtgctaagaccctctgaaccctgaggaggcccagacatcagcaggcagaggatgtctgaatgtctctggacttcaactcaagaagtaaggatccaatatggtttctgacttaaatcactttggatattgacttccaacatccaaaactttggccttcccctagcaagagagttgaggccaagtctttaatttctccctggggatttgaaaacgggatggtatttgtgcttatatttgaggcctccccacacagacagagatgattagagcttcttgatggagtgcgggtcactcccagtttaccatggaccaggtggtgaacagcctcagggagagtcaacatgatcctgggggttgtgaaggggagactccttgcctgtcctgtgtcctgccacaccacagaggaggctgtcgggaagcctggcaccccacaggacatggggtgacttctccgatatgaaccagccagacatagccctccctctctcaacccttccttttgggatccacctgcctggaacaccgggtgtttgcactgctcctcaccagtggtggggttagtgggaaggatcctgcctgtggaggcggcccacagccatatggtgtcacccatgcagcttgggtctggcatttgctctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacacttattggaatcctcaggtcccaacaggcagcagagcccagtgatcagagcagtgagactgctgagccttctgctgggatttctggaaataaagctctgtcttctgtggcacctggcatggaatggtgtgaagcggggttagtgctactataagagacaacctgggggctgcagggagtgggcaagtggcagaggtaggggcatgaccccaggggcctgaagcagggccccaggcctgcctgctcagtaccaggctcggagtgctagggcacctgctccaacatggaatggctggtccagagcccaggcccaggccctggggaccccgtggctgcacagacctgagtgcatggggctttgtcactgcaacctcagaggacccccctccccgccggacagggagatgtcctacaagccttctcccaaagctgggtggggggtcagggtccacagagaaggtgcccagcacacagtctccctatgggtggggctctgctggcataggggctgacgctgaccacagcagaaggtgggacctgccatcatcccgagtgccagacacagaaagcctagccaccctcatgagaacactgagactgctgcaaacatatgtcgagcccaactctgctccaccacttccccaagaagcccccagatcatcaccacccctcgagctgttcctgactgctgggggagtgagccacgcacagcgctcttctctgtcttccaatgagcagcccccagggcccgagaccaccagactgcaccagcatgtcaggggccaaggaccagccagaggctgagcaccacttccagctcccggagggccaactgaccgcgtaaattcccaaactctcctgaaggctgagagagctctaggtgcggagaaaccaccatctcctccataaaaacaagtcactttcaccttaaattctccaggccgctgacccagaataccatgagaatttagtggaacatttccttgtatttagaaatccaattacttcccaattcactgctgtcatcagaatgagggctgcgagctgcagaatctctaccaggacacatcaacaggaggaagcccaccaaggtaagaatcgtcaggacaccagcaccaaactcatcaaggactggcagggtgctgggcccaccaccctccaaggcggggttgctggggagcccgggggtgggggtgggggcagcattacctaggggtctctggggccactagccccaggggcctcaccctcagcagctgcctggctctctttgaggagcacctgacagcaggaagcctccccagctgctgtgagcacgaagggccgagcaggttttgcctctattcagatatctgcctgccctaatgtaaagtgtgatcctcacccgtgtggcttcttgattccataaaaccatcaggtacaaccaccctctcctcccacatctaagctgtaaatatggtagggtcttctttagttttttagccaaaaatgggaatttttccagtcagaatggttacttgatttgcactgataattcatgatacggaaggcattcacagcactgaccccaaacctgggggaccctcagaatcccctgctgagctttcttaatagcccaggtaaagacccaggtgcagcaaactggacacagaattccctggggtagagccaagaatcggcttttgtaaaaactctccagacttgaaaacgttgcttgggggatgcctgggtggctcagtggttgagcacctgccttcagctcagggagtaatcctggagtcccaggatcaagtcccacattaggctccctgcatggagcctgcttctctctctgcctatgtctctgcccgccccccccccccccccccccggtctcatgaatacataatctttaaaaaaaaaaaaaaaaaaaaggaaaacctcccttggtggctgagagggtccgtttcccatacattccacaagtaggatttgggagagagagagcgagccacaggggaagagaagagagagcacaggctgctttcattcacaatgactgtggctctgcacggggacacccgggggcagaggggaggcggatggagccctcaccaacatgctggccccccagcagctgagctctcctctggactcttgaaggcgcacggacctcatccgctccagcttgcgcctgcggacggcatccccctcacggcaagcaccttggggccccttgctcccatgttgcatgtgggtgaacagcatggcggccagctcacggtccacgtcagccagcttctgtgcttggacaacattttttactgctagaaggttcaaagagagaacaagaggttaaggcagagtctacattctttaaacagacctggcaacaagcttccagccacctcacccaaggagggagctcagcaggcaacctgccctttcctgcaccttctaagatgctgataaagatggtaatcctatagcctggtctatacagtgtcccccccctttaccagcctttcaaataaaaggattctgatccttagagacaaattcatacaccattggataactttccagaggaaaaaaaaaaagtcagtgttctagaagctgatgctttctggattcccaagggccttgccaccctgacccaggaccactagccctgtcaccctcacccaggaccaccgaccgtgctcacaaaagcccccaccagcgagagcctcctgatacaggcaccactgagcaatcaggcccacggccccaccagggcctcagacgcactcgtgtgctcaggtcaggagtcaggggctctgatgagctgctcacttggcctgaagccccctgagcagctcaggggggtggggtccagactaggcctggctcccaagctcatgcccctgtctctgccaccagctgcctctagacagcctggccccccggcactgactcctctgggagactatgctcaaatcctggacacacacaaaagccatgtgggcaaacagaagctgaagcaggccagacaccagcaggcagaggagctgcattttcttcaggtgatgaagccacacgcggcacatggccatgtcacaagctgacacacgagattcacagaccaccactcaagccagtgcaggcaggcagaaggtgcaccccaacttagcactggcccttcctgaccctgaggtaccagcagtgcctacaggaaatgggttgccgtcctgaatctgccaaggggttgcacatggtcaccgcatctccagcctcctactgaaggctccatgtggtggtaaagcagtccttcccccgggctcgcccacctcgtcctctgtcacatggaacaacaagtgctatttccatgttcctcaccaccattttaaagctgttcaactgcttttcagataccatgagtcagtttagacaagtaaaatgccaccccaaaaagactgttttcacgtctcaggtgcaagcatgctgagtgggcaaccccgcctaggttgggtttgagaggcccgggggaaggggccagggcaggagacaagcaaggctaggggtaagccaaggagctcacatgtcagattgcatcactctgctccaacaaaatcagaaatgaaatctccacactgctcagagcctggaagcactttctaaaactctgtacaacttcctacttgccaagcctgccctgtgggggaagacaggctctctgatggagcaccccagcctctgtctttgaccaaacagggagagaggaaccaatgactccagagactcaattctctcaacaaaccctcacaggttacagcaccctgcatttcgggcagagcccctgacctgctgggccctgaggccgtggctttcacaaatactccctgcctgttcttgggacagccagggatggatgggccctggccccaaagtgggaaatccagattctcacccaagttcacctccctctgccacacaattaggcaagtcacctgaccgtctctgccttggtttccttaacactaaacaggagaacagactctcagcctgccgggatcatggaccttaggcccgtctgagcatctgagcctccagggccatctcccagaagaacatttgagtacccacaaagtctgggctggagtttcagagaactctcgtggggtcctggaggcctcgcggctctcaccagccacgtccaggggcctcccctccctgggcacaggttcaaggacaacagtatccgaggaccatcaacacaggaagctcgaggtagctgcagatccacatgagatggcctgcaacatggtgacactacccaaaagacccaaagggcacaccccactttgcatcctacctacaactactttgggtcatttaatgggatgtaaatctttggatgccccctgctgtgaaattctccaagcccaggcctttatctctcaggctccgtgactggtttactcctcctcaccccaacatagggacttcaaatcggtttttaaaaaccagtgtttgttcatcctaaccttttcttcattttttgctcccctgcagatgggcagctaaccaaaaaccgatcccctcgaaggattattgtcagcccagacagcttccacagagcccgctctagagtgtcccgggtatgacacacatgcttaacccacccagcccaaggggccactgaggcagggcgctgagctggacacaacagcccccactcccgcccccagatcacacgtgtacctgcatgcacacagacacacacgcagccttccacctcttactccaccccacccccatgtcaatcacagctacacatggcccacctaccccaacccgctccccactcacatctcggacccccgcgcgagatgagactgcctccctcgaagtgtctggctccatcattcgaaatgacccgtgacctggaaggtggcaatgagctggaatctctcactctctgctcacacacatgacctaaattaaaaacatcccagtataaggtattaacaccaaaaagaagtgtgatgggacacatccatgtcccctggagggagttctgggtttccaagggcctgtccataccccctggcctgctaacaccccaaaactgcactctggttctgagtctgcttggcagttgagaggtgacgggcaggcgggtcctctcgtacatgggcagtggtgtctgctttcccacgggggttaaggctgcaggtactcaggacactttttcacacacactcagttcctggtctctggagcagcctagcagccctgcgacgtggggcagctcacacattcccacgccagcctttccaatgtcaccctgctcatggaaggcccttgggcatgtccgcaaatgtctgactttaaacatacttgaaggtaaaagagcctctgcaagctagaatcttcaggagctgttttcacacagaagccttggagagaagcatgcagcctagagggccaagcacaagtccagaggcagagcacacagcacacctgccccggctgaacgggaagcagtgtcccactaaccagccaacatgtgcaaggctcaggggataagaactgtttgcctgaagaaatgcctctcctaggaggacagaatcataccctcaggacccgtgacctctctctgtggacatgactcccaagagcacagctgaagtcagattccaaaatgaacccaagttccaaacacaaatgctcatgctgtgggcttccatgattccagcaggatggtggtgatctttctgggagctattagaacaggtaccccttcccctgggtgctcgtctcccgcctctctgcgtctcctcccacctggcctccactctgccctgctcagcttcatccgcctctgcctctgtcctgtgggacagtctgggtgcaagagcaagccccccatcacagccgtgggctggtcctcagcagtggtgaccaaggtgacattttcatatccagctgcctagcagaaatgtcttcctcccgactggttctgagctgagctggggaagggaatgcctatgctgccagggaggtctctgccctgctgccaatgggggccgcccctgcagaccccagggaatggcagctggagccccccaagacatgggtgtgacagctgaccgtcccttggcattgtggaaaatgctggattcaaaaatgagagactgacaccacctctggccaaaccaagaagtgatttacttggcatggcttaaagcctggccacaaacaggtgagcagagtcctcccctagggacttccattctcatcattttcaaactcccttggtcatcttgagcgttttctgagcatctcaaatcaggctggccttcccccagccttcctgcaacaagagccactcctggttatctgcccttctgcctgctaaatggtgttcacagctcagccagtagccctggcaagcgccagagcatcagctgtcatccacgctccccgtcaggaagaccactgtcgagggccttccctgacacagcaccacactccgggaatggggaagcatggagcccaagcacccacccaccacaggcagagaagtcggctccaaagcccaggttcgccatgagactccagatggcaggcctgtctcctctgtagcaggttccttacctgcctggccactgtggccagtgaccatcctacctggggtctctgcctagggtaacctcggtgccagcctggtctcttgctcttctgcccctacctggacgtcggctctatatgtacagcatacacttggacctgcaggcccagacttccgggcagacaccctgtcggccactcccacccctccctctagccctgtgtcttgagaacaagcttcttgagcccagatgttctcagcctctgctctgttgagaagcacggaacacttctccacaggggtctgcccgccacccacccacagctgcttctctgctcttcccctccagatgcaggggctgcagtgtcttctgccaaacatggtcatctcagcatcacaaggtaataaagctcgtccccaagagtcgagcctggtgttaacaccccttcactgggatcagctgctcacacctcggggcctgctttgtcacctgaagcccaagaggagcagaattcaaggcaagacaggctctcatacttgctcatccacccattcatgaacctctcatcaccaggccaggtcactgtggctgctgctgggtgagcatcacagcaggaaccaggatgtccctgagaaagcccccaggtccctagggggacagacaagccagtaggtggacaggcagggacagagagcctggctaagtgggcaggagacagccagctagtgccagggcccaagtcccagcactgacactacttgctctggtcctaccctgatttccaggtgagaggggaacaaagcaccatctggccccagctgcactcagttcctgccatgagctcagagacagtccctgctcctggctctcagctgcttcttcccagtgagggcaaacagggcctgcaagctctccatcccaccctgaggaccggctccatctgtcacctggcagggggcacataccacctccactcactgttggggacagcggattcatgccacacctgtgggaacctggggaccctccgtagccttccctgaagcacaggcagggaggcattcaccatgtcaagctgaaatgcacgcagcctacccttagcaggcaacagctctagggggagggacaggaaccagggaaatggctgcagatccggatctggtgcagacgggtgcactgccagggcacacaaccggggtgcatggccggcattgtcagagcccaggctctcaggactcacggctgtcatcgcagcatgtgggaccagcaggaccatgtagtcccctccagacagacaagggccatccctgagtgtcacagtttcctctaacagtatttgcactcagagcgtgggttcgtgtggaaggctgcacaataagctagatcagggcaacatacacctgcttactctgggcctggatgtggtgaaggtgctcacttcttcagggcaccatctgcaatctcaagttgctgtgggctttcttacccttccatgacaccggggttaagctggggacccacacatttgctgtgggagcggagactccctccagatgagcagggttcctgactcagctgtgcagctaccttcattccacggtggcagcagagcacatcaccccgtctgaccccaaacccaggctccctgtccacaccacccagctctccttcaaccctgagaatatggtgccaggggtcagggcagccccttgctcccctgaggagccctggaagggctgcagatacaagtgacaatagctccccgatgaaccacagccacaccgggatcccggggacagaaaagcaccagagacactgcagtcttgtctgcagggaccatcgcggaacctagaagccaccaggcacaagcaccccgccgcccccttcctatggcagattcatgggagatctgggctcagagagggcacctcgttctgcagcctcacacttgacagcatacactgggagtgcagagccatggagcgccgtccaaggcaaggccacgccaggcccatgatgctgtcctgaccctgcttctgtcccctgaagctcacacagcctcatccaatatgcaggcctgcacctgctgcttggttctaaacacccagttttaaaaaagaaatacaagacacaatgccattttttgttttttgtattttttttcaaaaaatgcaaaatttacagaggaaaccagaccatggctgtatggacagtgagctgctcgcgctcgtcctttgtcaatccccctccgctttggcgcctctgggcgggtgtctctgactctcccccgtgcgctatcgctcccttgcaggcaaagtatggaaatgacaatttaagataacgtataatagagctaatctttcttctgaagataagatttaaagcgcggagtcactgcacatttgtgggctgtgaataaatataaaaataaccaatgcaatctcagaagcagaacgcttcttgcagctccctcactcataggagcttctgaatgatgtctgcttctcacatacagttcctttttttctccagaacctaattgagttttgctgtttccatattgtctgttttcaaggcggcctcggatcttacaattgtcaacaagcaggcagaaacctccagcctaacttaacgagtatgcacgcctcctgctgctgcggccgccccagcctgcccgcctccaagctgcccgcacgagtgcatgtggcccatggggctccaggctttaaagccagcctgggctttgaggaatggaaaattcccccataaaccatgagaggtttgagtctcatctagacagacatggctccagaaacctcaaatggcttggtttcgtcctgctttccatttcaccccaaaaaacctcaatccttccatagtctcctcaagagagactgagtgggcttgtcccctggccccacaggggctcactccaggcccctacccagtgggaagcacaggaagacatctcaggccccatatcccctgtcgatcaggacctccgtggagcccccagaatggccagccaactccactgtagccacagccagccccataccctgccccaggcatgtgaacaacagcgcacagggctgcactcagtgtctgaagagtgaccctcagacatcattatatgtatggtcagagtataggctctaaagaagtgtgtttctctgttcccttatgcccaagctgcatcaaagttgagaaacattttgctttcctgtcctatgatgaagtaggctcataatctagatgaaaatctctcctggaaccagtcattttatctgaggattttcatagagatgtcacatagacaacaccaagaaacaaaatgtggacaagactgagatgactaatgattccaagtcctgatcagaaaccaagaacaccaaacaactagtgccaagacaggctcaggggtcaaggacagatctagtggtcaaagacaggtccagtagtcaaggacagatgctcagtggtcaaggacaggtgctcatggtcaaggacagatgctcagtggtcaagggcaggccaagtagtgaaggacagatgttcagtggccaaggacagatgccatatgatcaaggacagatgctcagtagtcaaatagtaagagtttttctgaattccggttgatcatatgatgcccacaataggaccactgccctgagggagctcatgcaccaactgggaaaatgaggggccacccaggagacagcataagaccaggtaaagctgccagggactcttacccacgttggccaacgtcaggtgcagccagccctttaccactgtgtagacaccgatgggcttgacacagccaaatctggtcacatctccactcaccaccacatcctgttcatattcagtcgccacgacctcaagctcatgcaggacctgcacagccggccggccttggcggaggagatgctgcagagaaaaaggcaccagtcagagggtagccgaggtagagcccatcttctactgaaaatctacactcttgatattgggtctttcccacaaattcttcaattaagtgcaccccaattcacccactgagaaaatatcatggatgcacaaacaggcctggcagccaccctcctggaaccccttctatggggagaacaatgggaaacgagcaataggaggaggctgggaatgctgagcagaccccatctcccagtcagtgagagcttcaagaaggaggtatgaaccaggccggggatgctaagggcacactaggcccagggtacagagcagaccccccacacccggggccaggagcatcaggcttcagcatggctgaaagacaggtgaggcctggttgtggaggtccacataaggcccaggttggtcagagccggcaagagcagatccaggacaggcctcctaaacccagtgctttctgctaatccagttcacccctcagtgccctgcctgagcctaagtcatagtgaggcctgcaatggcctcttcctgggaggccttgggaaaatcacctgacctttcaactagtgcttcccactcacaaatgggcatgtggaggtcagacaagcatcccaacttctggaattcttgcacttctactacagcaaacatgcccagctctgggagtggaagtgccccaaatgacccctctgtagtggctctggcagcttctggttgcttcaggaggatgaggacctggccggactccccagccacccacccgcctactctgcgtgccgaacctcccacacaggcacacctctgactcagaaccacacttctgatgagttctcatctggggagggcagagagtgacggtaataagcacaagcttccaagtcagaactgggctaaaaggctgggtctgctgtggtccgcgtgaccgtgggcctgtctctcagcagcacatgaagacagtgcagtacaactgggctgaggacaccgtccacgaagttcaagagcaatcagatgccgtggcaactgtcactattgtcctaacaacacccttgtagccttgggaatgagctgaagattgggcaaaatgtacgtccagatgttcactgcaacattgctcatggggaaaaaaaaatcactcagtgcccagcaacgagagcaagtgagacgagtcctagtgtatccataccttccagaaaatgtgcctacccaaggcacacagctagttcagcacctggagttaacaggcggttctctcttcttacctcatctcccatctcttctatataaattattaaggtaaagaccttatttttaaaaagtaaaatttaattttagtttatccaagttaagtgtcaataatcacaacagcatttgtttcaggggctctttgtggaggacaatctagagtaacgtcatctccagggacacagggacaccgggctccgcaagggatgaggcaggagccacagcgggtcccaaggtaacatgggctcctctctgcccctgcagtgcccttcctccccccactcctacctccctcttccctccactcccacttcccccactccctcctccctctatccctccccatccctcctcccccattccttcctcccttcactccctccatccaccttccctcctctctccatccctcctccctctatccctccactccctcccctcattccatcctccctccattcaccctccctcttccctccatctctcctccatccatccctcctcctccatctatctctcatccctcctcccccactccctcctccttccatccctcctctgtttatccctccactccctcctcccgtttttcctccctccatccctccttcctctatccttcctctccctcctccctcttttctctcctccctcactccctcctccctccattcctcacccttccccccacccccatcccctcaatgctccttccatttgctcattcccagctacacagcaccatgcccactgtgggggtcatgactgcactgaagatgaagagcagtcccattcctaccctctagaactttcaccagccagcatcagagacagacaagcaatggccacgatgaccccctggagttgcacggtgacccagaaaggcccaagggaccagggtgaggaatgagaacatcagccaatatctactgggtgctcaccacgggggacccctgccctcactcctcctgctcccctgggggcatcactcctgaggggtagacactgaatcgggtggaggagggatggtacaatccaggttagccctcagagggaagaggggaagctgtgtcctctcccacctccgtcccacagccgggacatcgcgtccccagtccctagcccaggagctgcggtgtaattacactgaccctctgggtagaagccccaggaagaatcttaaattaacatatggatttaggttttagtgtaggaggaagatggaaagcacaattagagaaagaattagattctaattacaattagaaaagcagagttgaagtaacagatagcaggcaccaggtgatctctgcacacccaatcgatgactcatcctaccctctgtgggccaggcccctgctctcactggtggggggcagaggcagaaggggactgcacctgtggtcagtctgtaggaggagctgatgactgaggaataatctcgcattacacagctgagactctgggaaaatcttttcctttggggaaggacggactttatgaacaaagacatcagatgacagcccacactggacgagaaccagcaactgagcagacccctcccccccccaggagggacaccccaccctaaccagcccgctgcagagcactgccaccttcatctggacggccgcagatcccacgagcagcaaggagtctccatcccagatgtcgatctggagggtctgagcagccatgtagtgggtgaaccagcgctgttctccgggtttcaggaacccaggttccaccaggtacttgagttgaaagccaggtgtccctgttcagagaggccccgaatgagcccaaaagtgatttgagtcattatcatcttcacattttgaagagactcctgctgtggtcacaatcactaatttctgcttccgtcataactacagtttaataaacacattaatcaaaaatatcataaaacatccacatttacaaaatacagagtcgtatgcagaagtctacatttctttccaatacatacaatgaactcctcccagaataccccagggaagaatcttgtgaaacagtctatcctgaatggggacagatatcctgacggggctgcccagggaggtcattattctgtgtgcagggtttaaggggaaacatggtctgccgtcaaaagtgggacagcacacatgaaagcacgcgcacacacatggagctcaggctaaggtcaggctgagctgccctcaacgcattcccagcggccactaccaggtcatctctgagacagcttctccaggtggcatcaccctcgattctaaactgacaaaaacaccactaaaatcattagttaaacatttcccccttcccatcaataacctattttcaggggcacctgggtgcctcagcggttgagcatctgtctttggctcaggtcatgataccggggtcccaggatagagtcccacattgggctccctgcatggagcctgctttttcctctgcctatgtctctgcctctctctgtgtgtctctcatgaataaataaataaataaaatcttaaaaaaaaaatattttcagagcctctgacgagtagaatactaccatgtttactcagcattgggaagcgcgacccaagtcagtctctgcctctagccacctcagcccctggggagcggggtgggggtggggggcacacagtcatgaacgtcccctctcctctcaagactagaacagtgcttctagttgaaggagggaagcgggttgggatgggacacagcaccactgagggcctcgcatgggcctacgggactaagccaggaccagttgccataggcctggctccgaccacagtgagaattcacagggacacctcattaagggagcccagcaataaggaatcaggaaaaaggctcagagagggggtggtaatgacatcatgcagcggcaggccagtccttagtatcacatatccagtgaccagacaaagtcacagggccaatggaggccaaggacacctaccacccttgccccagtgccacccaccagctgagacctaaagtggcctgtgacaactggtcattctcaggacaccgcctaaataatgggagccaaggctggctccttagatgcatggtgaggaccagacaaagacctataaacacccaagaccctgctcatggcccgaatggtatggcaccccataccaggcccacctgggctgcaggagaaggaagagctcagccactggcttctacactggtcatacttctgattcgaggagacagaacaggtttgacctctgtgggccaacttcccaaacgatgcctacagacaccagccttgggagctccatactccacctccagcatcgcccagatccacagaccctccatccagagttctacctgctccccagtgacgcatgtcccagatggaccctccatccttcccacaccagctccttgttacatcgtcccaaagtacttccttggcaccagacccttgcaacgcccatgctaggctggctcccgctccaccaccactgcatccactcagctgcctggccctttggcacaccattaactcacactcttcaaataactgcagtgcccccagcatgtgcaccaagcaccacgcagggataacttgagagagaaggacggacagggagggtccccacctgatggagctcatggtcacaggataacacactcagcccagagctaatggcgtgctgagtacctgggggaggcatgtggggctgtgggggccatcctggtctgggcccagcctcagggctctcatgctggaggctcccagttcctcctgccacaggcttgggcccctttacttctatccagctctgacaaccggctcctcagtggccttccctgaggtccacactccctgcagtccatcccactacacagccaaagcatccacaaagggaaccctgtgtcctccatcctgctcagaagtgagcaatgatactgaccatctactcacacaagacagacctcaggggcagtggcacctccccagggtccactcaggttcccaagcttacctccctcaccccctctcaaacaccaagccccagcccagctggctccattctgagcctcagtccagccacacccccacatccaagtgtggtaggtactcttcctccggttggatggtcctcagtctccatctctccttcatgacccacaaacacccctactctgcgaaggcatccacaactttcacagatgacaggcactttataccactatggtctacaatccacaatgaatccacaatgaatatacagcagctgggaatatctacacaccatatgacaaagcaaaaatctagagacgcaaggagaaacagataaaaacataataataggagagatcaactaatcactttgttttcagggcaggccaagagggcaataaacaacagcatggaaggccagccaagataatccatcacatagagcgtatacatgtacatatgacaccattccctgctaacattgaatatatctttcgagtctatctggaacattaataaaaattaactatatacctggccacgaagaaaacctgaatatgctccagaaggtgaaaataacacaaacaatattctctgaatacaatgcaacaaattagaaactggaaacaattttttaaaaactaatttacagcttacttttaaataacttgatgtcaaatggaaaacaaactccagggcagcccaggtggctcagcggtttagcaccaccttcagcccagggtgcaattctggagacctggaatcgagtcccacatcagactccctgcatggagcctgcttcctcctctgcctctgcctctgcctctctctctgtctctcatgaataaataaataaaatcttaaaaaaaaaatccaaacagattttctacagaataatgaaaatactacttatctgaatcaccaaagcataaagctgtaatcaaaggaaaattcatagccttgaatactttgcatccacaaaaacaaaagaattaaagtaagtgaattaagcaactcaaattagaaaaataacactgaagaatacaaaaaaaaaaaaaaggcaagaaagataaagatgaattttaattggttagaaaataagaaaagagcaaaacctgcatgaatgtgtctttggagagtcagggaagcaggagtgaggacatgagaggatgcccatatccctgtcccatgtcccattagggcagttctcccgaggttaacctggattcagcagaacccccataaagggcaaacagtttcacatggaaaaccaacccagccagaatgtggggtggtggtggggctgcagggaagaagagcatggtggagtggagggaaagctgtaagagcaaagttgcacctctagctccacagcacacccactgagtggacaggtggtctaatgcagagcttggcaaaccacagcccataggctgattcagctgtaagctaagaagagctttcacatttttaaagggttgttaaaaaagaagattccagagagaacatatataacccagaaagtctgaaatatttactatctggcccttaacagaaatcagtctgccaacctctggtctaatgaaacaaaatataaaatccagaaataaatctgagccataaaaaaagggtataaggtaagggtggctctcaagtcactttagaaaaagatggactttcttctaataaatggcactgggtcaaaagaacagacgtttgaaaaaaaagataaacctggactcctatgttccatagcacaacaggacaaactacaaatggaccagaagtctcatgtaaaaaatgaaacctataaggcccctggagaaaacgtgtgtggatctctttatgaaccaaggagaacaccttcgcatccatgactcaggaatttagaagcaataaaactaaagaccaccacattaaatggcataacagcaaaattttatggggtacctgcaagtctcagtctcagggttgtggttttgagccctaccctggatggagagattacttaaaaataaaaatcttgggatccctgggtgactcaaaggtttagcgcttgcgttcagcccagggtgtgatcctggagacctgggatcgagtcccacatggggctccctgcatggagcctgcttctccctctgcctgtgtctctgcccctctctctctctctctctgtctctcatgaataaataaataaaatcttttaaaaaaaatctttaaaaataaaactttacatagcaaaacacaccaagagcaaagtaaaaatgaccaaaacaggagaacatatttccaatttatatcacaagtaatagctaatctccccaagaaacagacttcccaaaatagaggacagaacaagaattggatttaaaaaatgggaaaaaatatgaatagaggtttgcagatagacaaatatccaagatgctcaatttcactcattataagagaaatgcaaactaaaacttcattaagatctatctcacatcgatcagattatacaaatcccgaagtttatgccacattttgtgggcaaggccctgaggaaataggcagccccatgcgctgcaagcggagcttggaaggccatcgaatgacaggggagtagctgacggctacaatctgattgttagtaacgccccccaaacacatgtccacacacagagaacatataaacaaggttacaagctactacattatctgaaactcaaagcctgaaagcagcctgaacgtccatcagaaggaggttgacctgaatcaatcacagggcacacgcccagtgccagacgatgtagctgtgaaaacagtgcagggggtccccacatggagtcaagctcaggacagaatgttaatggaaaaaagcaaagtgataaaagagcatatttggtatgctatctattgaataaggaaataaggagaaataaaaatacatttgcttacttgctgaaacaaacagaaacatcagaaggagaagccagaaactggaagcctggttacctccaggaggccctgcagggggcacatggtctagagggtacaacggttttagactgtgagccatgtaaatattccaagaaattataccagaagggaaagaaagccaataaaaaattgataacaaattgaaataaatgaatctgactgaatatcaaattggtaacagctccacagaggaaagcatgagctgcagggattctgacacgctgaactaactatgtccccagcgggatgtggctccgccctagagaatgccagaaacctcaaccctcttcactcagcacgcgtgcccttagcagtaacactggcaatgaaattctaaaagtacattatagaaatcataaggaaagcaagccatatgatcaggaagcatgattttcagtataagatggaaaagataaaaataaattaaggaaaaaaccctaaatctgaattagaaatatcaatgtgaactcatgtttgaatatatattctccagccctgcccagggaaaggcctggaagcaaggccatgcaggaccaaaagtagcccagccctcagtctccaaaagcaaatcccatgcaggagaagcggctggtccaggtcaaaggcagaggaagtccacaggagccgaggatgctgggccacccagaaggcaaggccatgcccaggctcctaggttaaccaggccaccagcagccaaacagaggggaagtagtagtaatgaatatatcacagtgaatagatatcagagtgattcaaaagccaacacaagaaacaaagaaaaaagacatataaaacctcaattgttaccaccagagtaactgtcacagcttctgactctgaaaactggaaattaaagggaaagtatcaaacatttgctctgttttggaggatgtatacatagttcactcttggttgataagggaaaactggttttttaataaaagaatgtcagtgaataaagggaaaccaaatgacaaattaagaaaaattaccattgcaaattccaattaaagacctggcccaggggatccctgggtggctcagcagtttagtgtctgcctttgtcccagggcgcgatcctgaagtcccgggatcaagtcccacatcgggctccctgcgtggagcctgcttctccctctgtctgtgtctctgcctctctctctctgtgtctctcatgaataaataaataaaatattttaaaaaaaagacctggcccaggacgtaaagaagtgggtaaaaggttgttgggtggatattcacatggagctaatgactggcctgagtagtccatgctgagagccaaggggaatggccccctgccccccagagggatgaggggtagatggagccagtgccctgagaataagaggcctgacctccacactgctgatgaaatccaggcgtgggggagtgtcagccctacctgcccgtgcacaagcccttcggggagaaaccagcagatgcacacagaatgagcgacgttctatgtgacaattggtctggtaccttccagtggccactgtccctggagagagaatgcaggggactattccagatgaaaggagcttggggaggtaactcccagcacaacccaggacccagactgaatgttggagtgaaaaagtcactgtcacaaacatacaaggaaaaaccaggaaaggaaacagtgagatcccgctcattgtcagtatgatggaggctggttttcaatagagcctgttgctatgttcagaggcaaaccatcacctcacctgctactgactttcaaatgattgagggaaaaagatgaacagttaactctcaagcatgtccaatggcctagacacgatctccatggcagaggacatgggactggatgcggagatccaccgaagaaaaagcaggcaaggcagcaccagatggacccagaggaggactggcaagccccagatgagactttagcccaggggggaggggggtctctcagatgacagctcaatgactccagcgcccttggtaataaggaaggtcactgaaacaccaggaggaacaagttagaaaaatccaactccaggcctgaaagcaaaagacacgaaaggaaggaaggtaccgtgtgtgtggaggacggcccactaacacctggacccctggaaggagcaggggcaccagcgagcatggaagccctaggggcacaccccagtcctaaggagaggagataacaagaaacaattctactcacaaccaggaacaccaagaaacaggagcctcgggcagaggccaccagacccagggcggcaggttccagaagcttctgttcaggaggccaccaagtctcctgctggaccagatctactccagcagatgtgccctgggagaaactgcttggcaaattgagtaaagatgaacatgtgggaccctatagcaatttcactcctgggcacacccccaacaccagtgcgggcacatatgcaccagaagaatgctcaccacagcattattcaaaagaaataagctgggaacagggggatccctgggtggcacagcggtttggcgcctgcctttggcccagggcgcgatcctggaaacccgggatcgagtcccacgtcgggctcccggtgcatggagcctgcttctccctctgcctatgtctctgccaatctctctctctctctccccctctctctgtgactatcataaataaataaatattaaaaaaaaaaaaagaaacaagctgggaacaatacaaatgtctatggacaaccaatcagataaatggtgtgcaatactcagagacttctatacagccaagataaaggacaaactactgccccacacaacaacatgcgtggacctcatagacacaatatccagtggaagtaagactgggaaaggcacacacaagatgatcctatttcagttcaaaactgcaggtgcactggtctacagggccagaattcaaagagtggtcacccctggggaagccaccgatggtgggaaaggagaaggctgggagaggcctggggatgggtctgcggccctatctgagtgggctggtgatcacacagatgtgttcactgtgtacaagttacagctgttataaaaattaactcaaaatgggtcacggacccagatttgaaacacaaaacttagatactcttgaaagaaagtagaggacaaaattccaggtgtggtgacgagtgtttacctctaacaccaaagacacgaacgatccatgacagaaataattgataaattggactccattaaaattacaaacctctgccctccgaaagacactggtaagaccgtgaaaaagatgagccacaagctgggagaagatattcgcaaagcacacatctgataatagtgcaggcaggcaaaatataagaaggatctcttaaaaaatacaataagaaaatccgttctacaaacaggtaaaatatctgaacagacacctccccaaagaaagactcatggcaagcaagcccatgagaagatgctccacatcacacgtcatcagggaaatggaaataaaaacaacagtgagatgtcaccatgtacatatgagaatgggcacaatccagaacgccatcaacaccaaatgctgggaggatgtgggatagcagggatgctcactcactgctggcagaaacccaacatggcacagccactcgggaagacagttgggcagtctcctacaaaaccaaacatactcttaccatacaaccatgcagccatccttctagatgttacccaaaggagttgaaaatttatgtctacacaagatcctgagcaaaaatgtccatgattgctctagtcataatcaccaaaaactggaagcaacgaagatgcccttcagtaagtaagcgaatggatgtccatctagacagtggagtattattcagcactaaaaagaaatgagctaccaagccacaaaaagacatggaagagtctcaaatgtgtactactaagtgaaagaagccagtcgggacaagctatatactatataattccaagtgtataacatcccaaaacaatgaggacagaggagggggtgatggggcaaagaggtggagctcagggcatatttgggtggtgaaattattctctacgatattgcacagggatacagatgctcggcatttgtcaaaatccatagaaggtacaccaccgagaataagccttgtgaactctgagctttagctaacaacaaagcatcaacactggttcagcaactaggggaacttctggggcaaggggtggatgggaactctgtacccccagctcaatttctccataaacctgaaactgttctaaaaagtaaaatccataaattttttaaaattcggggaactgaacactgtgttgcatgcatgttacatttcattaacaaaagaaaacacatcctaagtgagactctagtggggctaggcactgagctatggggacctgagctggggctgctggaggaagcctagccccagccaggtgcccaggcagtggggtggtggacctttggctgagcctcatcaggtaagacactgaaaagaacttgttttggacttcaacttaaggtttttggacttcgagtcttaatccaataatgacaaaatgtgattttggagtttaatttctttggacttcaacttacaaggaaatttaatttttggactaatttctctctgggaaattcctaatggaaaggatttatatttgaatcaggagtacctctccatggttggccttttgaattaaattacctgaacactattcatttaacctcatcacagaacctctggtgaaccactccagatagagcagatagatggggctgggaacaggaaatgaaagaggagacagtttctcagcaaaggtgcctgggagccttgaaatcgatgcactattctgatccatttcagagccttctgagggcatgagctaaactgtccaccaatggtccccaggagcatcagcaaagggaaggcagttggtttgggacaagtggtcccaggattgttaggaaacccagggagttgagaaaagaaaacctagagtattgcaagagagcatccttagctggccacacacttcatgtagtggggaccccctacagtcaccctctgcccagcacatgcctctcccagaccaaaagcagctcatgggctggagggtttgggagaaggaacagggcctgagctgtgcccccatctcctctctctttccagatttgtctgcaggaatcgagggctctaccccagggtctgtggccaggtggtcctacctctgtcggcagcaaagcccatctgatgaggtgagcaccagcaagcacacccaactcttgggctgaccagtgcctgcaatagctctgaccctcttgactgattctgaccttggacaagggacagcagcataacctgctgtacttatgactcccctaccccaagcccagacaaactctgtctactacctgttttctaagcatttatgcttattttaaaatttctctccttctacttatattgaatttgttctggttgttttcttcaaacttctggagttgaacaattagatcagaatttttaaaaatactttcttctttcccaatacaagcacttaaagctgtaaatttccctataaataacattttacctggatctcatttgttttgcaatatttgcaacatgattttgtgataccgttttcaataaccattcttattttgatttcttcattgacccacagaataaataaaagtatgctttaaaattaaaaaaaaaaaactagcagttcggagcctttttaggggagggagtggttgatttctgacatgtggtattacagtcagagaatgtcatgccattcagatcagatactggtctcctggaacatggtgatgcctgcacggtggcctaaaacatgcacacgtatacgtgactgatcagttcttgtaaattagtctcaagcgtgcttggaaagaatgcacatcctctaaattggttctataggttttataagattatttttttaaattgtatcttattacatccttgattattttctgcttgatgcgtcaattcacttggaggttcatcaccttccacaaccatagatttgtcaatttccccttctaaagtctgtcaatgtgtcaacatttcttttgtgttttgaagctgcttgttggcagttcacaggttcagaattatttattttcctagtgattattctcttgattatatttaataggcttctttatcctcattaccgcatttgccttttgtctcttttgtctgatgctaatgtcaccaaactactttcacttgattatcattttaatatggcatttcccacctgttctttctactgaaggagcagcaagggttcccagctctgcccctcaaaaaccctgccactggccaaagaggggtaactcatcattgataaaaacaatatatttggtatgttttgatccattgcagtgtggtttataatgatgccagaaacaaaagcccttggccccactttcaaacaatgctagggaactggctcattattttgaaaatttgtaaataaatgggaagaaatgaacaccattctgcctttcctacatggactacacctcaaagtaaccccatagttgaggagggcaagtttctctatatggaaggattccagttgagaaacaaaaaggtgttccaggtggggatccctgggtggctcagcagtttagcgcctgcctttggcccagggcgcgatcctggagtcccgggattgagtcccgggatcgagtcccgcgtcgggctcccagcatggagcctgcttctccctctgcctgtgtctctgcctctctctctctttctctctacgtctatcataaataaaaaaaaaaaaaaaaaaaaaaaggtgttccagaagtacaacatcaccactgttcaaccttgagcaaaataaccagcctatgggcaaccatcctaacagccactgactatccacagaaagaagggaccagcccctgcacagccccagccccagagagttcctgcccaaaacaaaaacatccctctgaccgtgcatagagatctccaatgtccaggaaaaacaggtgacagagaagggcactgtaaatgtcaccactgggaggcaaaacccaagtgtagatggtgcgaagctctgaaggatgaagcaatgtggctacttcaacaaataacacaacaaccaaggccaggggcggcaggggagctgcagacacacacactggagagtcatgtcaccgcaactcacaccttcccaagatcttgatctgaacaaactattaaaaactgtatcctccataaaaacaaacaaacaaaaatccttcttccatcaagttcgaaaaagttttttataaagttcctgggaaaatttgaacagtagctaaatatttgataacatttaagggattattattaattttaagcaggataatactgagtttatgtcctctcaaacaagactattttttttcagatatcttttttttttcttttcttttcttttctaatttgttcaagatgtttcctctctctggcatcccactaccctctatttcttcttcttcttttttttataaatttattttttattggtgttcaatttgccaacatatagaataatacccagtgcttatcccgtcaagtgcccacctcagtgcccatcacccaggcatccccacccccctcccacctccccttccaccacccctagttcgtttcccagagttgactctttactttttaaaaatacataaggaaatagtcacagattttgcttcgagggatctagggtctgggggggcggggcacagttgacagctgctggcacccactggcagaagcactggttcattacactattccttcaaattctgcacgtagggatccctgggtggcgcagcggtttggcgcctgcctttggcccagggcgcaatcctggagacccaggatcgaatcccacgtcgggctccaggtgcatggagcctgcttctccctctgcctgtgtctctgcctctctctctctctctgtgactatcataaataaaaaaaaattaaaaaaaaaaattctgcacgtgtttgaatattttcaccatacaatattttgtaagtaatacaatttataaacaaggctcaatgctggatttctcacatctttgccagacacataagcacttaccagcatcagaggagccatctttgtcagtgagaaccaggagatacaacagggaaccagagcctgtcctgcccgtctcgtccagcctgatcagctgcaagcgtggcgttgtcacgggtgggaagcgatagaactggaaggtgaaatacacggtctttggccacggtgctccctggcagtcctgggacactctgaatccaaaaaaagccccaaattaatcaagtatccatagcagagaacctgggttttcaagagcccagcaccacaggcaagacactgcctcatccacccacatgagcagctctactgggatctcggggagcccgtcagacagcaacccatttcagccccagctggagaaccctgctctcagagctacatgaataccaagaattggccaacaacactgtccatgtcaatgacagaagttcaggcaaagccagtttcacagcatctagagatggctgtgtgagcaccctaaccaaataaaaacaaaaacacatagcattccttaatccaggaagacctcagcatagagagtcctgccaactcagagccatacagaggggtttcttcaatgcaaggaaagtgaaagccaggtaaagagtaacataaatagaatatttaagatcgcacctgctaaaggcaagaaaatgcagcactatctcattgctttgtagacaatctgattcttccttttgggggttgaatttcacgggatctgtaggatttacagcctcggctggttgcttattggcatccagaatctcaggaaagccgcaggcttgcaggagcaccattgaggctcgtgagagcttacttccagagctaaaacagaaaagatcatggtggccggcctccagaaaagggccggcaaagaggtaggacctgccgacaacccctctctggggaaagcctgttgttcagagtcagtcttcccagcacagcatgccaggggcctcagggccacctctcacctcctggtctgggctcccacaacaatcggggtgtgcaccggcgtaaaaggcagctcctgcagctgttcagcaacagaggctcccaggaccaaggacgcctggctcagctgggcctccaggtgagagatcctgccctccgaggggggctcctgctgagcaaaatgcggccccaagtgaggccaacaaggatcccagcctgtaacaactgaggaggctgagatggggggggtgttcattgatgagccacctgtcggactgctagcttcagcaaaggaaaatacaggacgcccaagtgcctcttacactttaggcaaacaatgaataattatggtataatgtaagtatgtcccatgaaatatctggaacttacactaaaaagtcaccacttatctgaaattctaatttatctgggccaattcgtattttaggtgagtcaaaaagcaatgagggaggtatcatcaattttatgaactggcaccattgagagatctgctgtctgagaaggtcaattaacataaaaaccacattgatagatcagaaaaatgaaccctccttcaacgctgcaagacagaccactactcagctcccagctcgcctgtatagacatttctctgatcccatcacctgaatattctagtcattcctttgtattccaccttccacatccctccagagcagaagacgtgtctaaatgaagagactgtttggaaatcagaatgctctctttcacaccagctcacctagctttctgtttcagctcaaaaggaataccacaaaaccagcgataactccgacatgtctcagctgcctataataagaaatgcaggcttttcataaatacgtgagccctagacatgttctagaataaggcatctcacggtactgttttcaagtaaataatttaatggcgctatttttcctcaatagacttccagtacaggactgaagatacagtttgcaggtgagccagccaacttttgaaaccaaactctatgtgaattccccaaggtcagtggaaacacaggcctgttttccccactagatgcatccaaatccaggacggtgggcttcactacttctccttcagctgctgccaacaaggagagtgtcctggagcgctggaggcttggatcagcctctatttagaactctaacaaaactttccttcccccttcatgtacagagatctgaggggggtctaactttgtcttgtcaggcagagcaaaagcctaagtaagcctcatagcagggagagctcccacaccaaattcccaggtctgcaggacagaaaggtggcaagtacctggggcatggaagtaagccatgcccacccactccccctgccaccctgtccctacacccatgtcacagagaacccacgaaggtctcagccacctccccaaggaccagcacagctcacaaagtcaaagactccagcagatgtcaggagttcagagccattccctggaaggaggaagggtggccatgcttacctcctgggccggcgggggcttagagctgtcgggttgccgtggagggggtgtggctgagcgggagcgggtggtaggctgtggggaagctgcgagctgggaaagggacaactagagggggaggaagagtgggtgagaggtggaacacagcccccctacattccagcgcccccacacacacacatacactcacactggcataaacccaggagacatagcacagaaggcagtgaggtctgcaaccgcttgctgggtggctgctcctcgttcctttactagttactaagaggcaagattgtggtccctaccacgtggtaacactggcataaaaacaatagtaacaaggaacagcaggtagaaaagaggaagggccgaggaagacaggttctctgccacttatgggctgtgggggcagctggtctttgaacttctctggatttcacatccagccagggactgaacaggtgcaaggtcccttccaggtctcaccaccgctgtgcctgggacacaatcccgtcactgaggcagtgactggaatgggagccgacatgagctctagggacagctgctgagactgaatccccacaccactattactgtgtgactgagggcaggccattgagtcctttcttttcctctgtctcctcatctgcaaaatgggggtgaaaatagtacctaaatcacaggactatcacaggaattaaatgagaagtcgtggaaagggcttggccagaacctggtgcatgtaagcctcctgtcacgtcgcagatgtctggaaagtgaatgcagctgtaaggagggcctacgctccatggaagacctgcctctcaaatcttggactcactttatgacatatacagttagctggcccagcacagtactgggacagaggaacccaacacgagacagatcaaaggagtggctgggataggcatgtggcaatgggggaccccagcagcagccccaagggcagagaagagctgtggggtctgttcagctccattcagacccacaagctgttttgagactttcctgcctcaggaagtgggcagcactgttctgtggccctgaacacttagggtgacatggaaactgctgtaacgggccagtagggaagcttgggaggctgtctccagacctgtgtcttcaggcaaataaaacgttcacttctgagcgagtctccaagctgcttggtacacgttaaacccacttccttgagacagactttgatagtgagttattcgacttttctagataagtcattttccagggtgatgctggcaaagtcatcaggtagacaagccaactctgaccctgaacaaggtgatgccggaaccactggcaagcactgtgacaagcttggggttacaaaggggtggactcaatggccagccaacaaccagcacctctcttcctatgcaggagaccctcgcacacagaactatctgctgtctgacaggcggtggcccatctttaccagcaccagctgttcagcctatgggcctggctttggccccactgcccagggcagtggtcagtgccaggcaccctctcctggtagagacaagacaggaggaaatgaagaccacagggcagcctgggtggctcagcggtttagcgccaccttcagtccagggcgtgatcctggagacctgggatcgagtcccacatcgggctccctgcatggagcctgcttctccctctgcctgtgtctgtctttctctctctctctctctctgtttctcatgaataaataaaatcttttaaaaaactttaaaaaccgaacttaaaaaaaaaaaaaaaaaaaggaaatgaagaccacactgtggcacctcagaaaaaaacaaagtctactcggccctgaaagattctcagctatcagacataactgccagagagagcacatcattttttaaaagtactgaatgtgtcccagcaaaacaaaagagacaccactgttcttaatgatgcagagaatgttctgcccctggggcctttaattaatggattttggaattctgaaacaggcctctcagcccagacttacccctggtcccaccggcgagtcctggagggcagcgagcccctggggcggcagtgctggcaggtctgggggggaagcagacagaggtggggaggggcactcaggagccttgggaggcctgactcagggcctgacccatctgcccccggctgcgactggtgttgccgctggcgttgggcagtccaatcactctgcagtaaggtccagagccgctcccccaggtcacagcctcagaggacggagctgagctcgggctgctgtcactccagttcccctacccctggccaccccacctggcctcatactttctgactgtgtggtgtcaccaagaatgggcttcatgacccatgaagtcctcacacacccatcctgtgcctttccatcttcccatctgctcatactcccgactctggagagctggggccctgggtcagctggcctggcacccccactcctctgaccagggtggacttgctgttgtgctaagtaagcaactttgaagcagatgcacatgccgcaccctgctgttcctgcctccacccccgcagatgcccagcgcacacaacctgtcctgagtccagcgtactctagatgtgggcatgctgggcccaggaggggccggtggccatgcatgtccttgaccttggtgatgccaaggctggctcctgcaggctccaaggtgccatcctcctgccaagtccaggggccagccccatcctcacaagccaagtcagggcgcttcccagaaactgagaaacactctggcctgcaagccttcacccccgaaaccagctcacagcagatcatgaatccagctgtgctcccaagaaaggtgtcatctcaccagaagcaaagtgtggaggaggattccagcagccaagaacctctccctccacttgagagtgctggcggcagctcactgcccatcccaaggcggaaagcattacagagccactgagtcctccgtatcccaccctgataccgggactctggaaagccccacagactgactctggtcacaaggatgaaactgcaggaaatgggaagggaccagaccagtggctcccccaccaccagggctaatgctgtgcggacaggtccccactgactgcctgagacaaaggagcagctcacatttctgctccagcaagattcaccacctgtctgaagacactctgttactcccccatcagcctcaactcgctgaaaatcacagtcaacagaagctcaatcgtccaagcctccctcctgtcaattcagcttctagaagcccaaactgcctccactgacacctgggctatctgtgtcccacagggtcacataaagacctgtgaccaggtgttttctctgctgggagggcacacctgcagcatcacacagatgggaaacatcaacacggcccagaaagcactttggtgaggggaccagacaggatcatagaggatataaagacaagtgcacaggtgaacagaaggccaggcagtggtcttcctggagaaaactcccaggctggagggcacagaagtccccccagaagcactggggagcccacagtacaacttcctccctgcccatcatcaccaaccatgggccagacctgagtagcatggagcactgaagccctttgtcccaaatgccagctatctccctaggagtgcttctctgaaacatggctgtgcattctggggagaaactgaggcaggcctgcggaagggtgctcacaagtctgcctaggtctagctgcaggtcaggccttcactggcagaagcctcatcctgggagcaaggggtctggcgatcttagaggatgggggaacagctgccccaaaaggcagagggaagaagggctcatcgttgctcaagacagagtcactgtgacttaaagaaagatggggaaaaataagtgtcccactgggccctgaaaataaggctttaatcactatttaaaggttctttagacactaagaactcatatggcaaatccacctgaagaccctcatttctaactatttgggggtaaaaagccatcctccctaagacactgatcacaggaaatgagggacataaacctaatctgtgtagagataaagggacaggcagctcagtgacggggtggccatgggcctcgccgcagaagttgctcagagtgaaggtggctgaggccctccagcacacaggatgccgggtggcccagggcactagggggctgtagctggagaggggctggacttgcacacagaagagcccaggcctgcctgcagcatccacacctccagctgtaccagccccaccacaccggccacctgttgctcaggcaggcagggcaggcagggacagtgaggaacatgtgtcccaagcacacctgcgtgtcccaaggtcccatcgctgcccacaccagccgcttccagaatactgcgaagttaggcccagaacaatatgcactctggcacctgggaaatggcattgccctctggacctggggggtgcatggggctctttggggcaagggatgcgtgcacctgataaagctgctgctgtacccagatgtttcctaagcagaggacacaggagaagatgcacgtcccaggtgctgataagggagcgtcactacaaaggtgtcatggttctgtccatgctgctgctggcagtactataaaacccagacatagggcagcccctgtggcgcagcagtttagtgccgcctgcagcctggagtgtgatcctggagacccgggatcgagtcccacatcgggctccctgcatggagcctgcttctccctctggctgtgtctctgcctctctctctctctctctctatgaatgaatgaatgaatgaatgaatgaatgaatgaatgaataaataaataaataaatctatctttaaaaataaaaataaaaataaaaataaaaataaaaataaaaataaaaataaaaataaaataaaacccagacatgcaaggtgccctgcctcggcgaactaggccccagaggagtggggaatcccatggggcacccaaagcctaaggctggctgaagcagtgactcgtccccatcaaggcttctgctctgagccactcactaacatcctacagcagagcacacacaccctgggatccctttaaatctgcccattcacatggctccagctgggcggttcaaactcccggtcctgcatgactgcctgggtgacacgactgccccgggggcagggagccttccattggagaggacgtccccgcagcatcgacacgcagaaggtcacaaccatgccaagaacctgactcaggatattccgggaaaggccatggtgacatatttccctcgacccagctatttgtcaaatctactgaagcaaacataccaacattctgacaccaccagaacactgaagagcaaaatatgggtcagggaaattctgagtttccaaaaaccaagaccgagtgccctctagtggctctgtgcactgcttcctggagggtgaggagtggacctctgctccctcattaattctagagaggacataaagatcattcatgacagagagccgaagtccctcacttaacatgacagcctcatgtcatcaagatttccaacagaaccgactaagttggtaaatatcaaaggagaaaaagtttccatcaccaattaaacacgaatgaacctgggatgaaggcaatcacatcaatgtaacacaaatgcagcaataaaatccaaattaaataaagcaaatgcaatcaggatgggtcggtgcagacctgctggccccaaatggcagtggagaatggaggacgtcggtactagatcctgatccctccagcaacctgtcaatgggtggaatggcaagtctctttgcaaagaactcaagctctgcacaggggagacacactctccccccaaccatccagggagacacctgtcccgctggccaatggatggatctggagcccaaagtgagctgtctttgtggggaggggtccccttccccctgaacctccacaacatgccctggagcctaagaatgtctggcctcccatgggcgctgctctgtgcccagctgcccacgtggagctggaggtggccagcttccaggggacaggccagctctccctggtctgcatcctgatcatacagcacaggaggggcatgaagtgttggtatctgcccgtttgagcatctcaagatgcaggagcagctgccaagcctgggtgccaccacacacagaagcagcagagtgccagggatgaccttggacttgggagtcccgaggtcaggcactcaatccacggctgacacatctgctagctccatgccctgactccacactctctcccgagagcagcagcaatggcctccagttggagaagcaccctcccagctcttaaccacacgatgaatggtgggggacacagcagcgtgaaggggctccatctcaagacgttcatggaacacctgaacaggacttctctctgctgggcttcttaagccccagggtgacctcagagctcctggggtcgctatggggagaaacccttcctggaaggaagctaacaatgggagagctggatggtaaggggaccgggtgtggaaggcagacagagagacagaatccgagtgccaggacacagccagcctcacatcagctctgcaaacatgtgcccagtgctttcctggcccaggaccccggtgggcaatagaaagaggagtacacacagcccccattcgtgagatgctcctaggacagtgctcagggttggacaagtgcccacaccaaagatctgcacctcttcaccatcagggcacaaattagggcacttgccactagatggagccacccggctcactctcagccaggagctgggaggggtcctgtgtgtcacttccatgccacagcaactgactgtggggcaggtacctccgacatgtccctttcctcgtggtgctatggccagcagaactcagactacgccggccatctgtgaccacgaaagacagagttgttaccaacctgcaggggatgtggagcaagaacaggaaataaaagccttttgttttaaagcggtgagatctggggattatgtgtcaccatggcaaactggactgattcattctgagggcaacagaggaaggaggagacacacaacagtatggaataaacaacaggtgtcccaagatcagagagttgtggaagagcacactatgcctgggagatgcctgcgccgggctccaaacatgagcagtttactctgggtatttcaatgcaagactattttttctaaacttctaaattcaaggtcaaaggaaaatagaaattgaaaccaataatttagaaaataagaacactgtagatcaatatgtcagaaatagagtcaaagaagtacttggaaagaaaccacaggacctgaaatgttttattaaacagacaaatggagggacagtgactccatggttgagctgcctttggctcaggttgggatcccagggtcctgggatcgagtcccgcattgagctctccacagggagtctgcttctccctctgcctatgtctctgtctttctctctctgtgtgtgtcatgaataaataaataaaatttttttaaaaaggcaaatgaaaataagctaagcattcaacttctttaaaactttaaaaaaaatgaagggattaataaaggcaaaagcatataaaattaaattataattaattacattaaaattaaagacacaattagaaacttcaaaacagcacaggaattaatgaaggtatgcaagagccgactccctacagaagataaaaggtaaacacctctgatgaatctgcccaagagaaggagacactcaccaggagaccaggaatggcaaaggaatccacacagagatagaaattaggatatttacattacatacaagtttatcccaggatccattcatctatagactatatatatccccaaagttaacctgagctgagatagaaaacctaaatagagccataaccataataaaaacttgaaggtaggggatccctgggtggctcagctatttggcacctgccttcagcccagggtgtgatcctggagccccgggatcaagtcccacgtcgggctccctacatggagcctgcttctccctctgcctgtgtctctgcttctctctctctctctctctctctctctctctcgatctctcatgaataaataaaaaaccttaaaaaaaagaaactcgaaggtggtgaaaacgagctaaggaaaccccactgagctgggagaaggagtggagggtcagcaggtgccatcccccaccgggtcccatctgggtaccagcagggacccagaaccaatgagaagccgccacatggagttctcttttcctcacgaactcctgttcaaaaccatcccttccagcctcctccttccctctatcccctctattagtggacccttgctctctggtctctcaactggcctattggttcaccatggtctgcacatctccttttttttttttaatttttatttatttatgatagtcacagagagagagagagagagagagaggcagagacataggcagagggagaagcaggctccatgcaccgggagcctgacgtgggatttgatcctgggtctccaggatcacgccctgggccaaaggcaggcgctaaaccactgcgccacccagggatccctccacatctccttttgtaattcctctgccgttctggaataaaccaagttgctactcaataacttcgttgttttcaaggttggcatgatcagaaaatccttcccaaaggcactgagttcctgatggttttatagacaagttttaccaaaccttcaagaaacaagtcttgccttttgttgaaattattttaaagcccagaaaaagagagaaagcatcctaactcactgtgatgattaattttttttaagattttatttatttattcatgagaaacacagggagagaggcagagacacaggcagagggagaagcaggctccatgcaggcagcccaacgtgggactcgatcccaggtctccaggatcaggccctgggctgaaggcggcactaaaccactgatgattaattttatgtgtcaggtagactagatgaatcacaggggcccagacaggtggccaaccatcattctgagcgtgtctgtgaagatgattccagatgaaattcctatttgaatactccccctgagtgacgcgctctgccccccccaaccctgcccccgtgtgggtgggcatcatctcatgtgttgagggcttgaggaacaccagaggggaggaaggaagaactgggcgttcctgcctgtcaatgggggatgcacatctccttccctccatgctcctgctccgcaggccttcagacccagactggaacctgcatggtggctttcccccactctcaggcctctggatttggactaattccccagcagctttcctgggcctcttctcagcctccactgatggtgtgagccaatttcttataataaatctcttcctacatggatggcccactggttctgtttctctagagagccctagctacgcactcattttgcaaaacgagcactccaaataccaaagcctgataaagacatgacaaaacagaaagctatagatgagcgatctgcaggtgatggcccactcatcaaattccacccacaacctgtgtttgtgagtaagcttttattgtaacacagccaagctcattcatatggtttccactacacagcactggagatgaggagctacaacagggcatccggcccacaaagcctaaaatatttactatctggccttaaagtcagctgacctctgccacagatgcagctcaattatggagatgagacagaaatcctgaacacaggttaatcaaatcaagacagacatgattataaagcttcctaaaagtcattccctctgtttcttttcttaaacagactttatttttagggcagttttattatattcatagcaatattgaacagaaagtacagagaattcccatgtactccctgccccagatatgcatggcttccccagtatctacatcccgcaccagggtgctacactattctttttgctacctaaaatatttggtagttttttattgtctctttttaaaatcaatcttttaggtgcctgggtggctcagtcagttaagcatctatctgccttccactcgggtgatggtcccagggtcctgggatcaaatcccacatcaggctccctgctcaggggagagtgtgcttctccctctgctcgctgctccccatgctggtgggtatacatgctctctctctctctctctctaataaataaaatcaaaattaactaataaattaaattaaagcataagctctgtgagaacaggagcctaaaaagtacctggcacatcacagagtcccagtaaggatttgcagaatggatgagggagggaggagagaaagagagccactaagcaaaccctatcttagtaagtgttgccttaatttctggctaatgtaaaatgatagaaacaaacaaaaataatggtagaaacagagttaacactgaatgtgcggggcccaatgaggacccacttccctggatggtgtcatgggcactggtatgtgtccactgaagactctggggtcaaactctccatggagatgtgctgcatttgcacctgccctggggaccatcttgtttcactgggccaacggctaaaaggggaatgagcaggtggcactgcccagcgatggtgccagccagagatgacagcggcagccgtgaagcgtgcccagtgccaggccatcctgagcgcatccacagatgtttgcccactcaatcctccctacaacctctggtggaccatgtctgtgtttctttccagttgtttccttcttactgatctgcaggagctcttcctataatattgcttctttctgatattctaattccttcaattaacttctcatggttgtaggcactgtaactgtctttcaatttaaggtttgtctatcgacttgtttgtggagttttctctcttacaaaagttttatattttaatataatcctatttcttagtcttctttgtttatgcattgtctagcttgtttaagaaagctttcctaggtggctcagtcagctgagtgtctgacttttggtttcaactcaggtcatgatctcggggttgagatccagtcccaagcagggctcagtgctcagtggggagtctgcttgtctctctccctctgccctccctggatcatgcaaatgtgctctctcactctctctctctaaaataaataaataaaatatttaaaaagaagaaagaaagaaagaaagaaagaaagaaagaaagaaagaaagaaagaaagaaagaaagaaaggaaagaaagaaaaagaaaagaaagctttcctaaccccaaagtcacaaaaatatcctctaattttttttcgaaggcttttcaagttttattctttcctattcagttctttgttgcatcccattttacagctgaaaaaattgaggaagagagacagtcagcaaggggtccggggtcatccaattagcacacatgcaccagtcaatgacacatgcaactgttctcaaagttactgaaagccgccacgttgtacacttccttccagaacttgtcctcactcctcacatggtcttgaagattcacccctgaggacatgtaaagaccttcttggttcattctgactgctatacggcatctggtcatacaaacataccagctcgtttatccacccccagggggaggagtgagcaggccaccatgtacccaggaaccagctgctctcaacaacaggtgtctagaaacagcacttctgagtagcagggtatgaactaaatattatgaaattgctcttcaaagcagctgtccatatagcgttcctgcttgcgtggtaagacccttcttccccatgagcatcctcatgaaccttgtatatgaggcagctacatttctccagttgtgaagtatgagatgattcctcattttattgtgcatttctctgatcacgactgatactgagccctgtctcatgcttacgggcccctctggcttcttccatagaaatcccctattcatgtgttttgcccatttttctttcctgttaccgttatttcttattgatttggaacagctatttctgtaacctttcttctttctaatcttctaattctagagttttctaaaatttctaaagttttatttcataatccacctacaatttatttttgcacaagatatgaaattaggatctaattttatatgttccttatggataagcattcctgcactgttaagtgactggtccatccctattcctggtggtaagcagaacgatgtttcctaccccagaaatacccacatcctaacccccagagcctggcaatgtgatgttgcactgccggtaatcatggctgctgatcggagagaccatcctagatgtttgggtgggcccaatgctatcacagcagtcatagcggtcctggtaagtgccagggagccaggaggatcagagtcagagggagatttggaaactgcactgctgacttcgaagagaagagaatgagccaaaggctactagaggctggaaaggcaaggaggtggagcctgccctggagtctccggaaggaaccagccccaccaaaacctctaccttagcctgtgagaccccttttggacttctgacctccagaactgtgggacagcaagctcatgctattttaagccattttacatttatggtaatttgttacaacaataagagtaaagtaatacacttatctataatgtgtccagtcatcacactctgtcttcttatctgtccatttctgccccgatgccacactgttctagttactataactataaatctgccaattaggaaaactgaagcctcttttctacttcaaaattgccttggctattcttgaccatgtaattttttaattaaaaaaaaaaaagaatctatttcaaagtaaaataaatgcagtgcacgagggactacactgcagcatagagcatgagcccaactcagaagtcatgaaaacacacaacatagctggtgaatcacagtagcacagagcagccaggaaaactttcaaaaccactagaccatcaggcctgggtggaggatctcagcagcccaccgacatcaggcaaagacatctgtctcccaccaacgtagagtaagcaaatagggaccagactgaccgccccatgaaacaaatttttttaaagggacaaaatatatgaaacaactggcctcaagacattagaattcaggcaacaaaggacagtgatcctcagggacaggaaacaaaagaagtgggccctgaaaccgccccaactctcagcagctaatgagtctgtgggccacagtacagggagacggggcaccaggggcccccagtggagaaaacagagctgaatgtccaagaagaccgaggaagctcccatttgcagggccaagtgccagacaagacaaggtgcacagaaagagagctctggtcatttacagaggctcccctctagaattcagtaggtgactggtcaggaaagtacccaatgctggggccagagccattggaacagtccccagtgcttacatggggcagaaatagacttactgcacagacaggccaaactggcaaacctcataattcaggagatgtcaggcagagcacacaaaggggtcttgtcgtggggaataattagccctggactaaaagctggtctggtcccacctaacaacccttaagggcaagagcccaaaggatcaaactgtttccaaagctcaagagtattcatcaaatacaaacctatttagcactctcaatcaaagaaaattcacaatatctgcaatccaataaaaaatactgatggcagataaacaggaaaatacaacccaaattaggataaaatgactcaatcaaaactgagccagaattggggcacctggttggctcagttaagtgtctgactcttgattttggttcaggtcatgatctcagggtcctgggattgagttccaagtcgggctccctgctcagcggagggtctgcttctctctctctccctctgcccaccgcctacttatgggtgcactgcacactcctattctctccctctcaaataaataaaatctttttaaaataacttaaaaataaaataaaaataaaaataaattaacataattctgggaagcagagcaagcagcagactgggtttaaagcccagatctgctcacaacagtcaaggcaattgggaaaatgagacccaagcctgcagaccgcagagccacagcgggtacagccggtgttcgccccaagcagccaagtgataaggcaattccatcattgtttagaattaatttgattagaaggtcttcaaacaaaccagctgcttgcttccagcaccacagcggcaccataaactaacccttgaaaactcctttccaggaaaagtcagtatatcttccccataaaagctacctgtacaggcatgtcccccaaggacatgatctattctggcatttccacagacacaggtgaactacacatactacatttcactagtgtcctttttgagtagcttaatccaaaatatttaatcataacatttacctatttttgtaatctggaatactatctttcatatttacctttctcagagcttactatttgttgtatttttatttttatttttttaaagattttatttatttatttatttatttcagaaagagagagagcaaaagcagggtgaggggcagggggagaagcagacgccccactgagcagaaagcccaatgaggggctcaatcccaggacctgggattatgctctgactcaaaggcagatgcttaaccgactgagctgcctaggtaccccttgttttattttttaaatgtaactaagaatatatgtgaaggaagaagagacgctagatttacaggagagaatgagtgggaaataaattgtaccctttgtttagtatttttaagcgatatgttctatgccttcaatgcattcgactttattaagtaaaactggaaacactctaaacaaaccctgagagaaaatccatgtcttccgcccaccactgcagggactccatcagatatcctggcaacagcctcggggctgtggtgctggacgttttcagggctacactggtaagagtggttattagcggacacactggtggtaaacagcaatcccaagagcaatgtgacaagcacaatattcaaaaattgaaagatcaaaacaactaaaaccatcaaatgcactgcctttttgagaagaaggaaaatactcaatgatgtggtttgtcccaaagctacggattgtttggcacctgaatatgttccgatacctcagaaaaccttggaagatcatgaagacagtggccagcatgtccaggtccaggccaaagaactaaggggactgcttccaccaacagtaaattaaattcaacaataccaccttccattaactagattctcaactcagaaaaaatcagtcccaaatgagtcccagacccactagtgcctccgctccagcagccaggacctcagtaagtcctccctggggacagagaagcacccatggggaagatggataggattaagctccccactgtgggcttatacattagttcagtccctacctttctaactttctaagagactaacttcctatatctagaacattcttccagctggagcagtctctagaagcctcaggtgcctgaggctcagtgccctgccaagtgaacttcaccaacacccctctgacatcagccctgcagggacctgacccatgggcttctcagtcagatgctggcacaggggtccttaggggtccttatggtccttaggggtggtgaggcagaagccaagaggccctgaccagcaggtgctaggccagtcagacacaaggctgacagcaacgttcgctgctccgccacctgcacacatgcagacacatcactgcccccaggaagatgttgctctgagggtagccctcactctccccaaagtcgccagctgagatcagacaccccattcttggaatgcttcaagggagtaaaaagcaccttttctaaacaacaaatgtacagcacgttaacaacaacacccagagatggcagcccatggctctcggtcaaactcttactctcaacgctgcgtctataggactcccatgccaagtgcccattaggtgtcactggcagagccttccacaagcctctagtcagggggctgcaagacacaggtgacaacacacttggccaccctctgtcactgtccaattctacatccccgtattaagcaaaagctcaaccaactgactccaattaaagcaatctgtagcaattatctgataattgttattgtactggactaagtatttttccttaagaacagatggaagtcatgctatgtgctcaaaaagtcagttctctgcaaacacttacttgcaggagatgtgggtggcttcctggagggctgccgctccgcctcagggctgtggaccagccccgtgggtgtgtccaggtgtccctcagggctcagcgagaagtggaactggacagtccctgactccacttgctgcacctacaatcaaaacaagaacaatggcagctgcctcctttcatcggtgcccttctattacaatgcaaccagccccaaagtctacataaaaccagggattcctgaagccccgtgttctcagatcataatcaagtcactttctgagcagaaacaatcagccgtggcattgaaatcccagaagaccacattagcacagaatggctcttactcaaggagaaattacatttccccagttttgcacgtttttaaacaaacctatgcaaaactgaccttttaaatgttggtatgtagttctgttcaacacatgtactcactaccggagttggggtgcagcgtggctccatcacccaaacaatccctcgtgttgccccagtctgactcccccaaccccctgtttctgagctgctcctgtcatggcagcctcggctctccagaatgccacagacacggaacccaaggatatataattaagcctagcttcttacactccacataatgctgatgagactcatcccagtaagtagtacgtatcaatagttggttccttttcctgctgagcagtattctatggtaccatgtgcatggtttattcatccatttaagcacatttaggctcctttcagtttgggtggttacaaacaccggggtacatgtgtccatgtgaacctaagtcttcatatctctaggaacaggattgctgggtcacgtggtacgtgtatatttaatcctataggacaccatggtttacttctagagtactctgctcccaatttatatgccaatcccgcagcaacagcaccttacagtggttgtcgcagctctactccaagtgtcacactgtggggaaggattcctccaacatcactgctatcgtcacagttctttggactcttctaattcctgggactttctgtataaattttaggatcagtttctctacatgtagaccaaaaaaaaaaaaaatcctactggaattttgaataaaattacattatctgtacatgaattaggggagaatttctatcttcaacacactgaatattcccatgcataaagatgctgtgtctctccatttacttagagtcctctgtgatctcacccaagagtaaccacacgagaactgtgacatgcaccaaagggaatatgctacagttcacaacagacagatcctgtacacactgtgttagatttatcttactgtgggaaggccttgataagtaatactgttttttttttattttgttcatcaactgccctttcttaatatacagaaaagattgatttttctatcttgatctcctattgtgtcttgcaacctcaataaagtcactaattcattctagcaggtttttgtagatcccttgggattttccacacaatcatgctgcctgcaagtaagtacagttctgtttctctctttccaatctatgtgtcttttattttgtcttcttgccttaacgccctggctaggactttgagtatgatgctgaacactgagtgcacatccctcacttattcctgatcttggggcgggggggcacccaaactcttaccactccataggctgctcactgatgggtttgtgtggatgccccccaagcaggaagagggactgctcttctactgttagtatgctcacagtttttatcataagtcagtgttaaataaatcttatcaaatgcctttcatgtgtcatacagttcatctcccttaggctattactatggtgaattatatttattgatttcttgcatttctgggataaacccccacttggtcatggcctagtcttccttccacattgttgggttctatttgctagcattttgttggttgttttgcaactgtgtccatgaaggacacatgtccatggtcttcttatgctgtctctggttttattatcagggcagcacaagcctcattacatgaattaggagatgttcccatccttctatttcctgggggagaccgtattaaactactcttctgtctcttccttaagtcattctgtagaattcacagtgaataggcctggatgttctcaacagtgttttagtcatgaattcagtttctataatggtcataggactactcaggttgtctacatcatcctgagtgagttttgatcatttctgatttaagataaactgatccatttcatcgaaattgccaagtttacaggcacagaattgctcacagtgttcctttgctgtctgtttgatgcctgtggggccagtagtgctggtccttcttccattcctaacatcagcagtttgagtcctcctctcctgttgtcgttcttgctagacatttatcaattttattgatctttccaaagcctcagtttttggtttcattgatttcctctgttttcgttctctatttgttctgttctgtttcttctgctggatttagggttattttgttcttcttttttttaattttatttatttattcatgagagacacaaagagagagaggcagagacacaggcagggggagaagcgggctccatgcgggcagcccgatgtgggacttgatcccaggactccaggatcatgccctgagctgaaggcagacccttaactgctgagccacccaggcgtccctgtttctctttctcaagctcctaaaagtagaagataagattactgaatgaagaccattcttctgttccaacacaagcccttaatgctatcaatttccctggaagcacatgtcaagtgcatcctccaccttttgatgtattttcatttttgttcagtccaaaacatcctctatttcctcctgagtcttcctctttgatctatgggttatttagaaatgtattatttacttttcaaatgcctatactttttaatcttatctttgctaccgacttctggttaaattctgttatgaccgaaaaatgtaccctatataattccaattctttagaaatcatgaagatttgttttatggcccaggatgtggtctggctgagtgttccctgtgccctagaaaagactatgtgttctgctgctgttgggtgcagagttctgtagacgtcatgttgatccagacggttcatgatgtcctttagctgtttcatgtcctcagtgattttctctatgttccctggtttacttagagcgatgttaaagtctccaactataactatgagtttgtctatttgtcctttaacttctagtaattagtttttttgctttatgtattttgaagctctgcctatactggatgcacacacctttaggactgctatgtcttcttggttaattgacttttttatcatatgtcatatccttcttaattcctgaaaatttcctttgctccaaagtccactctgtctggtgttgatacagccactcctgttttcttttggtttttgttttcgtttttttttaaagatttatttattcatgagagacacacacacagagagagaggcagagacacaggcagagggagaagcagactccatgcaggaagcccgatgtgggactcaatcccggggctccaggatcacaccctgggtcaaaggcagcgctaaaccgctgagccacccgggctgccttcctgttttcttttgattggtgtttacattgaatgttttggtgtctgttgagtgtcttttctctgaaagttgagattttcctgcctctttgcatctcaattatgtttagattatatcacagacattttgaatactatgttaggagactgtgagtctcatttaaattctgtggagaatggtgatatttttgtttcgtcacgaatcagctcaatgggttcaggccacagtttccaaccagccttctatgcattgtcagcccctgtgccatgcttttcagatccctcctgcatgtgatccacccagggaaactctggaacggggctggaggcctagccacagtcagttctcagggtctttggaactgtagttcagggtcagactcaggcagcctgagggttaacccagcatttcttaatgttatgggattgctttccaagcctctccctctccatagcctccccaaacctgtctgggactttacaatcctccagccagaccccactgcgctgcacacttctgggaacaaacagtccctgaagccaagacccctcagggggaggacagagtagtttcttaaaagagctcaggagtgtatcaagcaagttctcatttatcattattggaagtcaatatataatgtctaaagttgataaacctaagaaatagcagaagcataatatttggaaacaaagtaaatatcataagtcagagcaaagagttgaaagtggctgcctctggagatagaaattttttttaaaagacaattatttgctcaaaggaaaaatacaggagttaagaaagaaaacataatcataatgtactacttagcttcacagtaacatcaaaataataacagaaacccccagatttacgtagccaaaaattatgggatgtttctaatatttatacttttctctgattagtatctgccaattccccttaactgtaagttcacaggatcggggatctttttccaagggtataagtgctcagtaaaagttcactgaatgggtaagtaagacacagaatgacctggcataggggcagagacaaggtgcacctcactcaggggcatctctgcccctaagcctccacactacctgcccagacatggcactgctgtagaagacacaatgggaagaagcagaggaggaagcagaattaagtttatggccagttttagttcattattttaattagttatgtattttagaatagtgatcctcgactagaggcaattttgcccttgagagacatttggcaacgtctgcagacaactcagtctatcatagttgggggcacgctactggcatccagcagacagaggccagggctggtaacgaacatcctccaatgcacaggacaacccccacagcaaagaattatctggcccaaaatgtcagcagtgctgagggtgagataacccaattagaagtgatattccttgtgaataatggcagaatttgatatatttgtgtcctcaagatagtcaagccttccatactcatccccaatgcagtttacacactcagggagtgaaggagatgtagtggcatgacaaggctaaagttaaatagaatagatatatatttaaatacatacacatatacatccagagagggaaagacagaggcaatggctccaggatctctcacggcagttttataataagggctcagactagtccatgggggaaggcagcaaaatgtgctcccataatgagatatccttataagaagggaagctgcacaatcagctgttcctgatccaggtccctaaagcatcacccacatcataaataacattgaaaataacatttgtggcgagagtcagcacacggatgatgcatggaataccagggatggtcctttcacctgagaaaaatgagacaactgagctgtagacggctcccagccacactgctgaaacagcagagtgtggtacaggagtgcaactgggagaaccaagcacccttgcgtgacactggcgtcaggatgggccagccagagcagtgctttcctgcgcggggtacctgcagctccctggcaggtgacccctcactcacacttcaactggccctgccaccgtttaccggtcttctccaccggggttttggagatagcttcattccaaacatggattttaaaagctcctgctgtggcccgccctccatgcaaagctggaaggagccaagggccagtgggcaagagtaacaagtcacagagatgcaccgggctcaacaccaactgccaggcactgaccacaaccctggtaccaccaaagctcattctccatagcaaaaggttccaagagtcctttttgaaaagacacatatagcacagaagcagatgctgtttgcacagtgcatacaaataaaagccacaggccccacaggccaccttgttgagacttatgtgtgaatggggatgtcctaagccccgagataaaacctgcctcagtgagttcacagccaactatgactcagcaatcatgcttgtcctttaagggcacgtccatgacatgaaaaaaattctgttcatcacatatccacaaacatatctgtcaacttctggttttgaaaatgtgtgaacttgctggaagctcacaggcaaagattcatacttataaagtatctgaacaacttacctcttcagagctcatgctggcaggggttactttatagaccaaacagtgtgaggggttgggccggatcccaccctgcagaggtaagatcaccttcccagagccggattccagcaaggggttccaaacagcccagcgaactatgtgcatacacgccacactggacaaagaggtgaccgaggctgcctaagaagggaaatgaggcacattggtacatgacttaccaccaaacccaccgtgacttaccaccaaaccaaccctgcaaacgtcactagggagcagtgcagccaagcagggatcacacagaaggaaccagaccaggaggagggcaacatggaagcttggggcagggggcagggaagcagcatccaacctaaccagtaccctttcctatactgatctttcttctggctcaaggaagtagcagcttttcttttaaagaccaacaccatccttctgatccctcctgttcccccgaggctcttgtgcagatctataagcatgctgtatcttgactacatctgcccacatccattaggaaaatactaggaatgacacttggtgttagtgacaggaaactggaaaataatgaagaatgcccatggaagttcaatttggtattatttgcacacattttctccaaccaccttttgctcataatgaatggtacatttcatatacgtgactctccatggatctgggaagttggccaggtccaattttctgcaccgtcccttcttccgccttctctatcactgaccaagtgaaagcaatgaggacactaaggaaaccggtttcagggctcaactcagcagttttaatggcaccaccttcatccattcacccagaaagaattaagaataaagtcacactccaggcctggaacccgtgctcagctgggcagtgagacttgcgattgccgccttgggggaggcaacatcagagaccccagctctgactgacagatgccgcaggttccttgggaggcagcacgtcaggaccccaggaagaggccatggggcacttggcaagagtccaggcaggtgttttccagcttcagagcacagctgtcctgaccagagtcacattgaaactatccagcccaaggaggcagcagccacacctgtcaccctgactcccacctcagtggtgatgtgggaggaggaaaacctgaaggtgtaggcagtacttggagcatgcacacagcattccacctcttaatgccctcttcacaggtggcagacagctgtctgtgcccctctgggatgaggacagcaaaggaactgggtgactccgtgtggtatccaggtggctgagggaccaaagccctctgatgctctaaaccaccagccacatctgtgaacccagacagccctctcaatgggggccacaggctagggaggctgggcagcctacaaggagggggcctcgagctcttagaaaggttggaagcccatacctcatctgatggtaaaggacatcatccccaaggagaagagagctgtatcagaagccacagagtaaggcaagactccagcagagctgctaaggctcccaacagaccccgctcacttgctgggcgtgggtgagctgccgtgccattgtggcacggacttgtggcatgggcaggactgactgagcttgagtccaaggcagactgtggcacacatacacaaaaggactctggccagaacccaatgcagatggttcaatggtcagccaggccacaccccagccgagcatatccatgagaagcatccaacatggaaaaggaagcacattcatctcacccaagtcagactatccagactgttccttagtcaggtagtctcccagcctgctgtatccctagtcacatggggataaatgtccagagtcagtgtgtggtcttgaattaatccctttcctgaagccccattgcaagtggcccaacctcacttcagcagggcctgttctcattctttactggagcccaacttcagatcataggctaggctcccatcacatatgcctgggtcacaggggtaaccaggtccctgccagttcaatgacaaaaaacaccaatcctgaaagtacaaagaaccagcaaagtaagaagcctaagcatatgtctctgccacacttcaaacgtgtcaaatacgtagccaatgcctgccgctcttcatccggttagctgatgacagtgacataaaatggtactgagcaactgtgcaaagttgactataaactcactgctctttaaaaacaaaattactataaatgtattggcaaaactggacactaaaaaaccatgtgattcgaccatttctggactacttttataaacagccatctcctataaaatccagtttattactggatcaaaaataaaatggcacacctgggtggctcagcggttaggcatttgccttccagctcagggcatgatcctggaatcccaggatcaagtcccacatcgggctccctgcatggagcctgcttctctctctgcctttctctctctgtgtctctaataaataaataaaatctttttaaaattttttaaatttaaaaaaaataataagataaagtatgtatccaagcataacacatgctattcgtgcaaatcttctggtttagtctttcaaattaggcgattctgatatgcagttttccaatttcacagtccctctggtttcctagttctcaatcgataatggctctcccctacgcggagtacctgtcagttatacacatacccatttcccctcactttcgtctctaacagctacaacaagaagagatggctagcgagtcactgatcactcctttgcaaagatttctgtgacaattaggacttgttggcaatttatcagcctgtgccctcttctgccaggctggggtgacacctgcaggtgagaacctcctcaatctcacacacctcgaatccccccagctaggtccctggctctgagcaggtcaggtgcatccaagcaggtgggcagaagaaaagcaactatttaactggtcaacaaaacaaggctcttcctgtcagtgaatctagtggttgagataaaatcagttgctttctaacatcttatccgcccaattgggtttggcttcaagaaaagaaatacaaactgaaaagagaaaaaaaatattgacttgttccccatttagacttttatttataacaccacataatatacaagaggccaaaagagtacacctgggttatatataaaaagccaggttcttcaggaacagaagaactgcaccaacagtcaccaggcaaagcccaaacagacaagggaccctgacctctgggaatgctgtaaaccaggttccctagggtgggtgccatggtggcttcaagaaggcaagtcctcagcctaatccctggaccctttgtgacctgatttggacaaagggtccacgtaaacgtaattaagtattatgagattaggagacctagattatccaggtaggcctcaaatccaacgacaagtgtccttagaaagcaggatagagatttgagacagagggaaggccctgtgaatgagaaggcagaagctgaagcaacatggccatgaacagagggaggacagcaggcgccagaacctgttagaggcaggagggaccctcactggagcctctggagggaaccccaccctgtaggcccactgctttcagcccccatttgcagctgattgtcaatctgtggcctccagagctgagagaataaatgtggattgctttaagacacatccagttcatatcaatgaacagcagacactcatgcaccacgacgaggcaccgaaaccacagtggcatgcgggatctccccagtaagtgaccctgggcggcaggatggcaccaacgcgctgagcataaaggtgaaagtggacacaatgaacccctgcaggaacaggatgggctctcctaggacacctcaaatcgagacataacagacatcaaggcaggcagatctgcctacaggaaaaatggtgctgaggtggccaagacaccctgggccaaggcccatgggtgaaaaccctgagttccagtgggacaccacttgcggtggggtggggggtgcctctgggaggtgacacaaatccgatgaaagtgttggggggtgtttttagtgtccttgcaagaaggggaggaggggagagagacctcccgccttccacacacacacaccaacacactcctacacacccctacacacacacaccagggcagcagctgcaaggtggggccccactgggaacccatctgcggctccagcacctggagggataggagtctgacgttcacatggcatttccttcggctgccacagcagataaagacaaggccaaacacaggggtgtgggtcaaaggcagaccgcagagcaggaagaggtgtcacacactgtcccgagggagaacaggtaccctcactcacaaaaatcaccacagaaaaacgggttaaagaagtgaccagacagacaacccacaacccgactgcacattccacttcacgaactgtccctcaccatgaggccatttctcccctccaagtagcagaacccaaagtatgagaggccactcctgcaaaggatcatgctcttgcccctggacccagcatctccttggggcgcacgcgcacagccctcaatccagcacacccttggggcacacacccctcgatcccgcatgccctcaggcgcatgctcacacccctcaatctagcatgccctcaaggtgcatgcctacaccccttgatccagctcaccctcggggcacatgctcacacccctcaatcccgcacaccctcagggtacaggctcacactcctcgatgtggcatgccctccgcgtgtgtgcctacacccctcgatccaggacctcctcagggcgcacagccatacccctggacccagcacccctcagagtgcatgctcacacctctcaatccagcacacccttggggtgcatgctcacaccccttgatcccacatgccctcaaggcacatgctcacacccctcaatctggcaccccctcagggcacatgctcacaccccctcaatctggcacctcctgggggcgcaccctggagacaccccagagcacaaggtgagccaacacacagacagttccacccacacctggtccacctataggccctgcaaattgctgagctatccaaccacaggaaatgcatgcacaagctggggggcacaagcagagtacatgccgttggaaaaggaggagacacgctctgtggccagccccgcctgatggccagggcaccccaagagtcacccacgcacatggccacttccctccctcaccccaagaagcagcccaggagggatgcactgggagcaacagcagcggctggctccagaatgaacagtggaagggacacagaggagcaatgcccttctgaaaccagctttgggtagaatttttactgaaaagcctatcagtgtcctacatgttccggaagtgaaatgaaatcactaggatgggagggaaataactctgaactagagcacagataaaaacaggatggtgaaagactcctaactctgggaaacaaactaggggtggtggaaggggaggtgggcgaggggtgggggtgactgggtgacgggcactgaggtgggcacttgacgggctgagcactgggtgttattctatatgttggcaaattgaacaccaataaaaaataaatttataataaaaacaggatggcccactgtgaccgagtgagcagtgaggctacaccattcgggccaggcctaggggctctggacactcttcccctccatctgggtggccagagccctgaacggctgggagggccatctggcagcagcagtgtgggtgaggcaaccctgcgctgaggctgatggtgccctgggcttctccctggggaaggaggctcggagagccttgccagtcggctgggatcggcaagaactgggttttttttttgttttgttttgtttaagaactgggttcttaaggtcaatctgtgtacatggcaagtgcacatgtgtgtgctcacatgtgtgcatatgcgcatatgtgtttctagaggtaagtctcccagctctgtccactgacatcagatctgggaaaccaaaacacaccagcacccagatctggattcaaatacatcccctttaaaaagaaccagggtgggagcagcccaggtgacgcagcggtttggcgctgccttcagcccagggcctgatcctggagaccctggatcaagtcctgcgtcgggctcccggcatggagcctgcttctccctctgcctgtgtctctgcctctctctctctctctgtctcttatgaataaataaaatctttaaaaaaaaaaaaaaaaagaaccagggttcatcaaaaacaaggaaagtcagaaactgtcacagtccagaggtgtgtgagaagacacgatgactacatgatgcatgacatcccaggtggggtcctagaacagaagaggactttaggaaaggaaattcaaataaagcatggacttaggttggtaacaatatatcgatactggttcattagttgtgacaaatgtaccatagtaaggaggctaataagaatggaactggggacaggtatgtgggatctctgtattatccacacagtttttctgtaaatctaaaacttttctgaaataaaaagtttattgagaaaaggcaggagtggggagaccacagctccccacagaaatggctgatgtcgaggctggaatggggaagctgtgagatgagcctggggcaccttgttctaccagaatgtaagaaactgctcggaaaacagaacaccatggaggatgtcacaaggacacagcctgaagggactacggacaactccaggacaatctgagcactaaaataatcaggaacagtagattggtttttttaaaaggtgtccttgagtccatcccaatgtagagctggaaagcctcactcagaggcagcaaggatgccaaccccggcagatgtctgtgtggcctgaagggtctccccacaaatttcttgtaaggacaaaatcagttactgtccagtggggacatcacatgacacccgactggtgatcaaaacaggccaaaaaggtgggagggctgccacgcacatctgatgtgggactctgcgggacacagcccaccccactgcagtgttctagcagagatgcagaggctgaccccattgggaggaagcatcactaaatccaacaggggaacatgtactttaaaacgtttttaccaaaatctataaagaacttattaaattcaacacccaaaaaacaaagaatccagtcaagaaatgggcagaagacaagaacagacatttctccaaagaagacatacaaatggccaaaagacacatgaaatcagggaaatacagatcaaaaccacgagataccacctcacactggtgagaatggctaaaattaacaaatcaggaaacaacagatgttggtaaggacgtggagaaagaggaaccctcttacgctgttggtgggaatccagactggtttagccattctggaaaacagcgtgggggttgctcaaaaagttaaaaacagggttaccctataatccagcaactgtactgccctaggtatttaccctaaggatacaaacattgtgtggcctgaaggggcacctggacccgaatctttatagcagccatgtccataatagccaaccaacagaaagagcccagatgcccatcgacaatgaatggataaagaagatgtggtgtatatatagaatggaatactaatcagccatcaaaaaatgaaatcttaccatttgcaatgacatggatggaaccagagggtattatgttaggtgaaataagtcagtcagagaaagacaattatatgatttcactcatatgtggaatttaggaaacaaaacaggggatcataagggaagggagggaaaaataaaataagatgaaatcagaggggtagacaaaccataaaagactcttaactctaagaaacaaactgaagaacactggagggaaggggggataggcattaagaagggcatgtgatgaaaaaaaaaaaaaaagaagggcacgtgatggaacgagcactgggtattatacatgcaactgatgaatccctgaactctatctgtaacactaaaaaataaattaattaaaaataaaatgtttccaaaaggagggaaaatgtcctataaaaataaaatcagtaaatccaaaaaaaggctgaggagctgctccagatcagaggagaccctgaaggcaggaaccgtaccaccatgagccctgcactggagagaaatgccaccaaggacacaccaggtcgctggcaggtctggattacagtggagattatggtcagactcagtgctgagccaggcaggctgctgtgtgtgcccatggaagggcatctcagctctgcagaaatgcacactgaagcatgtgaagtcaggggctgtgacgtctgtaagttacccccaaagagctcacaaaagataattgtatggatctgcacacacacgtacacatgcacagaggacaggtgggaggaagaaatactggccagaaggtatggataaagcaagtgtggcaggtgcgaacatctgaatctgggttaagaatagaccagtagtcctagtaattttgtaactggtctgtaagtttaaaatcatgtctaaataaaaagtttttaaagaaattatgcaatatctactatgccttttctttttccaagaactatgttaatatgaaaaggcaagtgacagaccatggaaaatattcacaatctatatatctgatgcaagatttatagcaaaaatatataaaggaaattcccatctatccaataataaaaagacacacaatccaaaacatggactggccacttcatgaaggaagaataggaatggccaacaaacatgggaacatatattcttgcctggtcattaaggaaaagcaaattaagaccacattcacacccactagaaagactgaagtcaaaaaatagaaaccaaaagctggtgaggatacggagaaactgtaagcttcaaacagtgctggtgcaagcaccttgggaaaatatttaacatgtatttacctaggacccagaacatggttccatagcactcaataacaagaaatgaaaatgtatgtccacaaagacttgcccaagaatgctgaaagcagctttatttattagctggaaaagcctaccaatggtaaaatgaaaaaacaaatttgcaacccacattatctgataagagattaacatccagggacacctggatggctcagtggttgaacatctgcctttggctcaggtcatgatcctggggtcctgggattgaatcccatatcacacttactgcggagagcctgcttctctgtctgcccatgtctctgtgtctctcatatataaataaaatattaaaaaaaaattaacatccagaacacagagagaacttcctaagcctgcaacaaaacaacaaaccacctgattcaaaaccaggcaaaggactcaaacagacatatctcctatgaagatacaggccaacaagcatgcaaaaagatgctcagcatcattgaccatcagggaaatgcaaatcaaaaccacagggaaaccccacctcacacccattcactgactattatcaaaaaacaaacaaacaaacaatgagtgatggcaagaacgtggagacaccagaaccctggtgtactatcggtaggaacacaaaatggcacggctgtggtggaaaacagtgtgggggttcctcaaaaaaattaaaaatagaatcaccacatgattcaataattcttctggtatataccccaaagaactgaaagtaggatctcgaagggatatgtgtatacacatgttcatgttaacattattcacaatgtttgaaatgtggaagccacccaaatccatcaatcgatgactaaatgagcaaaacgcagcatatccattcctcagtcataagaaggaaggaaattcctaaaatgcaacaacgtggatgggccatgagcacacaattctgagtgaaagaaaccagccacaaaaggacaaatactatatgattccacatacatgaggtccttagagttgtcataaccataaagctgtcaaatatacggtgggagccaggggctgggggtgaggaatgaggagtcagtgcttcatggggccagagttccagattcacatgatgtgaattctggggacagacggtggtgacagctgcgtaacaaatgtaaatgtccttgaagccaccaaattgcacacctaaaaatagttaagatggtaaactttgtgttatgtgtatttttacaagaaaaaagagacatgaaaaaaaaaaacaaaggtaagtcagaatgccactaatctatacactttaaaattattctaggccttgtgggtttcttagtaaaataaactacagttgaaaaagataaaccaaaaaaaaaaacaattttcaaaaagtgagttgagggcagcaccggtggcccagcggtttggcgccgcctgcagcccagggtgtgatcctggagacctgggatcgagtcccacgtcaggctccctgcatggagcctgcttctgtctctgcctctctctctctctctgtctctgtctctgtctctcatgaataaataaataaaatctttaaaaaaaaaagtgagttgatatctttcttaaagctataaggggagcctaagtctgaaagacttccagaaaaccctatagatatctatatttctaagttttgtctttttaaaaacgtttttttaagattttatttatttattcatgagagacacagagagagagaggcagagacatagacagagggacaaacaggcttcaatgcagggagtctgatatgggactcaatcccaggtctccaggatcaggccctgggctgaaggcggtgctaaaccgctgagccacccgggctcctctatttccaagttttacaagtttaactttacatcattttcagaataaatcgagacataactcttatgtgccaaatatgcaacatcaaaatattcagttctgaggattcccaggcacctggcaagcactcagatctttgttatatggacttacattgtccaaagaatgctagaaaatacataaataataaattagttaatcttcaccttctagagatctgggagttattttgctatgtgactccgaaaaaaaggactgttttgaccgctagcagccctgtggggatttgcacttattctgacataaaaatgcagcagaagacaccgggcctggttatttccacagctaccgtcattctgtgtcactctcttctgctccagggaatctgctttatcaactttcacaatcagtcaatgctgatggagcccacacctctgtggcttccagcattttcaggcctcctaatgcaggtgcactgtccccatgtattcaaattcctgggaaggaagctatctggatgggaagaaatactttctctgtctctttacagttcacccagagacaaattacaaatggctttagttgttctaagtaaaagatgcaaacattaccaacagattaccaacaaggagcatgtcttctcaaaatggtctttctagtttgttttccatgactaccaaaatactcaggaccaaaaaagttccctcagactcaggtaagcaggtctaaatgggatacactcaacctggatggtttctcccatttaccactcatcggtgaccaatctcacacagggccccaggacactgaggcttttggctcctagtccaccccgtggacaaggcctggcccattctgtgtctccaagcgttaggcacaaaagaacacaagacataaggaacatggcaaggtcatcctgagaacaggtcatggcacaagaggcatccgtgagggagcatacagctgccccacttaggaagattggttcagaaatcatttttcaaagaaaagtacatttactttttttggctcttctgattctgtacttgagtacttgagaaacatccaggcaggctgactaaatactgggtgacactggacaaatctgtgtcagtattttaggtgtgactatggtactgcagcagcacgcttaagccccattgttagagacacagagggacccgttcacagaggtggcagcgggctgtctgggactgacccagctggactggggaggagtgggggtgggcacctccttctctctatcttcatttgaaattttaagtaacaaaaaggttaaaagaagaaaagaacctatgtgccacaatgttcacagtaatcaaagcagaaaatgactggaatttgcttcaaaataaatcaaaatgggggatccctgggtggcgcagcagtttggcgcctgcctttggcccagggcgtgatcctggagacccgggatcgaatcccatgtcgggctcccggtgcatggagcctgcttctccctctgcctgtgtctctgcctctctctctctgtgtgactatcatgaataaataaataaaatctttaataaaaaaaaaaataaaaataaaaaataaataaataaatcaaaatgggtcgtgggagatggtacagacaatataggcctgcacatgcagttggtaactgctgagccagctggttcattttactacacggtctgccttggtttacctctgaaatgtccatcgtatgaagctctgacacagggaacaaaggggagaaagactggcaggcgggatgcaagcaaagagtgcgctccacggctctcctatctgccaagggatcggggtgtgctggctcagcctgtccccacaccccgggctgcgcccccacaacacacattcctggggaaggatgggggcccagccagcagcacccctgtgccctcccctctaatgtggagacctgtctgtgatgggagtgccctagtcaggccagccccatgcccacagcaggcagcctgggcggatcaccacctcacagccaggaccctccttcccagactgcacccaagaaagtgcacccaatggcagtccagtagaatccagtattctgtgtgaagagaaacctccccaccactaagaatcgatgagctagataagccctgtttcgtaaggcagcaacacacagtaaaaccacctgcaggttaagcactgcagggaagagttaaaaaaataataaagcatcatcaatgaaacgccgggacacctgcaccccgctgtttatagcagcaatgtctacaatagccaaactgtggaaggagccttggtgtccatcaaaatgcatggataaagaagatgtggtttatgtatacgatggaatattactcagccattagaaatgacaaatatccaccatttgcttcaacatggatggaactggagggtattatgctgagtgaaataagtcaatcggagaaggacaaacatatatggtctcattcatttggggaatataaataatagtgaaagggaatagaagggaagggagaagaaatgggtaggaaatatcagaaagggagacagaacatgaagactcctaactctgggaaacgaactaggggtggtggaaggggaggagggcggggggtgggggtgaatgggtgacaggcactgagaggggcacttgacgggatgagcactggatgttattctgtatgttggcaaattgaacaccaataaaaaataaatttattattaaataaataaataataataataataataataaagtgtcatgtgagacacaccagtttctgtatacatactctgtgatcaccagcagaccgtaattcaacattctagagtggatgctaatgacacgtccacagacgcagagagcgccaaacgcacacagcaacttggccggctccctccctgtctccttccaagggcagcacagggggccaccccaggagccccctccctcctggatgagtcccccacccaagcccagagtgccctccccacgcagccaggtgccttcacatacagttgcatttcagtggcaatttctggtaataaataaagcagagctggaaacgacggcttgattaaaacaactgtgaccaaaaaataaataaataaaacaactgtgaccatgtgttcagccataatcagaacacctgcttctagatttgttcagctgatcaggactctgaattatactgacctgaggtgtggacggcagagagcagatgagaaaataaaacagcacctccactcagcacccccttcaccttcctaaggctcccctcaagaccatacttggtcctggtcctccagcagcaaggctgcaatgctggcagaccgactgccatggaaggtgccttgtcatccttttatttctaatttacatgttaaattgatatttacttattcaaacagaaaattgaatcttaagggtagtatgatttgagtcttctaaagactctccatcagtgatacaatgtctaataaataaataaataaaattaaaaaaaaaaaaaaaccaatgtctatatgctatacagggacccatgaataacccggggctgaaggacagcaacccccacgcagtcaaaagcctgcatgtaacttctgacttctccaaaaacttaactactaacaccattcagttgaccagaggccttaccactaacagcaacagtcagtgaacacatatttcgtatgtcgtatgtattatatgctgtgttctcaccataaagtcagccagaggttagaaaatgtcattaagaaaaccatgagaagaaaaatacacttacagtactgtatctaacaaaaaaaaaaaaaaaaaatctgttgtaagtggccccacgcagttcaaacctatgttgctcaagggtcaaccacatttcttctaagtgaatcttacaaagtcttagctatgatacaggcacacacaaagttcactgtaagaaaacatttaccacttagtaatggtaaaaacagtaaagtgcatagtaaagctttactatccttaacatcatacgaagaatacattaaaatagactcatttttcacaattatcaacttgatatttgtgaattaatgctattatgcatgcctattgaactactgattaccatcggccagcagaacattccatcaccagaaagaggggcaatgattgcaacaaagccacaaccaggaagaagcgttttctataatctcattctacacccgttatgtacactgctgataatcacgctgctctgaacagaaacaatgttagtggtgactttgtttctccttggggcacggagcccagggccttctcaccttgctgtccactccagaagggctggtgaacacgtactccagttgaaagactatggcaaacgcagggtgatggaccatctcagggagtcggaggcggcttctcaaaactagagcctggtttccagagctgggtcaagaacacagagcagcagcacatcaggcagtgttcctgccaccctgcaccccccagggtgccaggccctgctgctgtggcaggttcccccagcaatgctgcagagctctgcactgtgtctggcccccaaggtagatcctaaaccagagcctctcatgcttcagtcgacactcattccaaatcgccagagcttacatcaggaggacagaaccacatggccaggtctccgaggaggctcgtcctccagggcacccctcggagaggtctctgatcctgacttgggaatagaggactgaacagaatggcagaacttcctgcctgctgcttctccaccacgtgagagactgatcacagacacagtgggggtataaagtgtgctagggtcccctcagatcctaagcagccacacctggagacacctataatgttagctccagaactgtcacagcacagttcaggatttttccaacctgataacactgaggtctgctcctaacagatataaatcagcaagaggaaacaaaggcaccagggaagaagtccaggtggacaagtgcatacttcacatgcaaaaccagctccatttccctttgatccccagtaatgtgcaccctagtacctcctaattccacgaggctggacatcacgaccctagggagaatacctggtcttcgaagaggagccaactttcctgctgaaactggcagagcgcgtcaaggccacatccatctcgggcaccagcaccaccacgtgtggcctctgcacgaagcccagaccattgtgcacgcccacacacaggcggcgctccaagatctccagcgtgctgccatctagtggactgtccccctagaagagcacatgtcacacacagtaaatccctgtgcagaagggcagaagctcccagtggagaggcaaacaaagagaagaaactgtaactgaagtctcaggcatttccagatcagatggtgctcagtgaaaatgaattcaggcaaataatcctatgagaaccgccagcctcagggcattttctaggtagttctgggtctttacacatagcacggtcttctcagagcaatatcaccctccccctagaagatacctgaatcaccaaagtcttactcaggtttccatggctgagctcaattcttaggatgcagcaaatgagcaaccagggccctagcgccccaaggcagagaagcccactgcccccaaggtgcctctatcctcccccagactcagacaccagaggcttggggtggaaggggcagcaggaaaagggagcacatttatgcagtaagtcataaaccaataaacacagaatctgccccaaagtgatcgctggatattaaagtttttcatcttaacaaaatatgtacaggcactttaacaaatgagtcaggatagtcctccaggctattactgtttattaaattacgtgatcgtggtgaaatgattcctgaattttcaagttttcttcactgcccattaatcaagctgaacagacctaaattcagcctattagtctctgagtaatgaaaacagagcgatggaggaaacagagtagctaccagttggataaggacaaaaatacaaagtagtaaaaatatgtaataaagtgattttggggggatacacaaaattcaacaatgatactcctaaagcatctctacccttatgtgacccactctgagcaataagaacacagcaggaaacacagtaagaaaatgtgctccgtcaatcctcttttctgtgtttctacttcacatgagacatcgtatagaactaccttgtcaagaacgggcagcaagcaaaaaaaagtccatctcatctatgtgtgctgtgcacatgcatctatgcatacatgtgcatgtgtgcatgtatgtgtgtgcttgtgtgcatgtgtgtgcagctaagactttgctgacttcccagactgctaggatgggcacctaccagtggaactgctcgttgtcacgatggagagatagcaaaggcctgtgacaggctgacactgaagcaccttgggcacagcagagaacccaaccacatccctgttttcctggtgcaaatactccagacagacaggcaacagagcaaggaataggtaaggaacccaggtgctcgtgggcagggccaagcacccggagaaaggaagcaggtgacatacttacagagacttggggggtgggcctcatcagcacattccagtggcctgcaggactgtcgagtttgcacagagacctgaagcctaaggggagccatgcaagggtctagacgcagagcatccaggaaaatgagctaccagcccaaaggccacagtggggacacactgtgacctgtgaccaccaagacttcatggcagcacatcagatgggtaggcagacctggaccctgtgaggggagtacctttcacagaaagtgggtcggccgggataaaatgactaagacttctagtcgtctccttggaatgagccaaatatcttgaaaatgtcctttataaatgctgataattttattggtctccctaagctcatttggtgatttaatttacttttacatgaagactgctttaagatacaacaaaactaacattagggtctcacggtaatagcaccatcatcgcaccaaagttaaaacaacgggttagacatcctcttctgccactaaaagaggaagcacgcaagactgtggctggaccgtccagcctggtggtgtcagagaccggcagcgtccccacctctgccgtccccacctctgccctgcccacatctctactgttcagattaagctccaccatgtaggacagtacctcccggaagttatcactgatgaggagttccagaagctcttcctcaaacttctcaagggaaggatatagcgtgaagaataaatcatccaagtaccatgtgacagacttctgaaggcgaggctttcggagggtgtcccctgaaaaaatgagggggttcgtgtagagtatccgtggggacaggaccaccagaagcacctagggcctctcacccacctaggtcaaggccatggtgccaggccctccaggcagagaagaaaccacaggcacagcactcagagccctaaatcagaagccacccccattcacaccaagacgccaagcccagatttcaattacaacccaatttctgcccccaccctcctccaggaacacattttataattcagcaatccaaagaacacagatcactgaatgcagggtaacatctgttcctatcagcttggtttccctttatcgctaacttcctctctagcaagaacacagccagtgatgccgtttcttatttttgtacgtttaccttgaaccatttggaaacatgaagaaatactcatgagcacattaagccaacaagcaaaaacaccgtctgggctggcaccacaagaaaagtccccatctgggtgggggagctgccctggactgtgagctccatgaggacagaaggggcggtagttcctggcacacagctgggtgcccggcacagagcaggtgcatcaaccctgggctgtgaaaggcatggtccagtctgagcagagacctcccctccaactcgggcacgaaggccaagccaaccacctccacaccagtacacaccccgcgctctgccctcccatcccgtcactgccctattgccctgtctcttccccagctttcacccgtaagaactaaggagcaaagccttagattttaagaactatttccaaagttagagaaaggaaacatcagcggaagactcagcgcagagggaactgtcatggtaagagcccttggtcgtcctgctaggaggtcaatcagcacagccagaggcgatcccatcttcgcctctgtgtgcactgagacccttcagaatctcctcagccgctctctgccccactaattccacattttgaacctattcagcaaaattaatcgaatgtgggagcaaagctttatgcgcaagaatacttattaaacattatttataacagaaaaaaataagaaacaaccaaatgctccctaggatggggaatgttaaacaagttaccatgcctccgtataaaacaggaatgaaactcctattgtcaaagatcacttaatgattgaaaaataatcataatattgcaggtaaaaacgatgtgtgtaacactgcgtatgagatcatttaaagccaattttatttgaaaatctacatttacatgcatacatggatacgtatgagaagtaatcacagatgactttatttccttctttatggttttctatcccttcctcatcacaaatatgtattagattctcatatttcacaatttcttatgcttctaagaaacgttgcacgttcctttctgaatttaaaaagaagtgttgaagctgctctgtggaggaatcagaacccacgcaaagtataggtatagttattcaccatggccaagggttaaagacaacccaagtgaccagcaacagatgaaaataaggaaaatgtggtccatccacacacagaatcttattcagcattaaaaaggaaggaaattgacacctggatggacctggaagacatggcgctcggtgaaatgagatagtcaccgaaagggcaaatcctagacaatctactcacaagaagccctaaggtggtaaaatcagagtcagaagatggaagggcgggtgccaggggctggggaggggctgggcactagtgtctcatggggctgagctgcagtttttgtgagatgagacattctggaggcaaacggtagtggtggccgcagactgcatggtttacatggttcaagtggcaagtgtgacactacatctatgttaccacaatttttctttaaagagcaaaacatagagtccaaactttcatgaaatggccaacaagtgtctgaggcatgtcctgaattgagatcctccaccagagaaagagaagggacgcctgggtggcccaggggttgagcatctgccttcggctcagggcgtgatcctggagtcccaggatcgagtcccacatcgggctccctgcatgcagcctgcttctcccactgcctatgtctctgcctctctgtgtgtgtgtgtctgtcttgaacaaataaataaaatcttaaaaaaaaaaaaaaggagaaagaggaccgacaaggaaagagaggaaggagctaaagcagcacacttccaccaccagtgccaagggcagcaagccaggtcctccatcatgggcctccctagcaacaggtcacagggcacacagcagcatgggggtgctcatggaggattttggggatgacgagacagggcagctgtggccctctgggcacagagaaggagcgagggaccatcatcaaaccagggcccatcagcaggaagaggccaggcccgagccaacaccactgcctgctgggggtgctgagtacatcctacccgcaggtagaaggcgtgtgtcctgaaacaagggaagacaacgggtggtcaggcaccctggggccagggcctaggggccaaccccagtttctaaacctgtgtcctcagctgtggaagccaggtgacagccccccactcaggacagtgctgtggggacagagctaataccattcacagccatgaacacagggtcatagctcccgaggaaaggcacacacatgtacacacatagctcatcagactaatcttaaagcctcggcatggctcatccctgtactggctcatccctgtacgttctagtctctactttacaagagaaacagggctcagacattctaagtgacttgcctgaggataccacacccacagaatgtgagtgagggagacccaaatgttgaagctgctaatacacatccccaaatgaccaggaaagtactgtgagaacttcagggagtgaatagattcacagatacagaacccaagagaaacaaagacgggctgctctgggagcaggggactcaggtgggaccccaggggcgccagccaatgccagcccttccagagatggggcagcagcagagacagacttgccggtatgctggggccactgcctgtccagcagggaagccacccgcccctcgccaatctccactactgtgctgttgcctgcccctctagacaacagtacagtaggatgagcaaatctgggcatccagaaggtggcggagtccagggcttctggaagaggctcagtcctggcaggaagcaggctctgcccagctccccattgacaggtggcccccaggcccacagccattagcaaaaaggagccttgactctagtctcacagagatgtgaggaagggagccccacacacagaagtcagacagggagaggaaagatggttcctccaccaggagatcagactaaggagccacatatgcacagggcagaaccttccagatcctgaggggcccaggaggcagggcagggccaggttgcatctggccggctttctccaggccatgtcctgtgaaaatcacaggcactggagaatggctctcactcagaggacagagcaggcctcccaggatgctccactgcagaggagcggccagggtgaggacagtatcccatgaataaagaacaaaggtcctggggccggtgcttcctaagttctcagtgctgagctccatcggcttacttctttgctcctcacatgcatctccaggggcaggtcccaagaaagtccccattttatgcaggaaagggcagcagcagaggctgggagaggttaggagcctggaggcccactccgtactcagaacctcttccctgctccctcctgcatccccctcacaacagcttcaccatctgaagtgtcaccctgaaatgtcacctcattcactctgaagggtcaccccgttcactctaccacccaacctcagcaaccggggttggggaactggggtgaggcgggggacggggactcatggcccgcacactctgtgactggggctatttttcatatggttgttgccaaggctctgggcaggaatgcaatccccaaaaacagcaatgttccttcaggaatccagactccctgccctcagggatccccacacatgcactgccgagaaaccagggattacaggcgcagacaggcaagaactccctgtagtgacatccttaagagaagcgttcagaaatgccctccaggtgcccaacaagctctccacgaggtcctaacagggtcccacagcctccccaggacacagctttcctggaagggcagtggattccccagaatgcaccccaccaaacagtgttgcaagaggaacaacagaatgagaccagttagagggtgacccaggcaggagcggaaagagtctgagccatccacattgctaaaacacctgttgcctttgatggctgtggcaagacgtgatgttgcccttgcttccatctgtccaagacaggagcactaagagccccatgctgtgccctcaccctggctcagtgtctgtgcacacggatccagccaatgagccttccgcggatgaatcatggacaaacccaagcatcggtgaacaagggagaggataaaaggatacaccgaagtcggagcactgctttctccagggaggttccccggggagcaagcctggaaggaggactttcagcaaagatgacctctgtcgtattaaaactggagctcacacagggcaggagtcagctctcctcttgtcaccccaccaccacccctgcctacccccacctacccacaacccacagtaacgtggcacaatccagaaggtgaatcccagcaaggaccgagcggcccagagaagcctaggagaaggcccacagccactgagatacttggatggagggtgggtagggtgggggaagcagcatttttaaggagcacctcaggagattcctgagcagggacttgggaggaccatgtgggataccaccagagtgggccacctcgtggcagactggcgggcatgaggttctgacaccccacttctcagctctaccgagctctggtgtaacagatgaggaggcagacctggaaaggagagcccacggcaaaggccgctttgtctgcttcagacgcctgcgtggtgtcagccagctcaggggagtgtgaggccctctgcttcctccagccctacagcactgtgagaagtgggcccaggagccctgcactccctgagcacccagtcaggaagtctcgctcaggactccaggcagtcctggcattcacctagccttcagggccactgttagaaaacaggcttccaacttatatttacaacgaagcctccagtgcactcagctactttatgcaaagctcgcacccactccagggcaggatgagcaatctgagccttcccccaaacgacagacatctttctggtcctgatctcctgagaccatccccccaatgacagaggcagcatgcagatccgcaaccaaggagcctccgtgcggagactcactctgagccaggccacgggagctctccttgaaagaagcagctttcaggagccacggcatgtgtgcaacgaccctggtgctgacacagctgtccaggtctgtcaaaccaaagcaagtgaaacggtcagaaccaggccgaggagaccacgagagggagggtgcgtggatttccaagatgcattggcaggacctgctaatctggccagatgctgagggcctccctctgagagccacccgaggggaaaggcctgcctcccttctggagagtgacaaagagccaccagcacaacacagctaagaccttcatttccagccattcccaggctgaaaatcactcttccaaaatcagccttcacagaactgagtgagtcaaagtggaagacaggaggaaggttggagactgtgtcaacatcggtatcctggtcgtgactttgtgctacaatttgggggaaaccaggcaaaggacacatggagtctctctgcgttattaaaaccacatgtgaatctagactgacctcaatataaatttcagttaacaactcaatcttcctgtattgtgtgatattttacaaatgaacaaatatccacatccccatatggcaccaaaataaaatcagaccctctccaaattgcacgtgaattccaccactgaagctggtagcagtcactgccttggccctctcagggctataaaatattattcagcaaagaaacacttttccaagagtgagtcacaaaagcccagaagaccctgggcaggatttataacacagacaagaacggcgtcagaaagcagggacagtgcctgcatggctgagagcacagatcagcctggcgtcctgcacatccagggaaggtggagcaaaaccagttccttttctcagatggtcaccggaggacttggtgagaagctgtgttccggtcctttctccccaagaagagggagtatgctcctgcccacccgggcactttccggctccacttctagggtccctggtcattagcttttatgcaaaatattagggacacctaggtggctcagcagtttggcacctgccttcagcccagggcatgatccaggagacccgggatcaagtctcacgtcaggctccctgcatggagcctgcttctctctctctgcctctctctctgcctgtgtctctgcctctctctgtatctctcatgaataaataaaatcttttataaataaataaataaataaataaataaataaataaataaataaataaataaaaattaacaacagattaaaacaacagcaatttcccagacgctgcctgcacccagtaagtgtcccagtgaccaactggccgctctacaggagggggctcagatggcttctgaagacatgacccttcagaatgaggagctggagtatgccccactctgcagctggcccctctgatgccccaggctgacacaagacactgagaataaactcggggccaccagagctgcccattgtcaccacaggaatgacataggaccaggctccagatgagcttcggcaggtccaggttttatctgcagctctactgacacccactggcaagtcatagagtgagggacacacctgagcccccaccaacactctgctcacccagaagcccaggtcagaaggactcatgtaagctgagttttaggcaggtgtgcacctaggttcttgacatcaggacatagcacagtggcacaatgggccctctctgatcgagagggccgaggcctcagagtcacacgcggattgtcatgttttcaatctcgaaggcagtgaccacgcaacattctatctccctgcttgaccccgtcttctctttcgagaggacataagcaaattctggagggtgaaaaggcttcctatatggggctggtggcagggggcagggggttgggggggctgggggagggtggttgcttggtaagccatggcaaatccagaggtgggaccctcctcagacttgctcctgttcatagctatcatttctggtgtgtcatcatctcttcttagcaacactggaaaaatagtgaccttgtaatgggctgaccaccatgggcccatcccagaaatttaccaactcagataacaatgtctgggccccagtattgactagaacactgagccatgtgcagggacgttgttactgaaaacattcccatactggagagctgagctacttcttttttggggggtcttgagtattcctattctaagtcaaaactcggcccttccagctcacgctcatgcccaatcctgctctccccagaacagaacagggagccagcccaccccacacaagaggttccagcagcaccaggtggggaaggatgggtgtggcaccttctcctggatacctcagacagccagacggagagcctccagaagtgcctggaaggagcatgaagttcagatcgaggcccgtcagcagaacccaggagcagagcatatctctgcagagacctcctggctaatccaccactggagaacctagttctaaacacaccaagacagagcccggaggctttcacgtggagttcggttttcaaaaatactcaagccatggctcacccgcattcacagcagcattatgcacgacagtcaaaaagcatcctgtccattgaaaggtaaacagagaaacaaaatgtggtccatccctgcaatggaatattattcagccttacaaaggaaggaaattccgacacatgacccaacaaggatggaccttgaggacacgatgccaagtggaataacccagccacagaagggcaaatatggactatttccctcagaggaggagcctggaggagtcaaattcacagaggcaggaagcagggcagtggggtggccagggctggggaggggacacggggagtcattattgagggattgacagagtttcagtcaggggacatgagaaagttctggagtggatggtggtggtggtgggtgtacaacaagatgaacatccttaatgcccctgaactgcatacttacaaacggttagaacagtagatttatgttatgtctattttgccacaatttaaagagaacgataatcaagcctctgatatggcagcccagcaagggtgggaactaacaacagtggttttcccgttttcccgaccttggtcatgaaaatgcgaacgatggatcttatgacagcatttcccaagtaaggacacagaagcaacttgagggatgagagacaccgctctgaagggacctgtgccctgccctcctccctcccctccctcccttcccctctcctccctcctctcctcctcccttcctcctctcctctctctcctcccatttgctaagggtacctagccacacccagcccacctcatagcacagagcctgatccctgcaaggtctgaccaccaaactaatacagactcaccaaatagagggagacagagcaacccttacccttttctccatgagctggaacgaggccagggatgtgctgcagaccagacaccagaaggttctggggaagaagatggaacacgggctccaggagcgggtgtggcttcagagtgtattgcaggctgcagctctcaatgagggtcatgtgtttattttctgtaggagaaaacacctatatgagagttggcactaaaggccctttcagtcaaaccagccaataaactgagcatctaagaaacaccacaaacaggaaacatatttttccatggagaggggggaatcgctctgttagttcagcctaaaaagcaaactaatccttactaaatccttagtaactctctactaaaactgcaataacagagatacttgcttttaatccacatagtgaaatctctcctgcttgcctctccgcccaaggtgtgaagtccaggcattgcctggtcctcagctcagcgggggactgcaagggaccctttcccctcagcacctgcaccctggagtcccacagagcatcgggactaggccctgggagaggcaggagaccaagacagagccccgcctcgaggacatgtgctcatctgggcaaccgacacacaaacacagatacccacagcacaaaggacaataggatcggaccctgagaaaggtatacataacgcacagcgagctaggggaagaaggagagcaccctgagaaagcagcagctgagaaaggcccagacacagtgctcagggagctggagggaacaaaaccaacagaacagaccctggggtgcaagaggcagagtccagaatcacagggcccagaagctgagagggaccaggacatgccctctactcatccccgccaaaaccttgcttggccttgatttcttccactgacaggagcctcaccacctcctgaggcaagctacatgatttcataagaaaagagctaaagtctgaagcttaattttattgataaagattggcttttaggttatttctcaatatgaccaactgcattcattaggaaaagcaattaatgaaaactatatttttattacctaatgactacacacatagggtctgctaactttaaatttttaagtgccctgagaggtaccctctcgtgtggccggctggagagacactggttgggcctccatggaaggcaaataggagaaatgttacaaactagcccccagaccgagctctgcagcttccaagagctcgacccacagttattgggcacatatacaaaagagaggtgtccacaccagcaacgtacccagccccagggccctggatctgcccggtatccactggcagggccactcagtcaacacagaacacacactcaacaactcaacaacataccatgaggtcatagggagaacgaggggctctctcaacaccaagaccaaacaaaccttctctaaaatgagaaaaatgagatggacatgcggctctgctcctgtcctggtacaaagaccccccagggtccaggagactcagaggggctgcctggggtatggaggaaggagagcagaacactgcaggggcaggggagctcactgaaccttgttacacttttttatgctatttttttaagattttatttatttgctcatgggagacacagagagaggcagagacataggcagaagaagaagcaggctccctgcagggagtctgatgtgggacttgatcccaggactccagggatcacaacctgagccaaaggcagacgctcaaccgctgagccacccaggcatccttttatgttattctgaatcatgtgaatatattgatcattcaggcaagaaatgtgtacatttctggaaatgcccacgagaaggtgagctaccagccacatagctcctcaggcactgccacagccacacaatcttccaatgctgcccgggacaaggtcctgatggtgtgtgtccttccaaaggtcctgaccctggggcactgcactaactcagtcaatggagcacgtgactcttgatctcagggttgtgagttcaagccccacattgggcatagagcctacttaacataaaattaaaaattaaatgaaaaaaaaaagccctgattctaaggatgctgtaggacacaaatactggcaaagtaacgatcagactcaattgagaaataatgtaaaagccaatctttatcaataacattaagcttcaaactaattgctcttctattgtgacatttccgtcaaattcagcaggcagtggaagggatggggcttgctcaaaattcccatcacagaatccaggcaacaactcaaagacacagaaagtcaccctccccaacagatctacctccaaccactggaccccctccacatcctggccctgggccaggtcaggtctccaccaggggcctccaagatggcttccacccggtgccccctccccatcgttcaagtggccaggagccagagtaggtcacatgcccttcaggtcacagaggcatggcaggaagccccctgctctgcctaagacaccccccacccccacccctgcagctgctgggcctcatggaagatggcaggcatccctgcaggacgttcctgtgcatttaggctttgccgtgctggacataaccaatgggcctcacagtgaaaggtttctggtactagcatggccatggccagtgatagacaacaactacttgtagtggttgtcaggtggacaggccaccaccccagaggccaggaggcccacatccctcccaccaggaggttctggggctgcctctcccagggccgtctgcacaaagcaacaccttcccctcaggctagtgcttacagttccttcagttacgtcaaccatccaagctcatgacactttttgtcctaatcccttaaataaatcagctaagctgataatggtttacacagagaaagagaacaagatggagtcaaagcagataacaccaggaaagtcatagggaggatgagaggacttcacagcaacacctcaggccaggccaggccttgtctcttacgttcgaaagggtcctggagcagtgggtgcaggagggctcggggagtgccgtgatacagcctcaacctgcaaagcaagaccacagagcgcatgtcaaccctccgcatcctgcatttagccaagaaacatcagtgatgcttctcactccacacctactagagctgtgtcaacaccatacaatcacatcctctgcaggagagacaggtgagcaaagcagcagccccccatgcccccgggccacatgcttccaacacacagacagtactcctcccgacacacgcaagacccaggcacctagaaaaagtacaccagggaaatttctaatatctgaaatgtcccaagaaagacagcatttgaggaatcttcatggttgtgtatctagagcactgttctcaaccagggatgataccatctccaagaggatatctggtaatgtccagagatatttttgattgtcatggctaggggtgggtgcagcggggagagggctttggcatctggtaggtaaggcctgggatgctctcagacaccccacagggcacgaggacggcccccacagcaaagacttgtcctggcccccatgtcagcatgccaaggctgaggaaccctgtccaaagtgacagtgtaaacttcagcactggctgacatggggacacactgtcttctccgtcctgctatatagccagctcctaaacagagcccggcacaatgtgtctgctgaacagatgcccactgcttactgggtgcctgttggtgaagaacatattatttagcacccacacaaccttatgacatgtgtagcgttctctctcatgccccgatgagtgaaatcagtcaagagtttgggtcactggcctaaaaccctacaggaataagtgagagctcacttcatcctacatcagttcctacatcagcctgaagaacacttccttggctcactgagaaatggcaaacagttaccacacagcagaaaagctcccagacaacacacggttaggaagaaaccacaacacaagctgacaattgcaaagttttctgttacagccaatgagggagcatgaaatctccacaaaatgatgctggaaaactccaaggatgaagaagacgaggcaggtaccgcttgtcctgggaggcagaggtaggagattccagtttgttgccaaagatccgaagaattccaaagccacatgacagagcctggagagagccatcctgcttccggccttcggtgaccacctcaaccacagccaccacgttagggtggttcaaggacgtgtgaaagtacaagggctgcaaaacagaaaccacaacttcggtgacagactgaccatccaccagctgcagagagcatacagaaaaggtacaggcgccctcgctctgccctcctatgaatgtgtgtgcccgtgtggaacaccgcgtccctcccagggcaccgcaggagagaacaggcatggtaaacttgctccctcggagtcaagaaacattccagagcacccaacacccaagagagcagactatttgcttcgctcctggagcttctgacccacaggaaatgacagatggcaggccctccacacaccacctgtcgcacacaccaccccattgcccaacaaggacccagtcactgcaaacacacacacctgctgagctacttcacagagttctcaccacgtaaaacttcaaaacaactcgccaatattggatctaacccgttttgtgggtaaccattatgaagttccacctaaactagttttgtttaaactaagcaggatccctagattttaggagatgctttgtaattatacccagttcagagcccgactaacatctttttatgacaaaaagcgcctaacatcattttcttacaagatttttttctttttgaatctaagccaagtagatagtattcaagtctctcttaaaaggttttccacctgtaacgttccctcggatgcaaatggacccccagctggggcggtggaaggggtcagatccacctgcgtccagcctccctgcgatgtgcactcctgctcaggagagacagtgcctctgctcagcggtcgcttccttcaacttcatcatgcccggtctggctgcttttatttaaaactaataaacgtcgtaggcacagaagatggggacagaggcgctgctggggtttgcagaacaaatctagatggaggcactgagctgtggctctggacacggaaggtccccagaatcgcatctgcactgggggagcatgggtgtggcacccgcccccaacacaaagaccacccccctccaagcaggtagcggtaaggccgggctgctgcgatgtgatttaaacgtggactaatcccccattaacgagtcattaccgtggtgtcaccaggaaaacgtttactgtaatcacagactgtcagcccaggataacagagagacacattaaaacatcagccatgagatataaatcgttattctattatcttttattgcaaaatgatgaaataatgaccacttacagctgacaggtggttggaccaaaagagaattatgtttctgtttctaagactcaagaactgttttggagagccccactgcagagttgggtttgtcactgtctcacctcttatatttatacctgtgcacatccgtgttaggaacactacccacttttcctttggcagtgaaacaaataccttaaaccgtgatgcaaagccacagtcatctcctacccagaggtcaccttgaaattgctctgcctggaggccctgggaccagctgcttccacacgtcccaggttcaaatttatggctgctccctctcctttccgtttaccccaaaaacaactgctctgagaccaaaacaacaggagcagagccctgctgttgtccatctgcaaagaccccatcttcaagtgggagggtcagtggtggaagtgccacttcctgaacacgacccgaggtgtgaggggtgttgccctggcactactggccatgaagggcacatggtctcttccaaatataatttatggaccctttggtgtgggaacagcatcgtgctaaagaatttctaaggagcctgaaaacacgatcccaccctcaagttcagagttcaaatgtaaaggctagattaggcagatagaaatggcaaattctgctcaggaccgaccagaatcacgtggaagtaaaagcctcccacgggtgagagcacagggcagctccacagcttgttaggaaaacccaacaggccctatagtcacctatgctaagccccagtcagcaaaccaagggacacagacccaactgcagttcccactctcccgggcttggagctctgagtgggtggatctggaattccctggtcagcaccagtgaggtgatctcctggtagaccctctgcacccccgcacacacaggaaggtaccttgcctgaaaaagtttctctttgctagaaacttcttccttccccattgtatctataaaagcctttcactttgtacagcttcctatagatcctttctggaggctaaatgggatgctgcccaaattatgagtcgtgcataaagccaattagatcttcaaatttactcagttgatttttggtgttgtgggttcatttagattttatttatttgagataagatcgagggagcaggaggaggggtgggggtgtggagggaaaaggaaaagcaggctccccgctgggcagggagcccaactcagggctgacatggagcttgatcccatgacccttaaatcacaacctgatccgaaagcaaatgcttaacccactgacccatgcagatgcccctgatttttggtttttgtctttttttttaagattttatttattcatgagagacacagagagagggagagacagagacacaggcagagggagaagcaggctctatgcaaggagcctgacgtgggacttgatcctgggactccaggatcacaccctgaaccaaaggcaggcactaaaccacttagccacccagggatctccatgatttttggtttttaacaagctttaatcccctggtcaccttgaggggaccgtccccaggagggtaaagttgggccagtccagctcacctggtacctaggcatattcccaccttacagcccaggaacaagctacactacaaatcctgccagcattcagctgagcaaaaactgcaaataagcccttcagtctaaggacatttaattaacaacatgcagcaagctttgagaggaaaaccctatgcatcaactgaaattgcatgattaaaagtttgagtcaagcccatcaaattacctgcaagagaaggcctctccagacagaagaggggaccctcgttcaccccagtcacatcccactcatctacatctgacaggtttcctccctgacaggataaacagattaataaatcatggaaatgctcctgtcttaatgccttgatcttcaaaaacatggacaccattttgcctcggggccaggtccccatttctcacccacatgacagaactgaaattgggcctctgatgatgtgcctctgtggagagactgcagccaatgtgtccccaacggcattgccactcgacctgcccccttcagtccccgagcagaaagctgatggcacggccagggaagagcagagcagagattctaacagtgccttccaaacaatttttttaaataaacctttaatagaatatcttcaggaaggcagaaagataacacttcctgcagctcctacctgctagctcagcctcccacctgagggcttgggtggttcctgtacacttcccacctcaagactgaatgctccaggcctggggaactctgctacatgaaactctgctgagagatgcaaagtctctagttccagagtcagaagcttcagtccactgttacccacaggaaacaacacgatagatccagccccactaggagactcgtccagccccccatgtcagcgtgccaaggctaaggagcctgtccagagtgacagtgtaaacttcagccctggctgacatggggacacactgtcttctccaacctgctacagccagctcctgaacagagcctggcatgatgcatctgctgaacagatgcccactgcttactgggtgtctgttggtgaagaacatattatttagcacccacgtaaaccttatgacatgcgtagtgttctctctcatgcccagatgagtgaaatcagagtcaagagtttgggtcactggcctaaaaccccacaggaataagtgagagctcacttccatcctacatcaacccctatatccaaggccagagccaatcccactccaacctaaaggacacttccttggcttactcagaagagtaagcaaacgcaaataacccacagtatatattattatgaatagacaacacaagctgacaaatttaaagctttctatcatcccccatggtagttaaatctccgtacgactaaggcccagtgctagaaacatccaggaaagtgatcattcctccatatctggctcggcgtcctgggggtactttccgtagagctggagaggaaggggccacggtgaaggacactgccctagggctcctgccatggtcacatatgcttgggagctgcatagaggacacatgagaggctggctattgagggagaaccagcacgtgttcaagaagtaagggattaatcaagtcacttggaagagcttccaggactttccttgggattacatggatattccttaacagcatccacttttaaagagggaattacatggttctcattactctaacaaataagaacatttgagaattgtttctcatggtgtcacttaagggaagcagaagccacccaattagctgtaccttgagctcaaacacctgggttacctactcctctaacaacacccggggccaaggcaagctctgggtaggtgcaagcctggtctgtgtggcggtggtcagtggagacccctaggactcctgcccagtcactcccataacataagaggaggaagaaaaatgagactgtcacaggaagcccgtgtgcggttagttaaggggaaaatttaaaggagaattatgtccatctcagaaactgcacggtactttggcttcccagattattttttaaaagatactttctgagaggctgaacaaattattaaccatctcagcagaagtgaaaggcaattcctctgcacgagaatggtctggaaggacggcccagaactgcgggctgggagctgagaggctccagcaggcagcacctaggaggccgagcctgagaggggaggaattcatcattcacggaaacatcagctcaagttcaaggcaaggtctttctgcaggacagtgtttgccaagtaataaaatcaggcccacatatgacacctaacgactcagtaaaaaccctgagcacaggagctgggttcattaatctacatttgactggacagccatggtgattccaaagatgcagcctgcccacctccagtcagatctccccacgccactgctggccagtaagaccagaaaacacaaccaataaaatcagagagagaccttcaccaaatttcagaatccccctgaaaaggggcacctggtattcacttccacgggtatgcgggcagagcgcacagaccctcctgtgcatcagtgccccgtgtgcgtggaagtacggatgtgcacataagcttgcgtgcatgcgccacacaggggttttgctttccttaactcaactcaagaagaacctatttccctgtgtgctgagcaccaaggaaaggagagtagtgctgggctcggtgaggacgcagctgaagggcgcggccgaccggagctcattcctgggccgccactggcctctgctgctcgccttgggcatagcagctggacggcagggctccctgagcaggtccgggctctggcctcacctcccagccgaagacactagggccagcccccggccctaatgcacactccacttcagcaactccaagactgaagggtgcagaattttccagaaacattaaaccctgaagtcctcactaacccagaatcccctatcaccctgcccagctcagcagaagacaaaatgtgagggcagccgaggcaaagcagggtggcacttggcaccctggacctgggacttggccgtgagggctcaggactgggagacacctgaagtgttgtggtcccacgagtggcacggcagccaaggtctctgggggtagcaatgctaaccctagacaagtccaggaggagcaggtctgctctgagggctgttttctaccgtgactgcagttctagagggtcaccatagccaagccaccctgggcagaggacatgaccctatgacagggacgtcccaccactcaccatgggccagggaagggtgggctgaggggaggatgtagaagaggaactcattccaaaactacaaccgcaaagcactcagtactgatttcagtgcacgcctccctccctctacccacaagactcttccaaacggaaagcccaggagcagacactggtcaggggaggctgcagagcaggtgcgctgccccaggcactcccagccccctaatgtgctgggttactcttcaaggaaggacatggaacaccagcctctggaggagaggggtgggaatggaatcacataaatgtgctgtgagccaacgctcagcccacagttaactccctggtaggggacagccatgtcaccatggccttcatcattcacttggtaactgtcacaacaccaggcttcccagggccagactgtacccacgacaaagagaaatttgaacagccatgcccttgagggccaccctcaaaggcagcagaggctgagctgaagccaggtgatcagccacaccggggccatgctgctcctggtgaaccctgacctgctggatcagaggcgtgaaggccactcatcacctgtgctgatgaaaccctgtagggagtcaccgacaggcccacaggtgactcacactcggcacggcaggccagaggcctttcagcagcacaacttctgtccccaggacaacattcctgacgtctaaggggtaggagcaccctccactaccctgtgatgggagggctcccatcagaatcaatccaagccccaaccacaatccaaccataatctggactctgggtggcagccaaatgctgagagcagcacatgctcagcttactttgtttcacgtctgggccgggtcagtccccagcagcaaagggctaccttcggctctggttacaggcagtgacacctccaagaatggctatggtcacagccacctccattactcacagcactacctgcccctcgagcccactctgcccatgagaaagatcacatcaacggggcctcagccctttccatacctgtgaccatcacccacatcaatcaagcagaccaactgtgttgggaagtgtgccccagcgctgggctctgcccctggtgtttgcagcctgtgagcgtgcaagccctgctccactcacagtgcctgtgctcacagacatccaaaggactagtccctcttgctgcaggtctggcttcaatgctcgctgccagggaggaccctccttctttgccctcccacgtcaggtctccccgttcacccccagggacagttctggtctccttcagaggctcaccacaatttgcaatgaccctgtcacctgcctttgtgtatcacccctgctatacaccatgagtgccacggccatgccacccaccatcaccactagacgctcagtccaagggctgagtagaggctcgtattgggtgccgccaaacaaagacaaaccatctagctgcagccagacacaaccatgcgggcaagaccatgtcaccaagcaatgccaatgcagccactcaggatggaaacagaaggcatgggttcgagtccaggctctgccacctgctacctgggcaagccaccctccttctaagctgcaagtgctcatctttaaaaacaaagaaaaaagccacaatgaaagaaggcagacaaggagagtgagggaaaacaggagaatcagatgagatcaataatttcctaactttgttccaagaaccccaagggtccttgaaggcgagaagacctccaggagccccctgtcctaccgcttcgaccagagccatgtgctctcatctgttcaggatgtttggcctccacacagattttgtttagaaatgaagtgccccagtgttaaaacaagctttgcaatgacccatctgctgctcagtggcatttctcgggtctgagatgcactggtcgtgtccccaggtcataagccccacacacctgctcttggccacacagctgccctgcctgccaccatggcccacctcatcaaagatgatcctagacggctgcctggaaagctgctcagccggcctcaccgcggtcttccatgtcctcccgaaaaagtgtcggtaggtgacatcaaagagagacacacgcagatggcagcccacctccgaaagcacctccagcactccctaggagacagaagggtacaggtcctgaagggttcttgacatcctgagccatcccatgtgtgctgactgaccagcttggtagaattggatcacttagcatcttttaggtgtcaatatccttggggggaacttttctctcccatggccagcggtcttcaggttatgcatgaaagtgcttggccctcccggctacttagagcactgaagccccgaggcacagacacgggcatatatgcccctcctggggccagctaattcctatacagagtaaatgacttgccctccagaacatctaggatgagcaaaccaaccaatccagagttcacactcccacctggccctccatctggctcttagactcggggagaccactgagattactcacaccagccatcctaagcctggtcatctgctcagcctgccttgcacattcctccccacagaaacaacaatgaaggctttcagccacagtctctccagctgctctgcctcccgatcgaccccatgccaccccgcgtggccctatgtggcctggtacacccccacctcttgggaatggcaataaacggtcttctctaggttgctctcactgggcctgaacaaacaaaatcccacacctgcctgtgtccctacagcagctgtgagaggtagagggagaagccaatgtgccatggaactcagcaggggtgcctcacaaagcacatgggtgtcaaatccccgcactgcatgcttacatagctctcaactattttatccattacacctccctccagctaccacatggaggtgactgggtgacaccagtgatgggccagatgtggggaggtgaggaacaacagctggaaggacggggtggccagctgcctccaggtgcccaggcctcacctgggcctagtgctgcaagtcctacatccctagcgaaggatgatggcatgttttctgccccgggctgctgggaatgaggcagtgttgggtacagaggtgtcagacagctgacaggtctggaagaggcatctgagccccgtttcaatcacagaggtatgggggccccttgggactttgcagacctttctcagacctctctcgacacacgggctgcctctctacccatatgttttctcaggaacctccccagctcactcccatgccttccctgagcactgccatctagacacctgcccatcctctgaccctggtttttttttttttttttttttaagattttatttatttattatttttttttaagattgtatttattttttaaaagattttatttattcatagacacacagaggcagagacacaggcagagggggaagcaggctcccctacaagagcctgatgcgggacttgatcccagaatgacaatctgagtcgaaggcagatcctcaaccactgagccacctggttaaactgtggtttctggggccaatttctttcagggatgtttgctatcatctaaagttgtcaattctcaaagtgactaaactcgtacaaacattgtaggactaaactttttcatgtgtttcctacttggtcttttttttttttttttttttgacttggttttcctttaaatgctatcttttcactgattttgtacgtacagctgacccttgaagaacacaggtttgaattgcacaggtccacttctatgtggtttcattctgttttgatgatgttttgaggatgatggtacagaaccgtagatgtgttttctcttccttacaccttaacaggattttctttcctatagctgactttattataagaacataggatatgatacatacagcataaaaaaatacatattagtcaactgtttacattcccaggaaggattctggaataatagtaggctattatttgttaagttttgggggagtcaaaagttatgtgtggatttttgactgcgttgcggggtcagtgcccctaacctcacattgttcaagagtcaacaataaaacttgtaaaatttagggttcctgtgttaaatcccatctgattgtgaggtactatcctttacgtatatcactagatctgatgtcctcatagtttaagaacttttgtgttcatattcatgagggatgtctgtactttcttgtgatatccttgcccaaactggttatcagttatggcagcctcaaaataacagctgggaagcattctctcctctgttttctgaatgagtttatcaaaaattcaaattaattaatcttcctcttatactttataaaatccaacaatgaagtcatcaaagtctggagtttctttgtaggaagctgtttaattatgaattcatttatttaacaaatataggactttttatattttctatttcttttagtgtcacttttggcaagtcgagcttttcaagaaatgtgtctatctcaactaagttatcaaatttgttggcacacaagttattcatatccctcgactttttaacatctgtaggatatatagcaatgctccttctttccttcctaatattggcaatctgtgtgttttcttgctcttttttcatgatcagtcttgtaggagtttatcaatcttattcatcttttcaaagaaccaacttctggctttgctgattttctctattatttgccctttctttactgatttctgctaaatgagcagaaatctaccattttcttcttctattctctttgggtttaacttgttcttctcatgtgatgcttaaagaggacatagaggtcatcgaatttataactttcttctcctcttacatagtcatataaagatgtaaatttccctacaagtactaaactactcacaccccataaattttggtctgttcttccattactcagttcaaattattttctaatttccctatgaatccttctttgacccaaggattagttgttagtatgctgtttaatttcctcctgtttgaggattttctaggtatcttcttgttcatttctaatttaacatcatacaagtcagtgaacatatatatactctttataaattccaatccttttaaatccattgagacttattttcatggcccagcatatggtctatcttggtgaatgtcccacatgcacttgagaaaaacgcgtattctatttgttggttatagtgttttagaaatgacagtcaggtcagagtggtggatggtgtttcaatcttccaaatccttatgagtgttctggttcatttgttctatcaattattgagagaggagggctaaaagtctccaattgtgatggtgaatatgaatggagctccctttagtcctacacaattttgattcacgtgtttggaagctgtgtcgctgggtgctgcgcacaaaggcgcaatcacagcttgcccaaccattatgccaaactattgccaaactattgtgctttgaaaggccagtataggggcacctgggtggctcaattggttaagtgtctgccatcagcttaggtcatgatctcagtgtcctgggatcaagagccgcattggactccctgctcagtggggactcggcttctccttcttcctctacccctcccccctccactcacacttgctctctctcagataaataaataaataaaatctcttaaaaaaaaaaaaaaaaaagccagtataggaggctggcccttggatggaggctgggcacttagcattcaggagggttcccatcaccatactgacaacggcaccctgtgcctaaactgtatacagtggtttacgctaaacacctcctttcttgctgggagtgtggaatctggaaacatgctaggcagaggatacccgcctgaccagtcctcaaacactgtggccactgagtgtctaacgagcttccttgctgggaacatttcatacgtgttgtcatgactcgttaccgagggaactgaatgtgtcctgtgaggacccttgaagcacatgcctagcccccccacactttcccccatatgcctttcctattgctgatcttactctatatcctttcactgcagtaaaccttttctatgaggctgattatatgctgacacatgtgaagtcttcccaaggaatcactgagtctggggggtggtctcacaggtctcaacagaggtacatgcacatttagaactgtcctatcttcctgactgcatcattatgaaatgccccttcatctctggtagtactcctcaccttgaaactgactgtctgacatcacagcacaaaggcttcttacaaggtctacatggcctgtattttctccatcctttaactttcataggtacagttcatttctttctgcttaaagtgtgtttttttataagcaacctataaatggtcttaacaataatttgaaacacgtaactctgagtcaggaactccatttggaggaagtattccacagacatatatgtacatatgtgtaaatgaagttaatcactgtagctgtgtctgtattgcaaatgactgagaaaaaatctacatgttcatcaataaaggtctggataaatacaggcaggttcatccagagaaatactatgttgtcttaagagagattgttcatgtcattacatgaacaatctataggacatattactaagaatattttttaagcaaaataatacaggtatatttacctgctgtgtttgcttagagtttccagaaagttacacaaaaaagcaggtcacactagctgagtctagggtgggacactgctgccttagggattctgtgtaattatacactctcctcacgtaccctagtgaattctgaacactgataattaccttttcaaaagtaaataaatacaaataaacaaaataaaataggaaaataaataaaatttccttcctaaaattcctaagagagaggacagcactgtccactactctgtgagctcctattaggtggaagtcactggcgctgggtgaccaggcaggagcccagctggccacctggattggggcaccccacgcacccggctgcagtcccctcaggctgtttcccgctctgtgaaatccagtagcgacggaccccacttcagagggctgctgcaaggagcaggccaactggaacaggaaagtgttgggctactagtaaggctggaagagcagcagccaccatggggcaagcacagtggccacacacagcagtcgctggattagagggcttacaagcgaatgctatagggtgtgagtgatatcaaagaagtgagctcttacagatccaggcccccaacaactaaatacatggagaatcacttgtctatctgcagaggagaacatagccttgccaagccaaaccctatgaaagtcacatgttcacactggcaggtctggatccttctcatgcgtcccactgggtgtcccactgggtctcactagaaccatccaattacatgggtgctctgtgggcagcgcagaagtaccagcttctctagcagttttcctgcccctgaaggacacagatgaagccagagccacagagcaagggcctgagtgtgcaggacgctatggcacatccaggcacacagcttcagccagagctctgcccttgtggttctaactgccaggctgcaagcaggctgcttgagtcgtgcatgagcccacctcctgcaaaccacccagctcagaaataatgcaggaaatcatattacctggattttaaaatggcctcactgtgggcagaaagaaaccttcacccagtgtcaaaagtgctgaccgagttgctgaatcacgcatgtgttctctttgctgatgagccactttgcctccaaataatgccaaggcccaaggtaatgggtatggtggtaagactcaaataaataagtgaaaatcaataccctctcagctgagagcaggagcaacatatataggaaaataaatagcaaatttctatacctgcttaattagtggcccgtcaagccacttcaggacacactgaaatgctgttgattcctttaaaagctggcgtgctctctgtgggtggggagggaccagcacgttttggtcaaaaatcctgagccagtcattcattctgccagtcccgagggtcacttcggcctctaggagtacctgcacaatttgtgttcaaataaataaaggagttacgaaggccatagtgcacagagcaattctgaaagtttcctacctccccttgcatcccagaggaaccaccatccccaaaacccacactttgcaattcctcccctgcatccccagtgcagcacactggtatctgtgccaccttcccctggtaaacacacccatcttgctccagatcatctctctacccctgctttcaggagccaactacagcatgaccatgctggcctcagtgactagtgcagaaataggtatatgcagggctgctttgtacagctgcagaggttgagcactacatgacagtgccaggccttggggactggggctatcttggaagggactaggaagcatgacccaatcagagccaaggagatgcaaatctcacagtttctgctggctctgctgagggaagggaatactcttctgcagccctcgaaagagaggtcatcagtatgaagctgtccggaaccaccgtgggagcagcttccttgagacacacatgaaaaaagcaagcagagcaaaagcacaagcaagggtcagtggatctaggtgtcacagttagagccctggcctgaggagaccctaaagcaccccaggactgctcagctacattaatacagtctctcgttgccaaagctggtttggaacagattttgagtgttatgggctttttggtgacctggctcactgaggacatcatccaccttcccaccagatagagatctgtaccttctctagcctcttttcggctgacatacaatcttaagatcttggctttgataggaggctgaattctgcacactaaaatgacaagcaagataaggcagcagatttggtctcctaaaacagaggtactttaaatgggttccccagactctgcggttccccggagttgtcaaaagctcaatgtcaaagctattttcataatattcagatgttatttgcctttttcaccgtgtcgatgtttgggctgatgaagcaaagggaagagtgggcacactggcctcagtgcaccgaggcaaacctctggccgtcgtgcattcttcaccactcacagcaagggcagagtccccagtgaagctgcaaaaactatgaattttattacatcttgatccttgagactggcatttttaatactctgtgatgagaagggaaagacagggtagccctggtggcgcagtggtttagcgccgcctgcagcccagggcgtgatcctggagccccgggatcaagtcccacgtcgggctctctgcatggagcctgcttctccctctgcctgtgtctctgcctctctctctagctgtgtctctatgaataaataaacttataaaaaaaaaaaaaagaaagaaaagaaaaaaagagaagggaaagacacagcaagcattgctgctgcaaaatgaagggtactggtcatctcagggtgaggctgctgtgagcctctgtggcacactcaggacccactgctcctgttgtctggtgatgctatcttttttttttttttttttaagattttatttgagaaagagagagagagagagagagagcatgagcaagggaaggggcagagaaagaagcagacgccccacagagcagggagtccgattcaggactcaatcccaggaccctggaatcatgacctgaaccaaaggcagacacttgactgagccacccaggtgcccctggcaataccatctttactaaaagagtagctgagagacagactgagcttactcaaactcgggcactttgcagtctctcaaacgtgaactaaatgagtctgtcactttcaaggaaaactggtaataagaattgggctttcaggcaaaaatcagaattgggaaaatctgtatcctccactgtgattctgacagcttcctcctccttaaagacgtgcttgacaagatggatggtgactgtgacaaaagtgatcttctgaaactacgattaaatgcacactatttggaagatcagcataccttagaagcagtgttctccaagtgagcagtgcatcatgatataaaaaagcctgcatgagtaaaagatccatccacagtgcaggacagggcagccctggtggtgcagcggtttggcgccgcctgcagcctggggtgtgatcctggagacccggaatcgagtcccacatcgggttccctgagtggagcctgcttctccctctccctgtgtctctgcctccctctgtgtgtctatgaataaataaataaaatctttaaaaaaaaaaaaaaagtgcaggacagaccaaaggactttaaagtaccggagcacgcaaacttcactggcagggtgccaaactccacactgcaacaaacttttaagaaactaccactcgcagtggcgctggcgttgcatttggatgaagaatcaaaggagaatgccacaattacctgagactctgctcccttctccgcatgaggctgtttctttcaaatacttcaaccaaaacaactttgctcaacaggttaaaaacagaagcctaagggcgcctggatggcttagatgagtatcagactcttgatttcagcttaggtcacaatctcagggttgcactcagtggggaatctgcttgagagtatctccctcttcctctccctctgcccctcccattgtgagtgctctcaaataaataaatcttaaaaacaaaaagaaagaagcctatatgagacccagctgtccattaagccagacattacagaaatgtgcaaaaatgcaaaaccaccccttttcctgctaatttttttattctggaaaacagctggttttcataaaaaatacattttatgctaacatgtaacaggtctattgttatctctacatgagcaataacattgctaacatctctcagttttcatttccaatacagttaaagctggtagacataataattaaagtaaacaaaaggtctctggagtcctcggcaattttttaaagggtaaaaggtacgagaccacaaagtgtgagaaaggctattctaacaggacccaaaagatgagggctttgtacagccaggccaccctccacctttgcaggctgtgtggggagcggagaggatcccccagagcacggacatggctggcgtgaaaagctctcccacacccagcatgcccaggcacaaaggcccagcacccacaccccacagcccctgcaggcaggcatgcagtgcagcatccagatctagactgtcaccatgtgagcagtgaggcacacatgcattgcgttttctgttctgagcagttacgtctgctcagtatccccttgttttaaggaatgaccctacaatccagtgtgggaaggctgtccacaaatctaggcagagcactgtagtgatgggctcaagatgaacacgtgacccacactgtgccaatcatcatctcccccaggactccagccgcggttccctctgaagaggctgtctctgctgagagggcaggagctgctgggccagcctgaattccaggggctaccctgccacttcagggagagcctgaggtcaaagggtgtcctgcccacagagcccagacccctagggggagagagcctgatggaaggcagcccccactcccatagcagccgaggtgtagcagccaagaaaagcccatggaggctttccctggtggctaaatggcccccaaagaagtcccggtcctcatccccaggatctgtgaatatgggacttcgcatggcaaaagggactctgggagtgtgattaagtgaagggtcttaaggaggggaatggatgacaggagccacctagatccacctggatgacacaggtgggcctgatgtagtcacaaggatcctcagaagagggaggcaggagggtcacagaacaggtcgagtgaccacgaaacagacagagaagggagggagggaggggtcacaggccaatgaccagctagaaaaggcaaggaaggcttctcccctggagctttcataaggaacacagccctgcccacaccttgatctgagcccagtgatattgaatttggacctctgacctccagacctggaagagggtaactgtgttgtctcaagctgcccagattgtggtatcattacaaaagccacaggcactcacacaggtgtctaccactagtaagcagcagaggcctgatacccagcaggagggccacatgcccaggagagcttcttggaggccctgggggccagtgactgggcccacctggcccctgtgcttcccatggttgcagcactcgccacctgtcctgtgagggtctgcctgctggccaatcacttgcacgaggggatctattttgttgtgtccttggtacctggctcaccatctgaactcaacaaattttcataaaatcaaagtctcaataaaattgactcccaagcattttttaaagagaaatttttgggatccctgggtggcgcagcggtttagcgcctgcctttggcccagggcgcgatcctggagatccgggatcgagtcccacatcgggcacctggtgcatggaggctgcttctccctctgcctatgtctctgcctctctctctctctgtgtgtgtgtgactatcataaataaatttttttaaaaaattttaaaaaattttaaagagaaatttttaaaagaaatgttttctcattgactttcattaaaaaaaaaggggggggggtgttaattcatacaaagtgagtgtcactacggattgaaagtccctgtcaggaagtccccctgcctagggagcaccgtagccaagcatcacctctgaaaggggcctctgtgcaaggcccgcactgtttccccggctccaggactgggtctcttttgcaggcccttccagactttgctcattgggcccagattcctgagccagcacctacctccccaccccctggctaccacacttggccagcagatgctgaaggacgctcctgttctagtccacctagcagaggaagcatgtccacaggactgccccaggctgtacccagtccccataggatgcacgctgggaagataggcctccactctgaatgggacagtcacccaaccaaaacagtgagccaggcagatggaggcgggtagcaacagtctgagtgggcgatagagaagagggcacccgccacataggctcctgggagtgggagacctagtgggcacagagcatttccctcccaacctcccaacccccacctaacactctgtcctctctgtggccactgtgcccccgctcggcccctgctccaattcctcctggacaccatgccccctaggatcctgtcacagaaagtcaccacaattgctcaggctccccatttctgttgtcctgcgagaatgagctcagccaggtcagggctagatcctcagtgaaaatcttttgatgactacgtaaataattaaattatgctgtgaaggagctaagactgctacagagagagagcagaaggcctgtagggctagatgaacagggagaaaacagtgaggccccatgcccaacacaaagggctctccaggggagaaacaacatgagaaaacacaccagctcccacagagagcctgctgcgagctgggtgggcactcacagacccattcatgtgtattagtccaacaaatgtgcagataagtatcacagacgaggacaaggagactaaggctaaacagccaggccaaagccacatggctgctcatgaccaggctcaaagccaggagaggctccggagcctacagcatgacactggctggagcacccaggctaagccccactctcctcagacttcagggaagtctcctgccctccctgaatctgctcccacctacaggatgagaggggactgtgaggtctcccagagggctctgctctgcacacctcttctgttccactgtgtgcaatggtgaacaatgccaaaggccaggtctttaagaatctaaaaatccagtggggaaaagacaagggttcgagtgtgagcagcacacagaagacaagcataattcagtctcctttttgggaggatgcggggcagagagaagaggaaatcccggcactgaagaagcaagctgggataaaatctcaacaacagtggaacaccagagaaagaggggggcaggagcagctgtgcaggcagggggatgtttccttccaagaagtgaacaagatctgggatgcctgagaccgaaaagctttgggaactttactgggagtaagtggcagttatctcaaccccgcctcgaagttttttggtctctcccaccccccccccccccccccctccgagcaagcaatcttctttgcacacctaccaggtgccaggacctggagaaaccatcctcaacatagcagacaagctccctgccctcagggaatggatct

SEQ ID NO: 2 - DNA, 4294 bp, Canis familiaris
cccttgtactttcacacgtccttgaaccaccctaacgtggtggctgtggttgaggtggtcaccgaaggccggaagcaggatggctctctccaggctctgtcatgtggctttggaattcttcggatctttggcaacaaactggaatctcctacctctgcctcccaggacaagcggtacctgcctcgtcttcttcatccttggagttttccagcatcattttgtggagatttcatgctccctcattggctgtaacagaaaactttgcaattgtcagcttgtgttgtggtttcttcctaaccgtgtgttgtctgggagcttttctgctgtgtggtaactgtttgccatttctcagtgagccaaggaagtgttcttcaggctgatgtaggaactgatgtaggatgaagtgagctctcacttattcctgtagggttttaggccagtgacccaaactcttgactgatttcactcatcggggcatgagagagaacgctacacatgtcataaggttgtgtgggtgctaaataatatgttcttcaccaacaggcacccagtaagcagtgggcatctgttcagcagacacattgtgccgggctctgtttaggagctggctatatagcaggacggagaagacagtgtgtccccatgtcagccagtgctgaagtttacactgtcactttggacagggttcctcagccttggcatgctgacatgggggccaggacaagtctttgctgtgggggccgtcctcgtgccctgtggggtgtctgagagcatcccaggccttacctaccagatgccaaagccctctccccgctgcacccacccctagccatgacaatcaaaaatatctctggacattaccagatatcctcttggagatggtatcatccctggttgagaacagtgctctagatacacaaccatgaagattcctcaaatgctgtctttcttgggacatttcagatattagaaatttccctggtgtactttttctaggtgcctgggtcttgcgtgtgtcgggaggagtactgtctgtgtgttggaagcatgtggcccgggggcatggggggctgctgctttgctcacctgtctctcctgcagaggatgtgattgtatggtgttgacacagctctagtaggtgtggagtgagaagcatcactgatgtttcttggctaaatgcaggatgcggagggttgacatgcgctctgtggtcttgctttgcaggttgaggctgtatcacggcactccccgagccctcctgcacccactgctccaggaccctttcgaacgtaagagacaaggcctggcctggcctgaggtgttgctgtgaagtcctctcatcctccctatgactttcctggtgttatctgctttgactccatcttgttctctttctctgtgtaaaccattatcagcttagctgatttatttaagggattaggacaaaaagtgtcatgagcttggatggttgacgtaactgaaggaactgtaagcactagcctgaggggaaggtgttgctttgtgcagacggccctgggagaggcagccccagaacctcctggtgggagggatgtgggcctcctggcctctggggtggtggcctgtccacctgacaaccactacaagtagttgttgtctatcactggccatggccatgctagtaccagaaacctttcactgtgaggcccattggttatgtccagcacggcaaagcctaaatgcacaggaacgtcctgcagggatgcctgccatcttccatgaggcccagcagctgcaggggtgggggtggggggtgtcttaggcagagcagggggcttcctgccatgcctctgtgacctgaagggcatgtgacctactctggctcctggccacttgaacgatggggagggggcaccgggtggaagccatcttggaggcccctggtggagacctgacctggcccagggccaggatgtggagggggtccagtggttggaggtagatctgttggggagggtgactttctgtgtctttgagttgttgcctggattctgtgatgggaattttgagcaagccccatcccttccactgcctgctgaatttgacggaaatgtcacaatagaagagcaattagtttgaagcttaatgttattgataaagattggcttttacattatttctcaattgagtctgatcgttactttgccagtatttgtgtcctacagcatccttagaatcagggcttttttttttcatttaatttttaattttatgttaagtaggctctatgcccaatgtggggcttgaactcacaaccctgagatcaagagtcacgtgctccattgactgagttagtgcagtgccccagggtcaggacctttggaaggacacacaccatcaggaccttgtcccgggcagcattggaagattgtgtggctgtggcagtgcctgaggagctatgtggctggtagctcaccttctcgtgggcatttccagaaatgtacacatttcttgcctgaatgatcaatatattcacatgattcagaataacataaaaggatgcctgggtggctcagcggttgagcgtctgcctttggctcaggttgtgatccctggagtcctgggatcaagtcccacatcagactccctgcagggagcctgcttcttcttctgcctatgtctctgcctctctctgtgtctcccatgagcaaataaataaaatcttaaaaaaatagcataaaaaagtgtaacaaggttcagtgagctcccctgcccctgcagtgttctgctctccttcctccataccccaggcagcccctctgagtctcctggaccctggggggtctttgtaccaggacaggagcagagccgcatgtccatctcatttttctcattttagagaaggtttgtttggtcttggtgttgagagagcccctcgttctccctatgacctcatggtatgttgttgagttgttgagtgtgtgttctgtgttgactgagtggccctgccagtggataccgggcagatccagggccctggggctgggtacgttgctggtgtggacacctctcttttgtatatgtgcccaataactgtgggtcgagctcttggaagctgcagagctcggtctgggggctagtttgtaacatttctcctatttgccttccatggaggcccaaccagtgtctctccagccggccacacgagagggtacctctcagggcacttaaaaatttaaagttagcagaccctatgtgtgtagtcattaggtaataaaaatatagttttcattaattgcttttcctaatgaatgcagttggtcatattgagaaataacctaaaagccaatctttatcaataaaattaagcttcagactttagctcttttcttatgaaatcatgtagcttgcctcaggaggtggtgaggctcctgtcagtggaagaaatcaaggccaagcaaggttttggcggggatgagtagagggcatgtcctggtccctctcagcttctgggccctgtgattctggactctgcctcttgcaccccagggtctgttctgttggttttgttccctccagctccctgagcactgtgtctgggcctttctcagctgctgctttctcagggtgctctccttcttcccctagctcgctgtgcgttatgtatacctttctcagggtccgatcctattgtcctttgtgctgtgggtatctgtgtttgtgtgtcggttgcccagatgagcacatgtcctcgaggcggggctctgtcttggtctcctgcctctcccagggcctagtcccgatgctctgtgggactccagggtgcaggtgctgaggggaaagggtcccttgcagtcccccgctgagctgaggaccaggcaatgcctggacttcacaccttgggcggagaggcaagcaggagagatttcactatgtggattaaaagcaagtatctctgttattgcagttttagtagagagttactaaggatttagtaaggattagtttgctttttaggctgaactaacagagcgattcccccctctccatggaaaaatatgtttcctgtttgtggtgtttcttagatgctcagtttattggctggtttgactgaaagggcctttagtgccaactctcatataggtgttttctcctacagaaaataaacacatgaccctcattgagagctgcagcctgcaatacactctgaagccacacccgctcctggagcccgtgttccatcttcttccccagaaccttctggtgtctggtctgcagcacatccctggcctcgttccagctcatggagaaaagg

SEQ ID NO: 3 - DNA, 115289 bp, Canis familiaris
  misc_feature (10684)..(10929): n is a, c, g, or t
ttactggtacacgaccttgacgcagaaggcttcttcatttttgtcttcgtggtcattgatgtaaatcagaatctcctcctccccggggctctgccgaggtgcgaaccacaggccgatggtgtaggtctctcctcccccaatctggaagcaagcatcagatcctagccctactgcagccctgctctccatgccagcatggtcaacacatgcaaacccaggctgacagagcagggtaaaccgaggctctggcccccactgctgccaggcacctgaggttctgaacacaacctccccgtgttacaggacaccactgctctgcccacgcctcaggggagattcacagctgaagaaacaccttccaatgacagacagactgccatctgctgtactgtcgttaaaatacttgcaacagagaaaaggctaacatgcaggacacttgcgaataaaatcgctgataaactgcagggtaaattggccaccagaagccctgtgacagtgcccagatgtgggggccctagccgcatgggcagaactgctgagagaggggtggccgcccctactcccagtaccaccggtgcagatcagcactgccccctcccggggctctggagacccctgtgcctcggggcccctgggacatgccgaggctgggtgtcccacatgcagtgcgcacaccctgtacgcagcgcttcccgcacctggaaggagtcctctctgaactgcagcaggtcagggtggtcgctgtacaggtgataggtcctctgggaggggtaagggttggtgtaggtgatccgcttgttggcgcctctgcctttccctgcagccagtgtgatctcgaaagcctgtgtgggcaggggtgtcagccttcctcagcctgctctgcacgtacagcccgacctatgtctccatagacatgacaagacacccgccgagggggctcactgtacaggagcaacccccgcccccacccccaggccatagactgcggaggagactcggtcacaggaccaggagacagcgaggtgacacagcacccataccacacacgacccagcaggttcccctccagaccttggagatgagaggcgggcggcaggagaggcacacgagccatgaggccaccagctggtggtagtccacgtcgaccaggttgaggtggatgaaccggctccctgccctgcggggcctcacaccgacatgcaggtcctgcaccccatgtggtggcagcacgaacacacctttggggtcagtctttcaaaacaacagcagagaggcgggtcagtatgcagtccctcatgctgtgcagaggggttccctgaacacctgcagccagtgcacgagaatcccaagggacctcagctattcagaggcagggggcacgtggcctgcaggaagtcagaaggggaccgagtgacaatgccaggtctgaagccagacgcagccacacagtcagggttgcagtgcatctcagtccgcatcaccacccgcatgggcaagtgcccctccatgtggcaacagtccccttagcaccagagctgcctcagcagagggaaatcttaacaggctccagctgtaagcttcagcaaggagtctgcattgccctcattacccagacgcatagttgtggctaaattaattccaaaattctattaatatttaccttccaataattctaaaaattggcctatagggtttatatgtcactcattccatgatggcagatccaataaaaggcagtttgaatgaagactctgccaagagtcttaaaggagctgtgagccgatcctcagccccacactggctgcagccagtctccacaccgtccgtgccccaggtggggtgaggatggggagaggccagagaacacccagcgacctaccttcagctcctgagggtgtgaggtgaaagcctgcactctcctaaccgtccgtgtccctcgaaggacaagggataggcgtgtccgctggcctgtgacacaggagacgtccacacgctgcaaggagtggaggtagacctgccatatttggatgggtgtcgccagccaacgatccctgtgtaggacagaaaccgtgatcaatggtggtgaggacagcagcactgaccaggaagaggccaagaaggtggccaggacaactggggcaggctggagcccagctgccatcatccctgcagccaccttccccactctgaaggcccactgtgactttgcaccacaacaccctgtgtgagtggtaatgtgggcttctaccagcaacaaaacagtcccaaaccaggtggtctgggacaaagcctgtggaaagagtgccgtggaagagtatgtgggttcagaggccacccagacctgagctcacaggccccgtcctagctgcctggtgctggaggccactggcccttccctggaaatggggctcgataaccctgcccctcgggcttctccacacggccagagaccagaaccgagcatggctacacctgccccaatcccagatgtcctattcagaagcccccagcctaccccagttttaaaagatcctcaagggggacacctgggtggctcagcagttgagtgtgtgcctttggctcagggagtgatcccagggccctgggatcaagttccacatcagtctccctgcagggagcctgcttctccctctgcctatgtctctgtccctctgtgtatctctcatgaataaataaataaaatctttaaaaaaaaaaaaaaaagaagatcctcaagggactggaagaggagacttcccagaacaaggtaaaggaacagggttatttcaactgagtgggagaggcaagaaggcgatgtgatcaccatctcaggcacggaggatgaagactgccaccctccctgtcccctgaacggagccaggcactgctctggggcagacgccaccccaagttcctttccttggtgggctctccatgcctctgcaagccgccgggccactctgatccgatggcgtcctttgtgaggcccctgccagccagccgtggagtctcacctcccgtggctgaaggccccacgacaatgtgccccatcaggaactcagcatgagcccaggcggcctctgggatgctgtggctgcctcccagtcccgcccaccctccagacctcccaggtttgggctcacagactcacgcgtaaacggcgacaaagaagtctctgctctctgggcttggaccgctggccaccttcaggaacacgtcccgaggctccctggggccctggcggacatgggcgccctgagaacaccacctgcctcccccccaagcccctggcaggtgaaagcgggcaggaactggctccctccctcccaacaggtgttgcattttcagtattaggcagaattcgtttttttcttcactgcaaaaaagaaaatcattaacttcttgatttcatattgagttcaataattatattctgcaggagattttatccagtttcctaaccagtcataacataattattgcctttattctttacacaatggcaattaataagtggcttaattaccactcactttattcactttgcttcatgcctttttttttttttttcatttgaattcaggtaatccaagaactgatctgccagcagaaaagatgaaaactcatgcataaagctcccacaaggatgtcaaaatagccccttagcaccttagaaagacaccacttaactcaaaagccgccgtgaagaggcctcaggaggccaggggctcagatgaccaggcactgactgggagacgctcggcagaacttaccacattctgggtctcgcagatgacgttggggtcgctacatcggacatagactgggggctcctcaccaggtcttcccacgggagcacctgctgcagggaatatcatagcaaacgtcttctggacaggcccgggccacatgatgttgctggcccatcacaggtaccccacagacaccagaagacagcaatgccacccttcctactgtcccgcccacgtgggcactggcactcccgccctaagccacacccgcaggcacttatccgaagagagtgaccaaaagtaaagagcggggggggctatggtgcagggcgccagacacggggacgctgctgcactctaaagcaccagaccagaagcagacaagtgacgcggcagcagaaggcgcgagcgcactccaggcacagccctcggggccgtgggctttaggaagtgggattagccagccaagggcataccctcgcatctcagaggacgcgaaaaggtcaccatttcaaaccataagggcgggtggcagactttacagatgggtcacaggttgggataagagcatcacaggagaatgtgatgatgccacttgtcaccctgagggaaaaacctttagccatctgtggaacctgcaaaatgccagccgcagggacagatgggacctgcctggaagcgtgtgccagggaggcaggcggatggccttcttcaggaaggtgagctctgggtggtagaagcggaaaacctggtccaccacatggggctgggtctccacggttaggcaaagcacggcgatgggcttaccgccactcccccggaacaagacctgccaggagagcagaggttgccagcatcagggctggcacgtgggctcatgaagagctgcctgcaccaagccagcatccacagggcccagtccccccaccccccgcaccgtgacgtgggaagcacaggtgttgtcagaaatggtgggctgaatctgcagcagcaggtccccacagaggggatggcacccagcacagggactgcatgtggacatgttcccgtgacacacatgcctcaaatgtgtcagcgctcaatttgacagccccaatgtcaaatactccttcagccaccgacccatctcccaacatgcctgccatgtgcatgctgggaagctgcccagagggtctgatgttaacctctccaagtcctcatgggtggtagtggagcacaagctctttaattctgaacctaaacaagaaaggctagagatacaaacttcctttctaaaaaaaaaaaaaaaaagttactgggacacctgagtggctcagtcagttaagcatccaactcttggtttcaactcagggtcatgagatggagccccacggcgggctctgcactcagcaaggagtctgcttgagattctctctgcccctgcagccctcccaagtgcatgcacatgcatgctctctctctcctccgcccctttctctctctctctctctcaaaaataaaaaatccttttaaaagaaaaaagaaagagagaaagggaaagaacgggtatctccctctggccctgagccctccaggccctgcatgaggccaggcatgcagcctgacacccaaggcaaccttcccacagcaacagaacccggtctgcaaaccttccacacgggtaagacgttcttctgctctggtacgttttattgtgaaacctttcagatgtacatatgtaaaaagctctatgtataagagcatgtttaagtcataaatcataacaggacagccagcaggagcccaaatccccacttgggatgaacccaccatccacactgttgagctctgggtgccccaccagccccccaccaaggaagccacccttcaagattctgtgggcctggtcccatcttttcttggctgtcttcccacctttggatttcacgcaaggtcacacttcacctgaaaggtcttcccccatcacatgtgctcccttggctgggagagaagctcccacaaacacacccaccaccccatcatctgtgccttgacactggccacatttgcaccgctagtctgagacttcgggtctaacaggagcacgtgtgtgttcagctgctatgcccactgcagaccaggcttctgccccaccccacccacctgctctcatggctgggcaccccctcagtccacatgtctgctccccctggccactcctggaagtgcagggaagagccaggcttgggaggacaaggactctctggtcagcctcacccagtgacccgccccggtccagcagtccctcccaagtggggcttgggtcctggggcttatttcccacactacagacccagcaccaacaggtgacaccctccttcagatggagtcttgggctcaaccgagtcctctagcacaagaaggggcagacagccacttcctgacatttccaccaccttaaaagcttgggagtgctggttttacccttccagtgacctggttagcacgttagcagttaacctaggtatcagtgtcagagagcgggcaggtctagcgtccaatgcctgtcacaggagaagcagcaaacctgaaaccacgtgttcataatgagactccaaaaacatgacctcctctggaggttgctagggtatagtttattatcttgaaaactgataaataaaatcaaagaatacaacattgtgatgcctgggtggctccgcagttgagcatctgcctttcactcaggtcgtgatcccagtcccaggatcaagttccacatcgggctccttgcatctccctctgcctatgtctctgcctctctctgtgtctctcatgaataaataaaatcttaaaaaaaaaaaaaaaagaatccaacatctatcctgactttctgatataacatatatttcagggcaaccaaactgctggtggggaaattctcattcattgccaaatcacagctaacaagtacagaaagaatgaaaatagttgaatgtccccatttcccagggtgaaaatgagcacaggcaatgaacccgaggtagaccacaaacacggggagccagggtcagtggaccccaactcacacacaccaaacatgagaagtgagcagtggggacaccaggagattcagaaggcctgctgctgaccagggcagggacagcggcatgtactaggctgaaagtcctcatcggccagggacacatcctggggtcattatgggtgaactcatgagctctctggggtttgccttcagactccagatccatatatggcaaatgtaccaggctctcagattccactgatagccctacatagagagcggacctactccttgaccaaggcaaagctgtgcctctacagtaagtgaaatacagcaggttccactagagatggggccgtgggcacaagctgacaccaccgtgatgtgggaacacaggcctgcaattgggaaccgtttcttatctgggacccactaataaaacccattctcagggggcagggacaaggcaaagtcttgttgcttttgtttttactttcaaactacttcacaaaggcatttgtctacccaaaaaagagcttcagaaattcctaaaaacaagtgtcctaagctctccctcaggatatcctaaaggcaggctgcaggtgtgtgtgcaaacatgccctccctcccaggcagtaatgaaggtttccagaagtaactgcacgccactcccactggtgctatgggatgtgggatgtggtggacggggtcagagggctagtctagccccctgtgtggtggccacgtgctgggacagctgtcctcccagggccatggggcaggcacgcagaccaccaaaggctacaaggcggccagtggctggggataggcttccatgcaggacacccacccagaggccataccaacatcgcctgaagcagtcactgccctatcaggacatgaaaagtccaccctggtaggaggtgcaataaaggacagagggtcaggtcctaggggcctgaggaacaacagaaggcaggaatgcttcaccgggtctgctcagaggacacctggctctcaggcaaaaccccaggagtgtctgggatcagagatctcctaccccttaccttggcatgcttagtgggcattgcactggatttctggggcaatccggtatccgtgtccttctcagatccgagctcagcagagggctgtgagaaaccaatcggtcatgagatgggagtccgagggtgatggccactacctgcaggacatctctttgtaggaacagctgccaccagggcagagggctcccgtgcgccgtctcatgggttctcactgggtggctgtctgggtgaccactgtcatgaaccagactcacagccccaaagagggaacaggggtggccatgaactatgaaggctcactggacatgtaactggctctgcctgaagtcaaagacctgacgtcttcactgtctttacgatctcttatcccaaaagtgcatctaaagagcctgttgaggattctgtgtgtctctgagagctcctcaagcccatttaagtgacaaccatcctacagaactgactccaaaagctcaggatgaaatgcatgccctgcacccttacccacccatcccaccagtcacacgggcatgtaccactcacacccatcactggaaacctccacgcctgcatttccagcagaggagcaaatgctacagaagacgtgggcacacagcattaactagtgaacacaaaatgcagcaaagtttcaacccatcatctcttctaacgtaagggagaggtcctatacccagagcactcagaggagacagtccagcctttcctgaagcccaatgcataggggctgggagagcaggtgacctgctgaaggctaacgggtctgagcagcacagggggacacatggaaatgccccactattatttagccaaatgggatttgcccacgtgcctgacaagtccatctggcattggaagaatggcctccccacctgcgccagggccagttgccctacagagaaggtctggtatttgaaggggatgtgagcggtctctcgtgggcgcaggaagatctgcggggcaaggctgccccgcagatggaacatgtcctcctccaccggcgtgtgcagcttggtcgcgtctttgaagtatctccactcctgactgtcctggatgatgctagatagcaggggagacacagcactcccagtgagtcctccgcccgcccactcagagctggcacctaggaccaggggtgaaagccaggaccagtgggaccccttcagggatgagagcagcccggctttcagggagaacctgtagacacccccagggaccctgtcggggctgccactcaagcctacctgagctcaggattgtccacctcaatggtcactgtgtgctgtgtgttgtgggggttcctaagggcaaactcgaagaactcggcagtgcccagtgtgacgtggattgtgtgcatcgtggtgatggccaggctcagcgcactggtgatgctctctgccttcgtacgctcccgataggcagcaatgacctgcaggtcacgtgcgtgctgtgcgcggatattctgctgaatctgtgggacacaaatgccaaaccaggccaccatcatggggctggctctccttctcaagggctgtttggccacctcccccactcccatgctgaataccaaatttcttctggaatcaccacctcctccttccccagacacccgggttggatgggactggcccatcctcaggtccagaggacagtaaacgggtctacacggcactcctggctccaatcagccctgcagtggtcttccctgtagaaggtggcatctgccctgcaggctcccaagagactacaaccacccatgaggccacctcttcccatgcctcaaggctggcatcagcatgatggggaattagaagcaaaggtttctaacaaaagggggggaaatgacctcaaagctactaagactagttatgtgctcattggccaggtccccagtgtggcgccgggagccccgtctgtgagcaggcgcctgtgtaacctgagccaggccacggtccagacagctggcacgctgccttagggggctcagccacagctcagcctgcggtccttcatccacctccttcctccatgccctgattcactgggcttggccaacacaaagtgttctagaggctggagactggcttaggtttcctgtgtgacttacactgaaaggtgaggtccttggaggctgccctgatctaggacctaggaggggatgccccgtgctggggaatgagccaggccccgtgctaagaccctctgaaccctgaggaggcccagacatcagcaggcagaggatgtctgaatgtctctggacttcaactcaagaagtaaggatccaatatggtttctgacttaaatcactttggatattgacttccaacatccaaaactttggccttcccctagcaagagagttgaggccaagtctttaatttctccctggggatttgaaaacgggatggtatttgtgcttatatttgaggcctccccacacagacagagatgattagagcttcttgatggagtgcgggtcactcccagtttaccatggaccaggtggtgaacagcctcagggagagtcaacatgatcctgggggttgtgaaggggagactccttgcctgtcctgtgtcctgccacaccacagaggaggctgtcgggaagcctggcaccccacaggacatggggtgacttctccgatatgaaccagccagacatagccctccctctctcaacccttccttttgggatccacctgcctggaacaccgggtgtttgcactgctcctcaccagtggtggggttagtgggaaggatcctgcctgtggaggcggcccacagccatatggtgtcacccatgcagcttgggtctggcatttgctctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacacttattggaatcctcaggtcccaacaggcagcagagcccagtgatcagagcagtgagactgctgagccttctgctgggatttctggaaataaagctctgtcttctgtggcacctggcatggaatggtgtgaagcggggttagtgctactataagagacaacctgggggctgcagggagtgggcaagtggcagaggtaggggcatgaccccaggggcctgaagcagggccccaggcctgcctgctcagtaccaggctcggagtgctagggcacctgctccaacatggaatggctggtccagagcccaggcccaggccctggggaccccgtggctgcacagacctgagtgcatggggctttgtcactgcaacctcagaggacccccctccccgccggacagggagatgtcctacaagccttctcccaaagctgggtggggggtcagggtccacagagaaggtgcccagcacacagtctccctatgggtggggctctgctggcataggggctgacgctgaccacagcagaaggtgggacctgccatcatcccgagtgccagacacagaaagcctagccaccctcatgagaacactgagactgctgcaaacatatgtcgagcccaactctgctccaccacttccccaagaagcccccagatcatcaccacccctcgagctgttcctgactgctgggggagtgagccacgcacagcgctcttctctgtcttccaatgagcagcccccagggcccgagaccaccagactgcaccagcatgtcaggggccaaggaccagccagaggctgagcaccacttccagctcccggagggccaactgaccgcgtaaattcccaaactctcctgaaggctgagagagctctaggtgcggagaaaccaccatctcctccataaaaacaagtcactttcaccttaaattctccaggccgctgacccagaataccatgagaatttagtggaacatttccttgtatttagaaatccaattacttcccaattcactgctgtcatcagaatgagggctgcgagctgcagaatctctaccaggacacatcaacaggaggaagcccaccaaggtaagaatcgtcaggacaccagcaccaaactcatcaaggactggcagggtgctgggcccaccaccctccaaggcggggttgctggggagcccgggggtgggggtgggggcagcattacctaggggtctctggggccactagccccaggggcctcaccctcagcagctgcctggctctctttgaggagcacctgacagcaggaagcctccccagctgctgtgagcacgaagggccgagcaggttttgcctctattcagatatctgcctgccctaatgtaaagtgtgatcctcacccgtgtggcttcttgattccataaaaccatcaggtacaaccaccctctcctcccacatctaagctgtaaatatggtagggtcttctttagttttttagccaaaaatgggaatttttccagtcagaatggttacttgatttgcactgataattcatgatacggaaggcattcacagcactgaccccaaacctgggggaccctcagaatcccctgctgagctttcttaatagcccaggtaaagacccaggtgcagcaaactggacacagaattccctggggtagagccaagaatcggcttttgtaaaaactctccagacttgaaaacgttgcttgggggatgcctgggtggctcagtggttgagcacctgccttcagctcagggagtaatcctggagtcccaggatcaagtcccacattaggctccctgcatggagcctgcttctctctctgcctatgtctctgcccgccccccccccccccccccccggtctcatgaatacataatctttaaaaaaaaaaaaaaaaaaaaggaaaacctcccttggtggctgagagggtccgtttcccatacattccacaagtaggatttgggagagagagagcgagccacaggggaagagaagagagagcacaggctgctttcattcacaatgactgtggctctgcacggggacacccgggggcagaggggaggcggatggagccctcaccaacatgctggccccccagcagctgagctctcctctggactcttgaaggcgcacggacctcatccgctccagcttgcgcctgcggacggcatccccctcacggcaagcaccttggggccccttgctcccatgttgcatgtgggtgaacagcatggcggccagctcacggtccacgtcagccagcttctgtgcttggacaacattttttactgctagaaggttcaaagagagaacaagaggttaaggcagagtctacattctttaaacagacctggcaacaagcttccagccacctcacccaaggagggagctcagcaggcaacctgccctttcctgcaccttctaagatgctgataaagatggtaatcctatagcctggtctatacagtgtcccccccctttaccagcctttcaaataaaaggattctgatccttagagacaaattcatacaccattggataactttccagaggaaaaaaaaaaagtcagtgttctagaagctgatgctttctggattcccaagggccttgccaccctgacccaggaccactagccctgtcaccctcacccaggaccaccgaccgtgctcacaaaagcccccaccagcgagagcctcctgatacaggcaccactgagcaatcaggcccacggccccaccagggcctcagacgcactcgtgtgctcaggtcaggagtcaggggctctgatgagctgctcacttggcctgaagccccctgagcagctcaggggggtggggtccagactaggcctggctcccaagctcatgcccctgtctctgccaccagctgcctctagacagcctggccccccggcactgactcctctgggagactatgctcaaatcctggacacacacaaaagccatgtgggcaaacagaagctgaagcaggccagacaccagcaggcagaggagctgcattttcttcaggtgatgaagccacacgcggcacatggccatgtcacaagctgacacacgagattcacagaccaccactcaagccagtgcaggcaggcagaaggtgcaccccaacttagcactggcccttcctgaccctgaggtaccagcagtgcctacaggaaatgggttgccgtcctgaatctgccaaggggttgcacatggtcaccgcatctccagcctcctactgaaggctccatgtggtggtaaagcagtccttcccccgggctcgcccacctcgtcctctgtcacatggaacaacaagtgctatttccatgttcctcaccaccattttaaagctgttcaactgcttttcagataccatgagtcagtttagacaagtaaaatgccaccccaaaaagactgttttcacgtctcaggtgcaagcatgctgagtgggcaaccccgcctaggttgggtttgagaggcccgggggaaggggccagggcaggagacaagcaaggctaggggtaagccaaggagctcacatgtcagattgcatcactctgctccaacaaaatcagaaatgaaatctccacactgctcagagcctggaagcactttctaaaactctgtacaacttcctacttgccaagcctgccctgtgggggaagacaggctctctgatggagcaccccagcctctgtctttgaccaaacagggagagaggaaccaatgactccagagactcaattctctcaacaaaccctcacaggttacagcaccctgcatttcgggcagagcccctgacctgctgggccctgaggccgtggctttcacaaatactccctgcctgttcttgggacagccagggatggatgggccctggccccaaagtgggaaatccagattctcacccaagttcacctccctctgccacacaattaggcaagtcacctgaccgtctctgccttggtttccttaacactaaacaggagaacagactctcagcctgccgggatcatggaccttaggcccgtctgagcatctgagcctccagggccatctcccagaagaacatttgagtacccacaaagtctgggctggagtttcagagaactctcgtggggtcctggaggcctcgcggctctcaccagccacgtccaggggcctcccctccctgggcacaggttcaaggacaacagtatccgaggaccatcaacacaggaagctcgaggtagctgcagatccacatgagatggcctgcaacatggtgacactacccaaaagacccaaagggcacaccccactttgcatcctacctacaactactttgggtcatttaatgggatgtaaatctttggatgccccctgctgtgaaattctccaagcccaggcctttatctctcaggctccgtgactggtttactcctcctcaccccaacatagggacttcaaatcggtttttaaaaaccagtgtttgttcatcctaaccttttcttcattttttgctcccctgcagatgggcagctaaccaaaaaccgatcccctcgaaggattattgtcagcccagacagcttccacagagcccgctctagagtgtcccgggtatgacacacatgcttaacccacccagcccaaggggccactgaggcagggcgctgagctggacacaacagcccccactcccgcccccagatcacacgtgtacctgcatgcacacagacacacacgcagccttccacctcttactccaccccacccccatgtcaatcacagctacacatggcccacctaccccaacccgctccccactcacatctcggacccccgcgcgagatgagactgcctccctcgaagtgtctggctccatcattcgaaatgacccgtgacctggaaggtggcaatgagctggaatctctcactctctgctcacacacatgacctaaattaaaaacatcccagtataaggtattaacaccaaaaagaagtgtgatgggacacatccatgtcccctggagggagttctgggtttccaagggcctgtccataccccctggcctgctaacaccccaaaactgcactctggttctgagtctgcttggcagttgagaggtgacgggcaggcgggtcctctcgtacatgggcagtggtgtctgctttcccacgggggttaaggctgcaggtactcaggacactttttcacacacactcagttcctggtctctggagcagcctagcagccctgcgacgtggggcagctcacacattcccacgccagcctttccaatgtcaccctgctcatggaaggcccttgggcatgtccgcaaatgtctgactttaaacatacttgaaggtaaaagagcctctgcaagctagaatcttcaggagctgttttcacacagaagccttggagagaagcatgcagcctagagggccaagcacaagtccagaggcagagcacacagcacacctgccccggctgaacgggaagcagtgtcccactaaccagccaacatgtgcaaggctcaggggataagaactgtttgcctgaagaaatgcctctcctaggaggacagaatcataccctcaggacccgtgacctctctctgtggacatgactcccaagagcacagctgaagtcagattccaaaatgaacccaagttccaaacacaaatgctcatgctgtgggcttccatgattccagcaggatggtggtgatctttctgggagctattagaacaggtaccccttcccctgggtgctcgtctcccgcctctctgcgtctcctcccacctggcctccactctgccctgctcagcttcatccgcctctgcctctgtcctgtgggacagtctgggtgcaagagcaagccccccatcacagccgtgggctggtcctcagcagtggtgaccaaggtgacattttcatatccagctgcctagcagaaatgtcttcctcccgactggttctgagctgagctggggaagggaatgcctatgctgccagggaggtctctgccctgctgccaatgggggccgcccctgcagaccccagggaatggcagctggagccccccaagacatgggtgtgacagctgaccgtcccttggcattgtggaaaatgctggattcaaaaatgagagactgacaccacctctggccaaaccaagaagtgatttacttggcatggcttaaagcctggccacaaacaggtgagcagagtcctcccctagggacttccattctcatcattttcaaactcccttggtcatcttgagcgttttctgagcatctcaaatcaggctggccttcccccagccttcctgcaacaagagccactcctggttatctgcccttctgcctgctaaatggtgttcacagctcagccagtagccctggcaagcgccagagcatcagctgtcatccacgctccccgtcaggaagaccactgtcgagggccttccctgacacagcaccacactccgggaatggggaagcatggagcccaagcacccacccaccacaggcagagaagtcggctccaaagcccaggttcgccatgagactccagatggcaggcctgtctcctctgtagcaggttccttacctgcctggccactgtggccagtgaccatcctacctggggtctctgcctagggtaacctcggtgccagcctggtctcttgctcttctgcccctacctggacgtcggctctatatgtacagcatacacttggacctgcaggcccagacttccgggcagacaccctgtcggccactcccacccctccctctagccctgtgtcttgagaacaagcttcttgagcccagatgttctcagcctctgctctgttgagaagcacggaacacttctccacaggggtctgcccgccacccacccacagctgcttctctgctcttcccctccagatgcaggggctgcagtgtcttctgccaaacatggtcatctcagcatcacaaggtaataaagctcgtccccaagagtcgagcctggtgttaacaccccttcactgggatcagctgctcacacctcggggcctgctttgtcacctgaagcccaagaggagcagaattcaaggcaagacaggctctcatacttgctcatccacccattcatgaacctctcatcaccaggccaggtcactgtggctgctgctgggtgagcatcacagcaggaaccaggatgtccctgagaaagcccccaggtccctagggggacagacaagccagtaggtggacaggcagggacagagagcctggctaagtgggcaggagacagccagctagtgccagggcccaagtcccagcactgacactacttgctctggtcctaccctgatttccaggtgagaggggaacaaagcaccatctggccccagctgcactcagttcctgccatgagctcagagacagtccctgctcctggctctcagctgcttcttcccagtgagggcaaacagggcctgcaagctctccatcccaccctgaggaccggctccatctgtcacctggcagggggcacataccacctccactcactgttggggacagcggattcatgccacacctgtgggaacctggggaccctccgtagccttccctgaagcacaggcagggaggcattcaccatgtcaagctgaaatgcacgcagcctacccttagcaggcaacagctctagggggagggacaggaaccagggaaatggctgcagatccggatctggtgcagacgggtgcactgccagggcacacaaccggggtgcatggccggcattgtcagagcccaggctctcaggactcacggctgtcatcgcagcatgtgggaccagcaggaccatgtagtcccctccagacagacaagggccatccctgagtgtcacagtttcctctaacagtatttgcactcagagcgtgggttcgtgtggaaggctgcacaataagctagatcagggcaacatacacctgcttactctgggcctggatgtggtgaaggtgctcacttcttcagggcaccatctgcaatctcaagttgctgtgggctttcttacccttccatgacaccggggttaagctggggacccacacatttgctgtgggagcggagactccctccagatgagcagggttcctgactcagctgtgcagctaccttcattccacggtggcagcagagcacatcaccccgtctgaccccaaacccaggctccctgtccacaccacccagctctccttcaaccctgagaatatggtgccaggggtcagggcagccccttgctcccctgaggagccctggaagggctgcagatacaagtgacaatagctccccgatgaaccacagccacaccgggatcccggggacagaaaagcaccagagacactgcagtcttgtctgcagggaccatcgcggaacctagaagccaccaggcacaagcaccccgccgcccccttcctatggcagattcatgggagatctgggctcagagagggcacctcgttctgcagcctcacacttgacagcatacactgggagtgcagagccatggagcgccgtccaaggcaaggccacgccaggcccatgatgctgtcctgaccctgcttctgtcccctgaagctcacacagcctcatccaatatgcaggcctgcacctgctgcttggttctaaacacccagttttaaaaaagaaatacaagacacaatgccattttttgttttttgtattttttttcaaaaaatgcaaaatttacagaggaaaccagaccatggctgtatggacagtgagctgctcgcgctcgtcctttgtcaatccccctccgctttggcgcctctgggcgggtgtctctgactctcccccgtgcgctatcgctcccttgcaggcaaagtatggaaatgacaatttaagataacgtataatagagctaatctttcttctgaagataagatttaaagcgcggagtcactgcacatttgtgggctgtgaataaatataaaaataaccaatgcaatctcagaagcagaacgcttcttgcagctccctcactcataggagcttctgaatgatgtctgcttctcacatacagttcctttttttctccagaacctaattgagttttgctgtttccatattgtctgttttcaaggcggcctcggatcttacaattgtcaacaagcaggcagaaacctccagcctaacttaacgagtatgcacgcctcctgctgctgcggccgccccagcctgcccgcctccaagctgcccgcacgagtgcatgtggcccatggggctccaggctttaaagccagcctgggctttgaggaatggaaaattcccccataaaccatgagaggtttgagtctcatctagacagacatggctccagaaacctcaaatggcttggtttcgtcctgctttccatttcaccccaaaaaacctcaatccttccatagtctcctcaagagagactgagtgggcttgtcccctggccccacaggggctcactccaggcccctacccagtgggaagcacaggaagacatctcaggccccatatcccctgtcgatcaggacctccgtggagcccccagaatggccagccaactccactgtagccacagccagccccataccctgccccaggcatgtgaacaacagcgcacagggctgcactcagtgtctgaagagtgaccctcagacatcattatatgtatggtcagagtataggctctaaagaagtgtgtttctctgttcccttatgcccaagctgcatcaaagttgagaaacattttgctttcctgtcctatgatgaagtaggctcataatctagatgaaaatctctcctggaaccagtcattttatctgaggattttcatagagatgtcacatagacaacaccaagaaacaaaatgtggacaagactgagatgactaatgattccaagtcctgatcagaaaccaagaacaccaaacaactagtgccaagacaggctcaggggtcaaggacagatctagtggtcaaagacaggtccagtagtcaaggacagatgctcagtggtcaaggacaggtgctcatggtcaaggacagatgctcagtggtcaagggcaggccaagtagtgaaggacagatgttcagtggccaaggacagatgccatatgatcaaggacagatgctcagtagtcaaatagtaagagtttttctgaattccggttgatcatatgatgcccacaataggaccactgccctgagggagctcatgcaccaactgggaaaatgaggggccacccaggagacagcataagaccaggtaaagctgccagggactcttacccacgttggccaacgtcaggtgcagccagccctttaccactgtgtagacaccgatgggcttgacacagccaaatctggtcacatctccactcaccaccacatcctgttcatattcagtcgccacgacctcaagctcatgcaggacctgcacagccggccggccttggcggaggagatgctgcagagaaaaaggcaccagtcagagggtagccgaggtagagcccatcttctactgaaaatctacactcttgatattgggtctttcccacaaattcttcaattaagtgcaccccaattcacccactgagaaaatatcatggatgcacaaacaggcctggcagccaccctcctggaaccccttctatggggagaacaatgggaaacgagcaataggaggaggctgggaatgctgagcagaccccatctcccagtcagtgagagcttcaagaaggaggtatgaaccaggccggggatgctaagggcacactaggcccagggtacagagcagaccccccacacccggggccaggagcatcaggcttcagcatggctgaaagacaggtgaggcctggttgtggaggtccacataaggcccaggttggtcagagccggcaagagcagatccaggacaggcctcctaaacccagtgctttctgctaatccagttcacccctcagtgccctgcctgagcctaagtcatagtgaggcctgcaatggcctcttcctgggaggccttgggaaaatcacctgacctttcaactagtgcttcccactcacaaatgggcatgtggaggtcagacaagcatcccaacttctggaattcttgcacttctactacagcaaacatgcccagctctgggagtggaagtgccccaaatgacccctctgtagtggctctggcagcttctggttgcttcaggaggatgaggacctggccggactccccagccacccacccgcctactctgcgtgccgaacctcccacacaggcacacctctgactcagaaccacacttctgatgagttctcatctggggagggcagagagtgacggtaataagcacaagcttccaagtcagaactgggctaaaaggctgggtctgctgtggtccgcgtgaccgtgggcctgtctctcagcagcacatgaagacagtgcagtacaactgggctgaggacaccgtccacgaagttcaagagcaatcagatgccgtggcaactgtcactattgtcctaacaacacccttgtagccttgggaatgagctgaagattgggcaaaatgtacgtccagatgttcactgcaacattgctcatggggaaaaaaaaatcactcagtgcccagcaacgagagcaagtgagacgagtcctagtgtatccataccttccagaaaatgtgcctacccaaggcacacagctagttcagcacctggagttaacaggcggttctctcttcttacctcatctcccatctcttctatataaattattaaggtaaagaccttatttttaaaaagtaaaatttaattttagtttatccaagttaagtgtcaataatcacaacagcatttgtttcaggggctctttgtggaggacaatctagagtaacgtcatctccagggacacagggacaccgggctccgcaagggatgaggcaggagccacagcgggtcccaaggtaacatgggctcctctctgcccctgcagtgcccttcctccccccactcctacctccctcttccctccactcccacttcccccactccctcctccctctatccctccccatccctcctcccccattccttcctcccttcactccctccatccaccttccctcctctctccatccctcctccctctatccctccactccctcccctcattccatcctccctccattcaccctccctcttccctccatctctcctccatccatccctcctcctccatctatctctcatccctcctcccccactccctcctccttccatccctcctctgtttatccctccactccctcctcccgtttttcctccctccatccctccttcctctatccttcctctccctcctccctcttttctctcctccctcactccctcctccctccattcctcacccttccccccacccccatcccctcaatgctccttccatttgctcattcccagctacacagcaccatgcccactgtgggggtcatgactgcactgaagatgaagagcagtcccattcctaccctctagaactttcaccagccagcatcagagacagacaagcaatggccacgatgaccccctggagttgcacggtgacccagaaaggcccaagggaccagggtgaggaatgagaacatcagccaatatctactgggtgctcaccacgggggacccctgccctcactcctcctgctcccctgggggcatcactcctgaggggtagacactgaatcgggtggaggagggatggtacaatccaggttagccctcagagggaagaggggaagctgtgtcctctcccacctccgtcccacagccgggacatcgcgtccccagtccctagcccaggagctgcggtgtaattacactgaccctctgggtagaagccccaggaagaatcttaaattaacatatggatttaggttttagtgtaggaggaagatggaaagcacaattagagaaagaattagattctaattacaattagaaaagcagagttgaagtaacagatagcaggcaccaggtgatctctgcacacccaatcgatgactcatcctaccctctgtgggccaggcccctgctctcactggtggggggcagaggcagaaggggactgcacctgtggtcagtctgtaggaggagctgatgactgaggaataatctcgcattacacagctgagactctgggaaaatcttttcctttggggaaggacggactttatgaacaaagacatcagatgacagcccacactggacgagaaccagcaactgagcagacccctcccccccccaggagggacaccccaccctaaccagcccgctgcagagcactgccaccttcatctggacggccgcagatcccacgagcagcaaggagtctccatcccagatgtcgatctggagggtctgagcagccatgtagtgggtgaaccagcgctgttctccgggtttcaggaacccaggttccaccaggtacttgagttgaaagccaggtgtccctgttcagagaggccccgaatgagcccaaaagtgatttgagtcattatcatcttcacattttgaagagactcctgctgtggtcacaatcactaatttctgcttccgtcataactacagtttaataaacacattaatcaaaaatatcataaaacatccacatttacaaaatacagagtcgtatgcagaagtctacatttctttccaatacatacaatgaactcctcccagaataccccagggaagaatcttgtgaaacagtctatcctgaatggggacagatatcctgacggggctgcccagggaggtcattattctgtgtgcagggtttaaggggaaacatggtctgccgtcaaaagtgggacagcacacatgaaagcacgcgcacacacatggagctcaggctaaggtcaggctgagctgccctcaacgcattcccagcggccactaccaggtcatctctgagacagcttctccaggtggcatcaccctcgattctaaactgacaaaaacaccactaaaatcattagttaaacatttcccccttcccatcaataacctattttcaggggcacctgggtgcctcagcggttgagcatctgtctttggctcaggtcatgataccggggtcccaggatagagtcccacattgggctccctgcatggagcctgctttttcctctgcctatgtctctgcctctctctgtgtgtctctcatgaataaataaataaataaaatcttaaaaaaaaaatattttcagagcctctgacgagtagaatactaccatgtttactcagcattgggaagcgcgacccaagtcagtctctgcctctagccacctcagcccctggggagcggggtgggggtggggggcacacagtcatgaacgtcccctctcctctcaagactagaacagtgcttctagttgaaggagggaagcgggttgggatgggacacagcaccactgagggcctcgcatgggcctacgggactaagccaggaccagttgccataggcctggctccgaccacagtgagaattcacagggacacctcattaagggagcccagcaataaggaatcaggaaaaaggctcagagagggggtggtaatgacatcatgcagcggcaggccagtccttagtatcacatatccagtgaccagacaaagtcacagggccaatggaggccaaggacacctaccacccttgccccagtgccacccaccagctgagacctaaagtggcctgtgacaactggtcattctcaggacaccgcctaaataatgggagccaaggctggctccttagatgcatggtgaggaccagacaaagacctataaacacccaagaccctgctcatggcccgaatggtatggcaccccataccaggcccacctgggctgcaggagaaggaagagctcagccactggcttctacactggtcatacttctgattcgaggagacagaacaggtttgacctctgtgggccaacttcccaaacgatgcctacagacaccagccttgggagctccatactccacctccagcatcgcccagatccacagaccctccatccagagttctacctgctccccagtgacgcatgtcccagatggaccctccatccttcccacaccagctccttgttacatcgtcccaaagtacttccttggcaccagacccttgcaacgcccatgctaggctggctcccgctccaccaccactgcatccactcagctgcctggccctttggcacaccattaactcacactcttcaaataactgcagtgcccccagcatgtgcaccaagcaccacgcagggataacttgagagagaaggacggacagggagggtccccacctgatggagctcatggtcacaggataacacactcagcccagagctaatggcgtgctgagtacctgggggaggcatgtggggctgtgggggccatcctggtctgggcccagcctcagggctctcatgctggaggctcccagttcctcctgccacaggcttgggcccctttacttctatccagctctgacaaccggctcctcagtggccttccctgaggtccacactccctgcagtccatcccactacacagccaaagcatccacaaagggaaccctgtgtcctccatcctgctcagaagtgagcaatgatactgaccatctactcacacaagacagacctcaggggcagtggcacctccccagggtccactcaggttcccaagcttacctccctcaccccctctcaaacaccaagccccagcccagctggctccattctgagcctcagtccagccacacccccacatccaagtgtggtaggtactcttcctccggttggatggtcctcagtctccatctctccttcatgacccacaaacacccctactctgcgaaggcatccacaactttcacagatgacaggcactttataccactatggtctacaatccacaatgaatccacaatgaatatacagcagctgggaatatctacacaccatatgacaaagcaaaaatctagagacgcaaggagaaacagataaaaacataataataggagagatcaactaatcactttgttttcagggcaggccaagagggcaataaacaacagcatggaaggccagccaagataatccatcacatagagcgtatacatgtacatatgacaccattccctgctaacattgaatatatctttcgagtctatctggaacattaataaaaattaactatatacctggccacgaagaaaacctgaatatgctccagaaggtgaaaataacacaaacaatattctctgaatacaatgcaacaaattagaaactggaaacaattttttaaaaactaatttacagcttacttttaaataacttgatgtcaaatggaaaacaaactccagggcagcccaggtggctcagcggtttagcaccaccttcagcccagggtgcaattctggagacctggaatcgagtcccacatcagactccctgcatggagcctgcttcctcctctgcctctgcctctgcctctctctctgtctctcatgaataaataaataaaatcttaaaaaaaaaatccaaacagattttctacagaataatgaaaatactacttatctgaatcaccaaagcataaagctgtaatcaaaggaaaattcatagccttgaatactttgcatccacaaaaacaaaagaattaaagtaagtgaattaagcaactcaaattagaaaaataacactgaagaatacaaaaaaaaaaaaaaggcaagaaagataaagatgaattttaattggttagaaaataagaaaagagcaaaacctgcatgaatgtgtctttggagagtcagggaagcaggagtgaggacatgagaggatgcccatatccctgtcccatgtcccattagggcagttctcccgaggttaacctggattcagcagaacccccataaagggcaaacagtttcacatggaaaaccaacccagccagaatgtggggtggtggtggggctgcagggaagaagagcatggtggagtggagggaaagctgtaagagcaaagttgcacctctagctccacagcacacccactgagtggacaggtggtctaatgcagagcttggcaaaccacagcccataggctgattcagctgtaagctaagaagagctttcacatttttaaagggttgttaaaaaagaagattccagagagaacatatataacccagaaagtctgaaatatttactatctggcccttaacagaaatcagtctgccaacctctggtctaatgaaacaaaatataaaatccagaaataaatctgagccataaaaaaagggtataaggtaagggtggctctcaagtcactttagaaaaagatggactttcttctaataaatggcactgggtcaaaagaacagacgtttgaaaaaaaagataaacctggactcctatgttccatagcacaacaggacaaactacaaatggaccagaagtctcatgtaaaaaatgaaacctataaggcccctggagaaaacgtgtgtggatctctttatgaaccaaggagaacaccttcgcatccatgactcaggaatttagaagcaataaaactaaagaccaccacattaaatggcataacagcaaaattttatggggtacctgcaagtctcagtctcagggttgtggttttgagccctaccctggatggagagattacttaaaaataaaaatcttgggatccctgggtgactcaaaggtttagcgcttgcgttcagcccagggtgtgatcctggagacctgggatcgagtcccacatggggctccctgcatggagcctgcttctccctctgcctgtgtctctgcccctctctctctctctctctgtctctcatgaataaataaataaaatcttttaaaaaaaatctttaaaaataaaactttacatagcaaaacacaccaagagcaaagtaaaaatgaccaaaacaggagaacatatttccaatttatatcacaagtaatagctaatctccccaagaaacagacttcccaaaatagaggacagaacaagaattggatttaaaaaatgggaaaaaatatgaatagaggtttgcagatagacaaatatccaagatgctcaatttcactcattataagagaaatgcaaactaaaacttcattaagatctatctcacatcgatcagattatacaaatcccgaagtttatgccacattttgtgggcaaggccctgaggaaataggcagccccatgcgctgcaagcggagcttggaaggccatcgaatgacaggggagtagctgacggctacaatctgattgttagtaacgccccccaaacacatgtccacacacagagaacatataaacaaggttacaagctactacattatctgaaactcaaagcctgaaagcagcctgaacgtccatcagaaggaggttgacctgaatcaatcacagggcacacgcccagtgccagacgatgtagctgtgaaaacagtgcagggggtccccacatggagtcaagctcaggacagaatgttaatggaaaaaagcaaagtgataaaagagcatatttggtatgctatctattgaataaggaaataaggagaaataaaaatacatttgcttacttgctgaaacaaacagaaacatcagaaggagaagccagaaactggaagcctggttacctccaggaggccctgcagggggcacatggtctagagggtacaacggttttagactgtgagccatgtaaatattccaagaaattataccagaagggaaagaaagccaataaaaaattgataacaaattgaaataaatgaatctgactgaatatcaaattggtaacagctccacagaggaaagcatgagctgcagggattctgacacgctgaactaactatgtccccagcgggatgtggctccgccctagagaatgccagaaacctcaaccctcttcactcagcacgcgtgcccttagcagtaacactggcaatgaaattctaaaagtacattatagaaatcataaggaaagcaagccatatgatcaggaagcatgattttcagtataagatggaaaagataaaaataaattaaggaaaaaaccctaaatctgaattagaaatatcaatgtgaactcatgtttgaatatatattctccagccctgcccagggaaaggcctggaagcaaggccatgcaggaccaaaagtagcccagccctcagtctccaaaagcaaatcccatgcaggagaagcggctggtccaggtcaaaggcagaggaagtccacaggagccgaggatgctgggccacccagaaggcaaggccatgcccaggctcctaggttaaccaggccaccagcagccaaacagaggggaagtagtagtaatgaatatatcacagtgaatagatatcagagtgattcaaaagccaacacaagaaacaaagaaaaaagacatataaaacctcaattgttaccaccagagtaactgtcacagcttctgactctgaaaactggaaattaaagggaaagtatcaaacatttgctctgttttggaggatgtatacatagttcactcttggttgataagggaaaactggttttttaataaaagaatgtcagtgaataaagggaaaccaaatgacaaattaagaaaaattaccattgcaaattccaattaaagacctggcccaggggatccctgggtggctcagcagtttagtgtctgcctttgtcccagggcgcgatcctgaagtcccgggatcaagtcccacatcgggctccctgcgtggagcctgcttctccctctgtctgtgtctctgcctctctctctctgtgtctctcatgaataaataaataaaatattttaaaaaaaagacctggcccaggacgtaaagaagtgggtaaaaggttgttgggtggatattcacatggagctaatgactggcctgagtagtccatgctgagagccaaggggaatggccccctgccccccagagggatgaggggtagatggagccagtgccctgagaataagaggcctgacctccacactgctgatgaaatccaggcgtgggggagtgtcagccctacctgcccgtgcacaagcccttcggggagaaaccagcagatgcacacagaatgagcgacgttctatgtgacaattggtctggtaccttccagtggccactgtccctggagagagaatgcaggggactattccagatgaaaggagcttggggaggtaactcccagcacaacccaggacccagactgaatgttggagtgaaaaagtcactgtcacaaacatacaaggaaaaaccaggaaaggaaacagtgagatcccgctcattgtcagtatgatggaggctggttttcaatagagcctgttgctatgttcagaggcaaaccatcacctcacctgctactgactttcaaatgattgagggaaaaagatgaacagttaactctcaagcatgtccaatggcctagacacgatctccatggcagaggacatgggactggatgcggagatccaccgaagaaaaagcaggcaaggcagcaccagatggacccagaggaggactggcaagccccagatgagactttagcccaggggggaggggggtctctcagatgacagctcaatgactccagcgcccttggtaataaggaaggtcactgaaacaccaggaggaacaagttagaaaaatccaactccaggcctgaaagcaaaagacacgaaaggaaggaaggtaccgtgtgtgtggaggacggcccactaacacctggacccctggaaggagcaggggcaccagcgagcatggaagccctaggggcacaccccagtcctaaggagaggagataacaagaaacaattctactcacaaccaggaacaccaagaaacaggagcctcgggcagaggccaccagacccagggcggcaggttccagaagcttctgttcaggaggccaccaagtctcctgctggaccagatctactccagcagatgtgccctgggagaaactgcttggcaaattgagtaaagatgaacatgtgggaccctatagcaatttcactcctgggcacacccccaacaccagtgcgggcacatatgcaccagaagaatgctcaccacagcattattcaaaagaaataagctgggaacagggggatccctgggtggcacagcggtttggcgcctgcctttggcccagggcgcgatcctggaaacccgggatcgagtcccacgtcgggctcccggtgcatggagcctgcttctccctctgcctatgtctctgccaatctctctctctctctccccctctctctgtgactatcataaataaataaatattaaaaaaaaaaaaagaaacaagctgggaacaatacaaatgtctatggacaaccaatcagataaatggtgtgcaatactcagagacttctatacagccaagataaaggacaaactactgccccacacaacaacatgcgtggacctcatagacacaatatccagtggaagtaagactgggaaaggcacacacaagatgatcctatttcagttcaaaactgcaggtgcactggtctacagggccagaattcaaagagtggtcacccctggggaagccaccgatggtgggaaaggagaaggctgggagaggcctggggatgggtctgcggccctatctgagtgggctggtgatcacacagatgtgttcactgtgtacaagttacagctgttataaaaattaactcaaaatgggtcacggacccagatttgaaacacaaaacttagatactcttgaaagaaagtagaggacaaaattccaggtgtggtgacgagtgtttacctctaacaccaaagacacgaacgatccatgacagaaataattgataaattggactccattaaaattacaaacctctgccctccgaaagacactggtaagaccgtgaaaaagatgagccacaagctgggagaagatattcgcaaagcacacatctgataatagtgcaggcaggcaaaatataagaaggatctcttaaaaaatacaataagaaaatccgttctacaaacaggtaaaatatctgaacagacacctccccaaagaaagactcatggcaagcaagcccatgagaagatgctccacatcacacgtcatcagggaaatggaaataaaaacaacagtgagatgtcaccatgtacatatgagaatgggcacaatccagaacgccatcaacaccaaatgctgggaggatgtgggatagcagggatgctcactcactgctggcagaaacccaacatggcacagccactcgggaagacagttgggcagtctcctacaaaaccaaacatactcttaccatacaaccatgcagccatccttctagatgttacccaaaggagttgaaaatttatgtctacacaagatcctgagcaaaaatgtccatgattgctctagtcataatcaccaaaaactggaagcaacgaagatgcccttcagtaagtaagcgaatggatgtccatctagacagtggagtattattcagcactaaaaagaaatgagctaccaagccacaaaaagacatggaagagtctcaaatgtgtactactaagtgaaagaagccagtcgggacaagctatatactatataattccaagtgtataacatcccaaaacaatgaggacagaggagggggtgatggggcaaagaggtggagctcagggcatatttgggtggtgaaattattctctacgatattgcacagggatacagatgctcggcatttgtcaaaatccatagaaggtacaccaccgagaataagccttgtgaactctgagctttagctaacaacaaagcatcaacactggttcagcaactaggggaacttctggggcaaggggtggatgggaactctgtacccccagctcaatttctccataaacctgaaactgttctaaaaagtaaaatccataaattttttaaaattcggggaactgaacactgtgttgcatgcatgttacatttcattaacaaaagaaaacacatcctaagtgagactctagtggggctaggcactgagctatggggacctgagctggggctgctggaggaagcctagccccagccaggtgcccaggcagtggggtggtggacctttggctgagcctcatcaggtaagacactgaaaagaacttgttttggacttcaacttaaggtttttggacttcgagtcttaatccaataatgacaaaatgtgattttggagtttaatttctttggacttcaacttacaaggaaatttaatttttggactaatttctctctgggaaattcctaatggaaaggatttatatttgaatcaggagtacctctccatggttggccttttgaattaaattacctgaacactattcatttaacctcatcacagaacctctggtgaaccactccagatagagcagatagatggggctgggaacaggaaatgaaagaggagacagtttctcagcaaaggtgcctgggagccttgaaatcgatgcactattctgatccatttcagagccttctgagggcatgagctaaactgtccaccaatggtccccaggagcatcagcaaagggaaggcagttggtttgggacaagtggtcccaggattgttaggaaacccagggagttgagaaaagaaaacctagagtattgcaagagagcatccttagctggccacacacttcatgtagtggggaccccctacagtcaccctctgcccagcacatgcctctcccagaccaaaagcagctcatgggctggagggtttgggagaaggaacagggcctgagctgtgcccccatctcctctctctttccagatttgtctgcaggaatcgagggctctaccccagggtctgtggccaggtggtcctacctctgtcggcagcaaagcccatctgatgaggtgagcaccagcaagcacacccaactcttgggctgaccagtgcctgcaatagctctgaccctcttgactgattctgaccttggacaagggacagcagcataacctgctgtacttatgactcccctaccccaagcccagacaaactctgtctactacctgttttctaagcatttatgcttattttaaaatttctctccttctacttatattgaatttgttctggttgttttcttcaaacttctggagttgaacaattagatcagaatttttaaaaatactttcttctttcccaatacaagcacttaaagctgtaaatttccctataaataacattttacctggatctcatttgttttgcaatatttgcaacatgattttgtgataccgttttcaataaccattcttattttgatttcttcattgacccacagaataaataaaagtatgctttaaaattaaaaaaaaaaaactagcagttcggagcctttttaggggagggagtggttgatttctgacatgtggtattacagtcagagaatgtcatgccattcagatcagatactggtctcctggaacatggtgatgcctgcacggtggcctaaaacatgcacacgtatacgtgactgatcagttcttgtaaattagtctcaagcgtgcttggaaagaatgcacatcctctaaattggttctataggttttataagattatttttttaaattgtatcttattacatccttgattattttctgcttgatgcgtcaattcacttggaggttcatcaccttccacaaccatagatttgtcaatttccccttctaaagtctgtcaatgtgtcaacatttcttttgtgttttgaagctgcttgttggcagttcacaggttcagaattatttattttcctagtgattattctcttgattatatttaataggcttctttatcctcattaccgcatttgccttttgtctcttttgtctgatgctaatgtcaccaaactactttcacttgattatcattttaatatggcatttcccacctgttctttctactgaaggagcagcaagggttcccagctctgcccctcaaaaaccctgccactggccaaagaggggtaactcatcattgataaaaacaatatatttggtatgttttgatccattgcagtgtggtttataatgatgccagaaacaaaagcccttggccccactttcaaacaatgctagggaactggctcattattttgaaaatttgtaaataaatgggaagaaatgaacaccattctgcctttcctacatggactacacctcaaagtaaccccatagttgaggagggcaagtttctctatatggaaggattccagttgagaaacaaaaaggtgttccaggtggggatccctgggtggctcagcagtttagcgcctgcctttggcccagggcgcgatcctggagtcccgggattgagtcccgggatcgagtcccgcgtcgggctcccagcatggagcctgcttctccctctgcctgtgtctctgcctctctctctctttctctctacgtctatcataaataaaaaaaaaaaaaaaaaaaaaaaggtgttccagaagtacaacatcaccactgttcaaccttgagcaaaataaccagcctatgggcaaccatcctaacagccactgactatccacagaaagaagggaccagcccctgcacagccccagccccagagagttcctgcccaaaacaaaaacatccctctgaccgtgcatagagatctccaatgtccaggaaaaacaggtgacagagaagggcactgtaaatgtcaccactgggaggcaaaacccaagtgtagatggtgcgaagctctgaaggatgaagcaatgtggctacttcaacaaataacacaacaaccaaggccaggggcggcaggggagctgcagacacacacactggagagtcatgtcaccgcaactcacaccttcccaagatcttgatctgaacaaactattaaaaactgtatcctccataaaaacaaacaaacaaaaatccttcttccatcaagttcgaaaaagttttttataaagttcctgggaaaatttgaacagtagctaaatatttgataacatttaagggattattattaattttaagcaggataatactgagtttatgtcctctcaaacaagactattttttttcagatatcttttttttttcttttcttttcttttctaatttgttcaagatgtttcctctctctggcatcccactaccctctatttcttcttcttcttttttttataaatttattttttattggtgttcaatttgccaacatatagaataatacccagtgcttatcccgtcaagtgcccacctcagtgcccatcacccaggcatccccacccccctcccacctccccttccaccacccctagttcgtttcccagagttgactctttactttttaaaaatacataaggaaatagtcacagattttgcttcgagggatctagggtctgggggggcggggcacagttgacagctgctggcacccactggcagaagcactggttcattacactattccttcaaattctgcacgtagggatccctgggtggcgcagcggtttggcgcctgcctttggcccagggcgcaatcctggagacccaggatcgaatcccacgtcgggctccaggtgcatggagcctgcttctccctctgcctgtgtctctgcctctctctctctctctgtgactatcataaataaaaaaaaattaaaaaaaaaaattctgcacgtgtttgaatattttcaccatacaatattttgtaagtaatacaatttataaacaaggctcaatgctggatttctcacatctttgccagacacataagcacttaccagcatcagaggagccatctttgtcagtgagaaccaggagatacaacagggaaccagagcctgtcctgcccgtctcgtccagcctgatcagctgcaagcgtggcgttgtcacgggtgggaagcgatagaactggaaggtgaaatacacggtctttggccacggtgctccctggcagtcctgggacactctgaatccaaaaaaagccccaaattaatcaagtatccatagcagagaacctgggttttcaagagcccagcaccacaggcaagacactgcctcatccacccacatgagcagctctactgggatctcggggagcccgtcagacagcaacccatttcagccccagctggagaaccctgctctcagagctacatgaataccaagaattggccaacaacactgtccatgtcaatgacagaagttcaggcaaagccagtttcacagcatctagagatggctgtgtgagcaccctaaccaaataaaaacaaaaacacatagcattccttaatccaggaagacctcagcatagagagtcctgccaactcagagccatacagaggggtttcttcaatgcaaggaaagtgaaagccaggtaaagagtaacataaatagaatatttaagatcgcacctgctaaaggcaagaaaatgcagcactatctcattgctttgtagacaatctgattcttccttttgggggttgaatttcacgggatctgtaggatttacagcctcggctggttgcttattggcatccagaatctcaggaaagccgcaggcttgcaggagcaccattgaggctcgtgagagcttacttccagagctaaaacagaaaagatcatggtggccggcctccagaaaagggccggcaaagaggtaggacctgccgacaacccctctctggggaaagcctgttgttcagagtcagtcttcccagcacagcatgccaggggcctcagggccacctctcacctcctggtctgggctcccacaacaatcggggtgtgcaccggcgtaaaaggcagctcctgcagctgttcagcaacagaggctcccaggaccaaggacgcctggctcagctgggcctccaggtgagagatcctgccctccgaggggggctcctgctgagcaaaatgcggccccaagtgaggccaacaaggatcccagcctgtaacaactgaggaggctgagatggggggggtgttcattgatgagccacctgtcggactgctagcttcagcaaaggaaaatacaggacgcccaagtgcctcttacactttaggcaaacaatgaataattatggtataatgtaagtatgtcccatgaaatatctggaacttacactaaaaagtcaccacttatctgaaattctaatttatctgggccaattcgtattttaggtgagtcaaaaagcaatgagggaggtatcatcaattttatgaactggcaccattgagagatctgctgtctgagaaggtcaattaacataaaaaccacattgatagatcagaaaaatgaaccctccttcaacgctgcaagacagaccactactcagctcccagctcgcctgtatagacatttctctgatcccatcacctgaatattctagtcattcctttgtattccaccttccacatccctccagagcagaagacgtgtctaaatgaagagactgtttggaaatcagaatgctctctttcacaccagctcacctagctttctgtttcagctcaaaaggaataccacaaaaccagcgataactccgacatgtctcagctgcctataataagaaatgcaggcttttcataaatacgtgagccctagacatgttctagaataaggcatctcacggtactgttttcaagtaaataatttaatggcgctatttttcctcaatagacttccagtacaggactgaagatacagtttgcaggtgagccagccaacttttgaaaccaaactctatgtgaattccccaaggtcagtggaaacacaggcctgttttccccactagatgcatccaaatccaggacggtgggcttcactacttctccttcagctgctgccaacaaggagagtgtcctggagcgctggaggcttggatcagcctctatttagaactctaacaaaactttccttcccccttcatgtacagagatctgaggggggtctaactttgtcttgtcaggcagagcaaaagcctaagtaagcctcatagcagggagagctcccacaccaaattcccaggtctgcaggacagaaaggtggcaagtacctggggcatggaagtaagccatgcccacccactccccctgccaccctgtccctacacccatgtcacagagaacccacgaaggtctcagccacctccccaaggaccagcacagctcacaaagtcaaagactccagcagatgtcaggagttcagagccattccctggaaggaggaagggtggccatgcttacctcctgggccggcgggggcttagagctgtcgggttgccgtggagggggtgtggctgagcgggagcgggtggtaggctgtggggaagctgcgagctgggaaagggacaactagagggggaggaagagtgggtgagaggtggaacacagcccccctacattccagcgcccccacacacacacatacactcacactggcataaacccaggagacatagcacagaaggcagtgaggtctgcaaccgcttgctgggtggctgctcctcgttcctttactagttactaagaggcaagattgtggtccctaccacgtggtaacactggcataaaaacaatagtaacaaggaacagcaggtagaaaagaggaagggccgaggaagacaggttctctgccacttatgggctgtgggggcagctggtctttgaacttctctggatttcacatccagccagggactgaacaggtgcaaggtcccttccaggtctcaccaccgctgtgcctgggacacaatcccgtcactgaggcagtgactggaatgggagccgacatgagctctagggacagctgctgagactgaatccccacaccactattactgtgtgactgagggcaggccattgagtcctttcttttcctctgtctcctcatctgcaaaatgggggtgaaaatagtacctaaatcacaggactatcacaggaattaaatgagaagtcgtggaaagggcttggccagaacctggtgcatgtaagcctcctgtcacgtcgcagatgtctggaaagtgaatgcagctgtaaggagggcctacgctccatggaagacctgcctctcaaatcttggactcactttatgacatatacagttagctggcccagcacagtactgggacagaggaacccaacacgagacagatcaaaggagtggctgggataggcatgtggcaatgggggaccccagcagcagccccaagggcagagaagagctgtggggtctgttcagctccattcagacccacaagctgttttgagactttcctgcctcaggaagtgggcagcactgttctgtggccctgaacacttagggtgacatggaaactgctgtaacgggccagtagggaagcttgggaggctgtctccagacctgtgtcttcaggcaaataaaacgttcacttctgagcgagtctccaagctgcttggtacacgttaaacccacttccttgagacagactttgatagtgagttattcgacttttctagataagtcattttccagggtgatgctggcaaagtcatcaggtagacaagccaactctgaccctgaacaaggtgatgccggaaccactggcaagcactgtgacaagcttggggttacaaaggggtggactcaatggccagccaacaaccagcacctctcttcctatgcaggagaccctcgcacacagaactatctgctgtctgacaggcggtggcccatctttaccagcaccagctgttcagcctatgggcctggctttggccccactgcccagggcagtggtcagtgccaggcaccctctcctggtagagacaagacaggaggaaatgaagaccacagggcagcctgggtggctcagcggtttagcgccaccttcagtccagggcgtgatcctggagacctgggatcgagtcccacatcgggctccctgcatggagcctgcttctccctctgcctgtgtctgtctttctctctctctctctctctgtttctcatgaataaataaaatcttttaaaaaactttaaaaaccgaacttaaaaaaaaaaaaaaaaaaaggaaatgaagaccacactgtggcacctcagaaaaaaacaaagtctactcggccctgaaagattctcagctatcagacataactgccagagagagcacatcattttttaaaagtactgaatgtgtcccagcaaaacaaaagagacaccactgttcttaatgatgcagagaatgttctgcccctggggcctttaattaatggattttggaattctgaaacaggcctctcagcccagacttacccctggtcccaccggcgagtcctggagggcagcgagcccctggggcggcagtgctggcaggtctgggggggaagcagacagaggtggggaggggcactcaggagccttgggaggcctgactcagggcctgacccatctgcccccggctgcgactggtgttgccgctggcgttgggcagtccaatcactctgcagtaaggtccagagccgctcccccaggtcacagcctcagaggacggagctgagctcgggctgctgtcactccagttcccctacccctggccaccccacctggcctcatactttctgactgtgtggtgtcaccaagaatgggcttcatgacccatgaagtcctcacacacccatcctgtgcctttccatcttcccatctgctcatactcccgactctggagagctggggccctgggtcagctggcctggcacccccactcctctgaccagggtggacttgctgttgtgctaagtaagcaactttgaagcagatgcacatgccgcaccctgctgttcctgcctccacccccgcagatgcccagcgcacacaacctgtcctgagtccagcgtactctagatgtgggcatgctgggcccaggaggggccggtggccatgcatgtccttgaccttggtgatgccaaggctggctcctgcaggctccaaggtgccatcctcctgccaagtccaggggccagccccatcctcacaagccaagtcagggcgcttcccagaaactgagaaacactctggcctgcaagccttcacccccgaaaccagctcacagcagatcatgaatccagctgtgctcccaagaaaggtgtcatctcaccagaagcaaagtgtggaggaggattccagcagccaagaacctctccctccacttgagagtgctggcggcagctcactgcccatcccaaggcggaaagcattacagagccactgagtcctccgtatcccaccctgataccgggactctggaaagccccacagactgactctggtcacaaggatgaaactgcaggaaatgggaagggaccagaccagtggctcccccaccaccagggctaatgctgtgcggacaggtccccactgactgcctgagacaaaggagcagctcacatttctgctccagcaagattcaccacctgtctgaagacactctgttactcccccatcagcctcaactcgctgaaaatcacagtcaacagaagctcaatcgtccaagcctccctcctgtcaattcagcttctagaagcccaaactgcctccactgacacctgggctatctgtgtcccacagggtcacataaagacctgtgaccaggtgttttctctgctgggagggcacacctgcagcatcacacagatgggaaacatcaacacggcccagaaagcactttggtgaggggaccagacaggatcatagaggatataaagacaagtgcacaggtgaacagaaggccaggcagtggtcttcctggagaaaactcccaggctggagggcacagaagtccccccagaagcactggggagcccacagtacaacttcctccctgcccatcatcaccaaccatgggccagacctgagtagcatggagcactgaagccctttgtcccaaatgccagctatctccctaggagtgcttctctgaaacatggctgtgcattctggggagaaactgaggcaggcctgcggaagggtgctcacaagtctgcctaggtctagctgcaggtcaggccttcactggcagaagcctcatcctgggagcaaggggtctggcgatcttagaggatgggggaacagctgccccaaaaggcagagggaagaagggctcatcgttgctcaagacagagtcactgtgacttaaagaaagatggggaaaaataagtgtcccactgggccctgaaaataaggctttaatcactatttaaaggttctttagacactaagaactcatatggcaaatccacctgaagaccctcatttctaactatttgggggtaaaaagccatcctccctaagacactgatcacaggaaatgagggacataaacctaatctgtgtagagataaagggacaggcagctcagtgacggggtggccatgggcctcgccgcagaagttgctcagagtgaaggtggctgaggccctccagcacacaggatgccgggtggcccagggcactagggggctgtagctggagaggggctggacttgcacacagaagagcccaggcctgcctgcagcatccacacctccagctgtaccagccccaccacaccggccacctgttgctcaggcaggcagggcaggcagggacagtgaggaacatgtgtcccaagcacacctgcgtgtcccaaggtcccatcgctgcccacaccagccgcttccagaatactgcgaagttaggcccagaacaatatgcactctggcacctgggaaatggcattgccctctggacctggggggtgcatggggctctttggggcaagggatgcgtgcacctgataaagctgctgctgtacccagatgtttcctaagcagaggacacaggagaagatgcacgtcccaggtgctgataagggagcgtcactacaaaggtgtcatggttctgtccatgctgctgctggcagtactataaaacccagacatagggcagcccctgtggcgcagcagtttagtgccgcctgcagcctggagtgtgatcctggagacccgggatcgagtcccacatcgggctccctgcatggagcctgcttctccctctggctgtgtctctgcctctctctctctctctctctatgaatgaatgaatgaatgaatgaatgaatgaatgaatgaataaataaataaataaatctatctttaaaaataaaaataaaaataaaaataaaaataaaaataaaaataaaaataaaaataaaataaaacccagacatgcaaggtgccctgcctcggcgaactaggccccagaggagtggggaatcccatggggcacccaaagcctaaggctggctgaagcagtgactcgtccccatcaaggcttctgctctgagccactcactaacatcctacagcagagcacacacaccctgggatccctttaaatctgcccattcacatggctccagctgggcggttcaaactcccggtcctgcatgactgcctgggtgacacgactgccccgggggcagggagccttccattggagaggacgtccccgcagcatcgacacgcagaaggtcacaaccatgccaagaacctgactcaggatattccgggaaaggccatggtgacatatttccctcgacccagctatttgtcaaatctactgaagcaaacataccaacattctgacaccaccagaacactgaagagcaaaatatgggtcagggaaattctgagtttccaaaaaccaagaccgagtgccctctagtggctctgtgcactgcttcctggagggtgaggagtggacctctgctccctcattaattctagagaggacataaagatcattcatgacagagagccgaagtccctcacttaacatgacagcctcatgtcatcaagatttccaacagaaccgactaagttggtaaatatcaaaggagaaaaagtttccatcaccaattaaacacgaatgaacctgggatgaaggcaatcacatcaatgtaacacaaatgcagcaataaaatccaaattaaataaagcaaatgcaatcaggatgggtcggtgcagacctgctggccccaaatggcagtggagaatggaggacgtcggtactagatcctgatccctccagcaacctgtcaatgggtggaatggcaagtctctttgcaaagaactcaagctctgcacaggggagacacactctccccccaaccatccagggagacacctgtcccgctggccaatggatggatctggagcccaaagtgagctgtctttgtggggaggggtccccttccccctgaacctccacaacatgccctggagcctaagaatgtctggcctcccatgggcgctgctctgtgcccagctgcccacgtggagctggaggtggccagcttccaggggacaggccagctctccctggtctgcatcctgatcatacagcacaggaggggcatgaagtgttggtatctgcccgtttgagcatctcaagatgcaggagcagctgccaagcctgggtgccaccacacacagaagcagcagagtgccagggatgaccttggacttgggagtcccgaggtcaggcactcaatccacggctgacacatctgctagctccatgccctgactccacactctctcccgagagcagcagcaatggcctccagttggagaagcaccctcccagctcttaaccacacgatgaatggtgggggacacagcagcgtgaaggggctccatctcaagacgttcatggaacacctgaacaggacttctctctgctgggcttcttaagccccagggtgacctcagagctcctggggtcgctatggggagaaacccttcctggaaggaagctaacaatgggagagctggatggtaaggggaccgggtgtggaaggcagacagagagacagaatccgagtgccaggacacagccagcctcacatcagctctgcaaacatgtgcccagtgctttcctggcccaggaccccggtgggcaatagaaagaggagtacacacagcccccattcgtgagatgctcctaggacagtgctcagggttggacaagtgcccacaccaaagatctgcacctcttcaccatcagggcacaaattagggcacttgccactagatggagccacccggctcactctcagccaggagctgggaggggtcctgtgtgtcacttccatgccacagcaactgactgtggggcaggtacctccgacatgtccctttcctcgtggtgctatggccagcagaactcagactacgccggccatctgtgaccacgaaagacagagttgttaccaacctgcaggggatgtggagcaagaacaggaaataaaagccttttgttttaaagcggtgagatctggggattatgtgtcaccatggcaaactggactgattcattctgagggcaacagaggaaggaggagacacacaacagtatggaataaacaacaggtgtcccaagatcagagagttgtggaagagcacactatgcctgggagatgcctgcgccgggctccaaacatgagcagtttactctgggtatttcaatgcaagactattttttctaaacttctaaattcaaggtcaaaggaaaatagaaattgaaaccaataatttagaaaataagaacactgtagatcaatatgtcagaaatagagtcaaagaagtacttggaaagaaaccacaggacctgaaatgttttattaaacagacaaatggagggacagtgactccatggttgagctgcctttggctcaggttgggatcccagggtcctgggatcgagtcccgcattgagctctccacagggagtctgcttctccctctgcctatgtctctgtctttctctctctgtgtgtgtcatgaataaataaataaaatttttttaaaaaggcaaatgaaaataagctaagcattcaacttctttaaaactttaaaaaaaatgaagggattaataaaggcaaaagcatataaaattaaattataattaattacattaaaattaaagacacaattagaaacttcaaaacagcacaggaattaatgaaggtatgcaagagccgactccctacagaagataaaaggtaaacacctctgatgaatctgcccaagagaaggagacactcaccaggagaccaggaatggcaaaggaatccacacagagatagaaattaggatatttacattacatacaagtttatcccaggatccattcatctatagactatatatatccccaaagttaacctgagctgagatagaaaacctaaatagagccataaccataataaaaacttgaaggtaggggatccctgggtggctcagctatttggcacctgccttcagcccagggtgtgatcctggagccccgggatcaagtcccacgtcgggctccctacatggagcctgcttctccctctgcctgtgtctctgcttctctctctctctctctctctctctctctctcgatctctcatgaataaataaaaaaccttaaaaaaaagaaactcgaaggtggtgaaaacgagctaaggaaaccccactgagctgggagaaggagtggagggtcagcaggtgccatcccccaccgggtcccatctgggtaccagcagggacccagaaccaatgagaagccgccacatggagttctcttttcctcacgaactcctgttcaaaaccatcccttccagcctcctccttccctctatcccctctattagtggacccttgctctctggtctctcaactggcctattggttcaccatggtctgcacatctccttttttttttttaatttttatttatttatgatagtcacagagagagagagagagagagagaggcagagacataggcagagggagaagcaggctccatgcaccgggagcctgacgtgggatttgatcctgggtctccaggatcacgccctgggccaaaggcaggcgctaaaccactgcgccacccagggatccctccacatctccttttgtaattcctctgccgttctggaataaaccaagttgctactcaataacttcgttgttttcaaggttggcatgatcagaaaatccttcccaaaggcactgagttcctgatggttttatagacaagttttaccaaaccttcaagaaacaagtcttgccttttgttgaaattattttaaagcccagaaaaagagagaaagcatcctaactcactgtgatgattaattttttttaagattttatttatttattcatgagaaacacagggagagaggcagagacacaggcagagggagaagcaggctccatgcaggcagcccaacgtgggactcgatcccaggtctccaggatcaggccctgggctgaaggcggcactaaaccactgatgattaattttatgtgtcaggtagactagatgaatcacaggggcccagacaggtggccaaccatcattctgagcgtgtctgtgaagatgattccagatgaaattcctatttgaatactccccctgagtgacgcgctctgccccccccaaccctgcccccgtgtgggtgggcatcatctcatgtgttgagggcttgaggaacaccagaggggaggaaggaagaactgggcgttcctgcctgtcaatgggggatgcacatctccttccctccatgctcctgctccgcaggccttcagacccagactggaacctgcatggtggctttcccccactctcaggcctctggatttggactaattccccagcagctttcctgggcctcttctcagcctccactgatggtgtgagccaatttcttataataaatctcttcctacatggatggcccactggttctgtttctctagagagccctagctacgcactcattttgcaaaacgagcactccaaataccaaagcctgataaagacatgacaaaacagaaagctatagatgagcgatctgcaggtgatggcccactcatcaaattccacccacaacctgtgtttgtgagtaagcttttattgtaacacagccaagctcattcatatggtttccactacacagcactggagatgaggagctacaacagggcatccggcccacaaagcctaaaatatttactatctggccttaaagtcagctgacctctgccacagatgcagctcaattatggagatgagacagaaatcctgaacacaggttaatcaaatcaagacagacatgattataaagcttcctaaaagtcattccctctgtttcttttcttaaacagactttatttttagggcagttttattatattcatagcaatattgaacagaaagtacagagaattcccatgtactccctgccccagatatgcatggcttccccagtatctacatcccgcaccagggtgctacactattctttttgctacctaaaatatttggtagttttttattgtctctttttaaaatcaatcttttaggtgcctgggtggctcagtcagttaagcatctatctgccttccactcgggtgatggtcccagggtcctgggatcaaatcccacatcaggctccctgctcaggggagagtgtgcttctccctctgctcgctgctccccatgctggtgggtatacatgctctctctctctctctctctaataaataaaatcaaaattaactaataaattaaattaaagcataagctctgtgagaacaggagcctaaaaagtacctggcacatcacagagtcccagtaaggatttgcagaatggatgagggagggaggagagaaagagagccactaagcaaaccctatcttagtaagtgttgccttaatttctggctaatgtaaaatgatagaaacaaacaaaaataatggtagaaacagagttaacactgaatgtgcggggcccaatgaggacccacttccctggatggtgtcatgggcactggtatgtgtccactgaagactctggggtcaaactctccatggagatgtgctgcatttgcacctgccctggggaccatcttgtttcactgggccaacggctaaaaggggaatgagcaggtggcactgcccagcgatggtgccagccagagatgacagcggcagccgtgaagcgtgcccagtgccaggccatcctgagcgcatccacagatgtttgcccactcaatcctccctacaacctctggtggaccatgtctgtgtttctttccagttgtttccttcttactgatctgcaggagctcttcctataatattgcttctttctgatattctaattccttcaattaacttctcatggttgtaggcactgtaactgtctttcaatttaaggtttgtctatcgacttgtttgtggagttttctctcttacaaaagttttatattttaatataatcctatttcttagtcttctttgtttatgcattgtctagcttgtttaagaaagctttcctaggtggctcagtcagctgagtgtctgacttttggtttcaactcaggtcatgatctcggggttgagatccagtcccaagcagggctcagtgctcagtggggagtctgcttgtctctctccctctgccctccctggatcatgcaaatgtgctctctcactctctctctctaaaataaataaataaaatatttaaaaagaagaaagaaagaaagaaagaaagaaagaaagaaagaaagaaagaaagaaagaaagaaagaaaggaaagaaagaaaaagaaaagaaagctttcctaaccccaaagtcacaaaaatatcctctaattttttttcgaaggcttttcaagttttattctttcctattcagttctttgttgcatcccattttacagctgaaaaaattgaggaagagagacagtcagcaaggggtccggggtcatccaattagcacacatgcaccagtcaatgacacatgcaactgttctcaaagttactgaaagccgccacgttgtacacttccttccagaacttgtcctcactcctcacatggtcttgaagattcacccctgaggacatgtaaagaccttcttggttcattctgactgctatacggcatctggtcatacaaacataccagctcgtttatccacccccagggggaggagtgagcaggccaccatgtacccaggaaccagctgctctcaacaacaggtgtctagaaacagcacttctgagtagcagggtatgaactaaatattatgaaattgctcttcaaagcagctgtccatatagcgttcctgcttgcgtggtaagacccttcttccccatgagcatcctcatgaaccttgtatatgaggcagctacatttctccagttgtgaagtatgagatgattcctcattttattgtgcatttctctgatcacgactgatactgagccctgtctcatgcttacgggcccctctggcttcttccatagaaatcccctattcatgtgttttgcccatttttctttcctgttaccgttatttcttattgatttggaacagctatttctgtaacctttcttctttctaatcttctaattctagagttttctaaaatttctaaagttttatttcataatccacctacaatttatttttgcacaagatatgaaattaggatctaattttatatgttccttatggataagcattcctgcactgttaagtgactggtccatccctattcctggtggtaagcagaacgatgtttcctaccccagaaatacccacatcctaacccccagagcctggcaatgtgatgttgcactgccggtaatcatggctgctgatcggagagaccatcctagatgtttgggtgggcccaatgctatcacagcagtcatagcggtcctggtaagtgccagggagccaggaggatcagagtcagagggagatttggaaactgcactgctgacttcgaagagaagagaatgagccaaaggctactagaggctggaaaggcaaggaggtggagcctgccctggagtctccggaaggaaccagccccaccaaaacctctaccttagcctgtgagaccccttttggacttctgacctccagaactgtgggacagcaagctcatgctattttaagccattttacatttatggtaatttgttacaacaataagagtaaagtaatacacttatctataatgtgtccagtcatcacactctgtcttcttatctgtccatttctgccccgatgccacactgttctagttactataactataaatctgccaattaggaaaactgaagcctcttttctacttcaaaattgccttggctattcttgaccatgtaattttttaattaaaaaaaaaaaagaatctatttcaaagtaaaataaatgcagtgcacgagggactacactgcagcatagagcatgagcccaactcagaagtcatgaaaacacacaacatagctggtgaatcacagtagcacagagcagccaggaaaactttcaaaaccactagaccatcaggcctgggtggaggatctcagcagcccaccgacatcaggcaaagacatctgtctcccaccaacgtagagtaagcaaatagggaccagactgaccgccccatgaaacaaatttttttaaagggacaaaatatatgaaacaactggcctcaagacattagaattcaggcaacaaaggacagtgatcctcagggacaggaaacaaaagaagtgggccctgaaaccgccccaactctcagcagctaatgagtctgtgggccacagtacagggagacggggcaccaggggcccccagtggagaaaacagagctgaatgtccaagaagaccgaggaagctcccatttgcagggccaagtgccagacaagacaaggtgcacagaaagagagctctggtcatttacagaggctcccctctagaattcagtaggtgactggtcaggaaagtacccaatgctggggccagagccattggaacagtccccagtgcttacatggggcagaaatagacttactgcacagacaggccaaactggcaaacctcataattcaggagatgtcaggcagagcacacaaaggggtcttgtcgtggggaataattagccctggactaaaagctggtctggtcccacctaacaacccttaagggcaagagcccaaaggatcaaactgtttccaaagctcaagagtattcatcaaatacaaacctatttagcactctcaatcaaagaaaattcacaatatctgcaatccaataaaaaatactgatggcagataaacaggaaaatacaacccaaattaggataaaatgactcaatcaaaactgagccagaattggggcacctggttggctcagttaagtgtctgactcttgattttggttcaggtcatgatctcagggtcctgggattgagttccaagtcgggctccctgctcagcggagggtctgcttctctctctctccctctgcccaccgcctacttatgggtgcactgcacactcctattctctccctctcaaataaataaaatctttttaaaataacttaaaaataaaataaaaataaaaataaattaacataattctgggaagcagagcaagcagcagactgggtttaaagcccagatctgctcacaacagtcaaggcaattgggaaaatgagacccaagcctgcagaccgcagagccacagcgggtacagccggtgttcgccccaagcagccaagtgataaggcaattccatcattgtttagaattaatttgattagaaggtcttcaaacaaaccagctgcttgcttccagcaccacagcggcaccataaactaacccttgaaaactcctttccaggaaaagtcagtatatcttccccataaaagctacctgtacaggcatgtcccccaaggacatgatctattctggcatttccacagacacaggtgaactacacatactacatttcactagtgtcctttttgagtagcttaatccaaaatatttaatcataacatttacctatttttgtaatctggaatactatctttcatatttacctttctcagagcttactatttgttgtatttttatttttatttttttaaagattttatttatttatttatttatttcagaaagagagagagcaaaagcagggtgaggggcagggggagaagcagacgccccactgagcagaaagcccaatgaggggctcaatcccaggacctgggattatgctctgactcaaaggcagatgcttaaccgactgagctgcctaggtaccccttgttttattttttaaatgtaactaagaatatatgtgaaggaagaagagacgctagatttacaggagagaatgagtgggaaataaattgtaccctttgtttagtatttttaagcgatatgttctatgccttcaatgcattcgactttattaagtaaaactggaaacactctaaacaaaccctgagagaaaatccatgtcttccgcccaccactgcagggactccatcagatatcctggcaacagcctcggggctgtggtgctggacgttttcagggctacactggtaagagtggttattagcggacacactggtggtaaacagcaatcccaagagcaatgtgacaagcacaatattcaaaaattgaaagatcaaaacaactaaaaccatcaaatgcactgcctttttgagaagaaggaaaatactcaatgatgtggtttgtcccaaagctacggattgtttggcacctgaatatgttccgatacctcagaaaaccttggaagatcatgaagacagtggccagcatgtccaggtccaggccaaagaactaaggggactgcttccaccaacagtaaattaaattcaacaataccaccttccattaactagattctcaactcagaaaaaatcagtcccaaatgagtcccagacccactagtgcctccgctccagcagccaggacctcagtaagtcctccctggggacagagaagcacccatggggaagatggataggattaagctccccactgtgggcttatacattagttcagtccctacctttctaactttctaagagactaacttcctatatctagaacattcttccagctggagcagtctctagaagcctcaggtgcctgaggctcagtgccctgccaagtgaacttcaccaacacccctctgacatcagccctgcagggacctgacccatgggcttctcagtcagatgctggcacaggggtccttaggggtccttatggtccttaggggtggtgaggcagaagccaagaggccctgaccagcaggtgctaggccagtcagacacaaggctgacagcaacgttcgctgctccgccacctgcacacatgcagacacatcactgcccccaggaagatgttgctctgagggtagccctcactctccccaaagtcgccagctgagatcagacaccccattcttggaatgcttcaagggagtaaaaagcaccttttctaaacaacaaatgtacagcacgttaacaacaacacccagagatggcagcccatggctctcggtcaaactcttactctcaacgctgcgtctataggactcccatgccaagtgcccattaggtgtcactggcagagccttccacaagcctctagtcagggggctgcaagacacaggtgacaacacacttggccaccctctgtcactgtccaattctacatccccgtattaagcaaaagctcaaccaactgactccaattaaagcaatctgtagcaattatctgataattgttattgtactggactaagtatttttccttaagaacagatggaagtcatgctatgtgctcaaaaagtcagttctctgcaaacacttacttgcaggagatgtgggtggcttcctggagggctgccgctccgcctcagggctgtggaccagccccgtgggtgtgtccaggtgtccctcagggctcagcgagaagtggaactggacagtccctgactccacttgctgcacctacaatcaaaacaagaacaatggcagctgcctcctttcatcggtgcccttctattacaatgcaaccagccccaaagtctacataaaaccagggattcctgaagccccgtgttctcagatcataatcaagtcactttctgagcagaaacaatcagccgtggcattgaaatcccagaagaccacattagcacagaatggctcttactcaaggagaaattacatttccccagttttgcacgtttttaaacaaacctatgcaaaactgaccttttaaatgttggtatgtagttctgttcaacacatgtactcactaccggagttggggtgcagcgtggctccatcacccaaacaatccctcgtgttgccccagtctgactcccccaaccccctgtttctgagctgctcctgtcatggcagcctcggctctccagaatgccacagacacggaacccaaggatatataattaagcctagcttcttacactccacataatgctgatgagactcatcccagtaagtagtacgtatcaatagttggttccttttcctgctgagcagtattctatggtaccatgtgcatggtttattcatccatttaagcacatttaggctcctttcagtttgggtggttacaaacaccggggtacatgtgtccatgtgaacctaagtcttcatatctctaggaacaggattgctgggtcacgtggtacgtgtatatttaatcctataggacaccatggtttacttctagagtactctgctcccaatttatatgccaatcccgcagcaacagcaccttacagtggttgtcgcagctctactccaagtgtcacactgtggggaaggattcctccaacatcactgctatcgtcacagttctttggactcttctaattcctgggactttctgtataaattttaggatcagtttctctacatgtagaccaaaaaaaaaaaaaatcctactggaattttgaataaaattacattatctgtacatgaattaggggagaatttctatcttcaacacactgaatattcccatgcataaagatgctgtgtctctccatttacttagagtcctctgtgatctcacccaagagtaaccacacgagaactgtgacatgcaccaaagggaatatgctacagttcacaacagacagatcctgtacacactgtgttagatttatcttactgtgggaaggccttgataagtaatactgttttttttttattttgttcatcaactgccctttcttaatatacagaaaagattgatttttctatcttgatctcctattgtgtcttgcaacctcaataaagtcactaattcattctagcaggtttttgtagatcccttgggattttccacacaatcatgctgcctgcaagtaagtacagttctgtttctctctttccaatctatgtgtcttttattttgtcttcttgccttaacgccctggctaggactttgagtatgatgctgaacactgagtgcacatccctcacttattcctgatcttggggcgggggggcacccaaactcttaccactccataggctgctcactgatgggtttgtgtggatgccccccaagcaggaagagggactgctcttctactgttagtatgctcacagtttttatcataagtcagtgttaaataaatcttatcaaatgcctttcatgtgtcatacagttcatctcccttaggctattactatggtgaattatatttattgatttcttgcatttctgggataaacccccacttggtcatggcctagtcttccttccacattgttgggttctatttgctagcattttgttggttgttttgcaactgtgtccatgaaggacacatgtccatggtcttcttatgctgtctctggttttattatcagggcagcacaagcctcattacatgaattaggagatgttcccatccttctatttcctgggggagaccgtattaaactactcttctgtctcttccttaagtcattctgtagaattcacagtgaataggcctggatgttctcaacagtgttttagtcatgaattcagtttctataatggtcataggactactcaggttgtctacatcatcctgagtgagttttgatcatttctgatttaagataaactgatccatttcatcgaaattgccaagtttacaggcacagaattgctcacagtgttcctttgctgtctgtttgatgcctgtggggccagtagtgctggtccttcttccattcctaacatcagcagtttgagtcctcctctcctgttgtcgttcttgctagacatttatcaattttattgatctttccaaagcctcagtttttggtttcattgatttcctctgttttcgttctctatttgttctgttctgtttcttctgctggatttagggttattttgttcttcttttttttaattttatttatttattcatgagagacacaaagagagagaggcagagacacaggcagggggagaagcgggctccatgcgggcagcccgatgtgggacttgatcccaggactccaggatcatgccctgagctgaaggcagacccttaactgctgagccacccaggcgtccctgtttctctttctcaagctcctaaaagtagaagataagattactgaatgaagaccattcttctgttccaacacaagcccttaatgctatcaatttccctggaagcacatgtcaagtgcatcctccaccttttgatgtattttcatttttgttcagtccaaaacatcctctatttcctcctgagtcttcctctttgatctatgggttatttagaaatgtattatttacttttcaaatgcctatactttttaatcttatctttgctaccgacttctggttaaattctgttatgaccgaaaaatgtaccctatataattccaattctttagaaatcatgaagatttgttttatggcccaggatgtggtctggctgagtgttccctgtgccctagaaaagactatgtgttctgctgctgttgggtgcagagttctgtagacgtcatgttgatccagacggttcatgatgtcctttagctgtttcatgtcctcagtgattttctctatgttccctggtttacttagagcgatgttaaagtctccaactataactatgagtttgtctatttgtcctttaacttctagtaattagtttttttgctttatgtattttgaagctctgcctatactggatgcacacacctttaggactgctatgtcttcttggttaattgacttttttatcatatgtcatatccttcttaattcctgaaaatttcctttgctccaaagtccactctgtctggtgttgatacagccactcctgttttcttttggtttttgttttcgtttttttttaaagatttatttattcatgagagacacacacacagagagagaggcagagacacaggcagagggagaagcagactccatgcaggaagcccgatgtgggactcaatcccggggctccaggatcacaccctgggtcaaaggcagcgctaaaccgctgagccacccgggctgccttcctgttttcttttgattggtgtttacattgaatgttttggtgtctgttgagtgtcttttctctgaaagttgagattttcctgcctctttgcatctcaattatgtttagattatatcacagacattttgaatactatgttaggagactgtgagtctcatttaaattctgtggagaatggtgatatttttgtttcgtcacgaatcagctcaatgggttcaggccacagtttccaaccagccttctatgcattgtcagcccctgtgccatgcttttcagatccctcctgcatgtgatccacccagggaaactctggaacggggctggaggcctagccacagtcagttctcagggtctttggaactgtagttcagggtcagactcaggcagcctgagggttaacccagcatttcttaatgttatgggattgctttccaagcctctccctctccatagcctccccaaacctgtctgggactttacaatcctccagccagaccccactgcgctgcacacttctgggaacaaacagtccctgaagccaagacccctcagggggaggacagagtagtttcttaaaagagctcaggagtgtatcaagcaagttctcatttatcattattggaagtcaatatataatgtctaaagttgataaacctaagaaatagcagaagcataatatttggaaacaaagtaaatatcataagtcagagcaaagagttgaaagtggctgcctctggagatagaaattttttttaaaagacaattatttgctcaaaggaaaaatacaggagttaagaaagaaaacataatcataatgtactacttagcttcacagtaacatcaaaataataacagaaacccccagatttacgtagccaaaaattatgggatgtttctaatatttatacttttctctgattagtatctgccaattccccttaactgtaagttcacaggatcggggatctttttccaagggtataagtgctcagtaaaagttcactgaatgggtaagtaagacacagaatgacctggcataggggcagagacaaggtgcacctcactcaggggcatctctgcccctaagcctccacactacctgcccagacatggcactgctgtagaagacacaatgggaagaagcagaggaggaagcagaattaagtttatggccagttttagttcattattttaattagttatgtattttagaatagtgatcctcgactagaggcaattttgcccttgagagacatttggcaacgtctgcagacaactcagtctatcatagttgggggcacgctactggcatccagcagacagaggccagggctggtaacgaacatcctccaatgcacaggacaacccccacagcaaagaattatctggcccaaaatgtcagcagtgctgagggtgagataacccaattagaagtgatattccttgtgaataatggcagaatttgatatatttgtgtcctcaagatagtcaagccttccatactcatccccaatgcagtttacacactcagggagtgaaggagatgtagtggcatgacaaggctaaagttaaatagaatagatatatatttaaatacatacacatatacatccagagagggaaagacagaggcaatggctccaggatctctcacggcagttttataataagggctcagactagtccatgggggaaggcagcaaaatgtgctcccataatgagatatccttataagaagggaagctgcacaatcagctgttcctgatccaggtccctaaagcatcacccacatcataaataacattgaaaataacatttgtggcgagagtcagcacacggatgatgcatggaataccagggatggtcctttcacctgagaaaaatgagacaactgagctgtagacggctcccagccacactgctgaaacagcagagtgtggtacaggagtgcaactgggagaaccaagcacccttgcgtgacactggcgtcaggatgggccagccagagcagtgctttcctgcgcggggtacctgcagctccctggcaggtgacccctcactcacacttcaactggccctgccaccgtttaccggtcttctccaccggggttttggagatagcttcattccaaacatggattttaaaagctcctgctgtggcccgccctccatgcaaagctggaaggagccaagggccagtgggcaagagtaacaagtcacagagatgcaccgggctcaacaccaactgccaggcactgaccacaaccctggtaccaccaaagctcattctccatagcaaaaggttccaagagtcctttttgaaaagacacatatagcacagaagcagatgctgtttgcacagtgcatacaaataaaagccacaggccccacaggccaccttgttgagacttatgtgtgaatggggatgtcctaagccccgagataaaacctgcctcagtgagttcacagccaactatgactcagcaatcatgcttgtcctttaagggcacgtccatgacatgaaaaaaattctgttcatcacatatccacaaacatatctgtcaacttctggttttgaaaatgtgtgaacttgctggaagctcacaggcaaagattcatacttataaagtatctgaacaacttacctcttcagagctcatgctggcaggggttactttatagaccaaacagtgtgaggggttgggccggatcccaccctgcagaggtaagatcaccttcccagagccggattccagcaaggggttccaaacagcccagcgaactatgtgcatacacgccacactggacaaagaggtgaccgaggctgcctaagaagggaaatgaggcacattggtacatgacttaccaccaaacccaccgtgacttaccaccaaaccaaccctgcaaacgtcactagggagcagtgcagccaagcagggatcacacagaaggaaccagaccaggaggagggcaacatggaagcttggggcagggggcagggaagcagcatccaacctaaccagtaccctttcctatactgatctttcttctggctcaaggaagtagcagcttttcttttaaagaccaacaccatccttctgatccctcctgttcccccgaggctcttgtgcagatctataagcatgctgtatcttgactacatctgcccacatccattaggaaaatactaggaatgacacttggtgttagtgacaggaaactggaaaataatgaagaatgcccatggaagttcaatttggtattatttgcacacattttctccaaccaccttttgctcataatgaatggtacatttcatatacgtgactctccatggatctgggaagttggccaggtccaattttctgcaccgtcccttcttccgccttctctatcactgaccaagtgaaagcaatgaggacactaaggaaaccggtttcagggctcaactcagcagttttaatggcaccaccttcatccattcacccagaaagaattaagaataaagtcacactccaggcctggaacccgtgctcagctgggcagtgagacttgcgattgccgccttgggggaggcaacatcagagaccccagctctgactgacagatgccgcaggttccttgggaggcagcacgtcaggaccccaggaagaggccatggggcacttggcaagagtccaggcaggtgttttccagcttcagagcacagctgtcctgaccagagtcacattgaaactatccagcccaaggaggcagcagccacacctgtcaccctgactcccacctcagtggtgatgtgggaggaggaaaacctgaaggtgtaggcagtacttggagcatgcacacagcattccacctcttaatgccctcttcacaggtggcagacagctgtctgtgcccctctgggatgaggacagcaaaggaactgggtgactccgtgtggtatccaggtggctgagggaccaaagccctctgatgctctaaaccaccagccacatctgtgaacccagacagccctctcaatgggggccacaggctagggaggctgggcagcctacaaggagggggcctcgagctcttagaaaggttggaagcccatacctcatctgatggtaaaggacatcatccccaaggagaagagagctgtatcagaagccacagagtaaggcaagactccagcagagctgctaaggctcccaacagaccccgctcacttgctgggcgtgggtgagctgccgtgccattgtggcacggacttgtggcatgggcaggactgactgagcttgagtccaaggcagactgtggcacacatacacaaaaggactctggccagaacccaatgcagatggttcaatggtcagccaggccacaccccagccgagcatatccatgagaagcatccaacatggaaaaggaagcacattcatctcacccaagtcagactatccagactgttccttagtcaggtagtctcccagcctgctgtatccctagtcacatggggataaatgtccagagtcagtgtgtggtcttgaattaatccctttcctgaagccccattgcaagtggcccaacctcacttcagcagggcctgttctcattctttactggagcccaacttcagatcataggctaggctcccatcacatatgcctgggtcacaggggtaaccaggtccctgccagttcaatgacaaaaaacaccaatcctgaaagtacaaagaaccagcaaagtaagaagcctaagcatatgtctctgccacacttcaaacgtgtcaaatacgtagccaatgcctgccgctcttcatccggttagctgatgacagtgacataaaatggtactgagcaactgtgcaaagttgactataaactcactgctctttaaaaacaaaattactataaatgtattggcaaaactggacactaaaaaaccatgtgattcgaccatttctggactacttttataaacagccatctcctataaaatccagtttattactggatcaaaaataaaatggcacacctgggtggctcagcggttaggcatttgccttccagctcagggcatgatcctggaatcccaggatcaagtcccacatcgggctccctgcatggagcctgcttctctctctgcctttctctctctgtgtctctaataaataaataaaatctttttaaaattttttaaatttaaaaaaaataataagataaagtatgtatccaagcataacacatgctattcgtgcaaatcttctggtttagtctttcaaattaggcgattctgatatgcagttttccaatttcacagtccctctggtttcctagttctcaatcgataatggctctcccctacgcggagtacctgtcagttatacacatacccatttcccctcactttcgtctctaacagctacaacaagaagagatggctagcgagtcactgatcactcctttgcaaagatttctgtgacaattaggacttgttggcaatttatcagcctgtgccctcttctgccaggctggggtgacacctgcaggtgagaacctcctcaatctcacacacctcgaatccccccagctaggtccctggctctgagcaggtcaggtgcatccaagcaggtgggcagaagaaaagcaactatttaactggtcaacaaaacaaggctcttcctgtcagtgaatctagtggttgagataaaatcagttgctttctaacatcttatccgcccaattgggtttggcttcaagaaaagaaatacaaactgaaaagagaaaaaaaatattgacttgttccccatttagacttttatttataacaccacataatatacaagaggccaaaagagtacacctgggttatatataaaaagccaggttcttcaggaacagaagaactgcaccaacagtcaccaggcaaagcccaaacagacaagggaccctgacctctgggaatgctgtaaaccaggttccctagggtgggtgccatggtggcttcaagaaggcaagtcctcagcctaatccctggaccctttgtgacctgatttggacaaagggtccacgtaaacgtaattaagtattatgagattaggagacctagattatccaggtaggcctcaaatccaacgacaagtgtccttagaaagcaggatagagatttgagacagagggaaggccctgtgaatgagaaggcagaagctgaagcaacatggccatgaacagagggaggacagcaggcgccagaacctgttagaggcaggagggaccctcactggagcctctggagggaaccccaccctgtaggcccactgctttcagcccccatttgcagctgattgtcaatctgtggcctccagagctgagagaataaatgtggattgctttaagacacatccagttcatatcaatgaacagcagacactcatgcaccacgacgaggcaccgaaaccacagtggcatgcgggatctccccagtaagtgaccctgggcggcaggatggcaccaacgcgctgagcataaaggtgaaagtggacacaatgaacccctgcaggaacaggatgggctctcctaggacacctcaaatcgagacataacagacatcaaggcaggcagatctgcctacaggaaaaatggtgctgaggtggccaagacaccctgggccaaggcccatgggtgaaaaccctgagttccagtgggacaccacttgcggtggggtggggggtgcctctgggaggtgacacaaatccgatgaaagtgttggggggtgtttttagtgtccttgcaagaaggggaggaggggagagagacctcccgccttccacacacacacaccaacacactcctacacacccctacacacacacaccagggcagcagctgcaaggtggggccccactgggaacccatctgcggctccagcacctggagggataggagtctgacgttcacatggcatttccttcggctgccacagcagataaagacaaggccaaacacaggggtgtgggtcaaaggcagaccgcagagcaggaagaggtgtcacacactgtcccgagggagaacaggtaccctcactcacaaaaatcaccacagaaaaacgggttaaagaagtgaccagacagacaacccacaacccgactgcacattccacttcacgaactgtccctcaccatgaggccatttctcccctccaagtagcagaacccaaagtatgagaggccactcctgcaaaggatcatgctcttgcccctggacccagcatctccttggggcgcacgcgcacagccctcaatccagcacacccttggggcacacacccctcgatcccgcatgccctcaggcgcatgctcacacccctcaatctagcatgccctcaaggtgcatgcctacaccccttgatccagctcaccctcggggcacatgctcacacccctcaatcccgcacaccctcagggtacaggctcacactcctcgatgtggcatgccctccgcgtgtgtgcctacacccctcgatccaggacctcctcagggcgcacagccatacccctggacccagcacccctcagagtgcatgctcacacctctcaatccagcacacccttggggtgcatgctcacaccccttgatcccacatgccctcaaggcacatgctcacacccctcaatctggcaccccctcagggcacatgctcacaccccctcaatctggcacctcctgggggcgcaccctggagacaccccagagcacaaggtgagccaacacacagacagttccacccacacctggtccacctataggccctgcaaattgctgagctatccaaccacaggaaatgcatgcacaagctggggggcacaagcagagtacatgccgttggaaaaggaggagacacgctctgtggccagccccgcctgatggccagggcaccccaagagtcacccacgcacatggccacttccctccctcaccccaagaagcagcccaggagggatgcactgggagcaacagcagcggctggctccagaatgaacagtggaagggacacagaggagcaatgcccttctgaaaccagctttgggtagaatttttactgaaaagcctatcagtgtcctacatgttccggaagtgaaatgaaatcactaggatgggagggaaataactctgaactagagcacagataaaaacaggatggtgaaagactcctaactctgggaaacaaactaggggtggtggaaggggaggtgggcgaggggtgggggtgactgggtgacgggcactgaggtgggcacttgacgggctgagcactgggtgttattctatatgttggcaaattgaacaccaataaaaaataaatttataataaaaacaggatggcccactgtgaccgagtgagcagtgaggctacaccattcgggccaggcctaggggctctggacactcttcccctccatctgggtggccagagccctgaacggctgggagggccatctggcagcagcagtgtgggtgaggcaaccctgcgctgaggctgatggtgccctgggcttctccctggggaaggaggctcggagagccttgccagtcggctgggatcggcaagaactgggttttttttttgttttgttttgtttaagaactgggttcttaaggtcaatctgtgtacatggcaagtgcacatgtgtgtgctcacatgtgtgcatatgcgcatatgtgtttctagaggtaagtctcccagctctgtccactgacatcagatctgggaaaccaaaacacaccagcacccagatctggattcaaatacatcccctttaaaaagaaccagggtgggagcagcccaggtgacgcagcggtttggcgctgccttcagcccagggcctgatcctggagaccctggatcaagtcctgcgtcgggctcccggcatggagcctgcttctccctctgcctgtgtctctgcctctctctctctctctgtctcttatgaataaataaaatctttaaaaaaaaaaaaaaaaagaaccagggttcatcaaaaacaaggaaagtcagaaactgtcacagtccagaggtgtgtgagaagacacgatgactacatgatgcatgacatcccaggtggggtcctagaacagaagaggactttaggaaaggaaattcaaataaagcatggacttaggttggtaacaatatatcgatactggttcattagttgtgacaaatgtaccatagtaaggaggctaataagaatggaactggggacaggtatgtgggatctctgtattatccacacagtttttctgtaaatctaaaacttttctgaaataaaaagtttattgagaaaaggcaggagtggggagaccacagctccccacagaaatggctgatgtcgaggctggaatggggaagctgtgagatgagcctggggcaccttgttctaccagaatgtaagaaactgctcggaaaacagaacaccatggaggatgtcacaaggacacagcctgaagggactacggacaactccaggacaatctgagcactaaaataatcaggaacagtagattggtttttttaaaaggtgtccttgagtccatcccaatgtagagctggaaagcctcactcagaggcagcaaggatgccaaccccggcagatgtctgtgtggcctgaagggtctccccacaaatttcttgtaaggacaaaatcagttactgtccagtggggacatcacatgacacccgactggtgatcaaaacaggccaaaaaggtgggagggctgccacgcacatctgatgtgggactctgcgggacacagcccaccccactgcagtgttctagcagagatgcagaggctgaccccattgggaggaagcatcactaaatccaacaggggaacatgtactttaaaacgtttttaccaaaatctataaagaacttattaaattcaacacccaaaaaacaaagaatccagtcaagaaatgggcagaagacaagaacagacatttctccaaagaagacatacaaatggccaaaagacacatgaaatcagggaaatacagatcaaaaccacgagataccacctcacactggtgagaatggctaaaattaacaaatcaggaaacaacagatgttggtaaggacgtggagaaagaggaaccctcttacgctgttggtgggaatccagactggtttagccattctggaaaacagcgtgggggttgctcaaaaagttaaaaacagggttaccctataatccagcaactgtactgccctaggtatttaccctaaggatacaaacattgtgtggcctgaaggggcacctggacccgaatctttatagcagccatgtccataatagccaaccaacagaaagagcccagatgcccatcgacaatgaatggataaagaagatgtggtgtatatatagaatggaatactaatcagccatcaaaaaatgaaatcttaccatttgcaatgacatggatggaaccagagggtattatgttaggtgaaataagtcagtcagagaaagacaattatatgatttcactcatatgtggaatttaggaaacaaaacaggggatcataagggaagggagggaaaaataaaataagatgaaatcagaggggtagacaaaccataaaagactcttaactctaagaaacaaactgaagaacactggagggaaggggggataggcattaagaagggcatgtgatgaaaaaaaaaaaaaaagaagggcacgtgatggaacgagcactgggtattatacatgcaactgatgaatccctgaactctatctgtaacactaaaaaataaattaattaaaaataaaatgtttccaaaaggagggaaaatgtcctataaaaataaaatcagtaaatccaaaaaaaggctgaggagctgctccagatcagaggagaccctgaaggcaggaaccgtaccaccatgagccctgcactggagagaaatgccaccaaggacacaccaggtcgctggcaggtctggattacagtggagattatggtcagactcagtgctgagccaggcaggctgctgtgtgtgcccatggaagggcatctcagctctgcagaaatgcacactgaagcatgtgaagtcaggggctgtgacgtctgtaagttacccccaaagagctcacaaaagataattgtatggatctgcacacacacgtacacatgcacagaggacaggtgggaggaagaaatactggccagaaggtatggataaagcaagtgtggcaggtgcgaacatctgaatctgggttaagaatagaccagtagtcctagtaattttgtaactggtctgtaagtttaaaatcatgtctaaataaaaagtttttaaagaaattatgcaatatctactatgccttttctttttccaagaactatgttaatatgaaaaggcaagtgacagaccatggaaaatattcacaatctatatatctgatgcaagatttatagcaaaaatatataaaggaaattcccatctatccaataataaaaagacacacaatccaaaacatggactggccacttcatgaaggaagaataggaatggccaacaaacatgggaacatatattcttgcctggtcattaaggaaaagcaaattaagaccacattcacacccactagaaagactgaagtcaaaaaatagaaaccaaaagctggtgaggatacggagaaactgtaagcttcaaacagtgctggtgcaagcaccttgggaaaatatttaacatgtatttacctaggacccagaacatggttccatagcactcaataacaagaaatgaaaatgtatgtccacaaagacttgcccaagaatgctgaaagcagctttatttattagctggaaaagcctaccaatggtaaaatgaaaaaacaaatttgcaacccacattatctgataagagattaacatccagggacacctggatggctcagtggttgaacatctgcctttggctcaggtcatgatcctggggtcctgggattgaatcccatatcacacttactgcggagagcctgcttctctgtctgcccatgtctctgtgtctctcatatataaataaaatattaaaaaaaaattaacatccagaacacagagagaacttcctaagcctgcaacaaaacaacaaaccacctgattcaaaaccaggcaaaggactcaaacagacatatctcctatgaagatacaggccaacaagcatgcaaaaagatgctcagcatcattgaccatcagggaaatgcaaatcaaaaccacagggaaaccccacctcacacccattcactgactattatcaaaaaacaaacaaacaaacaatgagtgatggcaagaacgtggagacaccagaaccctggtgtactatcggtaggaacacaaaatggcacggctgtggtggaaaacagtgtgggggttcctcaaaaaaattaaaaatagaatcaccacatgattcaataattcttctggtatataccccaaagaactgaaagtaggatctcgaagggatatgtgtatacacatgttcatgttaacattattcacaatgtttgaaatgtggaagccacccaaatccatcaatcgatgactaaatgagcaaaacgcagcatatccattcctcagtcataagaaggaaggaaattcctaaaatgcaacaacgtggatgggccatgagcacacaattctgagtgaaagaaaccagccacaaaaggacaaatactatatgattccacatacatgaggtccttagagttgtcataaccataaagctgtcaaatatacggtgggagccaggggctgggggtgaggaatgaggagtcagtgcttcatggggccagagttccagattcacatgatgtgaattctggggacagacggtggtgacagctgcgtaacaaatgtaaatgtccttgaagccaccaaattgcacacctaaaaatagttaagatggtaaactttgtgttatgtgtatttttacaagaaaaaagagacatgaaaaaaaaaaacaaaggtaagtcagaatgccactaatctatacactttaaaattattctaggccttgtgggtttcttagtaaaataaactacagttgaaaaagataaaccaaaaaaaaaaacaattttcaaaaagtgagttgagggcagcaccggtggcccagcggtttggcgccgcctgcagcccagggtgtgatcctggagacctgggatcgagtcccacgtcaggctccctgcatggagcctgcttctgtctctgcctctctctctctctctgtctctgtctctgtctctcatgaataaataaataaaatctttaaaaaaaaaagtgagttgatatctttcttaaagctataaggggagcctaagtctgaaagacttccagaaaaccctatagatatctatatttctaagttttgtctttttaaaaacgtttttttaagattttatttatttattcatgagagacacagagagagagaggcagagacatagacagagggacaaacaggcttcaatgcagggagtctgatatgggactcaatcccaggtctccaggatcaggccctgggctgaaggcggtgctaaaccgctgagccacccgggctcctctatttccaagttttacaagtttaactttacatcattttcagaataaatcgagacataactcttatgtgccaaatatgcaacatcaaaatattcagttctgaggattcccaggcacctggcaagcactcagatctttgttatatggacttacattgtccaaagaatgctagaaaatacataaataataaattagttaatcttcaccttctagagatctgggagttattttgctatgtgactccgaaaaaaaggactgttttgaccgctagcagccctgtggggatttgcacttattctgacataaaaatgcagcagaagacaccgggcctggttatttccacagctaccgtcattctgtgtcactctcttctgctccagggaatctgctttatcaactttcacaatcagtcaatgctgatggagcccacacctctgtggcttccagcattttcaggcctcctaatgcaggtgcactgtccccatgtattcaaattcctgggaaggaagctatctggatgggaagaaatactttctctgtctctttacagttcacccagagacaaattacaaatggctttagttgttctaagtaaaagatgcaaacattaccaacagattaccaacaaggagcatgtcttctcaaaatggtctttctagtttgttttccatgactaccaaaatactcaggaccaaaaaagttccctcagactcaggtaagcaggtctaaatgggatacactcaacctggatggtttctcccatttaccactcatcggtgaccaatctcacacagggccccaggacactgaggcttttggctcctagtccaccccgtggacaaggcctggcccattctgtgtctccaagcgttaggcacaaaagaacacaagacataaggaacatggcaaggtcatcctgagaacaggtcatggcacaagaggcatccgtgagggagcatacagctgccccacttaggaagattggttcagaaatcatttttcaaagaaaagtacatttactttttttggctcttctgattctgtacttgagtacttgagaaacatccaggcaggctgactaaatactgggtgacactggacaaatctgtgtcagtattttaggtgtgactatggtactgcagcagcacgcttaagccccattgttagagacacagagggacccgttcacagaggtggcagcgggctgtctgggactgacccagctggactggggaggagtgggggtgggcacctccttctctctatcttcatttgaaattttaagtaacaaaaaggttaaaagaagaaaagaacctatgtgccacaatgttcacagtaatcaaagcagaaaatgactggaatttgcttcaaaataaatcaaaatgggggatccctgggtggcgcagcagtttggcgcctgcctttggcccagggcgtgatcctggagacccgggatcgaatcccatgtcgggctcccggtgcatggagcctgcttctccctctgcctgtgtctctgcctctctctctctgtgtgactatcatgaataaataaataaaatctttaataaaaaaaaaaataaaaataaaaaataaataaataaatcaaaatgggtcgtgggagatggtacagacaatataggcctgcacatgcagttggtaactgctgagccagctggttcattttactacacggtctgccttggtttacctctgaaatgtccatcgtatgaagctctgacacagggaacaaaggggagaaagactggcaggcgggatgcaagcaaagagtgcgctccacggctctcctatctgccaagggatcggggtgtgctggctcagcctgtccccacaccccgggctgcgcccccacaacacacattcctggggaaggatgggggcccagccagcagcacccctgtgccctcccctctaatgtggagacctgtctgtgatgggagtgccctagtcaggccagccccatgcccacagcaggcagcctgggcggatcaccacctcacagccaggaccctccttcccagactgcacccaagaaagtgcacccaatggcagtccagtagaatccagtattctgtgtgaagagaaacctccccaccactaagaatcgatgagctagataagccctgtttcgtaaggcagcaacacacagtaaaaccacctgcaggttaagcactgcagggaagagttaaaaaaataataaagcatcatcaatgaaacgccgggacacctgcaccccgctgtttatagcagcaatgtctacaatagccaaactgtggaaggagccttggtgtccatcaaaatgcatggataaagaagatgtggtttatgtatacgatggaatattactcagccattagaaatgacaaatatccaccatttgcttcaacatggatggaactggagggtattatgctgagtgaaataagtcaatcggagaaggacaaacatatatggtctcattcatttggggaatataaataatagtgaaagggaatagaagggaagggagaagaaatgggtaggaaatatcagaaagggagacagaacatgaagactcctaactctgggaaacgaactaggggtggtggaaggggaggagggcggggggtgggggtgaatgggtgacaggcactgagaggggcacttgacgggatgagcactggatgttattctgtatgttggcaaattgaacaccaataaaaaataaatttattattaaataaataaataataataataataataataaagtgtcatgtgagacacaccagtttctgtatacatactctgtgatcaccagcagaccgtaattcaacattctagagtggatgctaatgacacgtccacagacgcagagagcgccaaacgcacacagcaacttggccggctccctccctgtctccttccaagggcagcacagggggccaccccaggagccccctccctcctggatgagtcccccacccaagcccagagtgccctccccacgcagccaggtgccttcacatacagttgcatttcagtggcaatttctggtaataaataaagcagagctggaaacgacggcttgattaaaacaactgtgaccaaaaaataaataaataaaacaactgtgaccatgtgttcagccataatcagaacacctgcttctagatttgttcagctgatcaggactctgaattatactgacctgaggtgtggacggcagagagcagatgagaaaataaaacagcacctccactcagcacccccttcaccttcctaaggctcccctcaagaccatacttggtcctggtcctccagcagcaaggctgcaatgctggcagaccgactgccatggaaggtgccttgtcatccttttatttctaatttacatgttaaattgatatttacttattcaaacagaaaattgaatcttaagggtagtatgatttgagtcttctaaagactctccatcagtgatacaatgtctaataaataaataaataaaattaaaaaaaaaaaaaaaccaatgtctatatgctatacagggacccatgaataacccggggctgaaggacagcaacccccacgcagtcaaaagcctgcatgtaacttctgacttctccaaaaacttaactactaacaccattcagttgaccagaggccttaccactaacagcaacagtcagtgaacacatatttcgtatgtcgtatgtattatatgctgtgttctcaccataaagtcagccagaggttagaaaatgtcattaagaaaaccatgagaagaaaaatacacttacagtactgtatctaacaaaaaaaaaaaaaaaaaatctgttgtaagtggccccacgcagttcaaacctatgttgctcaagggtcaaccacatttcttctaagtgaatcttacaaagtcttagctatgatacaggcacacacaaagttcactgtaagaaaacatttaccacttagtaatggtaaaaacagtaaagtgcatagtaaagctttactatccttaacatcatacgaagaatacattaaaatagactcatttttcacaattatcaacttgatatttgtgaattaatgctattatgcatgcctattgaactactgattaccatcggccagcagaacattccatcaccagaaagaggggcaatgattgcaacaaagccacaaccaggaagaagcgttttctataatctcattctacacccgttatgtacactgctgataatcacgctgctctgaacagaaacaatgttagtggtgactttgtttctccttggggcacggagcccagggccttctcaccttgctgtccactccagaagggctggtgaacacgtactccagttgaaagactatggcaaacgcagggtgatggaccatctcagggagtcggaggcggcttctcaaaactagagcctggtttccagagctgggtcaagaacacagagcagcagcacatcaggcagtgttcctgccaccctgcaccccccagggtgccaggccctgctgctgtggcaggttcccccagcaatgctgcagagctctgcactgtgtctggcccccaaggtagatcctaaaccagagcctctcatgcttcagtcgacactcattccaaatcgccagagcttacatcaggaggacagaaccacatggccaggtctccgaggaggctcgtcctccagggcacccctcggagaggtctctgatcctgacttgggaatagaggactgaacagaatggcagaacttcctgcctgctgcttctccaccacgtgagagactgatcacagacacagtgggggtataaagtgtgctagggtcccctcagatcctaagcagccacacctggagacacctataatgttagctccagaactgtcacagcacagttcaggatttttccaacctgataacactgaggtctgctcctaacagatataaatcagcaagaggaaacaaaggcaccagggaagaagtccaggtggacaagtgcatacttcacatgcaaaaccagctccatttccctttgatccccagtaatgtgcaccctagtacctcctaattccacgaggctggacatcacgaccctagggagaatacctggtcttcgaagaggagccaactttcctgctgaaactggcagagcgcgtcaaggccacatccatctcgggcaccagcaccaccacgtgtggcctctgcacgaagcccagaccattgtgcacgcccacacacaggcggcgctccaagatctccagcgtgctgccatctagtggactgtccccctagaagagcacatgtcacacacagtaaatccctgtgcagaagggcagaagctcccagtggagaggcaaacaaagagaagaaactgtaactgaagtctcaggcatttccagatcagatggtgctcagtgaaaatgaattcaggcaaataatcctatgagaaccgccagcctcagggcattttctaggtagttctgggtctttacacatagcacggtcttctcagagcaatatcaccctccccctagaagatacctgaatcaccaaagtcttactcaggtttccatggctgagctcaattcttaggatgcagcaaatgagcaaccagggccctagcgccccaaggcagagaagcccactgcccccaaggtgcctctatcctcccccagactcagacaccagaggcttggggtggaaggggcagcaggaaaagggagcacatttatgcagtaagtcataaaccaataaacacagaatctgccccaaagtgatcgctggatattaaagtttttcatcttaacaaaatatgtacaggcactttaacaaatgagtcaggatagtcctccaggctattactgtttattaaattacgtgatcgtggtgaaatgattcctgaattttcaagttttcttcactgcccattaatcaagctgaacagacctaaattcagcctattagtctctgagtaatgaaaacagagcgatggaggaaacagagtagctaccagttggataaggacaaaaatacaaagtagtaaaaatatgtaataaagtgattttggggggatacacaaaattcaacaatgatactcctaaagcatctctacccttatgtgacccactctgagcaataagaacacagcaggaaacacagtaagaaaatgtgctccgtcaatcctcttttctgtgtttctacttcacatgagacatcgtatagaactaccttgtcaagaacgggcagcaagcaaaaaaaagtccatctcatctatgtgtgctgtgcacatgcatctatgcatacatgtgcatgtgtgcatgtatgtgtgtgcttgtgtgcatgtgtgtgcagctaagactttgctgacttcccagactgctaggatgggcacctaccagtggaactgctcgttgtcacgatggagagatagcaaaggcctgtgacaggctgacactgaagcaccttgggcacagcagagaacccaaccacatccctgttttcctggtgcaaatactccagacagacaggcaacagagcaaggaataggtaaggaacccaggtgctcgtgggcagggccaagcacccggagaaaggaagcaggtgacatacttacagagacttggggggtgggcctcatcagcacattccagtggcctgcaggactgtcgagtttgcacagagacctgaagcctaaggggagccatgcaagggtctagacgcagagcatccaggaaaatgagctaccagcccaaaggccacagtggggacacactgtgacctgtgaccaccaagacttcatggcagcacatcagatgggtaggcagacctggaccctgtgaggggagtacctttcacagaaagtgggtcggccgggataaaatgactaagacttctagtcgtctccttggaatgagccaaatatcttgaaaatgtcctttataaatgctgataattttattggtctccctaagctcatttggtgatttaatttacttttacatgaagactgctttaagatacaacaaaactaacattagggtctcacggtaatagcaccatcatcgcaccaaagttaaaacaacgggttagacatcctcttctgccactaaaagaggaagcacgcaagactgtggctggaccgtccagcctggtggtgtcagagaccggcagcgtccccacctctgccgtccccacctctgccctgcccacatctctactgttcagattaagctccaccatgtaggacagtacctcccggaagttatcactgatgaggagttccagaagctcttcctcaaacttctcaagggaaggatatagcgtgaagaataaatcatccaagtaccatgtgacagacttctgaaggcgaggctttcggagggtgtcccctgaaaaaatgagggggttcgtgtagagtatccgtggggacaggaccaccagaagcacctagggcctctcacccacctaggtcaaggccatggtgccaggccctccaggcagagaagaaaccacaggcacagcactcagagccctaaatcagaagccacccccattcacaccaagacgccaagcccagatttcaattacaacccaatttctgcccccaccctcctccaggaacacattttataattcagcaatccaaagaacacagatcactgaatgcagggtaacatctgttcctatcagcttggtttccctttatcgctaacttcctctctagcaagaacacagccagtgatgccgtttcttatttttgtacgtttaccttgaaccatttggaaacatgaagaaatactcatgagcacattaagccaacaagcaaaaacaccgtctgggctggcaccacaagaaaagtccccatctgggtgggggagctgccctggactgtgagctccatgaggacagaaggggcggtagttcctggcacacagctgggtgcccggcacagagcaggtgcatcaaccctgggctgtgaaaggcatggtccagtctgagcagagacctcccctccaactcgggcacgaaggccaagccaaccacctccacaccagtacacaccccgcgctctgccctcccatcccgtcactgccctattgccctgtctcttccccagctttcacccgtaagaactaaggagcaaagccttagattttaagaactatttccaaagttagagaaaggaaacatcagcggaagactcagcgcagagggaactgtcatggtaagagcccttggtcgtcctgctaggaggtcaatcagcacagccagaggcgatcccatcttcgcctctgtgtgcactgagacccttcagaatctcctcagccgctctctgccccactaattccacattttgaacctattcagcaaaattaatcgaatgtgggagcaaagctttatgcgcaagaatacttattaaacattatttataacagaaaaaaataagaaacaaccaaatgctccctaggatggggaatgttaaacaagttaccatgcctccgtataaaacaggaatgaaactcctattgtcaaagatcacttaatgattgaaaaataatcataatattgcaggtaaaaacgatgtgtgtaacactgcgtatgagatcatttaaagccaattttatttgaaaatctacatttacatgcatacatggatacgtatgagaagtaatcacagatgactttatttccttctttatggttttctatcccttcctcatcacaaatatgtattagattctcatatttcacaatttcttatgcttctaagaaacgttgcacgttcctttctgaatttaaaaagaagtgttgaagctgctctgtggaggaatcagaacccacgcaaagtataggtatagttattcaccatggccaagggttaaagacaacccaagtgaccagcaacagatgaaaataaggaaaatgtggtccatccacacacagaatcttattcagcattaaaaaggaaggaaattgacacctggatggacctggaagacatggcgctcggtgaaatgagatagtcaccgaaagggcaaatcctagacaatctactcacaagaagccctaaggtggtaaaatcagagtcagaagatggaagggcgggtgccaggggctggggaggggctgggcactagtgtctcatggggctgagctgcagtttttgtgagatgagacattctggaggcaaacggtagtggtggccgcagactgcatggtttacatggttcaagtggcaagtgtgacactacatctatgttaccacaatttttctttaaagagcaaaacatagagtccaaactttcatgaaatggccaacaagtgtctgaggcatgtcctgaattgagatcctccaccagagaaagagaagggacgcctgggtggcccaggggttgagcatctgccttcggctcagggcgtgatcctggagtcccaggatcgagtcccacatcgggctccctgcatgcagcctgcttctcccactgcctatgtctctgcctctctgtgtgtgtgtgtctgtcttgaacaaataaataaaatcttaaaaaaaaaaaaaaggagaaagaggaccgacaaggaaagagaggaaggagctaaagcagcacacttccaccaccagtgccaagggcagcaagccaggtcctccatcatgggcctccctagcaacaggtcacagggcacacagcagcatgggggtgctcatggaggattttggggatgacgagacagggcagctgtggccctctgggcacagagaaggagcgagggaccatcatcaaaccagggcccatcagcaggaagaggccaggcccgagccaacaccactgcctgctgggggtgctgagtacatcctacccgcaggtagaaggcgtgtgtcctgaaacaagggaagacaacgggtggtcaggcaccctggggccagggcctaggggccaaccccagtttctaaacctgtgtcctcagctgtggaagccaggtgacagccccccactcaggacagtgctgtggggacagagctaataccattcacagccatgaacacagggtcatagctcccgaggaaaggcacacacatgtacacacatagctcatcagactaatcttaaagcctcggcatggctcatccctgtactggctcatccctgtacgttctagtctctactttacaagagaaacagggctcagacattctaagtgacttgcctgaggataccacacccacagaatgtgagtgagggagacccaaatgttgaagctgctaatacacatccccaaatgaccaggaaagtactgtgagaacttcagggagtgaatagattcacagatacagaacccaagagaaacaaagacgggctgctctgggagcaggggactcaggtgggaccccaggggcgccagccaatgccagcccttccagagatggggcagcagcagagacagacttgccggtatgctggggccactgcctgtccagcagggaagccacccgcccctcgccaatctccactactgtgctgttgcctgcccctctagacaacagtacagtaggatgagcaaatctgggcatccagaaggtggcggagtccagggcttctggaagaggctcagtcctggcaggaagcaggctctgcccagctccccattgacaggtggcccccaggcccacagccattagcaaaaaggagccttgactctagtctcacagagatgtgaggaagggagccccacacacagaagtcagacagggagaggaaagatggttcctccaccaggagatcagactaaggagccacatatgcacagggcagaaccttccagatcctgaggggcccaggaggcagggcagggccaggttgcatctggccggctttctccaggccatgtcctgtgaaaatcacaggcactggagaatggctctcactcagaggacagagcaggcctcccaggatgctccactgcagaggagcggccagggtgaggacagtatcccatgaataaagaacaaaggtcctggggccggtgcttcctaagttctcagtgctgagctccatcggcttacttctttgctcctcacatgcatctccaggggcaggtcccaagaaagtccccattttatgcaggaaagggcagcagcagaggctgggagaggttaggagcctggaggcccactccgtactcagaacctcttccctgctccctcctgcatccccctcacaacagcttcaccatctgaagtgtcaccctgaaatgtcacctcattcactctgaagggtcaccccgttcactctaccacccaacctcagcaaccggggttggggaactggggtgaggcgggggacggggactcatggcccgcacactctgtgactggggctatttttcatatggttgttgccaaggctctgggcaggaatgcaatccccaaaaacagcaatgttccttcaggaatccagactccctgccctcagggatccccacacatgcactgccgagaaaccagggattacaggcgcagacaggcaagaactccctgtagtgacatccttaagagaagcgttcagaaatgccctccaggtgcccaacaagctctccacgaggtcctaacagggtcccacagcctccccaggacacagctttcctggaagggcagtggattccccagaatgcaccccaccaaacagtgttgcaagaggaacaacagaatgagaccagttagagggtgacccaggcaggagcggaaagagtctgagccatccacattgctaaaacacctgttgcctttgatggctgtggcaagacgtgatgttgcccttgcttccatctgtccaagacaggagcactaagagccccatgctgtgccctcaccctggctcagtgtctgtgcacacggatccagccaatgagccttccgcggatgaatcatggacaaacccaagcatcggtgaacaagggagaggataaaaggatacaccgaagtcggagcactgctttctccagggaggttccccggggagcaagcctggaaggaggactttcagcaaagatgacctctgtcgtattaaaactggagctcacacagggcaggagtcagctctcctcttgtcaccccaccaccacccctgcctacccccacctacccacaacccacagtaacgtggcacaatccagaaggtgaatcccagcaaggaccgagcggcccagagaagcctaggagaaggcccacagccactgagatacttggatggagggtgggtagggtgggggaagcagcatttttaaggagcacctcaggagattcctgagcagggacttgggaggaccatgtgggataccaccagagtgggccacctcgtggcagactggcgggcatgaggttctgacaccccacttctcagctctaccgagctctggtgtaacagatgaggaggcagacctggaaaggagagcccacggcaaaggccgctttgtctgcttcagacgcctgcgtggtgtcagccagctcaggggagtgtgaggccctctgcttcctccagccctacagcactgtgagaagtgggcccaggagccctgcactccctgagcacccagtcaggaagtctcgctcaggactccaggcagtcctggcattcacctagccttcagggccactgttagaaaacaggcttccaacttatatttacaacgaagcctccagtgcactcagctactttatgcaaagctcgcacccactccagggcaggatgagcaatctgagccttcccccaaacgacagacatctttctggtcctgatctcctgagaccatccccccaatgacagaggcagcatgcagatccgcaaccaaggagcctccgtgcggagactcactctgagccaggccacgggagctctccttgaaagaagcagctttcaggagccacggcatgtgtgcaacgaccctggtgctgacacagctgtccaggtctgtcaaaccaaagcaagtgaaacggtcagaaccaggccgaggagaccacgagagggagggtgcgtggatttccaagatgcattggcaggacctgctaatctggccagatgctgagggcctccctctgagagccacccgaggggaaaggcctgcctcccttctggagagtgacaaagagccaccagcacaacacagctaagaccttcatttccagccattcccaggctgaaaatcactcttccaaaatcagccttcacagaactgagtgagtcaaagtggaagacaggaggaaggttggagactgtgtcaacatcggtatcctggtcgtgactttgtgctacaatttgggggaaaccaggcaaaggacacatggagtctctctgcgttattaaaaccacatgtgaatctagactgacctcaatataaatttcagttaacaactcaatcttcctgtattgtgtgatattttacaaatgaacaaatatccacatccccatatggcaccaaaataaaatcagaccctctccaaattgcacgtgaattccaccactgaagctggtagcagtcactgccttggccctctcagggctataaaatattattcagcaaagaaacacttttccaagagtgagtcacaaaagcccagaagaccctgggcaggatttataacacagacaagaacggcgtcagaaagcagggacagtgcctgcatggctgagagcacagatcagcctggcgtcctgcacatccagggaaggtggagcaaaaccagttccttttctcagatggtcaccggaggacttggtgagaagctgtgttccggtcctttctccccaagaagagggagtatgctcctgcccacccgggcactttccggctccacttctagggtccctggtcattagcttttatgcaaaatattagggacacctaggtggctcagcagtttggcacctgccttcagcccagggcatgatccaggagacccgggatcaagtctcacgtcaggctccctgcatggagcctgcttctctctctctgcctctctctctgcctgtgtctctgcctctctctgtatctctcatgaataaataaaatcttttataaataaataaataaataaataaataaataaataaataaataaataaataaaaattaacaacagattaaaacaacagcaatttcccagacgctgcctgcacccagtaagtgtcccagtgaccaactggccgctctacaggagggggctcagatggcttctgaagacatgacccttcagaatgaggagctggagtatgccccactctgcagctggcccctctgatgccccaggctgacacaagacactgagaataaactcggggccaccagagctgcccattgtcaccacaggaatgacataggaccaggctccagatgagcttcggcaggtccaggttttatctgcagctctactgacacccactggcaagtcatagagtgagggacacacctgagcccccaccaacactctgctcacccagaagcccaggtcagaaggactcatgtaagctgagttttaggcaggtgtgcacctaggttcttgacatcaggacatagcacagtggcacaatgggccctctctgatcgagagggccgaggcctcagagtcacacgcggattgtcatgttttcaatctcgaaggcagtgaccacgcaacattctatctccctgcttgaccccgtcttctctttcgagaggacataagcaaattctggagggtgaaaaggcttcctatatggggctggtggcagggggcagggggttgggggggctgggggagggtggttgcttggtaagccatggcaaatccagaggtgggaccctcctcagacttgctcctgttcatagctatcatttctggtgtgtcatcatctcttcttagcaacactggaaaaatagtgaccttgtaatgggctgaccaccatgggcccatcccagaaatttaccaactcagataacaatgtctgggccccagtattgactagaacactgagccatgtgcagggacgttgttactgaaaacattcccatactggagagctgagctacttcttttttggggggtcttgagtattcctattctaagtcaaaactcggcccttccagctcacgctcatgcccaatcctgctctccccagaacagaacagggagccagcccaccccacacaagaggttccagcagcaccaggtggggaaggatgggtgtggcaccttctcctggatacctcagacagccagacggagagcctccagaagtgcctggaaggagcatgaagttcagatcgaggcccgtcagcagaacccaggagcagagcatatctctgcagagacctcctggctaatccaccactggagaacctagttctaaacacaccaagacagagcccggaggctttcacgtggagttcggttttcaaaaatactcaagccatggctcacccgcattcacagcagcattatgcacgacagtcaaaaagcatcctgtccattgaaaggtaaacagagaaacaaaatgtggtccatccctgcaatggaatattattcagccttacaaaggaaggaaattccgacacatgacccaacaaggatggaccttgaggacacgatgccaagtggaataacccagccacagaagggcaaatatggactatttccctcagaggaggagcctggaggagtcaaattcacagaggcaggaagcagggcagtggggtggccagggctggggaggggacacggggagtcattattgagggattgacagagtttcagtcaggggacatgagaaagttctggagtggatggtggtggtggtgggtgtacaacaagatgaacatccttaatgcccctgaactgcatacttacaaacggttagaacagtagatttatgttatgtctattttgccacaatttaaagagaacgataatcaagcctctgatatggcagcccagcaagggtgggaactaacaacagtggttttcccgttttcccgaccttggtcatgaaaatgcgaacgatggatcttatgacagcatttcccaagtaaggacacagaagcaacttgagggatgagagacaccgctctgaagggacctgtgccctgccctcctccctcccctccctcccttcccctctcctccctcctctcctcctcccttcctcctctcctctctctcctcccatttgctaagggtacctagccacacccagcccacctcatagcacagagcctgatccctgcaaggtctgaccaccaaactaatacagactcaccaaatagagggagacagagcaacccttacccttttctccatgagctggaacgaggccagggatgtgctgcagaccagacaccagaaggttctggggaagaagatggaacacgggctccaggagcgggtgtggcttcagagtgtattgcaggctgcagctctcaatgagggtcatgtgtttattttctgtaggagaaaacacctatatgagagttggcactaaaggccctttcagtcaaaccagccaataaactgagcatctaagaaacaccacaaacaggaaacatatttttccatggagaggggggaatcgctctgttagttcagcctaaaaagcaaactaatccttactaaatccttagtaactctctactaaaactgcaataacagagatacttgcttttaatccacatagtgaaatctctcctgcttgcctctccgcccaaggtgtgaagtccaggcattgcctggtcctcagctcagcgggggactgcaagggaccctttcccctcagcacctgcaccctggagtcccacagagcatcgggactaggccctgggagaggcaggagaccaagacagagccccgcctcgaggacatgtgctcatctgggcaaccgacacacaaacacagatacccacagcacaaaggacaataggatcggaccctgagaaaggtatacataacgcacagcgagctaggggaagaaggagagcaccctgagaaagcagcagctgagaaaggcccagacacagtgctcagggagctggagggaacaaaaccaacagaacagaccctggggtgcaagaggcagagtccagaatcacagggcccagaagctgagagggaccaggacatgccctctactcatccccgccaaaaccttgcttggccttgatttcttccactgacaggagcctcaccacctcctgaggcaagctacatgatttcataagaaaagagctaaagtctgaagcttaattttattgataaagattggcttttaggttatttctcaatatgaccaactgcattcattaggaaaagcaattaatgaaaactatatttttattacctaatgactacacacatagggtctgctaactttaaatttttaagtgccctgagaggtaccctctcgtgtggccggctggagagacactggttgggcctccatggaaggcaaataggagaaatgttacaaactagcccccagaccgagctctgcagcttccaagagctcgacccacagttattgggcacatatacaaaagagaggtgtccacaccagcaacgtacccagccccagggccctggatctgcccggtatccactggcagggccactcagtcaacacagaacacacactcaacaactcaacaacataccatgaggtcatagggagaacgaggggctctctcaacaccaagaccaaacaaaccttctctaaaatgagaaaaatgagatggacatgcggctctgctcctgtcctggtacaaagaccccccagggtccaggagactcagaggggctgcctggggtatggaggaaggagagcagaacactgcaggggcaggggagctcactgaaccttgttacacttttttatgctatttttttaagattttatttatttgctcatgggagacacagagagaggcagagacataggcagaagaagaagcaggctccctgcagggagtctgatgtgggacttgatcccaggactccagggatcacaacctgagccaaaggcagacgctcaaccgctgagccacccaggcatccttttatgttattctgaatcatgtgaatatattgatcattcaggcaagaaatgtgtacatttctggaaatgcccacgagaaggtgagctaccagccacatagctcctcaggcactgccacagccacacaatcttccaatgctgcccgggacaaggtcctgatggtgtgtgtccttccaaaggtcctgaccctggggcactgcactaactcagtcaatggagcacgtgactcttgatctcagggttgtgagttcaagccccacattgggcatagagcctacttaacataaaattaaaaattaaatgaaaaaaaaaagccctgattctaaggatgctgtaggacacaaatactggcaaagtaacgatcagactcaattgagaaataatgtaaaagccaatctttatcaataacattaagcttcaaactaattgctcttctattgtgacatttccgtcaaattcagcaggcagtggaagggatggggcttgctcaaaattcccatcacagaatccaggcaacaactcaaagacacagaaagtcaccctccccaacagatctacctccaaccactggaccccctccacatcctggccctgggccaggtcaggtctccaccaggggcctccaagatggcttccacccggtgccccctccccatcgttcaagtggccaggagccagagtaggtcacatgcccttcaggtcacagaggcatggcaggaagccccctgctctgcctaagacaccccccacccccacccctgcagctgctgggcctcatggaagatggcaggcatccctgcaggacgttcctgtgcatttaggctttgccgtgctggacataaccaatgggcctcacagtgaaaggtttctggtactagcatggccatggccagtgatagacaacaactacttgtagtggttgtcaggtggacaggccaccaccccagaggccaggaggcccacatccctcccaccaggaggttctggggctgcctctcccagggccgtctgcacaaagcaacaccttcccctcaggctagtgcttacagttccttcagttacgtcaaccatccaagctcatgacactttttgtcctaatcccttaaataaatcccgtgatacagcctcaacctgcaaagcaagaccacagagcgcatgtcaaccctccgcatcctgcatttagccaagaaacatcagtgatgcttctcactccacacctactagagctgtgtcaacaccatacaatcacatcctctgcaggagagacaggtgagcaaagcagcagccccccatgcccccgggccacatgcttccaacacacagacagtactcctcccgacacacgcaagacccaggcacctagaaaaagtacaccagggaaatttctaatatctgaaatgtcccaagaaagacagcatttgaggaatcttcatggttgtgtatctagagcactgttctcaaccagggatgataccatctccaagaggatatctggtaatgtccagagatatttttgattgtcatggctaggggtgggtgcagcggggagagggctttggcatctggtaggtaaggcctgggatgctctcagacaccccacagggcacgaggacggcccccacagcaaagacttgtcctggcccccatgtcagcatgccaaggctgaggaaccctgtccaaagtgacagtgtaaacttcagcactggctgacatggggacacactgtcttctccgtcctgctatatagccagctcctaaacagagcccggcacaatgtgtctgctgaacagatgcccactgcttactgggtgcctgttggtgaagaacatattatttagcacccacacaaccttatgacatgtgtagcgttctctctcatgccccgatgagtgaaatcagtcaagagtttgggtcactggcctaaaaccctacaggaataagtgagagctcacttcatcctacatcagttcctacatcagcctgaagaacacttccttggctcactgagaaatggcaaacagttaccacacagcagaaaagctcccagacaacacacggttaggaagaaaccacaacacaagctgacaattgcaaagttttctgttacagccaatgagggagcatgaaatctccacaaaatgatgctggaaaactccaaggatgaagaagacgaggcaggtaccgcttgtcctgggaggcagaggtaggagattccagtttgttgccaaagatccgaagaattccaaagccacatgacagagcctggagagagccatcctgcttccggccttcggtgaccacctcaaccacagccaccacgttagggtggttcaaggacgtgtgaaagtacaagggctgcaaaacagaaaccacaacttcggtgacagactgaccatccaccagctgcagagagcatacagaaaaggtacaggcgccctcgctctgccctcctatgaatgtgtgtgcccgtgtggaacaccgcgtccctcccagggcaccgcaggagagaacaggcatggtaaacttgctccctcggagtcaagaaacattccagagcacccaacacccaagagagcagactatttgcttcgctcctggagcttctgacccacaggaaatgacagatggcaggccctccacacaccacctgtcgcacacaccaccccattgcccaacaaggacccagtcactgcaaacacacacacctgctgagctacttcacagagttctcaccacgtaaaacttcaaaacaactcgccaatattggatctaacccgttttgtgggtaaccattatgaagttccacctaaactagttttgtttaaactaagcaggatccctagattttaggagatgctttgtaattatacccagttcagagcccgactaacatctttttatgacaaaaagcgcctaacatcattttcttacaagatttttttctttttgaatctaagccaagtagatagtattcaagtctctcttaaaaggttttccacctgtaacgttccctcggatgcaaatggacccccagctggggcggtggaaggggtcagatccacctgcgtccagcctccctgcgatgtgcactcctgctcaggagagacagtgcctctgctcagcggtcgcttccttcaacttcatcatgcccggtctggctgcttttatttaaaactaataaacgtcgtaggcacagaagatggggacagaggcgctgctggggtttgcagaacaaatctagatggaggcactgagctgtggctctggacacggaaggtccccagaatcgcatctgcactgggggagcatgggtgtggcacccgcccccaacacaaagaccacccccctccaagcaggtagcggtaaggccgggctgctgcgatgtgatttaaacgtggactaatcccccattaacgagtcattaccgtggtgtcaccaggaaaacgtttactgtaatcacagactgtcagcccaggataacagagagacacattaaaacatcagccatgagatataaatcgttattctattatcttttattgcaaaatgatgaaataatgaccacttacagctgacaggtggttggaccaaaagagaattatgtttctgtttctaagactcaagaactgttttggagagccccactgcagagttgggtttgtcactgtctcacctcttatatttatacctgtgcacatccgtgttaggaacactacccacttttcctttggcagtgaaacaaataccttaaaccgtgatgcaaagccacagtcatctcctacccagaggtcaccttgaaattgctctgcctggaggccctgggaccagctgcttccacacgtcccaggttcaaatttatggctgctccctctcctttccgtttaccccaaaaacaactgctctgagaccaaaacaacaggagcagagccctgctgttgtccatctgcaaagaccccatcttcaagtgggagggtcagtggtggaagtgccacttcctgaacacgacccgaggtgtgaggggtgttgccctggcactactggccatgaagggcacatggtctcttccaaatataatttatggaccctttggtgtgggaacagcatcgtgctaaagaatttctaaggagcctgaaaacacgatcccaccctcaagttcagagttcaaatgtaaaggctagattaggcagatagaaatggcaaattctgctcaggaccgaccagaatcacgtggaagtaaaagcctcccacgggtgagagcacagggcagctccacagcttgttaggaaaacccaacaggccctatagtcacctatgctaagccccagtcagcaaaccaagggacacagacccaactgcagttcccactctcccgggcttggagctctgagtgggtggatctggaattccctggtcagcaccagtgaggtgatctcctggtagaccctctgcacccccgcacacacaggaaggtaccttgcctgaaaaagtttctctttgctagaaacttcttccttccccattgtatctataaaagcctttcactttgtacagcttcctatagatcctttctggaggctaaatgggatgctgcccaaattatgagtcgtgcataaagccaattagatcttcaaatttactcagttgatttttggtgttgtgggttcatttagattttatttatttgagataagatcgagggagcaggaggaggggtgggggtgtggagggaaaaggaaaagcaggctccccgctgggcagggagcccaactcagggctgacatggagcttgatcccatgacccttaaatcacaacctgatccgaaagcaaatgcttaacccactgacccatgcagatgcccctgatttttggtttttgtctttttttttaagattttatttattcatgagagacacagagagagggagagacagagacacaggcagagggagaagcaggctctatgcaaggagcctgacgtgggacttgatcctgggactccaggatcacaccctgaaccaaaggcaggcactaaaccacttagccacccagggatctccatgatttttggtttttaacaagctttaatcccctggtcaccttgaggggaccgtccccaggagggtaaagttgggccagtccagctcacctggtacctaggcatattcccaccttacagcccaggaacaagctacactacaaatcctgccagcattcagctgagcaaaaactgcaaataagcccttcagtctaaggacatttaattaacaacatgcagcaagctttgagaggaaaaccctatgcatcaactgaaattgcatgattaaaagtttgagtcaagcccatcaaattacctgcaagagaaggcctctccagacagaagaggggaccctcgttcaccccagtcacatcccactcatctacatctgacaggtttcctccctgacaggataaacagattaataaatcatggaaatgctcctgtcttaatgccttgatcttcaaaaacatggacaccattttgcctcggggccaggtccccatttctcacccacatgacagaactgaaattgggcctctgatgatgtgcctctgtggagagactgcagccaatgtgtccccaacggcattgccactcgacctgcccccttcagtccccgagcagaaagctgatggcacggccagggaagagcagagcagagattctaacagtgccttccaaacaatttttttaaataaacctttaatagaatatcttcaggaaggcagaaagataacacttcctgcagctcctacctgctagctcagcctcccacctgagggcttgggtggttcctgtacacttcccacctcaagactgaatgctccaggcctggggaactctgctacatgaaactctgctgagagatgcaaagtctctagttccagagtcagaagcttcagtccactgttacccacaggaaacaacacgatagatccagccccactaggagactcgtccagccccccatgtcagcgtgccaaggctaaggagcctgtccagagtgacagtgtaaacttcagccctggctgacatggggacacactgtcttctccaacctgctacagccagctcctgaacagagcctggcatgatgcatctgctgaacagatgcccactgcttactgggtgtctgttggtgaagaacatattatttagcacccacgtaaaccttatgacatgcgtagtgttctctctcatgcccagatgagtgaaatcagagtcaagagtttgggtcactggcctaaaaccccacaggaataagtgagagctcacttccatcctacatcaacccctatatccaaggccagagccaatcccactccaacctaaaggacacttccttggcttactcagaagagtaagcaaacgcaaataacccacagtatatattattatgaatagacaacacaagctgacaaatttaaagctttctatcatcccccatggtagttaaatctccgtacgactaaggcccagtgctagaaacatccaggaaagtgatcattcctccatatctggctcggcgtcctgggggtactttccgtagagctggagaggaaggggccacggtgaaggacactgccctagggctcctgccatggtcacatatgcttgggagctgcatagaggacacatgagaggctggctattgagggagaaccagcacgtgttcaagaagtaagggattaatcaagtcacttggaagagcttccaggactttccttgggattacatggatattccttaacagcatccacttttaaagagggaattacatggttctcattactctaacaaataagaacatttgagaattgtttctcatggtgtcacttaagggaagcagaagccacccaattagctgtaccttgagctcaaacacctgggttacctactcctctaacaacacccggggccaaggcaagctctgggtaggtgcaagcctggtctgtgtggcggtggtcagtggagacccctaggactcctgcccagtcactcccataacataagaggaggaagaaaaatgagactgtcacaggaagcccgtgtgcggttagttaaggggaaaatttaaaggagaattatgtccatctcagaaactgcacggtactttggcttcccagattattttttaaaagatactttctgagaggctgaacaaattattaaccatctcagcagaagtgaaaggcaattcctctgcacgagaatggtctggaaggacggcccagaactgcgggctgggagctgagaggctccagcaggcagcacctaggaggccgagcctgagaggggaggaattcatcattcacggaaacatcagctcaagttcaaggcaaggtctttctgcaggacagtgtttgccaagtaataaaatcaggcccacatatgacacctaacgactcagtaaaaaccctgagcacaggagctgggttcattaatctacatttgactggacagccatggtgattccaaagatgcagcctgcccacctccagtcagatctccccacgccactgctggccagtaagaccagaaaacacaaccaataaaatcagagagagaccttcaccaaatttcagaatccccctgaaaaggggcacctggtattcacttccacgggtatgcgggcagagcgcacagaccctcctgtgcatcagtgccccgtgtgcgtggaagtacggatgtgcacataagcttgcgtgcatgcgccacacaggggttttgctttccttaactcaactcaagaagaacctatttccctgtgtgctgagcaccaaggaaaggagagtagtgctgggctcggtgaggacgcagctgaagggcgcggccgaccggagctcattcctgggccgccactggcctctgctgctcgccttgggcatagcagctggacggcagggctccctgagcaggtccgggctctggcctcacctcccagccgaagacactagggccagcccccggccctaatgcacactccacttcagcaactccaagactgaagggtgcagaattttccagaaacattaaaccctgaagtcctcactaacccagaatcccctatcaccctgcccagctcagcagaagacaaaatgtgagggcagccgaggcaaagcagggtggcacttggcaccctggacctgggacttggccgtgagggctcaggactgggagacacctgaagtgttgtggtcccacgagtggcacggcagccaaggtctctgggggtagcaatgctaaccctagacaagtccaggaggagcaggtctgctctgagggctgttttctaccgtgactgcagttctagagggtcaccatagccaagccaccctgggcagaggacatgaccctatgacagggacgtcccaccactcaccatgggccagggaagggtgggctgaggggaggatgtagaagaggaactcattccaaaactacaaccgcaaagcactcagtactgatttcagtgcacgcctccctccctctacccacaagactcttccaaacggaaagcccaggagcagacactggtcaggggaggctgcagagcaggtgcgctgccccaggcactcccagccccctaatgtgctgggttactcttcaaggaaggacatggaacaccagcctctggaggagaggggtgggaatggaatcacataaatgtgctgtgagccaacgctcagcccacagttaactccctggtaggggacagccatgtcaccatggccttcatcattcacttggtaactgtcacaacaccaggcttcccagggccagactgtacccacgacaaagagaaatttgaacagccatgcccttgagggccaccctcaaaggcagcagaggctgagctgaagccaggtgatcagccacaccggggccatgctgctcctggtgaaccctgacctgctggatcagaggcgtgaaggccactcatcacctgtgctgatgaaaccctgtagggagtcaccgacaggcccacaggtgactcacactcggcacggcaggccagaggcctttcagcagcacaacttctgtccccaggacaacattcctgacgtctaaggggtaggagcaccctccactaccctgtgatgggagggctcccatcagaatcaatccaagccccaaccacaatccaaccataatctggactctgggtggcagccaaatgctgagagcagcacatgctcagcttactttgtttcacgtctgggccgggtcagtccccagcagcaaagggctaccttcggctctggttacaggcagtgacacctccaagaatggctatggtcacagccacctccattactcacagcactacctgcccctcgagcccactctgcccatgagaaagatcacatcaacggggcctcagccctttccatacctgtgaccatcacccacatcaatcaagcagaccaactgtgttgggaagtgtgccccagcgctgggctctgcccctggtgtttgcagcctgtgagcgtgcaagccctgctccactcacagtgcctgtgctcacagacatccaaaggactagtccctcttgctgcaggtctggcttcaatgctcgctgccagggaggaccctccttctttgccctcccacgtcaggtctccccgttcacccccagggacagttctggtctccttcagaggctcaccacaatttgcaatgaccctgtcacctgcctttgtgtatcacccctgctatacaccatgagtgccacggccatgccacccaccatcaccactagacgctcagtccaagggctgagtagaggctcgtattgggtgccgccaaacaaagacaaaccatctagctgcagccagacacaaccatgcgggcaagaccatgtcaccaagcaatgccaatgcagccactcaggatggaaacagaaggcatgggttcgagtccaggctctgccacctgctacctgggcaagccaccctccttctaagctgcaagtgctcatctttaaaaacaaagaaaaaagccacaatgaaagaaggcagacaaggagagtgagggaaaacaggagaatcagatgagatcaataatttcctaactttgttccaagaaccccaagggtccttgaaggcgagaagacctccaggagccccctgtcctaccgcttcgaccagagccatgtgctctcatctgttcaggatgtttggcctccacacagattttgtttagaaatgaagtgccccagtgttaaaacaagctttgcaatgacccatctgctgctcagtggcatttctcgggtctgagatgcactggtcgtgtccccaggtcataagccccacacacctgctcttggccacacagctgccctgcctgccaccatggcccacctcatcaaagatgatcctagacggctgcctggaaagctgctcagccggcctcaccgcggtcttccatgtcctcccgaaaaagtgtcggtaggtgacatcaaagagagacacacgcagatggcagcccacctccgaaagcacctccagcactccctaggagacagaagggtacaggtcctgaagggttcttgacatcctgagccatcccatgtgtgctgactgaccagcttggtagaattggatcacttagcatcttttaggtgtcaatatccttggggggaacttttctctcccatggccagcggtcttcaggttatgcatgaaagtgcttggccctcccggctacttagagcactgaagccccgaggcacagacacgggcatatatgcccctcctggggccagctaattcctatacagagtaaatgacttgccctccagaacatctaggatgagcaaaccaaccaatccagagttcacactcccacctggccctccatctggctcttagactcggggagaccactgagattactcacaccagccatcctaagcctggtcatctgctcagcctgccttgcacattcctccccacagaaacaacaatgaaggctttcagccacagtctctccagctgctctgcctcccgatcgaccccatgccaccccgcgtggccctatgtggcctggtacacccccacctcttgggaatggcaataaacggtcttctctaggttgctctcactgggcctgaacaaacaaaatcccacacctgcctgtgtccctacagcagctgtgagaggtagagggagaagccaatgtgccatggaactcagcaggggtgcctcacaaagcacatgggtgtcaaatccccgcactgcatgcttacatagctctcaactattttatccattacacctccctccagctaccacatggaggtgactgggtgacaccagtgatgggccagatgtggggaggtgaggaacaacagctggaaggacggggtggccagctgcctccaggtgcccaggcctcacctgggcctagtgctgcaagtcctacatccctagcgaaggatgatggcatgttttctgccccgggctgctgggaatgaggcagtgttgggtacagaggtgtcagacagctgacaggtctggaagaggcatctgagccccgtttcaatcacagaggtatgggggccccttgggactttgcagacctttctcagacctctctcgacacacgggctgcctctctacccatatgttttctcaggaacctccccagctcactcccatgccttccctgagcactgccatctagacacctgcccatcctctgaccctggtttttttttttttttttttttaagattttatttatttattatttttttttaagattgtatttattttttaaaagattttatttattcatagacacacagaggcagagacacaggcagagggggaagcaggctcccctacaagagcctgatgcgggacttgatcccagaatgacaatctgagtcgaaggcagatcctcaaccactgagccacctggttaaactgtggtttctggggccaatttctttcagggatgtttgctatcatctaaagttgtcaattctcaaagtgactaaactcgtacaaacattgtaggactaaactttttcatgtgtttcctacttggtcttttttttttttttttttttgacttggttttcctttaaatgctatcttttcactgattttgtacgtacagctgacccttgaagaacacaggtttgaattgcacaggtccacttctatgtggtttcattctgttttgatgatgttttgaggatgatggtacagaaccgtagatgtgttttctcttccttacaccttaacaggattttctttcctatagctgactttattataagaacataggatatgatacatacagcataaaaaaatacatattagtcaactgtttacattcccaggaaggattctggaataatagtaggctattatttgttaagttttgggggagtcaaaagttatgtgtggatttttgactgcgttgcggggtcagtgcccctaacctcacattgttcaagagtcaacaataaaacttgtaaaatttagggttcctgtgttaaatcccatctgattgtgaggtactatcctttacgtatatcactagatctgatgtcctcatagtttaagaacttttgtgttcatattcatgagggatgtctgtactttcttgtgatatccttgcccaaactggttatcagttatggcagcctcaaaataacagctgggaagcattctctcctctgttttctgaatgagtttatcaaaaattcaaattaattaatcttcctcttatactttataaaatccaacaatgaagtcatcaaagtctggagtttctttgtaggaagctgtttaattatgaattcatttatttaacaaatataggactttttatattttctatttcttttagtgtcacttttggcaagtcgagcttttcaagaaatgtgtctatctcaactaagttatcaaatttgttggcacacaagttattcatatccctcgactttttaacatctgtaggatatatagcaatgctccttctttccttcctaatattggcaatctgtgtgttttcttgctcttttttcatgatcagtcttgtaggagtttatcaatcttattcatcttttcaaagaaccaacttctggctttgctgattttctctattatttgccctttctttactgatttctgctaaatgagcagaaatctaccattttcttcttctattctctttgggtttaacttgttcttctcatgtgatgcttaaagaggacatagaggtcatcgaatttataactttcttctcctcttacatagtcatataaagatgtaaatttccctacaagtactaaactactcacaccccataaattttggtctgttcttccattactcagttcaaattattttctaatttccctatgaatccttctttgacccaaggattagttgttagtatgctgtttaatttcctcctgtttgaggattttctaggtatcttcttgttcatttctaatttaacatcatacaagtcagtgaacatatatatactctttataaattccaatccttttaaatccattgagacttattttcatggcccagcatatggtctatcttggtgaatgtcccacatgcacttgagaaaaacgcgtattctatttgttggttatagtgttttagaaatgacagtcaggtcagagtggtggatggtgtttcaatcttccaaatccttatgagtgttctggttcatttgttctatcaattattgagagaggagggctaaaagtctccaattgtgatggtgaatatgaatggagctccctttagtcctacacaattttgattcacgtgtttggaagctgtgtcgctgggtgctgcgcacaaaggcgcaatcacagcttgcccaaccattatgccaaactattgccaaactattgtgctttgaaaggccagtataggggcacctgggtggctcaattggttaagtgtctgccatcagcttaggtcatgatctcagtgtcctgggatcaagagccgcattggactccctgctcagtggggactcggcttctccttcttcctctacccctcccccctccactcacacttgctctctctcagataaataaataaataaaatctcttaaaaaaaaaaaaaaaaaagccagtataggaggctggcccttggatggaggctgggcacttagcattcaggagggttcccatcaccatactgacaacggcaccctgtgcctaaactgtatacagtggtttacgctaaacacctcctttcttgctgggagtgtggaatctggaaacatgctaggcagaggatacccgcctgaccagtcctcaaacactgtggccactgagtgtctaacgagcttccttgctgggaacatttcatacgtgttgtcatgactcgttaccgagggaactgaatgtgtcctgtgaggacccttgaagcacatgcctagcccccccacactttcccccatatgcctttcctattgctgatcttactctatatcctttcactgcagtaaaccttttctatgaggctgattatatgctgacacatgtgaagtcttcccaaggaatcactgagtctggggggtggtctcacaggtctcaacagaggtacatgcacatttagaactgtcctatcttcctgactgcatcattatgaaatgccccttcatctctggtagtactcctcaccttgaaactgactgtctgacatcacagcacaaaggcttcttacaaggtctacatggcctgtattttctccatcctttaactttcataggtacagttcatttctttctgcttaaagtgtgtttttttataagcaacctataaatggtcttaacaataatttgaaacacgtaactctgagtcaggaactccatttggaggaagtattccacagacatatatgtacatatgtgtaaatgaagttaatcactgtagctgtgtctgtattgcaaatgactgagaaaaaatctacatgttcatcaataaaggtctggataaatacaggcaggttcatccagagaaatactatgttgtcttaagagagattgttcatgtcattacatgaacaatctataggacatattactaagaatattttttaagcaaaataatacaggtatatttacctgctgtgtttgcttagagtttccagaaagttacacaaaaaagcaggtcacactagctgagtctagggtgggacactgctgccttagggattctgtgtaattatacactctcctcacgtaccctagtgaattctgaacactgataattaccttttcaaaagtaaataaatacaaataaacaaaataaaataggaaaataaataaaatttccttcctaaaattcctaagagagaggacagcactgtccactactctgtgagctcctattaggtggaagtcactggcgctgggtgaccaggcaggagcccagctggccacctggattggggcaccccacgcacccggctgcagtcccctcaggctgtttcccgctctgtgaaatccagtagcgacggaccccacttcagagggctgctgcaaggagcaggccaactggaacaggaaagtgttgggctactagtaaggctggaagagcagcagccaccatggggcaagcacagtggccacacacagcagtcgctggattagagggcttacaagcgaatgctatagggtgtgagtgatatcaaagaagtgagctcttacagatccaggcccccaacaactaaatacatggagaatcacttgtctatctgcagaggagaacatagccttgccaagccaaaccctatgaaagtcacatgttcacactggcaggtctggatccttctcatgcgtcccactgggtgtcccactgggtctcactagaaccatccaattacatgggtgctctgtgggcagcgcagaagtaccagcttctctagcagttttcctgcccctgaaggacacagatgaagccagagccacagagcaagggcctgagtgtgcaggacgctatggcacatccaggcacacagcttcagccagagctctgcccttgtggttctaactgccaggctgcaagcaggctgcttgagtcgtgcatgagcccacctcctgcaaaccacccagctcagaaataatgcaggaaatcatattacctggattttaaaatggcctcactgtgggcagaaagaaaccttcacccagtgtcaaaagtgctgaccgagttgctgaatcacgcatgtgttctctttgctgatgagccactttgcctccaaataatgccaaggcccaaggtaatgggtatggtggtaagactcaaataaataagtgaaaatcaataccctctcagctgagagcaggagcaacatatataggaaaataaatagcaaatttctatacctgcttaattagtggcccgtcaagccacttcaggacacactgaaatgctgttgattcctttaaaagctggcgtgctctctgtgggtggggagggaccagcacgttttggtcaaaaatcctgagccagtcattcattctgccagtcccgagggtcacttcggcctctaggagtacctgcacaatttgtgttcaaataaataaaggagttacgaaggccatagtgcacagagcaattctgaaagtttcctacctccccttgcatcccagaggaaccaccatccccaaaacccacactttgcaattcctcccctgcatccccagtgcagcacactggtatctgtgccaccttcccctggtaaacacacccatcttgctccagatcatctctctacccctgctttcaggagccaactacagcatgaccatgctggcctcagtgactagtgcagaaataggtatatgcagggctgctttgtacagctgcagaggttgagcactacatgacagtgccaggccttggggactggggctatcttggaagggactaggaagcatgacccaatcagagccaaggagatgcaaatctcacagtttctgctggctctgctgagggaagggaatactcttctgcagccctcgaaagagaggtcatcagtatgaagctgtccggaaccaccgtgggagcagcttccttgagacacacatgaaaaaagcaagcagagcaaaagcacaagcaagggtcagtggatctaggtgtcacagttagagccctggcctgaggagaccctaaagcaccccaggactgctcagctacattaatacagtctctcgttgccaaagctggtttggaacagattttgagtgttatgggctttttggtgacctggctcactgaggacatcatccaccttcccaccagatagagatctgtaccttctctagcctcttttcggctgacatacaatcttaagatcttggctttgataggaggctgaattctgcacactaaaatgacaagcaagataaggcagcagatttggtctcctaaaacagaggtactttaaatgggttccccagactctgcggttccccggagttgtcaaaagctcaatgtcaaagctattttcataatattcagatgttatttgcctttttcaccgtgtcgatgtttgggctgatgaagcaaagggaagagtgggcacactggcctcagtgcaccgaggcaaacctctggccgtcgtgcattcttcaccactcacagcaagggcagagtccccagtgaagctgcaaaaactatgaattttattacatcttgatccttgagactggcatttttaatactctgtgatgagaagggaaagacagggtagccctggtggcgcagtggtttagcgccgcctgcagcccagggcgtgatcctggagccccgggatcaagtcccacgtcgggctctctgcatggagcctgcttctccctctgcctgtgtctctgcctctctctctagctgtgtctctatgaataaataaacttataaaaaaaaaaaaaagaaagaaaagaaaaaaagagaagggaaagacacagcaagcattgctgctgcaaaatgaagggtactggtcatctcagggtgaggctgctgtgagcctctgtggcacactcaggacccactgctcctgttgtctggtgatgctatcttttttttttttttttttaagattttatttgagaaagagagagagagagagagagagcatgagcaagggaaggggcagagaaagaagcagacgccccacagagcagggagtccgattcaggactcaatcccaggaccctggaatcatgacctgaaccaaaggcagacacttgactgagccacccaggtgcccctggcaataccatctttactaaaagagtagctgagagacagactgagcttactcaaactcgggcactttgcagtctctcaaacgtgaactaaatgagtctgtcactttcaaggaaaactggtaataagaattgggctttcaggcaaaaatcagaattgggaaaatctgtatcctccactgtgattctgacagcttcctcctccttaaagacgtgcttgacaagatggatggtgactgtgacaaaagtgatcttctgaaactacgattaaatgcacactatttggaagatcagcataccttagaagcagtgttctccaagtgagcagtgcatcatgatataaaaaagcctgcatgagtaaaagatccatccacagtgcaggacagggcagccctggtggtgcagcggtttggcgccgcctgcagcctggggtgtgatcctggagacccggaatcgagtcccacatcgggttccctgagtggagcctgcttctccctctccctgtgtctctgcctccctctgtgtgtctatgaataaataaataaaatctttaaaaaaaaaaaaaaagtgcaggacagaccaaaggactttaaagtaccggagcacgcaaacttcactggcagggtgccaaactccacactgcaacaaacttttaagaaactaccactcgcagtggcgctggcgttgcatttggatgaagaatcaaaggagaatgccacaattacctgagactctgctcccttctccgcatgaggctgtttctttcaaatacttcaaccaaaacaactttgctcaacaggttaaaaacagaagcctaagggcgcctggatggcttagatgagtatcagactcttgatttcagcttaggtcacaatctcagggttgcactcagtggggaatctgcttgagagtatctccctcttcctctccctctgcccctcccattgtgagtgctctcaaataaataaatcttaaaaacaaaaagaaagaagcctatatgagacccagctgtccattaagccagacattacagaaatgtgcaaaaatgcaaaaccaccccttttcctgctaatttttttattctggaaaacagctggttttcataaaaaatacattttatgctaacatgtaacaggtctattgttatctctacatgagcaataacattgctaacatctctcagttttcatttccaatacagttaaagctggtagacataataattaaagtaaacaaaaggtctctggagtcctcggcaattttttaaagggtaaaaggtacgagaccacaaagtgtgagaaaggctattctaacaggacccaaaagatgagggctttgtacagccaggccaccctccacctttgcaggctgtgtggggagcggagaggatcccccagagcacggacatggctggcgtgaaaagctctcccacacccagcatgcccaggcacaaaggcccagcacccacaccccacagcccctgcaggcaggcatgcagtgcagcatccagatctagactgtcaccatgtgagcagtgaggcacacatgcattgcgttttctgttctgagcagttacgtctgctcagtatccccttgttttaaggaatgaccctacaatccagtgtgggaaggctgtccacaaatctaggcagagcactgtagtgatgggctcaagatgaacacgtgacccacactgtgccaatcatcatctcccccaggactccagccgcggttccctctgaagaggctgtctctgctgagagggcaggagctgctgggccagcctgaattccaggggctaccctgccacttcagggagagcctgaggtcaaagggtgtcctgcccacagagcccagacccctagggggagagagcctgatggaaggcagcccccactcccatagcagccgaggtgtagcagccaagaaaagcccatggaggctttccctggtggctaaatggcccccaaagaagtcccggtcctcatccccaggatctgtgaatatgggacttcgcatggcaaaagggactctgggagtgtgattaagtgaagggtcttaaggaggggaatggatgacaggagccacctagatccacctggatgacacaggtgggcctgatgtagtcacaaggatcctcagaagagggaggcaggagggtcacagaacaggtcgagtgaccacgaaacagacagagaagggagggagggaggggtcacaggccaatgaccagctagaaaaggcaaggaaggcttctcccctggagctttcataaggaacacagccctgcccacaccttgatctgagcccagtgatattgaatttggacctctgacctccagacctggaagagggtaactgtgttgtctcaagctgcccagattgtggtatcattacaaaagccacaggcactcacacaggtgtctaccactagtaagcagcagaggcctgatacccagcaggagggccacatgcccaggagagcttcttggaggccctgggggccagtgactgggcccacctggcccctgtgcttcccatggttgcagcactcgccacctgtcctgtgagggtctgcctgctggccaatcacttgcacgaggggatctattttgttgtgtccttggtacctggctcaccatctgaactcaacaaattttcataaaatcaaagtctcaataaaattgactcccaagcattttttaaagagaaatttttgggatccctgggtggcgcagcggtttagcgcctgcctttggcccagggcgcgatcctggagatccgggatcgagtcccacatcgggcacctggtgcatggaggctgcttctccctctgcctatgtctctgcctctctctctctctgtgtgtgtgtgactatcataaataaatttttttaaaaaattttaaaaaattttaaagagaaatttttaaaagaaatgttttctcattgactttcattaaaaaaaaaggggggggggtgttaattcatacaaagtgagtgtcactacggattgaaagtccctgtcaggaagtccccctgcctagggagcaccgtagccaagcatcacctctgaaaggggcctctgtgcaaggcccgcactgtttccccggctccaggactgggtctcttttgcaggcccttccagactttgctcattgggcccagattcctgagccagcacctacctccccaccccctggctaccacacttggccagcagatgctgaaggacgctcctgttctagtccacctagcagaggaagcatgtccacaggactgccccaggctgtacccagtccccataggatgcacgctgggaagataggcctccactctgaatgggacagtcacccaaccaaaacagtgagccaggcagatggaggcgggtagcaacagtctgagtgggcgatagagaagagggcacccgccacataggctcctgggagtgggagacctagtgggcacagagcatttccctcccaacctcccaacccccacctaacactctgtcctctctgtggccactgtgcccccgctcggcccctgctccaattcctcctggacaccatgccccctaggatcctgtcacagaaagtcaccacaattgctcaggctccccatttctgttgtcctgcgagaatgagctcagccaggtcagggctagatcctcagtgaaaatcttttgatgactacgtaaataattaaattatgctgtgaaggagctaagactgctacagagagagagcagaaggcctgtagggctagatgaacagggagaaaacagtgaggccccatgcccaacacaaagggctctccaggggagaaacaacatgagaaaacacaccagctcccacagagagcctgctgcgagctgggtgggcactcacagacccattcatgtgtattagtccaacaaatgtgcagataagtatcacagacgaggacaaggagactaaggctaaacagccaggccaaagccacatggctgctcatgaccaggctcaaagccaggagaggctccggagcctacagcatgacactggctggagcacccaggctaagccccactctcctcagacttcagggaagtctcctgccctccctgaatctgctcccacctacaggatgagaggggactgtgaggtctcccagagggctctgctctgcacacctcttctgttccactgtgtgcaatggtgaacaatgccaaaggccaggtctttaagaatctaaaaatccagtggggaaaagacaagggttcgagtgtgagcagcacacagaagacaagcataattcagtctcctttttgggaggatgcggggcagagagaagaggaaatcccggcactgaagaagcaagctgggataaaatctcaacaacagtggaacaccagagaaagaggggggcaggagcagctgtgcaggcagggggatgtttccttccaagaagtgaacaagatctgggatgcctgagaccgaaaagctttgggaactttactgggagtaagtggcagttatctcaaccccgcctcgaagttttttggtctctcccaccccccccccccccccccctccgagcaagcaatcttctttgcacacctaccaggtgccaggacctggagaaaccatcctcaacatagcagacaagctccctgccctcagggaatggatct

SEQ ID NO: 4 - DNA, 4819 bp, Canis familiaris
agatccattccctgagggcagggagcttgtctgctatgttgaggatggtttctccaggccctggcacctggtaatcctagaggccgaagtgaccctcgggactggcagaatgaatgactggctcaggatttttgaccaaaacgtgctggtccctccccacccacagagagcacgccagcttttaaaggaatcaacagcatttcagtgtgtcctgaagtggcttgacgggccactaattaagcagggagtgctggaggtgctttcggaggtgggctgccatctgcgtgtgtctctctttgatgtcacctaccgacactttttcgggaggacatggaagaccgcggtgaggccggctgagcagctttccaggcagccgtctaggatcatctttgatgagcccttgtactttcacacgtccttgaaccaccctaacgtggtggctgtggttgaggtggtcaccgaaggccggaagcaggatggctctctccaggctctgtcatgtggctttggaattcttcggatctttggcaacaaactggaatctcctacctctgcctcccaggacaagcggttgaggctgtatcacggcactccccgagccctcctgcacccactgctccaggaccctttcgaacaaaataaacacatgaccctcattgagagctgcagcctgcaatacactctgaagccacacccgctcctggagcccgtgttccatcttcttccccagaaccttctggtgtctggtctgcagcacatccctggcctcgttccagctcatggagaaaagggggacaccctccgaaagcctcgccttcagaagtctgtcacatggtacttggatgatttattcttcacgctatatccttcccttgagaagtttgaggaagagcttctggaactcctcatcagtgataacttccgggagggggacagtccactagatggcagcacgctggagatcttggagcgccgcctgtgtgtgggcgtgcacaatggtctgggcttcgtgcagaggccacacgtggtggtgctggtgcccgagatggatgtggccttgacgcgctctgccagtttcagcaggaaagttggctcctcttcgaagaccagctctggaaaccaggctctagttttgagaagccgcctccgactccctgagatggtccatcaccctgcgtttgccatagtctttcaactggagtacgtgttcaccagcccttctggagtggacagcaaggcagcctcggtcacctctttgtccagtgtggcgtgtatgcacatagttcgctgggctgtttggaaccccttgctggaatccggctctgggaaggtgatcttacctctgcagggtgggatccggcccaacccctcacactgtttggtctataaagtaacccctgccagcatgagctctgaagaggtgcagcaagtggagtcagggactgtccagttccacttctcgctgagccctgagggacacctggacacacccacggggctggtccacagccctgaggcggagcggcagccctccaggaagccacccacatctcctgcaaacctgccagcactgccgccccaggggctcgctgccctccaggactcgccggtgggaccagggttgtccctttcccagctcgcagcttccccacagcctaccacccgctcccgctcagccacaccccctccacggcaacccgacagctctaagcccccgccggcccaggaggagcccccctcggagggcaggatctctcacctggaggcccagctgagccaggcgtccttggtcctgggagcctctgttgctgaacagctgcaggagctgccttttacgccggtgcacaccccgattgttgtgggagcccagaccaggagctctggaagtaagctctcacgagcctcaatggtgctcctgcaagcctgcggctttcctgagattctggatgccaataagcaaccagccgaggctgtaaatcctacagatcccgtgaaattcaacccccaaaaggaagaatcagattgtctacaaagcaatgagatagtgctgcattttcttgcctttagcagagtgtcccaggactgccagggagcaccgtggccaaagaccgtgtatttcaccttccagttctatcgcttcccacccgtgacaacgccacgcttgcagctgatcaggctggacgagacgggcaggacaggctctggttccctgttgtatctcctggttctcactgacaaagatggctcctctgatgctgggacacctggctttcaactcaagtacctggtggaacctgggttcctgaaacccggagaacagcgctggttcacccactacatggctgctcagaccctccagatcgacatctgggatggagactccttgctgctcgtgggatctgcggccgtccagatgaagcatctcctccgccaaggccggccggctgtgcaggtcctgcatgagcttgaggtcgtggcgactgaatatgaacaggatgtggtggtgagtggagatgtgaccagatttggctgtgtcaagcccatcggtgtctacacagtggtaaagggctggctgcacctgacgttggccaacgtgggtcatgtgtgtgagcagagagtgagagattccagctcattgccaccttccaggtcacgggtcatttcgaatgatggagccagacacttcgagggaggcagtctcatctcgcgcgggggtccgagatcagtaaaaaatgttgtccaagcacagaagctggctgacgtggaccgtgagctggccgccatgctgttcacccacatgcaacatgggagcaaggggccccaaggtgcttgccgtgagggggatgccgtccgcaggcgcaagctggagcggatgaggtccgtgcgccttcaagagtccagaggagagctcagctgctggggggccagcatgttgattcagcagaatatccgcgcacagcacgcacgtgacctgcaggtcattgctgcctatcgggagcgtacgaaggcagagagcatcaccagtgcgctgagcctggccatcaccacgatgcacacaatccacgtcacactgggcactgccgagttcttcgagtttgcccttaggaacccccacaacacacagcacacagtgaccattgaggtggacaatcctgagctcagcatcatccaggacagtcaggagtggagatacttcaaagacgcgaccaagctgcacacgccggtggaggaggacatgttccatctgcggggcagccttgccccgcagatcttcctgcgcccacgagagaccgctcacatccccttcaaataccagaccttctctgtagggcaactggccctggcgcagccctctgctgagctcggatctgagaaggacacggataccggattgccccagaaatccagtgcaatgcccactaagcatgccaaggtcttgttccgggggagtggcggtaagcccatcgccgtgctttgcctaaccgtggagacccagccccatgtggtggaccaggttttccgcttctaccacccagagctcaccttcctgaagaaggccatccgcctgcctccctggcacacgcttccaggtgctcccgtgggaagacctggtgaggagcccccagtctatgtccgatgtagcgaccccaacgtcatctgcgagacccagaatgtgggccccagggagcctcgggacgtgttcctgaaggtggccagcggtccaagcccagagagcagagacttctttgtcgccgtttacgcggatcgttggctggcgacacccatccaaatatggcaggtctacctccactccttgcagcgtgtggacgtctcctgtgtcacaggccagcggacacgcctatcccttgtccttcgagggacacggacggttaggagagtgcaggctttcacctcacaccctcaggagctgaagactgaccccaaaggtgtgttcgtgctgccaccacatggggtgcaggacctgcatgtcggtgtgaggccccgcagggcagggagccggttcatccacctcaacctggtcgacgtggactaccaccagctggtggcctcatggctcgtgtgcctctcctgccgcccgcctctcatctccaaggctttcgagatcacactggctgcagggaaaggcagaggcgccaacaagcggatcacctacaccaacccttacccctcccagaggacctatcacctgtacagcgaccaccctgacctgctgcagttcagagaggactccttccagattgggggaggagagacctacaccatcggcctgtggttcgcacctcggcagagccccggggaggaggagattctgatttacatcaatgaccacgaagacaaaaatgaagaagccttctgcgtcaaggtcgtgtaccagtaaaagagcagccggaggtgctggtggggcttcctgtcctgggggctctgtagccttttgtcccctcgatgacctgctggggctgctggtgagcatggccgagggtggtgtgcgcgccgtgcccctgcccagtcctcctgcccaagtaagcgctgctgtcaccagctgtgccacgagccccgcgggcgtcctcagggtacgaggctcgtttcttctgctgcactctggctgttgtcactgtctatgacatgttgatttactttttaggaatatatattttatgaatcatcttaagtgataaaattatgtattcttactggattttgtacaaacattatttgctatgcaatattttatattggtgtcctttcatatctttttttaagttacgtatgaaatactaaacttttacctgttttctct

SEQ ID NO: 5 - DNA, 4754 bp, Canis familiaris
agatccattccctgagggcagggagcttgtctgctatgttgaggatggtttctccaggccctggcacctggtaatcctagaggccgaagtgaccctcgggactggcagaatgaatgactggctcaggatttttgaccaaaacatgctggtccctccccacccacagagagcacgccagcttttaaaggaatcaacagcatttcagtgtgtcctgaagtggcttgacgggccactaattaagcagggagtgctggaggtgctttcggaggtgggctgccatctgcgtgtgtctctctttgatgtcacctaccgacactttttcgggaggacatggaagaccgcggtgaggccggctgagcagctttccaggcagccgtctaggatcatctttgatgagcccttgtactttcacacgtccttgaaccaccctaacgtggtggctgtggttgaggtggtcaccgaaggccggaagcaggatggctctctccaggctctgtcatgtggctttggaattcttcggatctttggcaacaaactggaatctcctacctctgcctcccaggacaagcgaaaataaacacatgaccctcattgagagctgcagcctgcaatacactctgaagccacacccgctcctggagcccgtgttccatcttcttccccagaaccttctggtgtctggtctgcagcacatccctggcctcgttccagctcatggagaaaagggggacaccctccgaaagcctcgccttcagaagtctgtcacatggtacttggatgatttattcttcacgctatatccttcccttgagaagtttgaggaagagcttctggaactcctcatcagtgataacttccgggagggggacagtccactagatggcagcacgctggagatcttggagcgccgcctgtgtgtgggcgtgcacaatggtctgggcttcgtgcagaggccacacgtggtggtgctggtgcccgagatggatgtggccttgacgcgctctgccagtttcagcaggaaagttggctcctcttcgaagaccagctctggaaaccaggctctagttttgagaagccgcctccgactccctgagatggtccatcaccctgcgtttgccatagtctttcaactggagtacgtgttcaccagcccttctggagtggacagcaaggcagcctcggtcacctctttgtccagtgtggcgtgtatgcacatagttcgctgggctgtttggaaccccttgctggaatccggctctgggaaggtgatcttacctctgcagggtgggatccggcccaacccctcacactgtttggtctataaagtaacccctgccagcatgagctctgaagaggtgcagcaagtggagtcagggactgtccagttccacttctcgctgagccctgagggacacctggacacacccacggggctggtccacagccctgaggcggagcggcagccctccaggaagccacccacatctcctgcaaacctgccagcactgccgccccaggggctcgctgccctccaggactcgccggtgggaccagggttgtccctttcccagctcgcagcttccccacagcctaccacccgctcccgctcagccacaccccctccacggcaacccgacagctctaagcccccgccggcccaggaggagctcccctcggagggcaggatctctcacctggaggcccagctgagccaggcgtccttggtcctgggagcctctgttgctgaacagctgcaggagctgccttttacgccggtgcacaccccgattgttgtgggagcccagaccaggagctctggaagtaagctctcacgagcctcaatggtgctcctgcaagcctgcggctttcctgagattctggatgccaataagcaaccagccgaggctgtaaatcctacagatcccgtgaaattcaacccccaaaaggaagaatcagattgtctacaaagcaatgagatagtgctgcattttcttgcctttagcagagtgtcccaggactgccagggagcaccgtggccaaagaccgtgtatttcaccttccagttctatcgcttcccacccgtgacaacgccacgcttgcagctgatcaggctggacgagacgggcaggacaggctctggttccctgttgtatctcctggttctcactgacaaagatggctcctctgatgctgggacacctggctttcaactcaagtacctggtggaacctgggttcctgaaacccggagaacagcgctggttcacccactacatggctgctcagaccctccagatcgacatctgggatggagactccttgctgctcgtgggatctgcggccgtccagatgaagcatctcctccgccaaggccggccggctgtgcaggtcctgcatgagcttgaggtcgtggcgactgaatatgaacaggatgtggtggtgagtggagatgtgaccagatttggctgtgtcaagcccatcggtgtctacacagtggtaaagggctggctgcacctgacgttggccaacgtgggtcatgtgtgtgagcagagagtgagagattccagctcattgccaccttccaggtcacgggtcatttcgaatgatggagccagacacttcgagggaggcagtctcatctcgcgcgggggtccgagatcagtaaaaaatgttgtccaagcacagaagctggctgacgtggaccgtgagctggccgccatgctgttcacccacatgcaacatgggagcaaggggccccaaggtgcttgccgtgagggggatgccgtccgcaggcgcaagctggagcggatgaggtccgtgcgccttcaagagtccagaggagagctcagctgctggggggccagcatgttgattcagcagaatatccgcgcacagcacgcacgtgacctgcaggtcattgctgcctatcgggagcgtacgaaggcagagagcatcaccagtgcgctgagcctggccatcaccacgatgcacacaatccacgtcacactgggcactgccgagttcttcgagtttgcccttaggaacccccacaacacacagcacacagtgaccattgaggtggacaatcctgagctcagcatcatccaggacagtcaggagtggagatacttcaaagacgcgaccaagctgcacacgccggtggaggaggacatgttccatctgcggggcagccttgccccgcagatcttcctgcgcccacgagagaccgctcacatccccttcaaataccagaccttctctgtagggcaactggccctggcgcagccctctgctgagctcggatctgagaaggacacggataccggattgccccagaaatccagtgcaatgcccactaagcatgccaaggtcttgttccgggggagtggcggtaagcccatcgccgtgctttgcctaaccgtggagacccagccccatgtggtggaccaggttttccgcttctaccacccagagctcaccttcctgaagaaggccatccgcctgcctccctggcacacgcttccaggtgctcccgtgggaagacctggtgaggagcccccagtctatgtccgatgtagcgaccccaacgtcatctgcgagacccagaatgtgggccccagggagcctcgggacgtgttcctgaaggtggccagcggtccaagcccagagagcagagacttctttgtcgccgtttacgcggatcgttggctggcgacacccatccaaatatggcaggtctacctccactccttgcagcgtgtggacgtctcctgtgtcacaggccagcggacacgcctatcccttgtccttcgagggacacggacggttaggagagtgcaggctttcacctcacaccctcaggagctgaagactgaccccaaaggtgtgttcgtgctgccaccacatggggtgcaggacctgcatgtcggtgtgaggccccgcagggcagggagccggttcatccacctcaacctggtcgacgtggactaccaccagctggtggcctcatggctcgtgtgcctctcctgccgcccgcctctcatctccaaggctttcgagatcacactggctgcagggaaaggcagaggcgccaacaagcggatcacctacaccaacccttacccctcccagaggacctatcacctgtacagcgaccaccctgacctgctgcagttcagagaggactccttccagattgggggaggagagacctacaccatcggcctgtggttcgcacctcggcagagccccggggaggaggagattctgatttacatcaatgaccacgaagacaaaaatgaagaagccttctgcgtcaaggtcgtgtaccagtaaaagagcagccggaggtgctggtggggcttcctgtcctgggggctctgtagccttttgtcccctcgatgacctgctggggctgctggtgagcatggccgagggtggtgtgcgcgccgtgcccctgcccagtcctcctgcccaagtaagcgctgctgtcaccagctgtgccacgagccccgcgggcgtcctcagggtacgaggctcgtttcttctgctgcactctggctgttgtcactgtctatgacatgttgatttactttttaggaatatatattttatgaatcatcttaagtgataaaattatgtattcttactggattttgtacaaacattatttgctatgcaatattttatattggtgtcctttcatatctttttttaagttacgtatgaaatactaaacttttacctgttttctct

SEQ ID NO: 6 - protein, 1429 aa, Canis familiaris
MNDWLRIFDQNVLVPPHPQRARQLLKESTAFQCVLKWLDGPLIKQGVLEVLSEVGCHLRVSLFDVTYRHFFGRTWKTAVRPAEQLSRQPSRIIFDEPLYFHTSLNHPNVVAVVEVVTEGRKQDGSLQALSCGFGILRIFGNKLESPTSASQDKRLRLYHGTPRALLHPLLQDPFEQNKHMTLIESCSLQYTLKPHPLLEPVFHLLPQNLLVSGLQHIPGLVPAHGEKGDTLRKPRLQKSVTWYLDDLFFTLYPSLEKFEEELLELLISDNFREGDSPLDGSTLEILERRLCVGVHNGLGFVQRPHVVVLVPEMDVALTRSASFSRKVGSSSKTSSGNQALVLRSRLRLPEMVHHPAFAIVFQLEYVFTSPSGVDSKAASVTSLSSVACMHIVRWAVWNPLLESGSGKVILPLQGGIRPNPSHCLVYKVTPASMSSEEVQQVESGTVQFHFSLSPEGHLDTPTGLVHSPEAERQPSRKPPTSPANLPALPPQGLAALQDSPVGPGLSLSQLAASPQPTTRSRSATPPPRQPDSSKPPPAQEEPPSEGRISHLEAQLSQASLVLGASVAEQLQELPFTPVHTPIVVGAQTRSSGSKLSRASMVLLQACGFPEILDANKQPAEAVNPTDPVKFNPQKEESDCLQSNEIVLHFLAFSRVSQDCQGAPWPKTVYFTFQFYRFPPVTTPRLQLIRLDETGRTGSGSLLYLLVLTDKDGSSDAGTPGFQLKYLVEPGFLKPGEQRWFTHYMAAQTLQIDIWDGDSLLLVGSAAVQMKHLLRQGRPAVQVLHELEVVATEYEQDVVVSGDVTRFGCVKPIGVYTVVKGWLHLTLANVGHVCEQRVRDSSSLPPSRSRVISNDGARHFEGGSLISRGGPRSVKNVVQAQKLADVDRELAAMLFTHMQHGSKGPQGACREGDAVRRRKLERMRSVRLQESRGELSCWGASMLIQQNIRAQHARDLQVIAAYRERTKAESITSALSLAITTMHTIHVTLGTAEFFEFALRNPHNTQHTVTIEVDNPELSIIQDSQEWRYFKDATKLHTPVEEDMFHLRGSLAPQIFLRPRETAHIPFKYQTFSVGQLALAQPSAELGSEKDTDTGLPQKSSAMPTKHAKVLFRGSGGKPIAVLCLTVETQPHVVDQVFRFYHPELTFLKKAIRLPPWHTLPGAPVGRPGEEPPVYVRCSDPNVICETQNVGPREPRDVFLKVASGPSPESRDFFVAVYADRWLATPIQIWQVYLHSLQRVDVSCVTGQRTRLSLVLRGTRTVRRVQAFTSHPQELKTDPKGVFVLPPHGVQDLHVGVRPRRAGSRFIHLNLVDVDYHQLVASWLVCLSCRPPLISKAFEITLAAGKGRGANKRITYTNPYPSQRTYHLYSDHPDLLQFREDSFQIGGGETYTIGLWFAPRQSPGEEEILIYINDHEDKNEEAFCVKVVYQ

SEQ ID NO: 7 - protein, 155 aa, Canis familiaris
MNDWLRIFDQNMLVPPHPQRARQLLKESTAFQCVLKWLDGPLIKQGVLEVLSEVGCHLRVSLFDVTYRHFFGRTWKTAVRPAEQLSRQPSRIIFDEPLYFHTSLNHPNVVAVVEVVTEGRKQDGSLQALSCGFGILRIFGNKLESPTSASQDKRK

SEQ ID NO: 8 - protein, 23 aa, Canis familiaris
RLRLYHGTPRALLHPLLQDPFEQ

SEQ ID NO: 9 - DNA, 295 bp, Canis familiaris
  misc_feature (132)..(132): Exon 5 deletion is after nucleotide 132
gctaagctgataatggtttacacagagaaagagaacaagatggagtcaaagcagataacaccaggaaagtcatagggaggatgagaggacttcacagcaacacctcaggccaggccaggccttgtctcttacctgcaaagcaagaccacagagcgcatgtcaaccctccgcatcctgcatttagccaagaaacatcagtgatgcttctcactccacacctactagagctgtgtcaacaccatacaatcacatcctctgcaggagagacaggtgagcaaagcagcagccccccatg

SEQ ID NO: 10 - DNA, 4290 bp, Canis familiaris
atgaatgactggctcaggatttttgaccaaaacgtgctggtccctccccacccacagagagcacgccagcttttaaaggaatcaacagcatttcagtgtgtcctgaagtggcttgacgggccactaattaagcagggagtgctggaggtgctttcggaggtgggctgccatctgcgtgtgtctctctttgatgtcacctaccgacactttttcgggaggacatggaagaccgcggtgaggccggctgagcagctttccaggcagccgtctaggatcatctttgatgagcccttgtactttcacacgtccttgaaccaccctaacgtggtggctgtggttgaggtggtcaccgaaggccggaagcaggatggctctctccaggctctgtcatgtggctttggaattcttcggatctttggcaacaaactggaatctcctacctctgcctcccaggacaagcggttgaggctgtatcacggcactccccgagccctcctgcacccactgctccaggaccctttcgaacaaaataaacacatgaccctcattgagagctgcagcctgcaatacactctgaagccacacccgctcctggagcccgtgttccatcttcttccccagaaccttctggtgtctggtctgcagcacatccctggcctcgttccagctcatggagaaaagggggacaccctccgaaagcctcgccttcagaagtctgtcacatggtacttggatgatttattcttcacgctatatccttcccttgagaagtttgaggaagagcttctggaactcctcatcagtgataacttccgggagggggacagtccactagatggcagcacgctggagatcttggagcgccgcctgtgtgtgggcgtgcacaatggtctgggcttcgtgcagaggccacacgtggtggtgctggtgcccgagatggatgtggccttgacgcgctctgccagtttcagcaggaaagttggctcctcttcgaagaccagctctggaaaccaggctctagttttgagaagccgcctccgactccctgagatggtccatcaccctgcgtttgccatagtctttcaactggagtacgtgttcaccagcccttctggagtggacagcaaggcagcctcggtcacctctttgtccagtgtggcgtgtatgcacatagttcgctgggctgtttggaaccccttgctggaatccggctctgggaaggtgatcttacctctgcagggtgggatccggcccaacccctcacactgtttggtctataaagtaacccctgccagcatgagctctgaagaggtgcagcaagtggagtcagggactgtccagttccacttctcgctgagccctgagggacacctggacacacccacggggctggtccacagccctgaggcggagcggcagccctccaggaagccacccacatctcctgcaaacctgccagcactgccgccccaggggctcgctgccctccaggactcgccggtgggaccagggttgtccctttcccagctcgcagcttccccacagcctaccacccgctcccgctcagccacaccccctccacggcaacccgacagctctaagcccccgccggcccaggaggagcccccctcggagggcaggatctctcacctggaggcccagctgagccaggcgtccttggtcctgggagcctctgttgctgaacagctgcaggagctgccttttacgccggtgcacaccccgattgttgtgggagcccagaccaggagctctggaagtaagctctcacgagcctcaatggtgctcctgcaagcctgcggctttcctgagattctggatgccaataagcaaccagccgaggctgtaaatcctacagatcccgtgaaattcaacccccaaaaggaagaatcagattgtctacaaagcaatgagatagtgctgcattttcttgcctttagcagagtgtcccaggactgccagggagcaccgtggccaaagaccgtgtatttcaccttccagttctatcgcttcccacccgtgacaacgccacgcttgcagctgatcaggctggacgagacgggcaggacaggctctggttccctgttgtatctcctggttctcactgacaaagatggctcctctgatgctgggacacctggctttcaactcaagtacctggtggaacctgggttcctgaaacccggagaacagcgctggttcacccactacatggctgctcagaccctccagatcgacatctgggatggagactccttgctgctcgtgggatctgcggccgtccagatgaagcatctcctccgccaaggccggccggctgtgcaggtcctgcatgagcttgaggtcgtggcgactgaatatgaacaggatgtggtggtgagtggagatgtgaccagatttggctgtgtcaagcccatcggtgtctacacagtggtaaagggctggctgcacctgacgttggccaacgtgggtcatgtgtgtgagcagagagtgagagattccagctcattgccaccttccaggtcacgggtcatttcgaatgatggagccagacacttcgagggaggcagtctcatctcgcgcgggggtccgagatcagtaaaaaatgttgtccaagcacagaagctggctgacgtggaccgtgagctggccgccatgctgttcacccacatgcaacatgggagcaaggggccccaaggtgcttgccgtgagggggatgccgtccgcaggcgcaagctggagcggatgaggtccgtgcgccttcaagagtccagaggagagctcagctgctggggggccagcatgttgattcagcagaatatccgcgcacagcacgcacgtgacctgcaggtcattgctgcctatcgggagcgtacgaaggcagagagcatcaccagtgcgctgagcctggccatcaccacgatgcacacaatccacgtcacactgggcactgccgagttcttcgagtttgcccttaggaacccccacaacacacagcacacagtgaccattgaggtggacaatcctgagctcagcatcatccaggacagtcaggagtggagatacttcaaagacgcgaccaagctgcacacgccggtggaggaggacatgttccatctgcggggcagccttgccccgcagatcttcctgcgcccacgagagaccgctcacatccccttcaaataccagaccttctctgtagggcaactggccctggcgcagccctctgctgagctcggatctgagaaggacacggataccggattgccccagaaatccagtgcaatgcccactaagcatgccaaggtcttgttccgggggagtggcggtaagcccatcgccgtgctttgcctaaccgtggagacccagccccatgtggtggaccaggttttccgcttctaccacccagagctcaccttcctgaagaaggccatccgcctgcctccctggcacacgcttccaggtgctcccgtgggaagacctggtgaggagcccccagtctatgtccgatgtagcgaccccaacgtcatctgcgagacccagaatgtgggccccagggagcctcgggacgtgttcctgaaggtggccagcggtccaagcccagagagcagagacttctttgtcgccgtttacgcggatcgttggctggcgacacccatccaaatatggcaggtctacctccactccttgcagcgtgtggacgtctcctgtgtcacaggccagcggacacgcctatcccttgtccttcgagggacacggacggttaggagagtgcaggctttcacctcacaccctcaggagctgaagactgaccccaaaggtgtgttcgtgctgccaccacatggggtgcaggacctgcatgtcggtgtgaggccccgcagggcagggagccggttcatccacctcaacctggtcgacgtggactaccaccagctggtggcctcatggctcgtgtgcctctcctgccgcccgcctctcatctccaaggctttcgagatcacactggctgcagggaaaggcagaggcgccaacaagcggatcacctacaccaacccttacccctcccagaggacctatcacctgtacagcgaccaccctgacctgctgcagttcagagaggactccttccagattgggggaggagagacctacaccatcggcctgtggttcgcacctcggcagagccccggggaggaggagattctgatttacatcaatgaccacgaagacaaaaatgaagaagccttctgcgtcaaggtcgtgtaccagtaa

SEQ ID NO: 11 - DNA, 465 bp, Canis familiaris
atgaatgactggctcaggatttttgaccaaaacatgctggtccctccccacccacagagagcacgccagcttttaaaggaatcaacagcatttcagtgtgtcctgaagtggcttgacgggccactaattaagcagggagtgctggaggtgctttcggaggtgggctgccatctgcgtgtgtctctctttgatgtcacctaccgacactttttcgggaggacatggaagaccgcggtgaggccggctgagcagctttccaggcagccgtctaggatcatctttgatgagcccttgtactttcacacgtccttgaaccaccctaacgtggtggctgtggttgaggtggtcaccgaaggccggaagcaggatggctctctccaggctctgtcatgtggctttggaattcttcggatctttggcaacaaactggaatctcctacctctgcctcccaggacaagcgaaaa

SEQ ID NO: 12 - DNA, 69 bp, Canis familiaris
cggttgaggctgtatcacggcactccccgagccctcctgcacccactgctccaggaccctttcgaacaa

SEQ ID NO: 13 - DNA, 22 bp, Artificial
  PCR primer
gcatgctaatatgtgaggcact

SEQ ID NO: 14 - DNA, 20 bp, Artificial
  PCR primer
caatgtggccatcacttgac

SEQ ID NO: 15 - DNA, 20 bp, Artificial
  PCR primer
aggaatcccaacaaggttcc

SEQ ID NO: 16 - DNA, 20 bp, Artificial
  PCR primer
cctagccacatcccactgtt

SEQ ID NO: 17 - DNA, 20 bp, Artificial
  PCR primer
cccttgaatgagtcccctaa

SEQ ID NO: 18 - DNA, 20 bp, Artificial
  PCR primer
tcgtcaagaaggcagattca

SEQ ID NO: 19 - DNA, 20 bp, Artificial
  PCR primer
ctgtcttgcctttgcctttc

SEQ ID NO: 20 - DNA, 20 bp, Artificial
  PCR primer
aactcttccccctttgcact

SEQ ID NO: 21 - DNA, 20 bp, Artificial
  PCR primer
caccctctgtgttgcctttt

SEQ ID NO: 22 - DNA, 20 bp, Artificial
  PCR primer
tccagctcacactcatgctc

SEQ ID NO: 23 - DNA, 20 bp, Artificial
  PCR primer
atttgccccaccttccttta

SEQ ID NO: 24 - DNA, 20 bp, Artificial
  PCR primer
aggtgcctgccttcagtcta

SEQ ID NO: 25 - DNA, 20 bp, Artificial
  PCR primer
ggaggctctgagttggagaa

SEQ ID NO: 26 - DNA, 20 bp, Artificial
  PCR primer
ctgcatagggcttcttgctc

SEQ ID NO: 27 - DNA, 20 bp, Artificial
  PCR primer
gggtggagggtagattccat

SEQ ID NO: 28 - DNA, 20 bp, Artificial
  PCR primer
cagcctccctgaaaatgagt

SEQ ID NO: 29 - DNA, 20 bp, Artificial
  PCR primer
aagcagccagtgccactatt

SEQ ID NO: 30 - DNA, 20 bp, Artificial
  PCR primer
ccctctgcctatgtctctgc

SEQ ID NO: 31 - DNA, 20 bp, Artificial
  PCR primer
ggctgccctctttaggttct

SEQ ID NO: 32 - DNA, 20 bp, Artificial
  PCR primer
gacacaggcagagggagaag

SEQ ID NO: 33 - DNA, 20 bp, Artificial
  PCR primer
ggcgctgtattctgttggtt

SEQ ID NO: 34 - DNA, 19 bp, Artificial
  PCR primer
gccagtgacagctctctgc

SEQ ID NO: 35 - DNA, 20 bp, Artificial
  PCR primer
caaaatcatctgggcaggtc

SEQ ID NO: 36 - DNA, 21 bp, Artificial
  PCR primer
acactcctggctggtctcatc

SEQ ID NO: 37 - DNA, 20 bp, Artificial
  PCR primer
catgacaactgcccattttg

SEQ ID NO: 38 - DNA, 20 bp, Artificial
  PCR primer
catccaaccatccaaaccat

SEQ ID NO: 39 - DNA, 20 bp, Artificial
  PCR primer
tgcccgaaaataaggatttg

SEQ ID NO: 40 - DNA, 20 bp, Artificial
  PCR primer
catctgagttccctgcatga

SEQ ID NO: 41 - DNA, 20 bp, Artificial
  PCR primer
gacacaggcagagggagaag

SEQ ID NO: 42 - DNA, 20 bp, Artificial
  PCR primer
ctcttgtaaccctgggagca

SEQ ID NO: 43 - DNA, 20 bp, Artificial
  PCR primer
ggacgccaaatgctattgtt

SEQ ID NO: 44 - DNA, 20 bp, Artificial
  PCR primer
tacccccatccacaaggata

SEQ ID NO: 45 - DNA, 20 bp, Artificial
  PCR primer
ccattggtgcagagtaagca

SEQ ID NO: 46 - DNA, 19 bp, Artificial
  PCR primer
gatggtggatggatggaca

SEQ ID NO: 47 - DNA, 20 bp, Artificial
  PCR primer
agttccatccacatcattgc

SEQ ID NO: 48 - DNA, 20 bp, Artificial
  PCR primer
cctggcaaaggtcatgtttt

SEQ ID NO: 49 - DNA, 20 bp, Artificial
  PCR primer
tgccatgaaaatttgctgaa

SEQ ID NO: 50 - DNA, 20 bp, Artificial
  PCR primer
gccatccaaatcagaaagga

SEQ ID NO: 51 - DNA, 20 bp, Artificial
  PCR primer
ttgatggcctatggctgaat

SEQ ID NO: 52 - DNA, 20 bp, Artificial
  PCR primer
caggtgcagaagcacaaaaa

SEQ ID NO: 53 - DNA, 20 bp, Artificial
  PCR primer
aggagtgctgtgcaggagat

SEQ ID NO: 54 - DNA, 20 bp, Artificial
  PCR primer
cgtatcctgtctccctctgc

SEQ ID NO: 55 - DNA, 20 bp, Artificial
  PCR primer
ccatgaaggcaaagagtgct

SEQ ID NO: 56 - DNA, 20 bp, Artificial
  PCR primer
caaaaggcagaggagtcagg

SEQ ID NO: 57 - DNA, 20 bp, Artificial
  PCR primer
tgtcatggttctgtccatgc

SEQ ID NO: 58 - DNA, 20 bp, Artificial
  PCR primer
actcctctggggcctagttc

SEQ ID NO: 59 - DNA, 20 bp, Artificial
  PCR primer
gttgagatccagtcccaagc

SEQ ID NO: 60 - DNA, 20 bp, Artificial
  PCR primer
tgactggtgcatgtgtgcta

SEQ ID NO: 61 - DNA, 20 bp, Artificial
  PCR primer
ggtcctttctccccaagaag

SEQ ID NO: 62 - DNA, 20 bp, Artificial
  PCR primer
atctgagccccctcctgtag

SEQ ID NO: 63 - DNA, 20 bp, Artificial
  PCR primer
tggggaagtatgcatgaggt

SEQ ID NO: 64 - DNA, 20 bp, Artificial
  PCR primer
cactgatgggatgtcagtgc

SEQ ID NO: 65 - DNA, 20 bp, Artificial
  PCR primer
ctctccctgctgtgctctct

SEQ ID NO: 66 - DNA, 20 bp, Artificial
  PCR primer
gatctctggcttgctccttg

SEQ ID NO: 67 - DNA, 20 bp, Artificial
  PCR primer
atgagagacacggagggaga

SEQ ID NO: 68 - DNA, 20 bp, Artificial
  PCR primer
tgcaccaaaagcaatttcaa

SEQ ID NO: 69 - DNA, 20 bp, Artificial
  PCR primer
tatggctgaatggtgttcca

SEQ ID NO: 70 - DNA, 20 bp, Artificial
  PCR primer
agcagccactgtggaaaagt

SEQ ID NO: 71 - DNA, 20 bp, Artificial
  PCR primer
caggcaaggatgttgctttt

SEQ ID NO: 72 - DNA, 20 bp, Artificial
  PCR primer
ggagaaggagggaccatgag

SEQ ID NO: 73 - DNA, 20 bp, Artificial
  PCR primer
ctcccttagcctggaatggt

SEQ ID NO: 74 - DNA, 19 bp, Artificial
  PCR primer
gagactcggggtcaagtcc

SEQ ID NO: 75 - DNA, 20 bp, Artificial
  PCR primer
gaaattgcggtttccttgaa

SEQ ID NO: 76 - DNA, 20 bp, Artificial
  PCR primer
caaactcacgaccccaaaat

SEQ ID NO: 77 - DNA, 20 bp, Artificial
  PCR primer
agttggacccctaaccgact

SEQ ID NO: 78 - DNA, 19 bp, Artificial
  PCR primer
gagcctgcttctccctctg

SEQ ID NO: 79 - DNA, 20 bp, Artificial
  PCR primer
agacactgtgccaggtgcta

SEQ ID NO: 80 - DNA, 20 bp, Artificial
  PCR primer
tggccctcttctcagtttgt

SEQ ID NO: 81 - DNA, 20 bp, Artificial
  PCR primer
cacttccagaggctttcagc

SEQ ID NO: 82 - DNA, 20 bp, Artificial
  PCR primer
agtccactatggctgggatg

SEQ ID NO: 83 - DNA, 20 bp, Artificial
  PCR primer
gtggacaatctgctctgcaa

SEQ ID NO: 84 - DNA, 20 bp, Artificial
  PCR primer
cttctccctctccctctgct

SEQ ID NO: 85 - DNA, 25 bp, Artificial
  PCR primer
gaacaattcaatatgctttcaaaaa

SEQ ID NO: 86 - DNA, 20 bp, Artificial
  PCR primer
ccattctccaggtgatggac

SEQ ID NO: 87 - DNA, 24 bp, Artificial
  PCR primer
tgaatgtttatcaccaaagtgtca

SEQ ID NO: 88 - DNA, 20 bp, Artificial
  PCR primer
gctcttgatcttggctcagg

SEQ ID NO: 89 - DNA, 20 bp, Artificial
  PCR primer
atcaggatgcccactttctg

SEQ ID NO: 90 - DNA, 20 bp, Artificial
  PCR primer
cctgcggaatctgatgctat

SEQ ID NO: 91 - DNA, 18 bp, Artificial
  PCR primer
gcctgcttctccctcctc

SEQ ID NO: 92 - DNA, 24 bp, Artificial
  PCR primer
cacagacaaccgctaatacatctc

SEQ ID NO: 93 - DNA, 21 bp, Artificial
  PCR primer
gagtactcaagggccaaagtt

SEQ ID NO: 94 - DNA, 20 bp, Artificial
  PCR primer
atatgctgggtgtggagctt

SEQ ID NO: 95 - DNA, 20 bp, Artificial
  Primer used in amplifying exon of DAB1 gene
ggctgaaggtggcactaaac

SEQ ID NO: 96 - DNA, 20 bp, Artificial
  Primer used in amplifying exon of DAB1 gene
ggcataccaggaagggtttt

SEQ ID NO: 97 - DNA, 21 bp, Artificial
  Primer used in amplifying exon of DAB1 gene
ctcggtccaatgatgtgactt

SEQ ID NO: 98 - DNA, 20 bp, Artificial
  Primer used in amplifying exon of DAB1 gene
ccacacagtaggcgctcaat

SEQ ID NO: 99 - DNA, 20 bp, Artificial
  Primer used in amplifying exon of DAB1 gene
aggagaggcaaacacaccag

SEQ ID NO: 100 - DNA, 20 bp, Artificial
  Primer used in amplifying exon of DAB1 gene
cggggactgtttgacacttt

SEQ ID NO: 101 - DNA, 20 bp, Artificial
  Primer used in amplifying exon of DAB1 gene
gctcgttttccttccatctg

SEQ ID NO: 102 - DNA, 20 bp, Artificial
  Primer used in amplifying exon of DAB1 gene
tccgaggctatttgtgctct

SEQ ID NO: 103 - DNA, 20 bp, Artificial
  Primer used in amplifying exon of DAB1 gene
taggaaaaggaagggctgtg

SEQ ID NO: 104 - DNA, 20 bp, Artificial
  Primer used in amplifying exon of DAB1 gene
tctgttctcctccccagaaa

SEQ ID NO: 105 - DNA, 20 bp, Artificial
  Primer used in amplifying exon of DAB1 gene
tggggaggagaacagaaaga

SEQ ID NO: 106 - DNA, 20 bp, Artificial
  Primer used in amplifying exon of DAB1 gene
gatcccgtgttatggatgct

SEQ ID NO: 107 - DNA, 20 bp, Artificial
  Primer used in amplifying exon of DAB1 gene
agaaaagtcagggggagcat

SEQ ID NO: 108 - DNA, 20 bp, Artificial
  Primer used in amplifying exon of DAB1 gene
gccattgaacatttccacaa

SEQ ID NO: 109 - DNA, 20 bp, Artificial
  Primer used in amplifying exon of DAB1 gene
ctgctgcccctataattcca

SEQ ID NO: 110 - DNA, 20 bp, Artificial
  Primer used in amplifying exon of DAB1 gene
ccttgatccagactggcact

SEQ ID NO: 111 - DNA, 21 bp, Artificial
  Primer used in amplifying exon of DAB1 gene
cttaaccactgctggctttcc

SEQ ID NO: 112 - DNA, 20 bp, Artificial
  Primer used in amplifying exon of DAB1 gene
caggctgggtgaggagaata

SEQ ID NO: 113 - DNA, 20 bp, Artificial
  Primer used in amplifying exon of DAB1 gene
agcccagataaactgccaga

SEQ ID NO: 114 - DNA, 20 bp, Artificial
  Primer used in amplifying exon of DAB1 gene
attgctgatgcatggtttca

SEQ ID NO: 115 - DNA, 19 bp, Artificial
  Primer used in amplifying exon of DAB1 gene
accagcttgtggtctgcac

SEQ ID NO: 116 - DNA, 20 bp, Artificial
  Primer used in amplifying exon of DAB1 gene
ggcagcctctcaaagtcatc

SEQ ID NO: 117 - DNA, 20 bp, Artificial
  Primer used in amplifying exon of DAB1 gene
tggctctggaaggaacactc

SEQ ID NO: 118 - DNA, 20 bp, Artificial
  Primer used in amplifying exon of DAB1 gene
gcctgctgaatgtgaactga

SEQ ID NO: 119 - DNA, 20 bp, Artificial
  Primer used in amplifying exon of DAB1 gene
gtgtgcacccactctttcct

SEQ ID NO: 120 - DNA, 20 bp, Artificial
  Primer used in amplifying exon of DAB1 gene
agcagggcatattttcatgc

SEQ ID NO: 121 - DNA, 20 bp, Artificial
  Primer used in amplifying exon of DAB1 gene
ggcactagaggggctttctt

SEQ ID NO: 122 - DNA, 20 bp, Artificial
  Primer used in amplifying exon of DAB1 gene
ttctccactcctcaccccta

SEQ ID NO: 123 - DNA, 20 bp, Artificial
  Primer used in amplifying exon of DAB1 gene
ttgaaaaccaagggcaaaac

SEQ ID NO: 124 - DNA, 20 bp, Artificial
  Primer used in amplifying exon of DAB1 gene
cggtcaacaggtcgaaaaat

SEQ ID NO: 125 - DNA, 20 bp, Artificial
  Primer used in amplifying exon of AJAP1 gene
gtgcttctgagatgctgacg

SEQ ID NO: 126 - DNA, 18 bp, Artificial
  Primer used in amplifying exon of AJAP1 gene
ggggacgaggaggaggag

SEQ ID NO: 127 - DNA, 20 bp, Artificial
  Primer used in amplifying exon of AJAP1 gene
gagtctgaagagcggacagg

SEQ ID NO: 128 - DNA, 20 bp, Artificial
  Primer used in amplifying exon of AJAP1 gene
ccagaaaagaggcggttgta

SEQ ID NO: 129 - DNA, 18 bp, Artificial
  Primer used in amplifying exon of AJAP1 gene
cggagacggagttcatcg

SEQ ID NO: 130 - DNA, 20 bp, Artificial
  Primer used in amplifying exon of AJAP1 gene
ggacacacaagtccctggat

SEQ ID NO: 131 - DNA, 19 bp, Artificial
  Primer used in amplifying exon of AJAP1 gene
atgctgaggctgggactct

SEQ ID NO: 132 - DNA, 20 bp, Artificial
  Primer used in amplifying exon of AJAP1 gene
ctgcacacaaacccacattc

SEQ ID NO: 133 - DNA, 20 bp, Artificial
  Primer used in amplifying exon of AJAP1 gene
gagagggagcgaactgattg

SEQ ID NO: 134 - DNA, 20 bp, Artificial
  Primer used in amplifying exon of AJAP1 gene
gccgcaacaccttctatgtt

SEQ ID NO: 135 - DNA, 18 bp, Artificial
  Primer used in amplifying exon of AJAP1 gene
acacggagcttggccttc

SEQ ID NO: 136 - DNA, 20 bp, Artificial
  Primer used in amplifying exon of AJAP1 gene
tggacagcagcactattgga

SEQ ID NO: 137 - DNA, 20 bp, Artificial
  Primer used in amplifying exon of VAMP3 gene
ttcacttgagaccctgctca

SEQ ID NO: 138 - DNA, 20 bp, Artificial
  Primer used in amplifying exon of VAMP3 gene
ctcgggaggagtatttgtgg

SEQ ID NO: 139 - DNA, 19 bp, Artificial
  Primer used in amplifying exon of VAMP3 gene
cctgccccatcctttctaa

SEQ ID NO: 140 - DNA, 20 bp, Artificial
  Primer used in amplifying exon of VAMP3 gene
atgtgcagcacgtcaaactc

SEQ ID NO: 141 - DNA, 20 bp, Artificial
  Primer used in amplifying exon of NPHP4 gene
taatgggctccacttccaac

SEQ ID NO: 142 - DNA, 20 bp, Artificial
  Primer used in amplifying exon of NPHP4 gene
tcaacaacagtggaacacca

SEQ ID NO: 143 - DNA, 20 bp, Artificial
  Primer used in amplifying exon of NPHP4 gene
ggagcaagatgggtgtgttt

SEQ ID NO: 144 - DNA, 20 bp, Artificial
  Primer used in amplifying exon of NPHP4 gene
tttgctgatgagccactttg

SEQ ID NO: 145 - DNA, 20 bp, Artificial
  Primer used in amplifying exon of NPHP4 gene
ttctggagggcaagtcattt

SEQ ID NO: 146 - DNA, 20 bp, Artificial
  Primer used in amplifying exon of NPHP4 gene
tttgttccaagaaccccaag

SEQ ID NO: 147 - DNA, 20 bp, Artificial
  Primer used in amplifying exon of NPHP4 gene
ttcctgtgggtcagaagctc

SEQ ID NO: 148 - DNA, 20 bp, Artificial
  Primer used in amplifying exon of NPHP4 gene
agcagaaaagctcccagaca

SEQ ID NO: 149 - DNA, 20 bp, Artificial
  Primer used in amplifying exon of NPHP4 gene
ttctaggtgcctgggtcttg

SEQ ID NO: 150 - DNA, 20 bp, Artificial
  Primer used in amplifying exon of NPHP4 gene
tcccctcaggctagtgctta

SEQ ID NO: 151 - DNA, 20 bp, Artificial
  Primer used in amplifying exon of NPHP4 gene
atgcctggacttcacacctt

SEQ ID NO: 152 - DNA, 20 bp, Artificial
  Primer used in amplifying exon of NPHP4 gene
tcccttcctcctctcctctc

SEQ ID NO: 153 - DNA, 20 bp, Artificial
  Primer used in amplifying exon of NPHP4 gene
gggcagaaattgggttgtaa

SEQ ID NO: 154 - DNA, 20 bp, Artificial
  Primer used in amplifying exon of NPHP4 gene
accatcatcgcaccaaagtt

SEQ ID NO: 155 - DNA, 20 bp, Artificial
  Primer used in amplifying exon of NPHP4 gene
tcttctagggggagggtgat

SEQ ID NO: 156 - DNA, 20 bp, Artificial
  Primer used in amplifying exon of NPHP4 gene
aaaggcaccagggaagaagt

SEQ ID NO: 157 - DNA, 20 bp, Artificial
  Primer used in amplifying exon of NPHP4 gene
gcaggaagttctgccattct

SEQ ID NO: 158 - DNA, 20 bp, Artificial
  Primer used in amplifying exon of NPHP4 gene
atgattgcaacaaagccaca

SEQ ID NO: 159 - DNA, 20 bp, Artificial
  Primer used in amplifying exon of NPHP4 gene
ttctgtgtgatccctgcttg

SEQ ID NO: 160 - DNA, 20 bp, Artificial
  Primer used in amplifying exon of NPHP4 gene
tgaatggggatgtcctaagc

SEQ ID NO: 161 - DNA, 20 bp, Artificial
  Primer used in amplifying exon of NPHP4 gene
gtgcaaaactggggaaatgt

SEQ ID NO: 162 - DNA, 20 bp, Artificial
  Primer used in amplifying exon of NPHP4 gene
caccctctgtcactgtccaa

SEQ ID NO: 163 - DNA, 20 bp, Artificial
  Primer used in amplifying exon of NPHP4 gene
tgtgaggacttcatgggtca

SEQ ID NO: 164 - DNA, 20 bp, Artificial
  Primer used in amplifying exon of NPHP4 gene
cactgtggcacctcagaaaa

SEQ ID NO: 165 - DNA, 20 bp, Artificial
  Primer used in amplifying exon of NPHP4 gene
acctcactgccttctgtgct

SEQ ID NO: 166 - DNA, 20 bp, Artificial
  Primer used in amplifying exon of NPHP4 gene
aaactttccttcccccttca

SEQ ID NO: 167 - DNA, 20 bp, Artificial
  Primer used in amplifying exon of NPHP4 gene
cacttgggcgtcctgtattt

SEQ ID NO: 168 - DNA, 20 bp, Artificial
  Primer used in amplifying exon of NPHP4 gene
attcttccttttgggggttg

SEQ ID NO: 169 - DNA, 20 bp, Artificial
  Primer used in amplifying exon of NPHP4 gene
ctgcaggagctgccttttac

SEQ ID NO: 170 - DNA, 20 bp, Artificial
  Primer used in amplifying exon of NPHP4 gene
tgtgtgagcaccctaaccaa

SEQ ID NO: 171 - DNA, 20 bp, Artificial
  Primer used in amplifying exon of NPHP4 gene
tgaaatgggttgctgtctga

SEQ ID NO: 172 - DNA, 20 bp, Artificial
  Primer used in amplifying exon of NPHP4 gene
ctggagacccaggatcgaat

SEQ ID NO: 173 - DNA, 20 bp, Artificial
  Primer used in amplifying exon of NPHP4 gene
ggggtattctgggaggagtt

SEQ ID NO: 174 - DNA, 20 bp, Artificial
  Primer used in amplifying exon of NPHP4 gene
ggaggagctgatgactgagg

SEQ ID NO: 175 - DNA, 20 bp, Artificial
  Primer used in amplifying exon of NPHP4 gene
ggtgaattggggtgcactta

SEQ ID NO: 176 - DNA, 19 bp, Artificial
  Primer used in amplifying exon of NPHP4 gene
aagggcaggccaagtagtg

SEQ ID NO: 177 - DNA, 20 bp, Artificial
  Primer used in amplifying exon of NPHP4 gene
tgctccagagaccaggaact

SEQ ID NO: 178 - DNA, 20 bp, Artificial
  Primer used in amplifying exon of NPHP4 gene
ctgcatgcacacagacacac

SEQ ID NO: 179 - DNA, 19 bp, Artificial
  Primer used in amplifying exon of NPHP4 gene
aggtggctggaagcttgtt

SEQ ID NO: 180 - DNA, 20 bp, Artificial
  Primer used in amplifying exon of NPHP4 gene
ccaggatcaagtcccacatt

SEQ ID NO: 181 - DNA, 20 bp, Artificial
  Primer used in amplifying exon of NPHP4 gene
cccgtttactgtcctctgga

SEQ ID NO: 182 - DNA, 20 bp, Artificial
  Primer used in amplifying exon of NPHP4 gene
ggagacacagcactcccagt

SEQ ID NO: 183 - DNA, 20 bp, Artificial
  Primer used in amplifying exon of NPHP4 gene
gcacacagtgaccattgagg

SEQ ID NO: 184 - DNA, 20 bp, Artificial
  Primer used in amplifying exon of NPHP4 gene
aggtgacctgctgaaggcta

SEQ ID NO: 185 - DNA, 20 bp, Artificial
  Primer used in amplifying exon of NPHP4 gene
ttcaggcagagccagttaca

SEQ ID NO: 186 - DNA, 20 bp, Artificial
  Primer used in amplifying exon of NPHP4 gene
ataccaacatcgcctgaagc

SEQ ID NO: 187 - DNA, 20 bp, Artificial
  Primer used in amplifying exon of NPHP4 gene
caaattgagcgctgacacat

SEQ ID NO: 188 - DNA, 20 bp, Artificial
  Primer used in amplifying exon of NPHP4 gene
atgggtcacaggttgggata

SEQ ID NO: 189 - DNA, 20 bp, Artificial
  Primer used in amplifying exon of NPHP4 gene
tgtctgcttctggtctggtg

SEQ ID NO: 190 - DNA, 21 bp, Artificial
  Primer used in amplifying exon of NPHP4 gene
tcactttgcttcatgcctttt

SEQ ID NO: 191 - DNA, 21 bp, Artificial
  Primer used in amplifying exon of NPHP4 gene
aaaggcatgaagcaaagtgaa

SEQ ID NO: 192 - DNA, 19 bp, Artificial
  Primer used in amplifying exon of NPHP4 gene
agccgtggagtctcacctc

SEQ ID NO: 193 - DNA, 20 bp, Artificial
  Primer used in amplifying exon of NPHP4 gene
aagtcacagtgggccttcag

SEQ ID NO: 194 - DNA, 20 bp, Artificial
  Primer used in amplifying exon of NPHP4 gene
acccagacgcatagttgtgg

SEQ ID NO: 195 - DNA, 20 bp, Artificial
  Primer used in amplifying exon of NPHP4 gene
actcggtccccttctgactt

SEQ ID NO: 196 - DNA, 20 bp, Artificial
  Primer used in amplifying exon of NPHP4 gene
tgatctcgaaagcctgtgtg

SEQ ID NO: 197 - DNA, 20 bp, Artificial
  Primer used in amplifying exon of NPHP4 gene
gtctcctccgcagtctatgg

SEQ ID NO: 198 - DNA, 20 bp, Artificial
  Primer used in amplifying exon of NPHP4 gene
ctgtgacagtgcccagatgt

SEQ ID NO: 199 - DNA, 20 bp, Artificial
  Primer used in amplifying exon of NPHP4 gene
agctgtgaatctcccctgag

SEQ ID NO: 200 - DNA, 20 bp, Artificial
  Primer used in amplifying exon of NPHP4 gene
gtgacagcagcgcttacttg

SEQ ID NO: 201 - DNA, 20 bp, Artificial
  Primer used in amplifying exon of NPHP4 gene
cggatctttggcaacaaact

SEQ ID NO: 202 - DNA, 21 bp, Artificial
  Primer used in amplifying exon of NPHP4 gene
ctgatgaggagttccagaagc

SEQ ID NO: 203 - DNA, 20 bp, Artificial
  Primer used in amplifying exon of NPHP4 gene
agctgtgaatctcccctgag

SEQ ID NO: 204 - DNA, 20 bp, Artificial
  Primer used in amplifying exon of NPHP4 gene
gtgacagcagcgcttacttg

SEQ ID NO: 205 - DNA, 240 bp, Canis familiaris
ctctctccaggctctgtcatgtggctttggaattcttcggatctttgggaacaaactggaatctcctacctctgcctcccaggacaagcggttgaggctgtatcacggcactccccgagccctcctgcacccactgctccaggaccctttcgaacaaaataaacacatgaccctcattgagagctgcagcctgcaatacactctgaagccacacccgctcctggagcccgtcttccatct

SEQ ID NO: 206 - DNA, 175 bp, Canis familiaris
ctctctccaggctctgtcatgtggctttggaattcttcggatctttgggaacaaactggaatctcctacctctgcctcccaggacaagcgaaaataaacacatgaccctcattgagagctgcagcctgcaatacactctgaagccacacccgctcctggagcccgtcttccatct

SEQ ID NO: 207 - DNA, 301 bp, Canis familiaris
gcgctctgtggtcttgctttgcaggttgaggctgtatcacggcactccccgagccctcctgcacccactgctccaggaccctttcgaacgtaagagacaaggcctggcctggcctgaggtgttgctgtgaagtcctctcatcctccctatgactttcctggtgttatctgctttgactccatcttgttctctttctcctgtgtaaaccattatcagcttaggtgatttatttaagggattaggacaaaaagtgtcatgagcttggatggttgacgtaactgaaggaactgtaagcactagc

SEQ ID NO: 208 - DNA, 119 bp, Canis familiaris
gcgctctgtggtcttgctttgcaggttgaggctgtatcacgggatttatttaagggattaggacaaaaagtgtcatgagcttggatggttgacgtaactgaaggaactgtaagcactag

SEQ ID NO: 209 - DNA, 269 bp, Canis familiaris
gttgaggctgtatcacggcactccccgagccctcctgcacccactgctccaggaccctttcgaacgtaagagacaaggcctggcctggcctgaggtgttgctgtgaagtcctctcatcctccctatgactttcctggtgttatctgctttgactccatcttgttctctttctctgtgtaaaccattatcagcttagctgatttatttaagggattaggacaaaaagtgtcatgagcttggatggttgacgtaactgaaggaactgtaag

(19) United States
(12) Patent Application Publication
(10) Pub. No.: US 2009/0235371 A1
(43) Pub. Date: Sep. 17, 2009

(54) Diagnostic method

(76) Inventors: Frode Lingaas, Fagernes (NO); Anne Caroline Wiik, Oslo (NO); Claire Margaret Wade, Como (AU); Kerstin Lindblad-Toh, Malden, MA (US)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/315,403

(22) Filed: Dec. 3, 2008

(30) Foreign Application Priority Data
Feb. 8, 2008 (GB) GB0802456.4

(51) Int. Cl. A01K 67/00; C12Q 1/68; G01N 33/00; G01N 33/566; C07K 16/00; C07H 21/00; C12N 15/74; C07K 14/00

(52) U.S. Cl. 800/8; 435/6; 436/94; 436/501; 530/387.1; 536/23.1; 435/320.1; 530/300

(57) ABSTRACT

The present invention relates to a method of diagnosing or detecting cone-rod dystrophy in a mammal, particularly in a canine species. In particular, the invention relates to methods involving the detection of a deletion in the NPHP4 (nephroretinin 4) gene and associated biomarkers. The invention also provides primers, nucleic acid molecules, polypeptides, antibodies, as well as kits for use in such methods.

### BACKGROUND OF THE INVENTION

1. Field of the Invention

The present invention relates to a method of diagnosing or detecting cone-rod dystrophy in a mammal, particularly in a canine species. In particular, the invention relates to methods involving the detection of a deletion in the NPHP4 (nephroretinin 4) gene and biomarkers associated with this deletion. The invention also provides primers, nucleic acid molecules, polypeptides, antibodies, as well as kits for use in such methods.

2. Description of Related Art

Cone-rod dystrophy (CRD) is a progressive retinal degenerative disease that causes deterioration of the cones and rods in the retina and frequently leads to blindness. The cone-rod dystrophies are a heterogeneous group of disorders that are known to occur naturally in man and dogs. The disorders are characterized by a predominant loss of cone function, with relative preservation of rod function [1, 2].

Cone-rod dystrophy is classified as a retinal ciliopathy, together with retinitis pigmentosa, macular degeneration, cone-dystrophy, Leber congenital amaurosis, and retinal degenerations associated with Usher syndrome, primary ciliary dyskinesia, Senior-Loken syndrome, Joubert syndrome, Bardet-Biedl syndrome, Laurence-Moon syndrome, McKusick-Kaufman syndrome and Biemond syndrome. Mutations for these latter disorders have been found in the genes: retinitis pigmentosa-1 (RP1), retinitis pigmentosa GTPase regulator (RPGR), retinitis pigmentosa GTPase regulator interacting protein (RPGRIP1), as well as the Usher, Bardet-Biedl, and nephronophthisis genes [3]. These genes are expressed in the outer segment of the photoreceptors, which is a specialized and modified cilium, where they have a structural role and are crucial for transport function. They also are involved in protein-protein interaction with transport-like proteins. [3]

Nephroretinin-4 has been found to be abundantly present at the subcortical plasma membrane region of polarized renal epithelial cells, in the centrosomes of dividing cells and, to a lesser extent in the primary cilia of highly confluent cell cultures [6 ]. NPHP4 is localized in the connecting cilium of photoreceptors, form a functional complex and interact with other proteins at the ciliary base.

The number of genes or loci currently identified for CRD in humans is low, compared to those for retinitis pigmentosa, RP (e.g. RetNet™). A few genes have been associated with autosomal recessive inherited CRD in man including ABCA4 [4, 5] and RPGRIP1 [10] and the mapped loci CORD8 [7, 8] and CORD9 [9].

Nephronophthisis (NPHP) comprises a group of autosomal recessive cystic kidney diseases, which constitute the most frequent genetic cause for end-stage renal failure in children and young adults. Six genes, NPHP1-6, have been identified as being mutated in human NPHP. The disease has additionally been associated with various extrarenal disorders. In Joubert syndrome (JBTS), NPHP may be associated with cerebellar vermis aplasia/hypoplasia, retinal degeneration and mental retardation. Mutations in NPHP6 and RPGRIP1L, a nephrocystin-4 interactor, are involved in Joubert syndrome [11, 12]. In Senior-Loken syndrome (SLSN), NPHP mutations are associated with retinitis pigmentosa, tapetoretinal degeneration or retinal dysplasia [13]. SLSN is present in about 10% of persons with mutations in NPHP1, 2, 3 or 4 [14-17] and >80% of persons with mutations in NPHP5 or NPHP6 [18, 19].

NPHP4 encodes nephroretinin-4 (also known as nephrocystin-4) that is conserved in evolution [17]. RPGRIP1 and NPHP4 interact strongly in vitro and in vivo, and they co-localize in the retina. Their interaction is disrupted by either mutations in RPGRIP1, found in patients with LCA, or by mutations in NPHP4, found in patients with SLSN [20].

At least 35 NPHP4 sequence variants are known in man, all causing nephronophthisis. A number of these display extrarenal manifestations, such as retinitis pigmentosa (RP), loss of hearing, bronchitis, Cogan syndrome, Usher syndrome and ophthalmologic abnormalities (unspecified).

No mutations in NPHP4 have previously been known to cause CRD. To date, only some unspecified ophthalmologic abnormalities have been described as being correlated with mutations in NPHP4, but these are always found in combination with kidney disease.

Mutations in NPHP6 are found to be a frequent cause of Leber congenital amaurosis without any kidney disease. Furthermore, a mutation in NPHP6 (CEP290) has been reported in low frequency Abyssinian cats, causing autosomal recessive late onset photoreceptor degeneration (rdAc).

So far, the only dog breeds in which CRD has been detected are the standard wire haired dachshund (SWHD), the miniature long haired dachshund (MLDH) and the pit bull terrier (PBT) [21-23]. A mutation in canine RPGRIP1 has been demonstrated to cause autosomal recessive CRD in miniature longhaired dachshunds [22], while the genetic basis of the cone-rod dystrophy found in the pit bull terrier is still unknown [21].

### SUMMARY OF THE INVENTION

The inventors have now found that a deletion in the NPHP4 gene is associated with the presence of CRD in dogs. This discovery is of significance not only in the diagnosis and detection of CRD in dogs, but also in other mammals, particularly humans. Phenotypic similarities between humans and dogs caused by mutations in orthologous genes make the dog a valuable model for the study of human retinal disease [24]. Dog populations display a number of different inherited retinal disorders and the genetic basis of at least 11 diseases have been identified to date [22, 25-34]. An example where an identical mutation was found for Retinitis Pigmentosa (RP17) in man and an orthologous progressive rod-cone dystrophy (PRCD) gene in the dog [33] shows the close relationship between the genes in the two species.

The molecular tools described herein can efficiently be used to reduce the frequency of the specific mutation or other mutations in this gene by obtaining a biological sample containing DNA and/or RNA or protein, and using the information about polymorphisms in the gene (e.g. the disease-associated mutation, SNPs in the same gene or the SNPs or microsatellites closely linked to the gene on each side, including promoters and enhancers) to define the haplotype and the disease/carrier-status of the mammal.

In one embodiment, the invention provides a method for diagnosing or detecting cone-rod dystrophy (CRD) in a mammal, the method comprising determining the presence or absence in a sample of nucleic acid that has been obtained from the mammal of: (i) a deletion in the NPHP4 gene, the deletion comprising all or part of exon 5, and (ii) a wild-type exon 5 in the NPHP4 gene, wherein the presence of (i) and the absence of (ii) is indicative of the presence of CRD in the mammal, and wherein the presence of (i) and the presence of (ii) is indicative of the mammal being a carrier for CRD.

Preferably, the deletion in exon 5 is one which results in the production, upon translation of the NPHP4 gene, of a polypeptide which is truncated at the C-terminal end compared to a wild-type NPHP polypeptide. In other embodiments, the deletion in exon 5 is one which results in the production, upon translation of the NPHP4 gene, of a NPHP4 polypeptide which does not bind to RPGRIP1.

In yet other embodiments, the deletion in exon 5 is one which results in the production, upon translation of the NPHP4 gene, of a NPHP4 polypeptide which does not bind to RPGRIP1 but does bind to NPHP1.

As discussed above, mutations in genes other than NPHP4 are known to cause CRD in at least some mammals. Hence the absence of (i) and the presence of (ii) cannot be said to be indicative of a normal (i.e. wild-type CRD phenotype) mammal in all cases.

The skilled person will understand that the mammal will possess two alleles of the NPHP4 gene and that the deletion in exon 5 and the wild-type NPHP4 gene exon 5 may be present in one or both alleles.

As used herein, the term “nucleic acid” encompasses both DNA and RNA. The nucleic acid may be single-stranded or double-stranded. Examples of nucleic acid include genomic DNA, cDNA and mRNA. In particular, the invention encompasses nucleic acid which has been amplified from nucleic acid obtained from the mammal, e.g. PCR'd DNA, and also cDNA which has been produced from RNA obtained from the mammal.

As used herein, references to cDNA relate to the cDNA which has been obtained from NPHP4 RNA, either wild-type NPHP4 RNA or mutated NPHP4 RNA having the deletion of the invention.

As used herein, the term “cone-rod dystrophy (CRD)” refers to a progressive retinal degenerative disease that causes deterioration of the cones and rods in the retina and frequently leads to blindness. The invention particularly relates to a form of CRD which is not associated with clinically-detectable kidney disease.

The NPHP4 gene is well known in the art [17]. Furthermore, the complete sequence of the Canis familiaris NPHP4 gene is disclosed herein; and FIG. 6 discloses a partial intron/exon map of this gene. Corresponding genes in other mammals are also known or can readily be identified. The performance of this invention in mammals other than Canis familiaris can therefore readily be carried out.

As used herein, the term “wild-type” NPHP4 gene refers to the NPHP4 gene which is present in most members of the population. The wild-type form of the gene contains all of the exons. In particular it does not have a deletion in exon 5.

The term “mammal” includes, inter alia, domestic mammals (dogs, cats, rabbits, etc.) and farm mammals (such as cows, pigs, sheep, donkeys, goats, etc) and well as humans. Preferably, the mammal is a dog or a human. In other embodiments of the invention, the mammal is a non-human mammal.

Preferred dog species include the dachshund and the pit bull terrier, particularly preferably the Standard Wirehaired Dachshund (SWHD) and the miniature long haired dachshund (MLDH).

The deletion of all or a part of exon 5 is preferably a deletion which results, during transcription into mRNA, in the skipping of exon 5, i.e. a mRNA is produced in which the 3′-end of exon 4 is joined to the 5′-end of exon 6.

Hence in embodiments of the invention where the nucleic acid sample is RNA, the deletion is preferably all of exon 5.

Exon 5 of the NPHP4 gene is generally about 65 bp in length, although some variation might exist from species to species.

Preferably, the deletion of part of exon 5 is one which results in the deletion of 1-65, 1-60, 1-50, 1-40, 1-30, 1-20 or 1-10 nucleotides from exon 5.

In some embodiments, the deletion is measured from the 5′-end in a 5′-3′ direction, possibly including the (5′-end) intron/exon boundary. In other embodiments, the deletion is measured from the 3′-end in a 3′-5′ direction, possibly including the (3′-end) exon/intron boundary. In yet other embodiments, the deletion will be in the middle part of the exon, leaving the both 5′- and 3′-intron/exon boundaries unaffected.

In some preferred embodiments where the nucleic acid is DNA, the deletion in exon 5 extends into the intron between exons 5 and 6 of the NPHP4 gene (i.e. into intron 5-6).

In some particularly preferred embodiments, the deletion starts at and includes nucleotide 19 of exon 5 and extends in a 3′ direction into intron 5, resulting in a deletion of about 180 nucleotides of genomic DNA.

The invention also provides a method for diagnosing or detecting cone-rod dystrophy (CRD) in a mammal, the method comprising determining the presence or absence in a sample of DNA that has been obtained from the mammal of:
(i) a deletion in a first allele of the NPHP4 gene, the deletion starting at and including nucleotide 19 of exon 5 and extending in a 3′ direction into intron 5,
(ii) a wild-type exon 5 in a second allele of the NPHP4 gene,
   wherein the presence of (i) and the absence of (ii) is indicative of the presence of CRD in the mammal, and wherein the presence of (i) and the presence of (ii) is indicative of the mammal being a carrier for CRD.

In some particularly preferred embodiments, the deletion results in a deletion of about 180 bp of genomic DNA.

The invention also provides a method for diagnosing or detecting cone-rod dystrophy (CRD) in a mammal, the method comprising determining the presence or absence in a sample of RNA that has been obtained from the mammal or of cDNA obtained therefrom of:
(i) a deletion in the NPHP4 RNA, the deletion consisting of all of exon 5,
(ii) a wild-type exon 5 in the NPHP4 RNA,
   wherein the presence of (i) and the absence of (ii) is indicative of the presence of CRD in the mammal, and wherein the presence of (i) and the presence of (ii) is indicative of the mammal being a carrier for CRD.

The skilled person will understand that, in mammals having a nucleic acid deletion in an exon, a mutated polypeptide will be produced. The complete amino acid sequence of the mutated dog NPHP4 polypeptide is given in SEQ ID NO: 7. Hence the presence of all of this sequence or a non-wild-type part of this amino acid sequence in a sample obtained from a mammal will be indicative of the mammal being either heterozygous or homozygous for the deletion.

The invention therefore also provides a method for diagnosing or detecting cone-rod dystrophy (CRD) in a mammal, the method comprising determining the presence or absence in a sample of nucleic acid that has been obtained from the mammal of:
(i) a nucleic acid molecule coding for a mutant or truncated NPHP4 polypeptide whose amino acid sequence consists of the amino acid sequence given in SEQ ID NO: 7 or an amino acid sequence which has at least 70% sequence identity thereto, preferably at least 75%, 80%, 85%, 90% or 95% sequence identity thereto, and
(ii) a nucleic acid molecule coding for a NPHP4 polypeptide whose amino acid sequence comprises SEQ ID NO: 8 or whose amino acid sequence comprises an amino acid sequence which has at least 70% sequence identity to SEQ ID NO: 8, preferably at least 75%, 80%, 85%, 90% or 95% sequence identity to SEQ ID NO: 8,
   wherein the presence of (i) and the absence of (ii) is indicative of the presence of CRD in the mammal, and wherein the presence of (i) and the presence of (ii) is indicative of the mammal being a carrier for CRD.

Preferably, the nucleic acid molecule encoding (i) comprises the nucleotide sequence given in SEQ ID NO: 3 or 5 or 9, or comprises a nucleotide sequence which has at least 70% sequence identity, preferably at least 80%, 85%, 90% or 95% sequence identity, to SEQ ID NO: 9.

Preferably, the NPHP4 polypeptide referred to in step (ii) is a polypeptide whose amino acid sequence consists of the amino acid sequence given in SEQ ID NO: 6 or an amino acid sequence which has at least 70%, preferably at least 75%, 80%, 85%, 90%, 95% or 99% sequence identity thereto and which also encodes a mammalian NPHP4 polypeptide.

In other embodiments, the nucleic acid molecule of step (ii) comprises the nucleotide sequence given in SEQ ID NO: 1 or 2, or comprises a nucleotide sequence which has at least 95% sequence identity to SEQ ID NO: 2, or comprises the nucleotide sequence given in SEQ ID NO: 4 or comprises a nucleotide sequence which has at least 75%, preferably at least 80%, 85%, 90% or 95%, sequence identity to SEQ ID NO: 4.

Preferably, the nucleic acid molecule of (ii) comprises the nucleotide sequence given in SEQ ID NO: 2.

In some embodiments of the invention, the term “method for detecting cone-rod dystrophy in a mammal” means a method for detecting probable cone-rod dystrophy in a mammal or a method of determining an increased likelihood of cone-rod dystrophy in a mammal.

The skilled person will be aware that the deletion may be detected either by direct means (such as those described above) or by indirect means, for example, by establishing a relationship between the deletion and a co-segregating polymorphic site (e.g. a Single Nucleotide Polymorphism or SNP). A number of polymorphic sites in the dog genome are already known.

In a further embodiment, therefore, the invention provides a method for diagnosing or detecting cone-rod dystrophy (CRD) in a mammal or for determining the likelihood of a mammal possessing a CRD genotype, the method comprising determining the presence or absence in a sample of nucleic acid which has been obtained from the mammal of:
(i) a polymorphic site which co-segregates with a deletion in the NPHP4 gene, the deletion comprising all or part of exon 5, and
(ii) a polymorphic site which co-segregates with the wild-type exon 5 of the NPHP4 gene,
   wherein the presence of (i) and the absence of (ii) is indicative of the presence of CRD in the mammal, and wherein the presence of (i) and the presence of (ii) is indicative of the mammal being a carrier for CRD.

The invention also provides a method for diagnosing or detecting cone-rod dystrophy (CRD) in a mammal or for determining the likelihood of a mammal possessing a CRD genotype, the method comprising determining the presence or absence in a sample of nucleic acid that has been obtained from the mammal of:
(i) a polymorphic site which co-segregates with a deletion in the NPHP4 gene, the deletion comprising all or part of exon 5, and
(ii) the wild-type exon 5 of the NPHP4 gene,
   wherein the presence of (i) and the absence of (ii) is indicative of the presence of CRD in the mammal, and wherein the presence of (i) and the presence of (ii) is indicative of the mammal being a carrier for CRD.

The invention further provides a method for diagnosing or detecting cone-rod dystrophy (CRD) in a mammal or for determining the likelihood of a mammal possessing a CRD genotype, the method comprising determining the presence or absence in a sample of nucleic acid that has been obtained from the mammal of:
(i) a deletion in the NPHP4 gene, the deletion comprising all or part of exon 5, and
(ii) a polymorphic site which co-segregates with the wild-type exon 5 of the NPHP4 gene,
   wherein the presence of (i) and the absence of (ii) is indicative of the presence of CRD in the mammal, and wherein the presence of (i) and the presence of (ii) is indicative of the mammal being a carrier for CRD.

The presence or absence of the polymorphic site may be detected using any of the methods described herein for the detection of the deletion, mutatis mutandis.

The polymorphic site may, for example, be a site having 1, 2, 3, 4, 5, 6, 7, 8, 9 or 10 contiguous or non-contiguous nucleotides, some or all of which nucleotides are different from the corresponding wild-type mammalian NPHP4 gene sequence.

Preferably the polymorphic site is a repeated motif of polynucleotides, wherein the motif is 1-50, 1-30, 1-20, 1-10, 1-5 or 1-2 nucleotides in length, and is repeated 1-100, 1-50, 1-40, 1-30, 1-20, or 1-10 times. Most preferably, the polymorphic site is a microsatellite or SNP.

In some preferred embodiments, the polymorphic site is a dinucleotide or tetranucletide motif, for example (AT)ₙ or (CG)_{n.}

The distance between the polymorphic site and the deletion may, for example, be 1-10⁷, 1-10⁶, 1-10⁵ or 1-10⁴, 1-10³ or 1-10² nucleotides. Preferably, the polymorphic site is as close as possible to the deletion, for example 1-50, 1-25 or 1-10 nucleotides.

Alternatively, the distance between the polymorphic site and the deletion may, for example, less than 50 cM, less than 20 cM, less than 10 cM, less than 5 cM or less than 1 cM.

The skilled person will understand that the closer the polymorphic site is to the deletion, the greater the probability will be that the polymorphic site and the deletion will co-segregate in any one mammal.

Preferably, the polymorphic site is within the NPHP4 gene, for example in an intron or exon or promoter, or a SNP or a microsatellite. Most preferably, the polymorphic site is a SNP.

The coding sequence of the wild-type canine NPHP4 gene is given herein in SEQ ID NOs: 1 (genomic) and 4 (cDNA); the corresponding polypeptide sequence is given in SEQ ID NO: 6.

The gene and polypeptide sequences of the mutant NPHP4 sequences are given herein as SEQ ID NOs: 3 (genomic) and 5 (cDNA), and 7 (polypeptide). It will be appreciated, however, by the person skilled in the art that the invention relates primarily to the presence or absence of a deletion in exon 5 of the coding sequence of the NPHP4 gene. Consequently, the precise nucleotide sequence of the NPHP4 gene of the mammal being tested is not of primary importance in the context of the present invention, i.e. the sequence of the NPHP4 gene of the mammal being tested might differ from that of the sequences disclosed herein due to natural sequence variation and inter-species variation. However, these natural sequence variations (if present) should not prevent the skilled person from identifying exon 5 in the NPHP4 gene/polypeptide in the mammal being tested. It is not a requirement of the invention, therefore, that the mammal being tested has a NPHP4 gene which has the precise sequences disclosed herein. The sequences given herein are included merely for reference purposes.

The person skilled in the art will readily be able to establish the nucleotide sequence corresponding to exon 5 of the mammalian NPHP4 gene in a sample of nucleic acid obtained from the mammal being tested. This may be done either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/), the Megalign program of the LASERGENE bioinformatics computing suite (DNASTAR Inc., Madison, Wis.) or using the Clustal method of alignment (Higgins and Sharp (1989) CABIOS. 5: 151-153). If using the Clustal method, default parameters for pairwise alignments may be KTUPLE 1, GAP PENALTY=3, WINDOW=5 and DIAGONALS SAVED=5.

The mammalian DNA to be tested may be obtained from any suitable cell type or tissue sample from the mammal. In a preferred embodiment, the DNA is obtained from a bodily fluid, e.g. blood, obtained by known techniques (e.g. venipuncture) or saliva. Alternatively, DNA can be obtained from dry samples (e.g. hair follicles or skin) or from germ cells, e.g. sperm or egg cells.

mRNA may be obtained from any cell type of the mammal which ordinarily expresses NPHP4, i.e. cell types which express NPHP4 in wild-type mammals. In mammals in which the deletion is present, a mutant form of NPHP4 mRNA should be expressed in muscle, kidney, lung, eye, nerve, brain, testis, thymus, trachea, uterus, bone and connective tissues. Preferably, the mRNA for testing is obtained from muscle, kidney or lung.

Numerous methods are known in the art for the detection of deletions such as the exon 5 deletion referred to herein. The invention is not limited to any one detection method. These methods include nucleic acid sequencing; microsequencing; allele specific oligonucleotide hybridization; size analysis; hybridization; 5′ nuclease digestion; single-stranded conformation polymorphism; allele specific hybridization; primer specific extension; oligonucleotide ligation assay; and restriction enzyme analysis.

Polymerase chain reaction (PCR) may or may not be used as part of the detection method of the invention. Using PCR, the presence or absence of the deletion in the mammalian NPHP4 gene may be detected by amplifying a region of nucleic acid from the mammalian genome which includes the deletion, using first and second nucleic acid primers. The primers will preferably have sequences and lengths so as to hybridize specifically under appropriate conditions to the region of mammalian nucleic acid which includes the deletion in exon 5. The primers in this case will not hybridize to the deleted part of exon 5, but will flank this region so that the deletion or wild-type sequence will be included in the fragment that is amplified. Primers may be designed, for example, using the SNP analysis program of the WatCut package found at http://watcut.uwaterloo.ca/watcut/watcut/template.php. The primers are preferably DNA primers. Preferred primers are disclosed herein.

The invention particularly relates therefore to a method as disclosed herein which additionally comprises the step of amplifying the region of the mammalian NPHP4 nucleic acid which includes exon 5 of the coding sequence of the mammalian NPHP4 gene. Preferably, the amplifying is by an exponential amplification method, e.g. the polymerase chain reaction (PCR). The length of the amplified nucleic acid which includes exon 5 of the coding sequence of the mammalian NPHP4 gene will generally be a length which will allow for the detection of the presence or absence of exon 5 by any suitable method. Preferably, the length of the region of amplified nucleic acid will be 20-3,000 nucleotides, more preferably 25-500 nucleotides and most preferably 50-250 nucleotides.

Preferably, the primers used in the amplification process are between 15-50 nucleotides in length, more preferably 18-35 nucleotides and most preferably 20-25 nucleotides in length. The upstream and downstream primers might have different lengths.

SEQ ID NO: 2 shows the nucleotide sequence of exons 4-6 of the wild-type canine NPHP4 gene. Exon 5, the start of intron 5-6 and the deletion in the mutant canine NPHP4 gene are shown below (with the deletion underlined):

Exon 5
GTTGAGGCTGTATCACGGCACTCCCCGAGCCCTCCTGCACCCACTGCTCC
AGGACCCTTTCGAAC
Intron 5-6

(SEQ ID NO: 209)

gtaagagacaaggcctggcctggcctgaggtgttgctgtgaagtcctctc
atcctccctatgactttcctggtgttatctgctttgactccatcttgttc
tctttctctgtgtaaaccattatcagcttagctgatttatttaagggatt
aggacaaaaagtgtcatgagcttggatggttgacgtaactgaaggaactg
taag . . .

Thus it will be clear to the skilled person where suitable primers may be located in order to amplify the region of DNA which is shown above. For example, the sequence of the upstream primer might be found within the end of intron 4/start of exon 5, within intron 4-5, or indeed further upstream. Similarly, the downstream primer may be located in the non-underlined region of intron 5-6 or further downstream.

Primers are preferably placed in intron 4 and in the undeleted part of intron 5. This provides a short PCR product in subjects with the deletion. The relative distance between the sizes of the PCR products in the deleted genotype versus the wild-type genotype is therefore large.

Preferred primers are those whose sequences are shown in the Figures. Primers for amplification of cDNA are preferably placed in exons 3 and 6.

The nucleic acid primers of the invention may additionally comprise, at the 5′-end of the primer, the nucleotide sequence of a restriction enzyme, thus facilitating the cloning of any amplified fragments. The nucleotide sequences of the primers of the invention might also be different from the naturally-occurring NPHP4 sequence in 1, 2 or 3 nucleotides, allowing for the production of artificial restriction enzyme sites within the sequence of the primers.

A further embodiment of the invention relates to nucleic acid primers as disclosed herein.

All nucleic acid fragments of the invention can be prepared according to methods well known in the art (e.g. [35]). For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides and primers of the invention may also be synthesized by standard methods known in the art, e.g. by use of an automated DNA synthesizer (such as is commercially available from Biosearch, Applied Biosystems, etc.).

Using methods well known in the art, nucleic acid sequencing of the region of nucleic acid which includes exon 5 of the NPHP4 gene may be used to determine the presence or absence of deletion compared to that of the wild-type coding sequence.

Examples of standard DNA sequencing methods include those based on techniques developed by Maxam and Gilbert [36] or Sanger [37]. A variety of automated sequencing procedures may also be utilized, as may sequencing by mass spectrometry.

Microsequencing methods may be used to determine the identity of a single nucleotide at a predetermined site. Hence these methods may be used to determine presence or absence of polymorphisms in the NPHP4 gene. Such microsequencing methods are discussed in Boyce-Jacino et al. (U.S. Pat. No. 6,294,336).

Single base variations in DNA sequences can be detected by a variety of techniques including Southern blot analysis for restriction fragment length polymorphisms, allele-specific oligonucleotide hybridization [38], denaturing gradient gel electrophoresis [39], chemical cleavage of mismatched heteroduplexes [40], conformational changes in single strands [41], and allele-specific priming of the polymerase chain reaction (PCR) [42-44].

An alternative strategy for DNA diagnosis, the oligonucleotide ligation assay (OLA), employs two adjacent oligonucleotides (20-mers), a 5′ biotinylated probe (with its 3′ end at the nucleotide to be assayed) and a 3′ reporter probe [45-47].

The invention also provides a method of testing a nucleic acid molecule, comprising determining the presence or absence of a nucleotide sequence in the nucleic acid molecule which encodes a truncated NPHP4 polypeptide or a mutant NPHP4 as disclosed herein.

As mentioned above, the skilled person will understand that, in mammals having a nucleic acid deletion in an exon, a mutated polypeptide will be produced. The complete amino acid sequence of the mutated dog NPHP4 polypeptide is given in SEQ ID NO: 7. Hence the presence of this amino acid sequence in a sample obtained from a mammal will be indicative of the mammal having at least carrier status, i.e. being heterozygous or possibly homozygous for the exon 5 deletion.

The invention therefore provides a method for diagnosing or detecting cone-rod dystrophy (CRD) in a mammal, the method comprising determining the presence or absence in a sample of polypeptide obtained from the mammal of:
(i) a mutant NPHP4 polypeptide whose amino acid sequence consists of the amino acid sequence given in SEQ ID NO: 7 or an amino acid sequence which has at least 70% sequence identity thereto, preferably at least 75%, 80%, 85%, 90% or 95% sequence identity thereto, and
(ii) a NPHP4 polypeptide whose amino acid sequence comprises SEQ ID NO: 8 or whose amino acid sequence comprises an amino acid sequence which has at least 70% sequence identity to SEQ ID NO: 8, preferably at least 75%, 80%, 85%, 90% or 95% sequence identity to SEQ ID NO: 8,
   wherein the presence of (i) and the absence of (ii) is indicative of the presence of CRD in the mammal, and wherein the presence of (i) and the presence of (ii) is indicative of the mammal being a carrier for CRD.

Preferably, the polypeptide sequence of step (ii) above consists of the amino acid sequence of SEQ ID NO: 6 or an amino acid sequence which has at least 80% sequence identity thereto, preferably at least 85%, 90% or 95% sequence identity thereto.

The detection of the polypeptides in the methods of the invention may be carried out by any suitable method known in the art. Examples of suitable methods include the fractionation of the polypeptides in the sample by one or two dimensional SDS-PAGE, optionally followed by Western blotting with an appropriate antibody; and various chromatographic methods including HPLC and protein identification using mass spectrometric (MS)-based methods [48]. Alternatively, part or all of a purified NPHP4-containing fraction or fragments thereof may be sequenced. Alternatively, the protein truncation test (PTT) may be used [49-50].

Due to the presence of the exon 5 deletion, the mutant NPHP4 polypeptide of SEQ ID NO: 7 is only 155 amino acids in length, as opposed to the wild-type polypeptide (SEQ ID NO: 6) which is 1429 amino acids in length. As a result of this, the mutant NPHP4 will present different epitopes compared to the wild-type polypeptide and hence the two polypeptides may be distinguished on this basis. Similar issues apply to mutated NPHP4 polypeptides from other species.

In yet a further embodiment, the invention provides a method for diagnosing or detecting cone-rod dystrophy (CRD) in a mammal, the method comprising:
(i) detecting whether a first antibody binds to a polypeptide sample obtained from the said mammal, wherein said first antibody is an antibody which binds to a polypeptide whose amino acid sequence consists of the sequence given in SEQ ID NO: 7 but does not bind to a polypeptide whose amino acid sequence consists of the sequence given in SEQ ID NO: 6, and
(ii) detecting whether a second antibody binds to a sample obtained from the said mammalian, wherein said second antibody is an antibody which binds to a polypeptide whose amino acid sequence consists of the sequence given in SEQ ID NO: 6 but does not bind to a polypeptide whose amino acid sequence consists of the sequence given in SEQ ID NO: 7,
   wherein the detection of binding of (i) and the absence of binding in (ii) is indicative of the presence of CRD in the mammal, and wherein the detection of binding in (i) and the detection of binding in (ii) is indicative of the mammal being a carrier for CRD.

In yet a further embodiment, the invention provides a method for diagnosing or detecting cone-rod dystrophy (CRD) in a mammal, the method comprising:
(i) detecting whether a first antibody binds to a polypeptide sample obtained from the said mammal, wherein said first antibody is an antibody which binds to a polypeptide whose amino acid sequence consists of the sequence given in SEQ ID NO: 7 or a sequence which has at least 80% sequence identity thereto (preferably at least 85%, 90% or 95% sequence identity thereto), but does not bind to a polypeptide whose amino acid sequence consists of the sequence given in SEQ ID NO: 6 or a sequence which has at least 80% sequence identity thereto (preferably at least 85%, 90% or 95% sequence identity thereto), and
(ii) detecting whether a second antibody binds to a sample obtained from the said mammal, wherein said second antibody is an antibody which binds to a polypeptide whose amino acid sequence consists of the sequence given in SEQ ID NO: 6 or a sequence which has at least 80% sequence identity thereto (preferably at least 85%, 90% or 95% sequence identity thereto), but does not bind to a polypeptide whose amino acid sequence consists of the sequence given in SEQ ID NO: 7 or a sequence which has at least 80% sequence identity thereto (preferably at least 85%, 90% or 95% sequence identity thereto),
   wherein the detection of binding of (i) and the absence of binding in (ii) is indicative of the presence of CRD in the mammal, and wherein the detection of binding in (i) and the detection of binding in (ii) is indicative of the mammal being a carrier for CRD.

The protein sample may be taken from any tissue which expresses NPHP4. Many NPHP4-expressing tissues are known (see http://www.ncbi.nlm.nih.gov/UniGene/ESTProfileViewer.cgi?uglist=Hs.462348). In particular, the tissue may be retina or lung, particularly preferably a tissue which would be expected to contain NPHP4 polypeptide of SEQ ID NO: 6 and/or SEQ ID NO: 7. The sample will, in general, be a protein sample, which may be purified or fractionated, as desired.

In some embodiments of the invention, the method for diagnosing or detecting cone-rod dystrophy includes the additional step of obtaining a sample from the mammal. The sample will be a sample of nucleic acid, DNA, RNA, polypeptide, etc. which is appropriate for the method in question.

In other embodiments, the method additionally comprises prescribing or administering a medicament to the mammal for the treatment of cone-rod dystrophy.

In the context of the present invention, the term “determining” may mean “obtaining an indication of the likelihood of” or “obtaining an indication of the probability of”.

The invention also provides a method for diagnosing or detecting cone-rod dystrophy (CRD) in a mammal, comprising the steps:
(i) sending one or more biological samples which have been obtained from the mammal to a remote location;
(ii) receiving data on the presence or absence of a deletion in all or part of exon 5 of the NPHP4 gene of the mammal,
   wherein the data has been obtained by a method of the invention.

In the above context, the biological sample is one which comprises appropriate nucleic acid or polypeptides upon which the methods of the invention can be used. Appropriate sources of nucleic acids and polypeptides are discussed above.

The sample may be sent to the remote location by any suitable means, e.g. by post or by courier.

The data may be received by any suitable means, including electronic means (e.g. by email, text-message, electronic file) and paper means (e.g. a letter or computer print-out).

In some embodiments, the above method additionally comprises the step: (iii) selecting a mammal which has a particular genotype or phenotype on the basis of the data received, and optionally breeding that mammal with a second mammal of the opposite sex by in vivo or in vitro means.

A further embodiment of the invention relates to an antibody which binds to a polypeptide whose amino acid sequence consists of the sequence given in SEQ ID NO: 7 but does not bind to a polypeptide whose amino acid sequence consists of the sequence given in SEQ ID NO: 6.

A further embodiment of the invention relates to an antibody which binds to a polypeptide whose amino acid sequence consists of the sequence given in SEQ ID NO: 6 but does not bind to a polypeptide whose amino acid sequence consists of the sequence given in SEQ ID NO: 7.

A further embodiment of the invention relates to an antibody which binds to a polypeptide whose amino acid sequence consists of the sequence given in SEQ ID NO: 7 or a sequence having at least 80% sequence identity thereto (preferably at least 85%, 90% or 95% sequence identity thereto) but does not bind to a polypeptide whose amino acid sequence consists of the sequence given in SEQ ID NO: 6 or a sequence having at least 80% sequence identity thereto (preferably at least 85%, 90% or 95% sequence identity thereto).

A further embodiment of the invention relates to an antibody which binds to a polypeptide whose amino acid sequence consists of the sequence given in SEQ ID NO: 6 or a sequence having at least 80% sequence identity thereto (preferably at least 85%, 90% or 95% sequence identity thereto) but does not bind to a polypeptide whose amino acid sequence consists of the sequence given in SEQ ID NO: 7 or a sequence having at least 80% sequence identity thereto (preferably at least 85%, 90% or 95% sequence identity thereto).

The antibodies of the invention may be of any suitable source, for example but not limited to monoclonal, polyclonal, chimeric, bispecific, single-chain or fragments thereof.

Antibody fragments include, but are not limited to, Fab, Fab′ and F(ab′)₂, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals, or derived from phage or ribosome display libraries. Preferably, the antibodies are human, murine (e.g. mouse or rat), donkey, rabbit, goat, guinea pig, camel, horse, or chicken. The antibodies of the invention may be monospecific, bispecific, trispecific or of greater multispecificity.

The antibodies of the invention may be labelled in any suitable manner, thus allowing for detection in a suitable assay.

Subtraction methods for the isolation of antibodies of the invention which bind to a polypeptide of SEQ ID NO: 7 but not to a polypeptide of SEQ ID NO: 6, or vice versa, are well known in the art.

In a further aspect, the invention provides a method for assaying for the presence of a mutated form of NPHP4 polypeptide in a sample, the method comprising:
(i) providing a substrate to which is bound NPHP1 polypeptide;
(ii) contacting said substrate with the sample, and optionally determining the binding of any NPHP4 polypeptide in the sample to the bound NPHP1;
(iii) washing the substrate in order to remove any unbound sample;
(iv) contacting the substrate with labelled RPGRIP1 polypeptide;
(v) washing the substrate; and
(vi) detecting binding of any labelled RPGRIP1 polypeptide to the NPHP4;
   wherein the binding of NPHP4 in step (ii) with the absence of binding of labelled RPGRIP1 polypeptide to the NPHP4 in step (vi) is indicative of the presence of mutated NPHP4 in the sample.

The source of the sample may be any tissue which normally expresses NPHP4, preferably retina or lung.

Preferably, the methods of the invention are carried out ex vivo or in vitro.

A further embodiment of the invention provides a kit for diagnosing or detecting cone-rod dystrophy (CRD) in a mammal, the kit comprising first and second nucleic acid primers for amplifying the region of nucleic acid which includes exon 5 of the mammalian NPHP4 gene. Preferably, the primers are ones disclosed in the Figures.

A yet further embodiment of the invention provides a kit for diagnosing or detecting cone-rod dystrophy (CRD) in a mammal, the kit comprising an antibody which binds to a polypeptide of SEQ ID NO: 7 but not to a polypeptide of SEQ ID NO: 6.

A yet further embodiment of the invention provides a kit for diagnosing or detecting cone-rod dystrophy (CRD) in a mammal, the kit comprising an antibody which binds to a polypeptide of SEQ ID NO: 6 but not to a polypeptide of SEQ ID NO: 7.

Also provided is a kit for diagnosing or detecting cone-rod dystrophy (CRD) in a mammal, the kit comprising:
(i) an antibody which binds to a polypeptide whose amino acid sequence consists of the sequence given in SEQ ID NO: 7 or a sequence having at least 80% sequence identity thereto but does not bind to a polypeptide whose amino acid sequence consists of the sequence given in SEQ ID NO: 6 or a sequence having at least 80% sequence identity thereto; and/or
(ii) an antibody which binds to a polypeptide whose amino acid sequence consists of the sequence given in SEQ ID NO: 6 or a sequence having at least 80% sequence identity thereto but does not bind to a polypeptide whose amino acid sequence consists of the sequence given in SEQ ID NO: 7 or a sequence having at least 80% sequence identity thereto.

A further embodiment of the invention relates to a nucleic acid molecule whose nucleotide sequence consists of the sequence given in SEQ ID NO: 3 or 5 or 9, of the complement thereof.

A yet further embodiment of the invention provides a nucleic acid molecule which encodes a polypeptide of SEQ ID NO: 7.

The invention also relates to the aforementioned nucleic acid molecules when present in a vector, preferably an expression vector, and an isolated host cell comprising said vector. Such a nucleic acid molecule may be used to produce the polypeptide of SEQ ID NO: 7, which is useful for producing the antibodies of the invention.

The isolated host cell is preferably a bacterial cell, yeast cell, animal cell or mammalian cell, for example a CHO cell or human cell. In some embodiments of the invention, the host cell is not a human embryonic cell or a human embryonic stem cell.

A further aspect of the invention provides a method for producing vectors or constructs, the method comprising the steps:
i) producing by extraction, amplification or synthesis a wild type or mutated NPHP4-gene sequence for the purpose of gene transfer or research in mammals, most preferably dogs or humans
(ii) determining the NPHP4 genotype of DNA in a vector or construct
(iii) selecting a vector or construct containing the wild type or mutated NPHP4 gene
(iii) transferring the NPHP4 gene to a mammal artificially, using vector/constructs.

In particular, the invention provides a process for producing a vector, comprising the steps:
(i) obtaining a wild-type or mutant NPHP4 gene;
(ii) determining the genotype of the NPHP4 gene using a method as disclosed herein; and
(iii) inserting the NPHP4 gene into a vector.
   Optionally, the vector is transformed, transfected or injected into a mammalian cell.

The deletion in exon 5 of the NPHP4 gene has been shown herein to be correlated with cone-rod dystrophy. The resulting polyptide has only 155 amino acids (in the canine form of the mutant polypeptide). This mutant polypeptide contains the binding site for Nephrocystin (NPHP1); NPHP1 interacts with NPHP4 in kidney. However, the mutant polypeptide has lost the binding site for RPGRIP1; the latter polypeptide interacts with wild-type NPHP4 in retina. The invention therefore provides a mutant NPHP4 polypeptide which binds NPHP1 but does not bind RPGRIP1.

A yet further embodiment of the invention relates to a polypeptide whose amino acid sequence consists of the sequence given in SEQ ID NO: 7 or a sequence having at least 75%, preferably at least 80%, 85%, 90% or 95% sequence identity thereto. Such polypeptides may be used to produce antibodies for use in the methods described herein which require such antibodies.

A yet further embodiment of the invention relates to a polypeptide whose amino acid sequence consists of the sequence given in SEQ ID NO: 8 or a sequence having at least 75% sequence identity thereto, preferably at least 80%, 85%, 90% or 95% sequence identity thereto.

The skilled person will appreciate that an indication of the genotypic/phenotypic status of a particular mammal regarding its susceptibility to cone-rod dystrophy (CRD) may be obtained by testing the parent or progeny of the mammal, instead of the mammal per se.

Sequence alignments and percent similarity calculations may be determined using a variety of comparison methods designed to detect homologous sequences including, but not limited to, the Megalign program of the LASARGENE bioinformatics computing suite (DNASTAR Inc., Madison, Wis.). Multiple alignment of the sequences are performed using the Clustal method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

The methods of the invention may be used to select mammals for specific breeding programmes. In some cases, it may be desirable to identify mammals which are heterozygous for the exon 5 deletion or mammals which are homozygous for the exon 5 deletion and selectively use these mammals in breeding programmes. In other embodiments, it will be desirable to select mammals which are homozygous for not having the exon 5 deletion.

In the following, the first and second mammals will generally be of the same species. In preferred embodiments of the methods of producing a mammal and mammal breeding systems, the mammal is a non-human mammal.

The invention therefore provides a method of producing a mammal, the method comprising the steps: (i) selecting a first mammal whose NPHP4 genotype has previously been determined using a method of the invention, and (ii) breeding said first mammal with a second mammal of the opposite sex, in order to produce progeny mammals.

Yet another embodiment of the invention provides a method of producing a mammal, the method comprising the steps: (i) determining the NPHP4 genotype of a group of one or more mammals using a method of the invention, and selecting a first mammal from within this group, and (ii) breeding the first mammal with a second mammal of the opposite sex, in order to produce progeny mammals.

The history of any one mammal and whether its NPHP4 genotype has previously been determined using a method of the invention will be readily apparent from the farmer's or owner's records on that mammal.

The invention further provides a method for producing a mammal, the method comprising the steps: (i) selecting a male first mammal whose NPHP4 genotype has previously been determined using a method of the invention, and (ii) inseminating a female second mammal with sperm from the male first mammal, in order to produce progeny mammals.

The invention further provides a method for producing a mammal, the method comprising the steps: (i) determining the NPHP4 genotype of a group of one or more male mammals using a method of the invention and selecting a male first mammal from within this group, and (ii) inseminating a female second mammal with sperm from the male first mammal, in order to produce progeny mammals.

The invention further provides a method for producing a mammal, the method comprising the steps: (i) selecting a female first mammal whose NPHP4 genotype has previously been determined using a method of the invention, and (ii) inseminating the female first mammal with sperm from a male second mammal, in order to produce progeny mammal.

The invention further provides a method for producing a mammal, the method comprising the steps: (i) determining the NPHP4 genotype of a group of one or more female mammals using a method of the invention and selecting a female first mammal from within this group, and (ii) inseminating the female first mammal with sperm from a male second mammal,

in order to produce progeny mammals.

In some embodiments of the invention, step (i) additionally comprises the step of selecting the second mammal from within the group.

Preferably, the first and/or second mammal is one which has previously been or is selected as being homozygous or heterozygous for the NPHP4 wild-type genotype or which has been or is selected as not having the NPHP4 exon 5 deletion genotype.

In some embodiments, the first mammal is homozygous or heterozygous for the NPHP4 wild-type genotype. In other embodiments, the second mammal is homozygous or heterozygous for the NPHP4 wild-type genotype. In yet other embodiments, the first and second mammals are either both homozygous or both heterozygous for the NPHP4 wild-type genotype.

The invention also provides a method of producing a mammal which is homozygous for the NPHP4 wild-type genotype, the method comprising the steps: (i) determining the NPHP4 genotype of a group of two or more mammals using a method as disclosed herein, (ii) selecting a first mammal from the group which is homozygous for the NPHP4 wild-type genotype, (iii) selecting a second mammal of the opposite sex to the first from the group which is homozygous for the NPHP4 wild-type genotype, (iv) breeding said first mammal with said second mammal, in order to produce progeny which are homozygous for the NPHP4 wild-type genotype.

The invention further provides a method of producing a mammal which is homozygous for a NPHP4 mutant genotype, the method comprising the steps: (i) determining the NPHP4 genotype of a group of two or more mammals using a method as disclosed herein, (ii) selecting a first mammal from the group which is homozygous for the NPHP4 mutant genotype, (iii) selecting a second mammal of the opposite sex to the first from the group which is homozygous for the NPHP4 mutant genotype, and (iv) breeding said first mammal with said second mammal, in order to produce progeny which are homozygous for the NPHP4 mutant genotype.

The invention also relates to mammals (preferably non-human mammals) produced by the above methods and to progeny thereof. The invention particularly relates to germ cells, for example sperm and eggs, from the aforementioned mammals.

The invention further provides a method of producing a zygote, the method comprising the steps: (i) combining germ cells from a first mammal whose NPHP4 genotype has previously been determined using a method of the invention with germ cells from a second mammal of the opposite sex, and (ii) culturing the combined germ cells under conditions suitable for producing a zygote.

The invention also provides a method of producing a zygote, the method comprising the steps: (i) determining the NPHP4 genotype of a group of one or more mammals using a method of the invention, and selecting a first mammal from within this group, (ii) combining germ cells from the first mammal with germ cells from a second mammal of the opposite sex, and (iii) culturing the combined germ cells under conditions suitable for producing a zygote.

The invention also provides a method for producing sperm, the method comprising the steps: (i) determining the NPHP4 genotype of a group of one or more mammals using a method as disclosed herein, (ii) selecting a male mammal from the group, and (iii) obtaining sperm from the male mammal.

Preferably, the obtained sperm is then stored, most preferably under refrigerated conditions. The invention also relates to sperm produced by the above method.

The invention further provides a method for producing eggs, the method comprising the steps: (i) determining the NPHP4 genotype of a group of one or more mammals using a method as disclosed herein, (ii) selecting a female mammal from the group, and (iii) obtaining one or more eggs from the female mammal.

Preferably, the obtained eggs are then stored, most preferably under refrigerated conditions. The invention also relates to eggs produced by the above method.

In some embodiments of the invention, the selected male mammal will be homozygous for the NPHP4 wild-type genotype. In other embodiments of the invention, the selected mammal will be homozygous for not having the NPHP4 exon 5 deletion.

The invention additionally provides a mammal breeding system comprising the steps: (i) maintaining a first population of mammals which are homozygous for the NPHP4 wild-type genotype and/or a second population which are homozygous for not having the NPHP4 exon 5 deletion genotype.

Optionally, the NPHP4 genotype of the first population and/or the second population has previously been determined using a method of the invention.

Alternatively, the mammal breeding system additionally comprises the step: determining the NPHP4 genotype of one or more mammals in the first population using a method of the invention and/or one or more mammals in the second population using a method of the invention.

Preferably, the mammal breeding system additionally includes the step:
(ii) selectively breeding a first mammal from the first population with a second mammal from the second population, wherein the first and second mammal are of the opposite sex; and producing progeny therefrom.

The invention also provides a mammal breeding system comprising the steps: (i) maintaining a first population of mammals which are homozygous for the NPHP4 wild-type genotype and a second population which are heterozygous for a deletion in NPHP4 exon 5 in one allele and wild type NPHP4 in the other allele, and optionally, (ii) selectively breeding a first mammal from the first population with a second mammal from the second population, wherein the first and second mammal are of the opposite sex; and producing progeny therefrom.

The invention also relates to mammals produced by the mammal breeding systems of the invention, and also to germ cells, for example sperm and egg cells, obtained from the mammal.

The invention also relates to processes for producing transgenic mammals (preferably non-human mammals) which are homozygous or heterozygous for not having the NPHP4 exon 5 deletion. Such mammals may, for example, be produced using nuclear transfer from cultured somatic cells (McCreath et al., Nature (2000), 405, 1066-1069) or by using mammalian embryonic stem cells. The exon 5 deletion may be removed from the mammalian genome by any known means, e.g. homologous recombination using an appropriate vector containing the wild-type exon 5 and adjacent sequences, and optionally a suitable selection marker (e.g. antibiotic resistance). Methods of homologous recombination are well known in the art. Alternatively, appropriate vectors may be used to introduce the wild-type NPHP4 gene into the genome of the mammal at a location other than the wild-type location.

Mammal somatic cells may be isolated by any appropriate means. Generally such cells are grown in culture and then transformed with the homologous recombination vector. Transformed cells are then selected before the nucleus is transferred to a enucleated mammal egg cell.

A further aspect of the invention therefore relates to a process for producing a modified mammalian cell, comprising the step of introducing a wild-type NPHP4 gene into the genome of the mammalian cell.

Preferably, the mammalian cell is a somatic mammalian cell or a mammalian embryonic stem cell.

In some embodiments, the mammalian cell is a somatic mammalian cell and the process additionally comprises the step of transferring the nucleus of the somatic mammalian cell to a mammalian enucleated egg cell. In embodiments wherein the mammalian cell is a somatic cell, it is preferably a fibroblast. The invention also includes mammals made using the above process.

The invention also relates to a mammal which is homozygous or heterozygous for a wild-type NPHP4 gene or non-exon 5 deleted NPHP4 gene or a functional NPHP4 gene, wherein the mammal additionally comprise a selective marker in its genome adjacent to the NPHP4 gene. Preferably, the selective marker is an antibiotic resistance marker.

A further aspect of the invention relates to a process of transferring the wild type NPHP4 gene to an affected patient in an attempt to cure the disease or in an attempt to learn more about gene therapy for eye disease in general or specifically retinal eye disease, using dogs with the NPHP4 mutation described herein.

A number of methods would be known for the skilled person in the field, and would often include the use of different vectors, first of all viral vectors. In some embodiments, the process could be performed by injecting wild type versions of the gene, or trying to introduce genes that express the wild-type protein (and maintain the expression of the gene in a long period) in the affected cells.

The process could be performed in mammals, after typing and detecting mutations in the NPHP4 gene, preferably in the first part of the gene that interacts with RPGRIP1, or more preferably with individuals with mutations in exon 5. This could be performed in any mammals, but most preferable in dogs and humans.

The invention therefore provides a method of treating cone rod dystrophy in a patient, comprising administering to the patient a vector comprising a wild-type NPHP4 gene. Preferably, the vector is administered directly or indirectly to the cells which are affected by cone-rod dystrophy, particularly preferably to the eye or the retina of the patient. Preferably, the vector comprises the NPHP4 gene in a form which facilitates the transfer of the NPHP4 gene to affected cells. Most preferably, the NPHP4 gene is stably transferred to the genome of the affected cells, at a position which allows expression of the NPHP4 gene.

A number of single nucleotide polymorphisms (SNPs) and microsatellite markers which are found in the region of the NPHP4 gene are disclosed herein. Individual SNPs/markers disclosed herein or combinations of SNPs/markers (e.g. haplotypes) disclosed herein form part of the invention and can be used as tools for the identification of individual mammals. For example, the haplotype of an individual mammal which has particular value in a breeding programme can be used for the identification of that individual and/or for the identification of the offspring of that individual.

The invention therefore provides the use of one or more SNPs or microsatellite markers as disclosed herein for the identification of a mammal or family of mammals, or offspring thereof.

The invention also provides the use of one or more SNPs or microsatellite markers as disclosed herein for the identification of mutations in the NPHP4 gene. Furthermore, the invention provides the use of one or more SNPs or microsatellite markers as disclosed herein for the identification of mutations in a gene associated with eye disorders.

### BRIEF DESCRIPTION OF THE FIGURES

FIG. 1. Pedigree of the dachshund family segregating crd that was used for genome-wide association. Discordant sibs of half sibs, marked *, were genotyped on the canine SNP-array and sibTDT genome-wide association was performed. Microsatellite markers in the associated CFA5 region was used for identification of recombinant breakpoints and haplotypes are shown for each dog.

FIG. 2. Primers used for amplifying all microsatellite markers used.

FIG. 3. Primers used in amplifying the exons of the DAB1, AJAP1 and VAMP3 candidate genes.

FIG. 4. Primers used for amplifying all NPHP4 exons.

FIG. 5. Genome-wide association mapping identified a locus on CFA5.

5A. The best association was identified by sibTDT analysis (pgenomeTDT=0.00060) but case-control association was also significant (PgenomeASSOC=5.2×10-3) using plink [51].

5B. Comparison of sibTDT and single SNP association statistics across the CFA5 region.

FIG. 6. Illustration of the 180 bp deletion in exon 5 of the NPHP4 gene on DNA, RNA and protein level. The mutation leads to exon-skipping of exon 5 and a stop codon in exon 6 which truncates the affected protein at 155aa. The truncated protein contains the nephrocystin interacting domain but not the RPGRIP1 domain.

FIG. 7. Location of SNP's in the genomic sequence of NPHP4.

FIG. 8. A deletion in exon 5 is visualized on the genomic and transcript level.

A. PCR products of exon 5 in affected dogs (338 bp, lane 1 and 2) carrier dogs (heterozygotes, lanes 3, 4, and 5) and unaffected dog (518 bp, lane 6).

B. PCR amplification of cDNA of one affected (329 bp, lane 1) and one healthy dog (392 bp, lane 2) using primers localized in exon 3 and exon 6.

C. Alignment of sequenced PCR product of mutant and wildtype cDNA and

D. DNA.

E. Testing for the mutation in human exon 5 showing a normal PCR-product from a wild type individual.

### DETAILED DESCRIPTION

Brief Description of the Sequences

SEQ ID NO: 1 is the genomic sequence of the wild-type Canis familiaris NPHP4 gene. (5 dna:chromosome chromosome:BROADD2:5:62820170:62935638:1)

SEQ ID NO: 2 is part of the genomic sequence of Canis familiaris NPHP4 gene, from and including exon 4 to exon 6.

SEQ ID NO: 3 is the genomic sequence of the mutant Canis familiaris NPHP4 gene, with the 180 nucleotide deletion starting from and including nucleotide 19 of exon 5. In the “NNNN” sequence, N can be any of the nucleotides A,C,G or T. (In this sequence, all the NNNN-nucleotide stretches are in the introns). (5 dna:chromosome chromosome:BROADD2:5:62820170:62935638:1)

SEQ ID NO: 4 is the cDNA sequence of the wild-type Canis familiaris NPHP4 gene.

SEQ ID NO: 5 is the cDNA sequence of the mutant Canis familiaris NPHP4 gene with the deletion of exon 5.

SEQ ID NO: 6 is the full amino acid sequence of the wild-type Canis familiaris NPHP4 gene.

SEQ ID NO: 7 is the full amino acid sequence of the mutant Canis familiaris NPHP4 polypeptide which results from the deletion of exon 5.

SEQ ID NO: 8 is the amino acid sequence encoded by wild-type NPHP4 exon 5. (only).

SEQ ID NO: 9 is a fragment of SEQ ID NO: 3 which spans, but excludes, the exon 5 deletion. The contiguous sequence is present in mutant Canis familiaris genomic DNA.

SEQ ID NO: 10 is the nucleotide sequence which encodes the amino acid sequence of SEQ ID NO: 6.

SEQ ID NO: 11 is the nucleotide sequence which encodes the amino acid sequence of SEQ ID NO: 7.

SEQ ID NO: 12 is the nucleotide sequence which encodes the amino acid sequence of SEQ ID NO: 8.

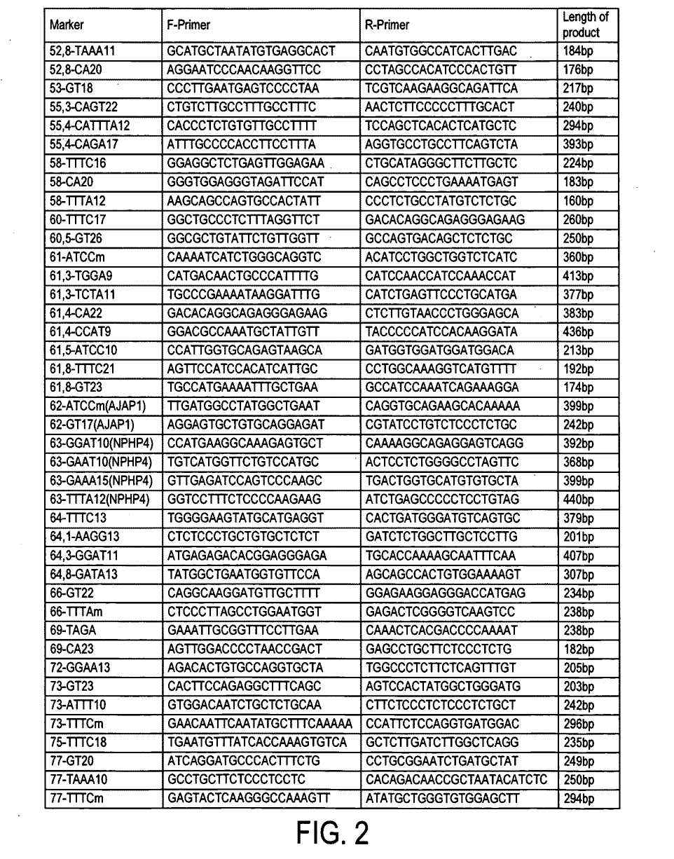

SEQ ID NOs: 13-94 are the primer sequences given in FIG. 2.

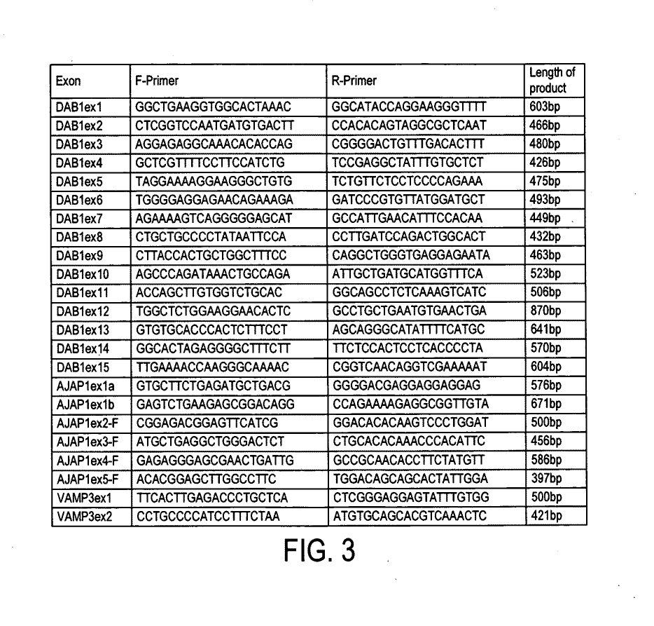

SEQ ID NOs: 95-140 are the primer sequences given in FIG. 3.

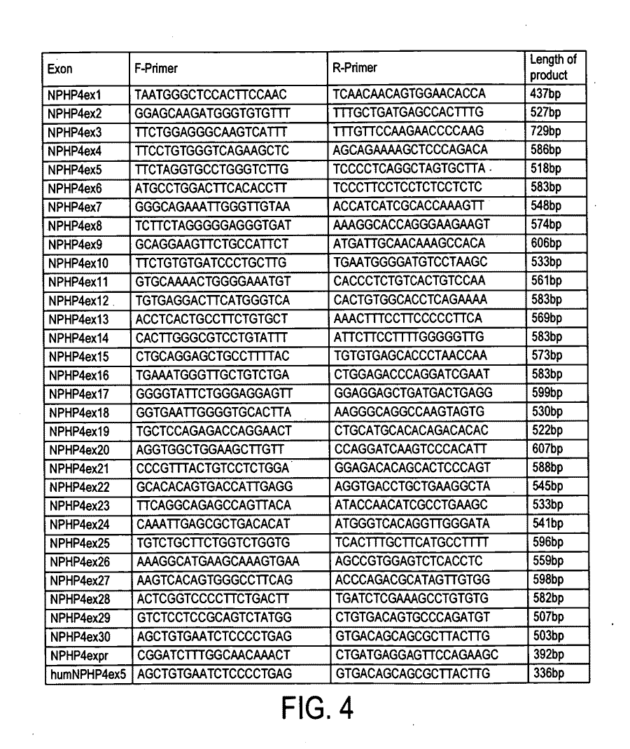

SEQ ID NOs: 141-204 are the primer sequences given in FIG. 4.

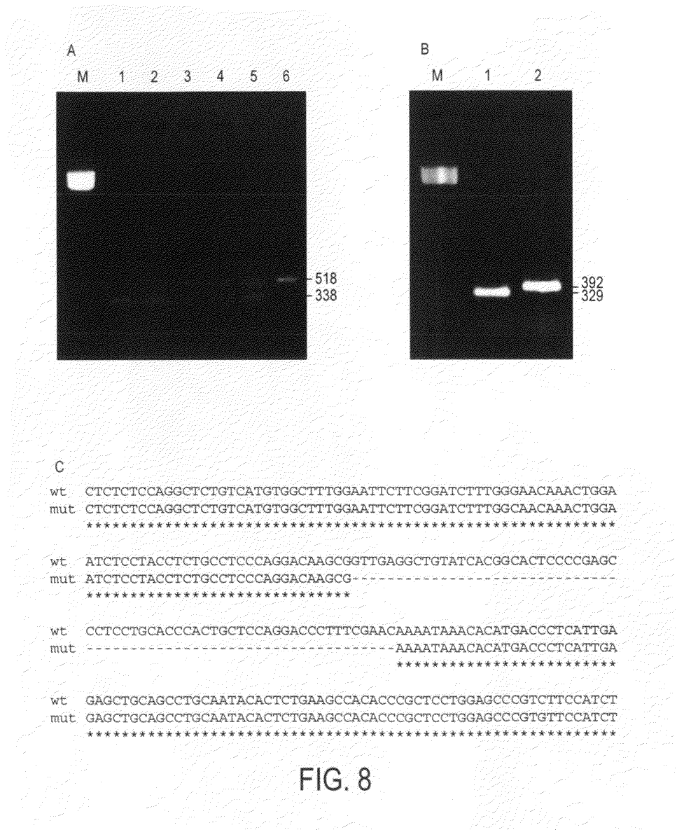

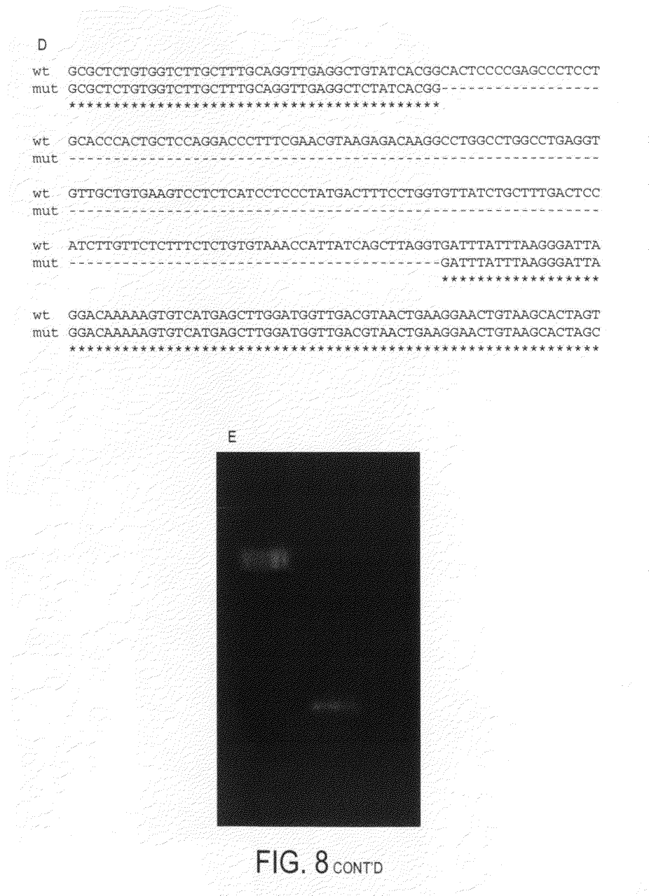

SEQ ID NOs: 205-208 are the sequences given in FIG. 8.

SEQ ID NO: 209 shows exon 5 and the start of intron 5-6 in the mutant canine NPHP4 gene, as disclosed in the description.

Sequence Listing Free Text

SEQ ID NO: 3 <223> 180 nucleotide deletion after nucleotide 93419.

SEQ ID NO: 9 <223> Exon 5 deletion is after nucleotide 132

SEQ ID NOs: 13-94 <223> PCR primer

SEQ ID NOs: 94-124 <223> Primer used in amplifying exon of DAB1 gene

SEQ ID NOs: 125-136 <223> Primer used in amplifying exon of AJAP1 gene

SEQ ID NOs: 137-140 <223> Primer used in amplifying exon of VAMP3 gene

SEQ ID NOs: 141-204 <223> Primer used in amplifying exon of NPHP4 gene

The invention will now be illustrated further by the following non-limiting Examples. Other features and advantages of the present invention will become apparent from the above detailed description. It should be understood, however, that the above detailed description and the following specific examples, while indicating preferred embodiments of the invention, are given by way of illustration only, since various changes and modifications within the spirit and scope of the invention will become apparent to those skilled in the art from the detailed description.

All documents mentioned herein are incorporated herein by reference.

### EXAMPLES

### Example 1

### Materials and Methods

### Animals and Diagnosis

Day blindness was diagnosed in a wire haired standard dachshund and a purpose bred family of dogs confirmed that this most likely was a simple recessively inherited phenotype. Detailed clinical descriptions of the affected family have been reported previously [52-53], establishing the disease as an early onset cone-rod dystrophy. All procedures used in this study were in accordance with the Norwegian Animal Research Authority (“Forsøksdyrutvalget”) and adhered to the Association for Research in Vision and Opthalmology Statement for the Use of Animals in Opthalmic Vision and Research.

### Samples

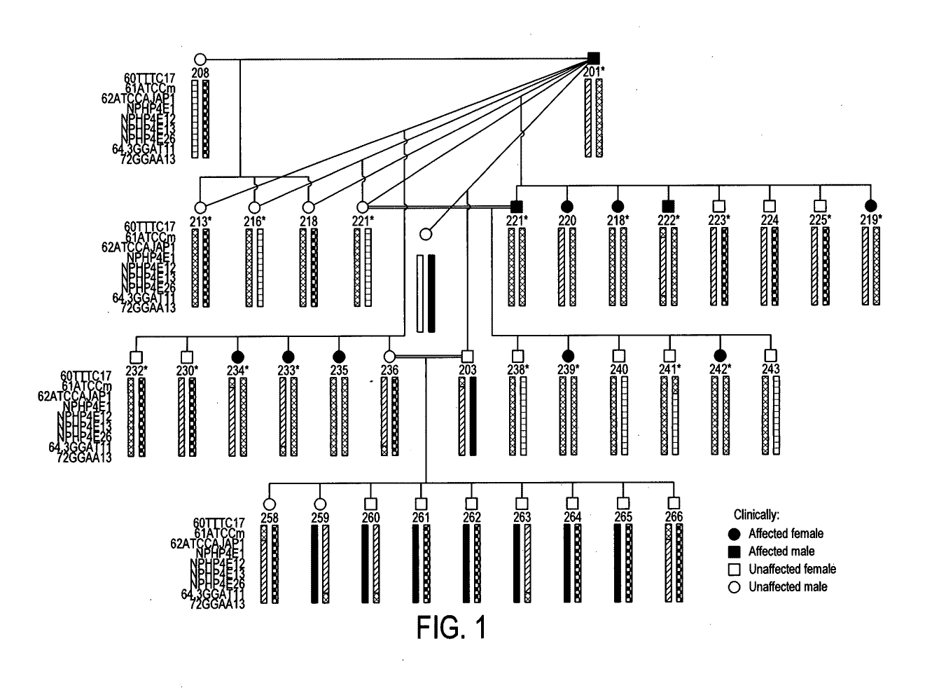

Blood samples were collected in EDTA-coated tubes from all the families segregating the disease and from two non-affected, unrelated dogs. Genomic DNA was isolated from the blood samples using DNeasy Tissue kit (Qiagen, GmbH, Germany). Thirteen affected and thirteen sex- and age matched controls (preferably full sibs) were selected for whole genome SNP analysis (FIG. 1). As some of the dogs were euthanized a number of samples from various tissues (including retina and muscle) were collected immediately post mortem and frozen on liquid nitrogen for RNA analysis. Total RNA was isolated from tissue using RNeasy Fibrous Tissue kit (Qiagen). mRNA for further use in the RACE protocol was isolated from the total RNA using Dynabeads oligodT particles (Invitrogen, Carlsbad, Calif., U.S.A.).

### Whole Genome Association Analysis

From the SWHD family 13 pairs of cases and controls were chosen. Where possible, mismatched full sibs (n=10) were used in the analysis to equally distribute population stratification effects among cases and controls. Where a full sib pair was not available, mismatched half-sibs were used (n=2 pairs). The founding sire (case 201) and dam (control 208) were included in the analysis.

Genomewide association analysis was undertaken using the Affymetrix version 2 Dog SNP Array “Platinum Panel” containing 49 663 SNP markers (Affymetrix, Santa Clara, Calif., U.S.A.). SNP genotypes were obtained by following the Affymetrix protocol for the human 500K array protocol but with a smaller hybridization volume to allow for the smaller surface area of the canine array as described in [54]. Detailed information on the arrays is available at http://www.broad.mit.edu/mammals/dog/caninearray/.

The Sibling Transmission Disequilibrium Test (sibTDT) was carried out on all 49 663 SNP using the “Dfam” option in the analysis package Plink [51]. Data were filtered to exclude individual calls with BRLMM calling probabilities lower than 0.01, loci with low minor allele frequency (<0.05), loci with low genotyping success rate (failed calls >0.1), and individual dogs with greater than 25% of genotypes missing. After applying these filters, 24 798 SNPs and 25 dogs (13 cases and 12 controls) remained in the analysis. The mean genotyping rate in the individual SWHDs included in the analysis was 96%. Genome-wide significance was ascertained through permutation of phenotypes (n=10 000 permutations) over all 24 798 SNPs in the analysis (EMP2 in Plink).

Case-control association mapping using Plink was applied to the same data with the same filtering parameters to test the efficacy of case-control association strategies in related samples. A region of association spanning from 57.6 and 72.8 Mb was identified using the array analysis.

### Segregation Studies

Primers for microsatellite analysis in the region of CFA5 (52.8 and 77 Mb), were identified by searching selected sequence windows of approximately 200,000 bp for microsatellite motifs. Tetra- and dinucleotide repeat microsatellites with more than ten and nineteen repeats, respectively, were selected. Primers for amplification of selected microsatellites were designed using primer3 (http://frodo.wi.mit.edu/cgi-bin/primer3/primer3_www.cgi) (FIG. 2). One primer in each pair was labelled with fluorescein. For genotyping, PCR was carried out as described above with 28 cycles. The sizes of the alleles were estimated with an automated genotyper (ABI PRISM® 3100 Genetic Analyzer, Applied Biosystems, Foster City, Calif., U.S.A.) with software for fragment analysis.

### Mutation Screening

Chromosomal locations of the predicted canine orthologs of the candidate genes were determined by using the http://genome.ucsc.edu web site. Primers were designed in introns, using primer3 (FIG. 3), at a distance of approximately 100-200 bp from the intron/exon borders, to provide optimal PCR products for bi-directional sequencing. The primers for amplification on each of the canine NPHP4-exons were positioned in introns according to the described human sequence NM_{—}015102 (FIG. 4). The exon specific primers for amplification of cDNA were based on the predicted canine sequence XM_{—}546745.

PCR amplification reactions were performed using 83 μM of each primer in a 15 μl reaction containing 1.5 μl DNA prepared as described above, 1× Ammonium PCR buffer containing 1.5 mM MgCl2, 83 μM each of dATP, dCTP, dGTP and dTTP, and 0.4 units Taq DNA polymerase (Ambion, Austin, Tex., U.S.A.). After an initial denaturation at 95° C. for 2 min and 30 sec, samples were amplified for 34 cycles at 95° C. for 30 sec, 58° C. for 40 sec and 72° C. for 50 sec, followed by a final extension of 72° C. for 5 min and 30 sec. All PCR products were bidirectionally sequenced using the PCR primers and the BigDye Terminator v3.1 Cycle Sequencing Kit (Applied Biosystems). Amplification was done in a 10 μl reaction mixture containing 2 μl BigDye sequencing buffer, 1.5 μl BigDye sequencing enzyme, 1 μl PCR product, 0.5 μl sequencing primer (5 pmol/μl) and 5 μl dH₂O. The sequencing reaction was performed by cycle sequencing with the following protocol: initially 95° C. for 1 minute then 28 cycles each of 95° C. for 10 sec, 50° C. for 15 sec and 60° C. for 2 min. Sequence reaction products were purified by ammonium acetate precipitation and sequenced on the ABI3100.

### Transcript Analysis

One affected family member was compared to an unaffected English cocker spaniel (ECS) at the mRNA level. Total RNA was isolated from 30 mg of muscle tissue using RNeasy fibrous tissue kit (Qiagen, GmbH, Germany) following the manufacturers instructions. The RNA was precipitated using standard procedures with ethanol, sodium acetate and glycogen. The RNA was dissolved in 10 μl RNase free water and first strand cDNA synthesis was performed using Superscript II RT following the manufacturer's descriptions. The final cDNA product was diluted 1:1 with RNase free water. Exon specific primers were designed based on the predicted canine NPHP4 sequence XM_{—}546745. PCR with these primers were performed as described above with 1 μl cDNA cDNA as template.

3′- and 5′-RACE was performed to amplify the ends of the NPHP4 transcript in both dogs. RNA was isolated from 30 mg muscle tissue as described above, precipitated and dissolved in RNase free water. 5′- and 3′-end RACE was performed with GeneRacer Kit (Invitrogen, Carlsbad, Calif., U.S.A.) and Marathon cDNA Amplification Kit (Clontech, Mountain View, Calif., U.S.A.), according to the manufacturer's protocols. PCR and nested PCR was performed using Kit specific 5′- and 3′-primers with Reverse end Forward gene specific primers and cloning of PCR products was performed using TOPO cloning kit. The inserts of the clones were amplified with M13 forward and reverse primer and sequenced with T3 and T7 primers.

### Example 2

### Genome-Wide Association Analysis

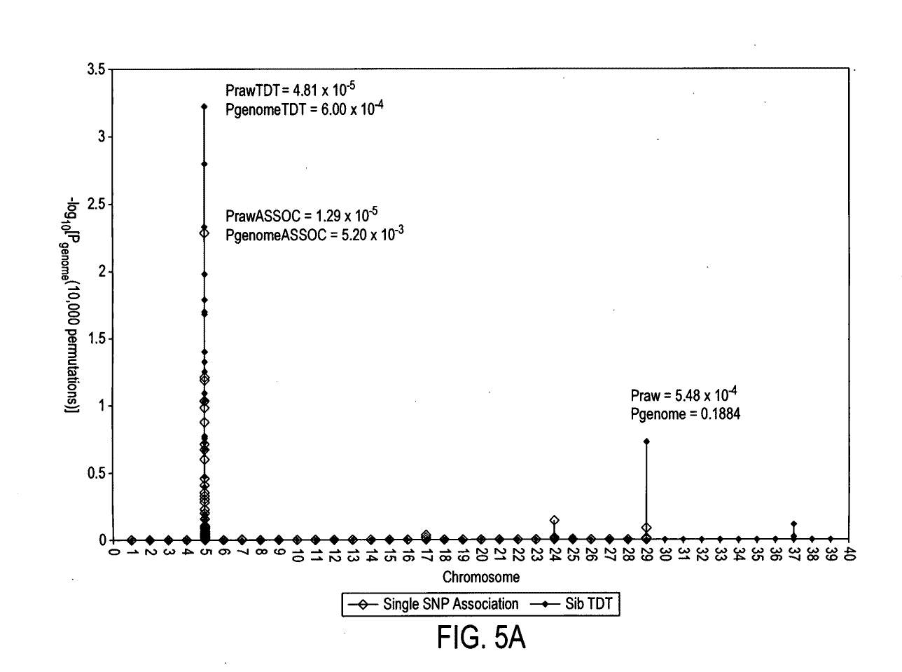

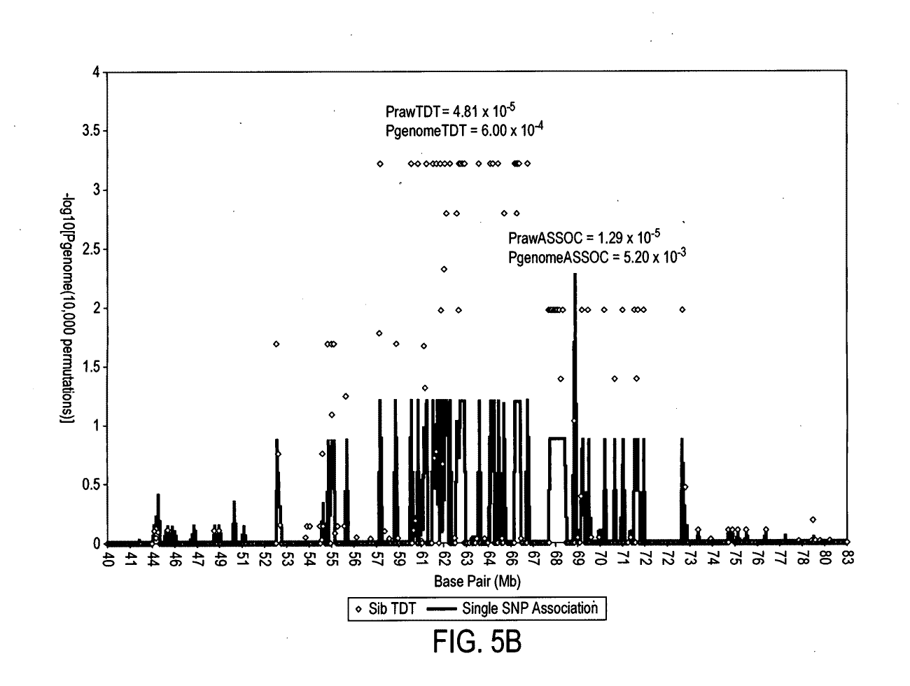

A significant association was detected to CFA5 using sib TDT analysis of ˜50,000 SNPs in 13 cases and 13 controls selected as discordant sib-pairs from a pedigree of standard wire-haired dachshund (FIG. 1). All samples were genotyped on the Affymetrix v2 canine SNP array. One control was excluded from further analysis based on low call rate (<75%). Genome-wide significance (prawTDT=4.8e-05 and pgenomeTDT=0.00060) was achieved over the region CFA5: 61,122,335-67,576,960 using a sibTDT analysis as part of the Plink software [51]. Homozygosity was detected in affected cases from 64.82 Mb-68.95 Mb (FIGS. 5A and 5B). Case-control association identified the same region, but reported the highest association to a spurious SNP in the “shoulder” of the linkage region (P rawASSOC=1.3×10−5, PgenomeASSOC=5.2×10−3 FIG. 2B). In this region, allelic frequencies differed maximally between cases and controls, however one control dog was homozygous for the “affected” allele at the point of association (Chr5:69351798) and the foundation sire (201) was heterozygous in the near vicinity (although not at this SNP).

### Fine-Mapping and Mutation Detection

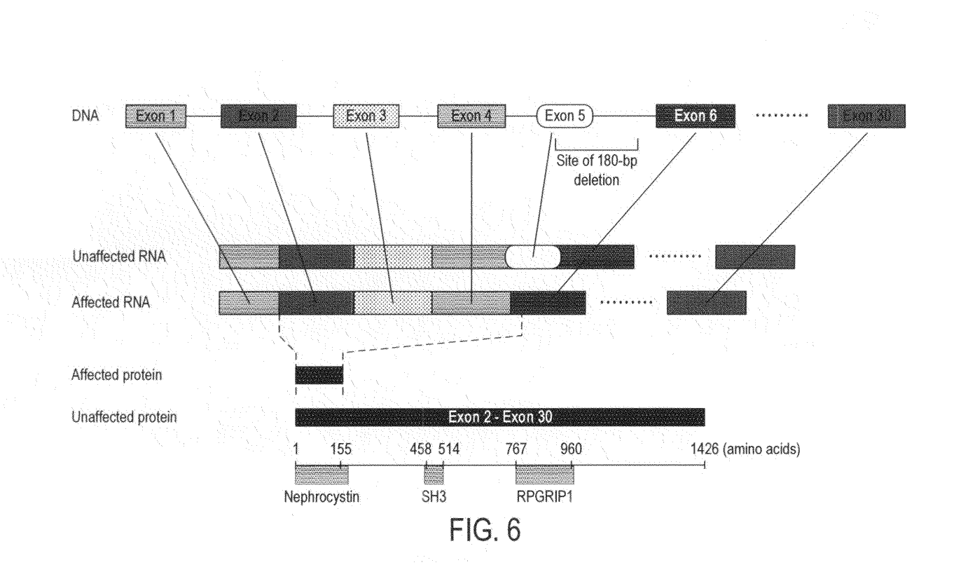

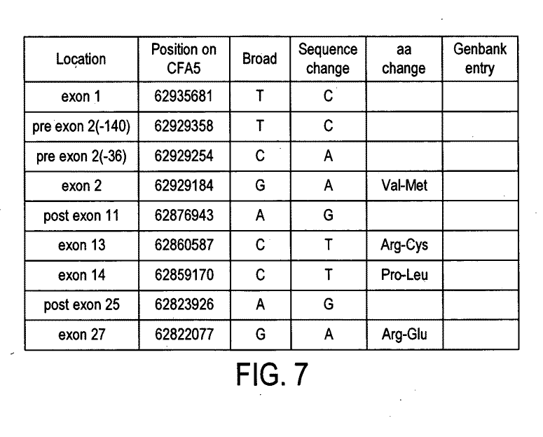

Fine-mapping and haplotype analysis of microsatellites of the region between 52.8 Mb to 77.0 Mb revealed crossovers at 61.4 Mb and 72 Mb (FIG. 1). This region contains 130 genes that were examined to find possible candidate genes expressed in the retina. DAB1 (55.3 Mb, outside region), AJAP1 (62.0 Mb), VAMP3 (64.4 Mb) and NPHP4 (62.9 Mb) were identified as potential candidate genes. Exon sequencing, including exon-intron boundaries, showed no mutations in the first three genes. PCR products from exon 5 of NPHP4 showed a different size in unaffected (519 bp) and affected (339 bp) individuals and with both bands seen in carriers (FIG. 8). Exon sequencing showed a deletion of 180 bp in exon 5/intron 5 (location 62,913,591-62,913,770) in the affected animal, where only the first 18 bases of exon 5 were retained (FIG. 6). Screening of exon 5 in 72 unrelated SWHD revealed 5 carriers, giving a gene frequency of the mutated gene of approximately 4% in the SWHD population. The exon screening also revealed 4 SNPs within exons and 4 SNPs were found in intronic sequence close to exons. All of the expressed SNPs give rise to amino acid changes, but all are present as polymorphisms in multiple breeds and are not associated to disease within the dachshunds (FIG. 7). No mutations were found in the exon/intron junctions.

PCR products from human exon 5 of NPHP4 using the primers in FIG. 4 (“humNPHP4ex5”-F-primer and R-primer) show the testing for the presence of a normal PCR product in a human. This shows that the method of the invention is easily adapted to the testing of human sampled (FIG. 8C).

### Transcript Analysis

Comparison of cDNA from affected and unaffected dogs, revealed complete exon-skipping of exon 5 in the affected animals. Translation of the 4819 bp long cDNA sequence of the healthy dogs gives a 1429 amino acid (AA) protein, while translation of the cDNA of the affected dog contains an early stop codon in exon 6 resulting in a short protein of 155 AA (FIG. 8). The RACE procedure of the 3′- and 5′-ends gave a complete mRNA sequence, with an extra untranslated exon 1 of 73 bp located at 6312 bp upstream (CanFam2.0 62,935,566-62,935,638) of exon 2. In addition exon 2 was found to have a 5′-UTR of 35 bp and exon 29 a 3′-UTR of 420 bp (CamFam2.0 62,819,750-62,820,169). The amplification of the transcript ends revealed no differences between the affected and the non-affected dogs. The amplified cDNA also revealed a difference with the previously predicted transcript XM_{—}546745. The cDNA sequence, amplified in this study, contains an additional CAG-triplet in front of exon 20 in all dogs.

### REFERENCES

[1] N. Udar, et al., Identification of GUCY2D gene mutations in CORD5 families and evidence of incomplete penetrance, Hum. Mutat. 21 (2003) 170-171.
[2] M. Michaelides, et al., A detailed study of the phenotype of an autosomal dominant cone-rod dystrophy (CORD7) associated with mutation in the gene for RIM1, Br. J Ophthalmol. 89 (2005) 198-206.
[3] N. A. Adams, A. Awadein, H. S. Toma, The retinal ciliopathies, Ophthalmic Genet. 28 (2007) 113-125.
[4] R. Allikmets, et al., A photoreceptor cell-specific ATP-binding transporter gene (ABCR) is mutated in recessive Stargardt macular dystrophy, Nat. Genet. 15 (1997) 236-246.
[5] R. Allikmets, et al., Mutation of the Stargardt disease gene (ABCR) in age-related macular degeneration, Science 277 (1997) 1805-1807.
[6] Mollet G, Silbermann F, Delous M, Salomon R, Antignac C, Saunier S. Characterization of the nephrocystin/nephrocystin-4 complex and subcellular localization of nephrocystin-4 to primary cilia and centrosomes. Hum Mol Genet. Mar. 1, 2005;14(5):645-56.
[7] M. Ismail, A. Abid, K. Anwar, M. S. Qasim, S. Khaliq, Refinement of the locus for autosomal recessive cone-rod dystrophy (CORD8) linked to chromosome 1q23-q24 in a Pakistani family and exclusion of candidate genes, J Hum. Genet. 51 (2006) 827-831.
[8] S. Khaliq, et al., Novel locus for autosomal recessive cone-rod dystrophy CORD8 mapping to chromosome 1q12-Q24, Invest Ophthalmol. Vis. Sci 41 (2000) 3709-3712.
[9] M. Danciger, et al., CORD9 a new locus for arCRD: mapping to 8p11, estimation of frequency, evaluation of a candidate gene, Invest Ophthalmol. Vis. Sci 42 (2001) 2458-2465.
[10] A. Hameed, et al., Evidence of RPGRIP1 gene mutations associated with recessive cone-rod dystrophy, J Med. Genet. 40 (2003) 616-619.
[11] H. H. Arts, et al., Mutations in the gene encoding the basal body protein RPGRIP1L, a nephrocystin-4 interactor, cause Joubert syndrome, Nat. Genet. 39 (2007) 882-888.
[12] E. M. Valente, et al., Mutations in CEP290, which encodes a centrosomal protein, cause pleiotropic forms of Joubert syndrome, Nat. Genet. 38 (2006) 623-625.
[13] F. Hildebrandt, P. Jungers, C. Robino, J.-P. Grunfeld, Nephronophthisis, medullary cystic kidney disease and medullary spong kidney disease, in: R. Schrier (Ed.), Diseases of the kidney and urinary tract, Lippincott Williams & Wilkins, Philadelphia, 2001.
[14] G. Caridi, et al., Renal-retinal syndromes: association of retinal anomalies and recessive nephronophthisis in patients with homozygous deletion of the NPH1 locus, Am. J. Kidney Dis. 32 (1998) 1059-1062.
[15] J. F. O'Toole, E. A. Otto, J. Hoefele, J. Helou, F. Hildebrandt, Mutational analysis in 119 families with nephronophthisis, Pediatr. Nephrol. 22 (2007) 366-370.
[16] H. Olbrich, et al., Mutations in a novel gene, NPHP3, cause adolescent nephronophthisis, tapeto-retinal degeneration and hepatic fibrosis, Nat. Genet. 34 (2003) 455-459.
[17] E. Otto, et al., A gene mutated in nephronophthisis and retinitis pigmentosa encodes a novel protein, nephroretinin, conserved in evolution, Am. J. Hum. Genet. 71 (2002) 1161-1167.
[18] E. A. Otto, et al., Nephrocystin-5, a ciliary IQ domain protein, is mutated in Senior-Loken syndrome and interacts with RPGR and calmodulin, Nat. Genet. 37 (2005) 282-288.
[19] J. A. Sayer, et al., The centrosomal protein nephrocystin-6 is mutated in Joubert syndrome and activates transcription factor ATF4, Nat. Genet. 38 (2006) 674-681.
[20] R. Roepman, et al., Interaction of nephrocystin-4 and RPGRIP1 is disrupted by nephronophthisis or Leber congenital amaurosis-associated mutations, Proc. Natl. Acad. Sci. U.S.A 102 (2005) 18520-18525.
[21] J. W. Kijas, et al., Cloning of the canine ABCA4 gene and evaluation in canine cone-rod dystrophies and progressive retinal atrophies, Mol. Vis. 10 (2004) 223-232.
[22] C. S. Mellersh, et al., Canine RPGRIP1 mutation establishes cone-rod dystrophy in miniature longhaired dachshunds as a homologue of human Leber congenital amaurosis, Genomics 88 (2006) 293-301.
[23] E. O. Ropstad, E. Bjerkas, K. Narfstrom, Electroretinographic findings in the Standard Wire Haired Dachshund with inherited early onset cone-rod dystrophy, Doc. Ophthalmol. 114 (2007) 27-36.
[24] G. D. Aguirre, G. M. Acland, Models, mutants and man: searching for uniquephenotypes and genes in the dog model of inherited retinal degeneration, in: E. A. Ostrander, U. Giger, K. Lindblad-Toh (Eds.), The dog and Its Genome, Cold Spring Harbor Laboratory Press, Cold Spring Harbor, N.Y., 2006, pp. 291-325.
[25] G. D. Aguirre, et al., Congenital stationary night blindness in the dog: common mutation in the RPE65 gene indicates founder effect, Mol. Vis. 4 (1998) 23.
[26] G. Dekomien, M. Runte, R. Godde, J. T. Epplen, Generalized progressive retinal atrophy of Sloughi dogs is due to an 8-bp insertion in exon 21 of the PDE6B gene, Cytogenet. Cell Genet. 90 (2000) 261-267.
[27] K. E. Guziewicz, et al., Bestrophin gene mutations cause canine multifocal retinopathy: a novel animal model for best disease, Invest Ophthalmol. Vis. Sci. 48 (2007) 1959-1967.
[28] J. W. Kijas, et al., Naturally occurring rhodopsin mutation in the dog causes retinal dysfunction and degeneration mimicking human dominant retinitis pigmentosa, Proc. Natl. Acad. Sci. U.S.A 99 (2002) 6328-6333.
[29] C. S. Mellersh, L. Pettitt, O. P. Forman, M. Vaudin, K. C. Barnett, Identification of mutations in HSF4 in dogs of three different breeds with hereditary cataracts, Vet. Ophthalmol. 9 (2006) 369-378.
[30] S. M. Petersen-Jones, D. D. Entz, D. R. Sargan, cGMP phosphodiesterase-alpha mutation causes progressive retinal atrophy in the Cardigan Welsh corgi dog, Invest Ophthalmol. Vis. Sci. 40 (1999) 1637-1644.
[31] D. J. Sidjanin, et al., Canine CNGB3 mutations establish cone degeneration as orthologous to the human achromatopsia locus ACHM3, Hum. Mol. Genet. 11 (2002) 1823-1833.
[32] M. L. Suber, et al., Irish setter dogs affected with rod/cone dysplasia contain a nonsense mutation in the rod cGMP phosphodiesterase beta-subunit gene, Proc. Natl. Acad. Sci. U.S.A 90 (1993) 3968-3972.
[33] B. Zangerl, et al., Identical mutation in a novel retinal gene causes progressive rod-cone degeneration in dogs and retinitis pigmentosa in humans, Genomics 88 (2006) 551-563.
[34] Q. Zhang, et al., Different RPGR exon ORF15 mutations in Canids provide insights into photoreceptor cell degeneration, Hum. Mol. Genet. 11 (2002) 993-1003.
[35] Sambrook, J. Fritsch, E. F., and Maniatis, T. (1989) “Molecular Cloning: A Laboratory Manual”, Cold Spring Harbor Laboratory Press, Cold Spring Harbor, N.Y.).
[36] Maxam and Gilbert (Proc. Natl Acad Sci USA (1977) 74:560)
[37] Sanger et al. (1977) Proc. Nat. Acad. Sci 74:5463.
[38] Conner, B. J., Reyes, A. A., Morin, C., Itakura, K., Teplitz, R. L. & Wallace, R. B. (1983) Proc. Natl. Acad. Sci. USA 80, 278-282.
[39] Meyers, R. M., Maniatis, T. & Lerman, L. S. (1987) Methods Enzymol. 155, 501-527.
[40] Cotton, R. G. H., Rodriques, N. R. & Campbell, R. D. (1988) Proc. Natl. Acad. Sci. USA 85, 4397-4401.
[41] Orita, M., Iwahana, H., Kanazawa, H., Hayashi, H. & Sekiya, T. (1989) Proc. Natl. Acad. Sci. USA 86, 2766-2770.
[42] Chehab, F. F. & Kan, Y. W. (1989) Proc. Natl. Acad. Sci. USA 86, 9178-9182.
[43] Newton, C. R., Graham, A., Heptinstall, L. C., Powell, S. J., Summers, C., Kalsheker, N., Smith, J. C. & Markham, A. F. (1989) Nucleic Acids Res. 17, 2503-2516.
[44] Wu, D. Y. & Wallace, R. B. (1989) Gene 76, 245-254.
[45] Landegren, U., Kaiser, R., Sanders, J. & Hood, L. (1988) Science 241, 1077-1080.
[46] Alves, A. M. & Carr, F. J. (1988) Nucleic Acids Res. 16, 8723.
[47] Wu, D. Y., Ugozzoli, L., Pal, B. K. & Wallace, R. B. (1989) Proc. Natl. Acad. Sci. USA 86, 2757-2760.
[48] Rappsilber and Mann, Trends Biochem Sci. February (2002);27(2):74-8.
[49] Roest, et. al., (1993) Hum. Mol. Genet. 2:1719-21;
[50] Van der Luijt, et. al., (1994) Genomics 20:1-4
[51] Purcell, S., Neale, B., Todd-Brown, K., Thomas, L., Ferreira, M. A., Bender, D., Maller, J., Sklar, P., de Bakker, P. I., Daly, M. J., et al. 2007. PLINK: a tool set for whole-genome association and population-based linkage analyses. Am. J. Hum. Genet. 81: 559-575.
[52] Ropstad, E. O., Bjerkas, E., and Narfstrom, K. 2007b. Clinical findings in early onset cone-rod dystrophy in the Standard Wire-haired Dachshund. Vet. Ophthalmol. 10: 69-75.
[53] Ropstad, E. O., Bjerkas, E., and Narfstrom, K. 2007a. Electroretinographic findings in the Standard Wire Haired Dachshund with inherited early onset cone-rod dystrophy. Doc. Ophthalmol. 114: 27-36.
[54] Karlsson, E. K., Baranowska, I., Wade, C. M., Salmon Hillbertz, N. H., Zody, M. C., M. C., Anderson, N., Biagi, T. M., Patterson, N., Pielberg, G. R., Kulbokas, E. J., III, et al. 2007. Efficient mapping of mendelian traits in dogs through genome-wide association. Nat. Genet. 39: 1321-1328.

1. A method for diagnosing or detecting cone-rod dystrophy (CRD) in a mammal, the method comprising determining the presence or absence in a sample of nucleic acid that has been obtained from the mammal of:
(i) a deletion in a first allele of the NPHP4 gene, the deletion comprising all or part of exon 5, and
(ii) a wild-type exon 5 in a second allele of the NPHP4 gene,
wherein the presence of (i) and the absence of (ii) is indicative of the presence of CRD in the mammal, and
wherein the presence of (i) and the presence of (ii) is indicative of the mammal being a carrier for CRD.

2. A method as claimed in claim 1, wherein the deletion in exon 5 in (i) is one which results in the production, upon translation of the NPHP4 gene, of a polypeptide which is truncated at the C-terminal end compared to a wild-type NPHP4 polypeptide.

3. A method as claimed in claim 1, wherein the deletion in exon 5 in (i) is one which results in the production, upon translation of the NPHP4 gene, of a NPHP4 polypeptide which does not bind to RPGRIP1.

4. A method as claimed in claim 1, wherein the deletion in exon 5 in (i) is one which results in the production, upon translation of the NPHP4 gene, of a NPHP4 polypeptide which does not bind to RPGRIP1 but does bind to NPHP1.

5. A method as claimed in claim 1, wherein the deletion of all or a part of exon 5 is a deletion which results, during transcription into mRNA, in the skipping of exon 5.

6. A method as claimed in claim 1, wherein the deletion of part of exon 5 is one which results in the deletion of 1-65, 1-60, 1-50, 1-40, 1-30, 1-20 or 1-10 nucleotides from exon 5.

7. A method for diagnosing or detecting cone-rod dystrophy (CRD) in a mammal, the method comprising determining the presence or absence in a sample of DNA that has been obtained from the mammal of:
(i) a deletion in a first allele of the NPHP4 gene, the deletion starting at and including nucleotide 19 of exon 5 and extending in a 3′ direction into intron 5,
(ii) a wild-type exon 5 in a second allele of the NPHP4 gene,
wherein the presence of (i) and the absence of (ii) is indicative of the presence of CRD in the mammal, and
wherein the presence of (i) and the presence of (ii) is indicative of the mammal being a carrier for CRD.

8. A method for diagnosing or detecting cone-rod dystrophy (CRD) in a mammal, the method comprising determining the presence or absence in a sample of RNA that has been obtained from the mammal or of cDNA obtained therefrom of:
(i) a deletion in the NPHP4 gene, the deletion consisting of all of exon 5,
(ii) a wild-type exon 5 in the NPHP4 gene,
wherein the presence of (i) and the absence of (ii) is indicative of the presence of CRD in the mammal, and
wherein the presence of (i) and the presence of (ii) is indicative of the mammal being a carrier for CRD.

9. A method for diagnosing or detecting cone-rod dystrophy (CRD) in a mammal, the method comprising determining the presence or absence in a sample of nucleic acid that has been obtained from the mammal of:
(i) a nucleic acid molecule coding for a mutant NPHP4 polypeptide whose amino acid sequence consists of the amino acid sequence given in SEQ ID NO: 7 or an amino acid sequence which has at least 70% sequence identity thereto, and
(ii) a nucleic acid molecule coding for a NPHP4 polypeptide whose amino acid sequence comprises SEQ ID NO: 8 or whose amino acid sequence comprises an amino acid sequence which has at least 70% sequence identity to SEQ ID NO: 8,
wherein the presence of (i) and the absence of (ii) is indicative of the presence of CRD in the mammal, and
wherein the presence of (i) and the presence of (ii) is indicative of the mammal being a carrier for CRD.

10. A method as claimed in claim 9, wherein the nucleic acid molecule encoding (i) comprises the nucleotide sequence given in SEQ ID NO: 3 or 5 or 9, or comprises a nucleotide sequence which has at least 70% sequence identity to SEQ ID NO: 9.

11. A method as claimed in claim 9, wherein the NPHP4 polypeptide in step (ii) is a polypeptide whose amino acid sequence consists of the amino acid sequence given in SEQ ID NO: 6 or an amino acid sequence which has at least 70% sequence identity thereto and which also encodes a mammalian NPHP4 polypeptide.

12. A method as claimed in claim 9, wherein the nucleic acid molecule of step (ii) comprises the nucleotide sequence given in SEQ ID NO: 1 or 2, or comprises a nucleotide sequence which has at least 95% sequence identity to SEQ ID NO: 2, or comprises the nucleotide sequence given in SEQ ID NO: 4 or comprises a nucleotide sequence which has at least 75% sequence identity to SEQ ID NO: 4.

13. A method for diagnosing or detecting cone-rod dystrophy (CRD) in a mammal or for determining the likelihood of a mammal possessing a CRD genotype, the method comprising determining the presence or absence in a sample of nucleic acid which has been obtained from the mammal of:
(i) a polymorphic site which co-segregates with a deletion in the NPHP4 gene, the deletion comprising all or part of exon 5, and
(ii) a polymorphic site which co-segregates with the wild-type exon 5 of the NPHP4 gene,
wherein the presence of (i) and the absence of (ii) is indicative of the presence of CRD in the mammal, and
wherein the presence of (i) and the presence of (ii) is indicative of the mammal being a carrier for CRD.

14. A method for diagnosing or detecting cone-rod dystrophy (CRD) in a mammal or for determining the likelihood of a mammal possessing a CRD genotype, the method comprising determining the presence or absence in a sample of nucleic acid that has been obtained from the mammal of:
(i) a polymorphic site which co-segregates with a deletion in the NPHP4 gene, the deletion comprising all or part of exon 5, and
(ii) the wild-type sequence-of exon 5 of the NPHP4 gene,
wherein the presence of (i) and the absence of (ii) is indicative of the presence of CRD in the mammal, and
wherein the presence of (i) and the presence of (ii) is indicative of the mammal being a carrier for CRD.

15. A method for diagnosing or detecting cone-rod dystrophy (CRD) in a mammal or for determining the likelihood of a mammal possessing a CRD genotype, the method comprising determining the presence or absence in a sample of nucleic acid that has been obtained from the mammal of:
(i) a deletion in the NPHP4 gene, the deletion comprising all or part of exon 5, and
(ii) a polymorphic site which co-segregates with the wild-type exon 5 of the NPHP4 gene,
wherein the presence of (i) and the absence of (ii) is indicative of the presence of CRD in the mammal, and
wherein the presence of (i) and the presence of (ii) is indicative of the mammal being a carrier for CRD.

16. A method as claimed in claim 13, wherein the polymorphic site is a microsatellite or SNP.

17. A method for testing a nucleic acid molecule, comprising determining the presence or absence of a nucleotide sequence in the nucleic acid molecule which encodes a truncated NPHP4 polypeptide.

18. A method as claimed in claim 1, wherein the presence or absence of the deletion or polymorphic site is determined by nucleic acid sequencing; microsequencing; allele specific oligonucleotide hybridization; size analysis; hybridization; 5′ nuclease digestion; single-stranded conformation polymorphism; allele specific hybridization; primer specific extension; oligonucleotide ligation assay; or restriction enzyme analysis.

19. A method as claimed in claim 1, wherein the method additionally comprises the step of amplifying the region of the mammalian NPHP4 nucleic acid which includes exon 5 of the NPHP4 gene.

20. A method for diagnosing or detecting cone-rod dystrophy (CRD) in a mammal, the method comprising determining the presence or absence in a sample of polypeptide obtained from the mammal of:
(i) a mutant NPHP4 polypeptide whose amino acid sequence consists of the amino acid sequence given in SEQ ID NO: 7 or an amino acid sequence which has at least 70% sequence identity thereto,
and
(ii) a NPHP4 polypeptide whose amino acid sequence comprises SEQ ID NO: 8 or whose amino acid sequence comprises an amino acid sequence which has at least 70%. sequence identity to SEQ ID NO: 8,
wherein the presence of (i) and the absence of (ii) is indicative of the presence of CRD in the mammal, and
wherein the presence of (i) and the presence of (ii) is indicative of the mammal being a carrier for CRD.

21. A method as claimed in claim 20, wherein the polypeptide sequence of step (ii) above consists of the amino acid sequence of SEQ ID NO: 6 or an amino acid sequence which has at least 80% sequence identity thereto.

22. A method for diagnosing or detecting cone-rod dystrophy (CRD) in a mammal, the method comprising:
(i) detecting whether a first antibody binds to a polypeptide sample obtained from the said mammal, wherein said first antibody is an antibody which binds to a polypeptide whose amino acid sequence consists of the sequence given in SEQ ID NO: 7 but does not bind to a polypeptide whose amino acid sequence consists of the sequence given in SEQ ID NO: 6, and
(ii) detecting whether a second antibody binds to a sample obtained from the said mammalian, wherein said second antibody is an antibody which binds to a polypeptide whose amino acid sequence consists of the sequence given in SEQ ID NO: 6 but does not bind to a polypeptide whose amino acid sequence consists of the sequence given in SEQ ID NO: 7,
wherein the detection of binding of (i) and the absence of binding in (ii) is indicative of the presence of CRD in the mammal, and
wherein the detection of binding in (i) and the detection of binding in (ii) is indicative of the mammal being a carrier for CRD.

23. A method for diagnosing or detecting cone-rod dystrophy (CRD) in a mammal, the method comprising:
(i) detecting whether a first antibody binds to a polypeptide sample obtained from the said mammal, wherein said first antibody is an antibody which binds to a polypeptide whose amino acid sequence consists of the sequence given in SEQ ID NO: 7 or a sequence which has at least 80% sequence identity thereto, but does not bind to a polypeptide whose amino acid sequence consists of the sequence given in SEQ ID NO: 6 or a sequence which has at least 80% sequence identity thereto, and
(ii) detecting whether a second antibody binds to a sample obtained from the said mammal, wherein said second antibody is an antibody which binds to a polypeptide whose amino acid sequence consists of the sequence given in SEQ ID NO: 6 or a sequence which has at least 80% sequence identity thereto, but does not bind to a polypeptide whose amino acid sequence consists of the sequence given in SEQ ID NO: 7 or a sequence which has at least 80% sequence identity thereto,
wherein the detection of binding of (i) and the absence of binding in (ii) is indicative of the presence of CRD in the mammal, and
wherein the detection of binding in (i) and the detection of binding in (ii) is indicative of the mammal being a carrier for CRD.

24. A method as claimed in claim 23, which additionally comprises the step of obtaining a sample from the mammal.

25. A method for diagnosing or detecting cone-rod dystrophy (CRD) in a mammal, comprising the steps:
(i) sending one or more biological samples which have been obtained from the mammal to a remote location;
(ii) receiving data on the presence or absence of a deletion in all or part of exon 5 of the NPHP4 gene of the mammal,
wherein the data has been obtained by a method as claimed in claim 1.

26. A method as claimed in claim 25 which additionally comprises the step of selecting a mammal which has a particular genotype or phenotype on the basis of the information obtained in the method, and optionally breeding that mammal with a second mammal of the opposite sex by in vivo or in vitro means.

27. A method as claimed in claim 25 wherein the mammal is a domestic mammal, a farm mammal or a human.

28. A method as claimed in claim 27, wherein the mammal is a dog or a human.

29. A method as claimed in claim 28, wherein the dog is a Standard Wirehaired Dachshund (SWHD).

30. An antibody which binds to a polypeptide whose amino acid sequence consists of the sequence given in SEQ ID NO: 7 or a sequence having at least 80% sequence identity thereto but does not bind to a polypeptide whose amino acid sequence consists of the sequence given in SEQ ID NO: 6 or a sequence having at least 80% sequence identity thereto.

31. An antibody which binds to a polypeptide whose amino acid sequence consists of the sequence given in SEQ ID NO: 6 or a sequence having at least 80% sequence identity thereto but does not bind to a polypeptide whose amino acid sequence consists of the sequence given in SEQ ID NO: 7 or a sequence having at least 80% sequence identity thereto.

32. An antibody as claimed in claim 30 which comprises a detectable label.

33. A method for assaying for the presence of a mutated form of NPHP4 polypeptide in a sample, the method comprising:
(i) providing a substrate to which is bound NPHP1 polypeptide;
(ii) contacting said substrate with the sample, and optionally determining the binding of any NPHP4 polypeptide in the sample to the bound NPHP1;
(iii) washing the substrate in order to remove any unbound sample;
(iv) contacting the substrate with labelled RPGRIP1 polypeptide;
(v) washing the substrate; and
(vi) detecting binding of any labelled RPGRIP1 polypeptide to the NPHP4 polypeptide;
wherein the binding of NPHP4 polypeptide in step (ii) with the absence of binding of labelled RPGRIP1 polypeptide to the NPHP4 polypeptide in step (vi) is indicative of the presence of mutated NPHP4 polypeptide in the sample.

34. A kit for diagnosing or detecting cone-rod dystrophy (CRD) in a mammal, the kit comprising first and second nucleic acid primers for amplifying the region of nucleic acid which includes exon 5 of the mammalian NPHP4 gene.

35. A kit for diagnosing or detecting cone-rod dystrophy (CRD) in a mammal, the kit comprising an antibody as claimed in claim 30.

36. A nucleic acid molecule whose nucleotide sequence consists of the sequence given in SEQ ID NO: 3 or 5 or 9, of the complement thereof.

37. A nucleic acid molecule which encodes a polypeptide of SEQ ID NO: 7 or 8.

38. A vector comprising the nucleic acid molecule of claim 36.

39. An isolated host cell comprising the nucleic acid molecule of claim 36.

40. A process for producing a vector, comprising the steps:
(i) obtaining a wild-type or mutant NPHP4 gene;
(ii) determining the genotype of the NPHP4 gene using a method as claimed in claim 1; and
(iii) inserting the NPHP4 gene into a vector.

41. A polypeptide whose amino acid sequence consists of the sequence given in SEQ ID NO: 7 or a sequence having at least 75% sequence identity thereto.

42. A process for producing a modified mammalian cell, comprising the step of introducing a wild-type NPHP4 gene into the genome of the mammalian cell.

43. A mammal which is homozygous or heterozygous for a wild-type NPHP4 gene or functional NPHP4 gene, wherein the mammal additionally comprise a selective marker in its genome adjacent to the NPHP4 gene.

44. Use of one or more SNPs as defined in FIG. 7 for the identification of a mammal or family of mammals, or offspring thereof.

45. Use of one or more SNPs as defined in FIG. 7 for the identification of mutations in the NPHP4 gene.

46. Use of one or more SNPs as defined in FIG. 7 for the identification of mutations in a gene associated with eye disorders.

47. Use of one or more microsatellite markers as defined in FIG. 2 for the identification of a mammal or family of mammals, or offspring thereof.

48. Use of one or more microsatellite markers as defined in FIG. 2 for the identification of mutations in the NPHP4 gene.

49. Use of one or more microsatellite markers as defined in FIG. 2 for the identification of mutations in a gene associated with eye disorders.
